(19) United States
(12) Patent Application Publication
(10) Pub. No.: US 2023/0000960 A1
(43) Pub. Date: Jan. 5, 2023

(54) NEOANTIGEN COMPOSITIONS AND USES THEREOF

(71) Applicant: BIONTECH US INC., Cambridge, MA (US)

(72) Inventors: Vikram Juneja, Newton, MA (US); Zhengxin Dong, Holliston, MA (US); Robyn Jessica Eisert, Somerville, MA (US); Mahboubeh Kheirabadi, Cambridge, MA (US)

(21) Appl. No.: 17/618,067

(22) PCT Filed: Jun. 10, 2020
(86) PCT No.: PCT/US20/37019

§ 371 Date: Dec. 10, 2021

Related U.S. Application Data
(60) Provisional application No. 62/860,493, filed on Jun. 12, 2019.

(51) Int. Cl. A61K 39/00; A61P 37/04

(52) U.S. Cl. CPC A61K 39/0011; A61P 37/04; A61K 2039/5154; A61K 2039/54

(57) ABSTRACT

Disclosed herein relates to immunotherapeutic polypeptides comprising neoepitopes, antigen presenting cells comprising the immunotherapeutic polypeptides, and a pharmaceutical composition comprising the immunotherapeutic polypeptides. Also disclosed herein is use of the immunotherapeutic polypeptides in treating a disease or condition.

### CROSS-REFERENCE TO RELATED APPLICATIONS

This application claims the benefit of U.S. Provisional Application No. 62/860,493 filed on Jun. 12, 2019, which is hereby incorporated by reference in its entirety. This application relates to International Application No. PCT/US2020/031898, filed on May 7, 2020, which is hereby incorporated by reference in its entirety.

### BACKGROUND

Cancer immunotherapy is the use of the immune system to treat cancer. Immunotherapies exploit the fact that cancer cells often have molecules on their surface that can be detected by the immune system, known as tumor antigens, which are often proteins or other macromolecules (e.g., carbohydrates). Active immunotherapy directs the immune system to attack tumor cells by targeting tumor antigens. Passive immunotherapies enhance existing anti-tumor responses and include the use of monoclonal antibodies, lymphocytes, and cytokines. Tumor vaccines are typically composed of tumor antigens and immunostimulatory molecules (e.g., adjuvants, cytokines, or Toll-Like Receptor (TLR) ligands) that work together to induce antigen-specific cytotoxic T cells (CTLs) that recognize and lyse tumor cells. Tumor neoantigens, which arise as a result of genetic change (e.g., inversions, translocations, deletions, missense mutations, splice site mutations, etc.) within malignant cells, represent the most tumor-specific class of antigens and can be patient-specific or shared. Tumor neoantigens are unique to the tumor cell as the mutation and its corresponding protein are present only in the tumor. They also avoid central tolerance and are therefore more likely to be immunogenic. Therefore, tumor neoantigens provide an excellent target for immune recognition including by both humoral and cellular immunity.

To elicit a T cell response from vaccination, antigen-presenting cells (APCs) must process epitope-containing peptide and present epitopes on major histocompatibility complex (MHC) I or MHC II. One of the critical barriers to developing curative and tumor-specific immunotherapy is insufficient processing and release of minimal epitopes for antigen presentation to generate adequate immune responses. Accordingly, there is a need for developing additional cancer therapeutic vaccines to ensure efficient and sufficient epitope processing and presentation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, provided herein is a polypeptide comprising an epitope presented by a class I MHC or a class II MHC of an antigen presenting cell (APC), the polypeptide having a structure of Formula (I):

Yₙ-Bₜ-Aᵣ-Xₘ-Aₛ-Cᵤ-Zₚ Formula (I),

or a pharmaceutically acceptable salt thereof,

(i) wherein Xₘ is the epitope, wherein each X independently represents an amino acid of a contiguous amino acid sequence encoded by a nucleic acid sequence in a genome of a subject,

and wherein, (a) the MHC is a class I MHC and m is an integer from 8 to 12, or
(b) the MHC is a class II MHC and m is an integer from 9 to 25;

(ii) wherein each Y is independently an amino acid, analog, or derivative thereof, and wherein:
(A) when variable r of Ar in Formula (I) is 0, Yₙ is not encoded by a nucleic acid sequence immediately upstream of the nucleic acid sequence in the genome of the subject that encodes Bₜ-Aᵣ-Xₘ,
(B) when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or
(C) when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and

further wherein n is an integer from 0 to 1000;

(iii) wherein each Z is independently an amino acid, analog, or derivative thereof, and wherein:
(A) when variable s of A, in Formula (I) is 0, Zₚ is not encoded by a nucleic acid sequence immediately downstream of the nucleic acid sequence in the genome of the subject that encodes Xₘ-Aₛ-Cᵤ,
(B) when variable s of A, in Formula (I) is 1 and variable u of Cu in Formula (I) is 0, Zₚ is not encoded by a nucleic acid sequence immediately downstream of the nucleic acid sequence in the genome of the subject that encodes Xₘ, or
(C) when variable s of Aₛ in Formula (I) is 1 and variable u of Cu in Formula (I) is 1 or more, Zₚ is not encoded by a nucleic acid sequence immediately downstream of the nucleic acid sequence in the genome of the subject that encodes Cᵤ; and

further wherein p is an integer from 0 to 1000;

and further wherein,

when n is 0, p is an integer from 1 to 1000; and

when p is 0, n is an integer from 1 to 1000;

(iv) wherein Aᵣ is a linker, and r is 0 or 1;

(v) wherein Aₛ is a linker, and s is 0 or 1;
(vi) wherein each B independently represents an amino acid encoded by a nucleic acid sequence in the genome of the subject that is immediately upstream of the nucleic acid sequence in the genome of the subject that encodes Xₘ,

and wherein t is an integer from 0 to 1000; and

(vii) wherein each C independently represents an amino acid encoded by a nucleic acid sequence in the genome of the subject that is immediately downstream of the nucleic acid sequence in the genome of the subject that encodes Xₘ,

and wherein, u is an integer from 0 to 1000;

and further wherein,
(a) the polypeptide does not consist of four different epitopes presented by a class I MHC;
(b) the polypeptide comprises at least two different polypeptide molecules;
(c) the epitope comprises at least one mutant amino acid; and/or
(d) Yₙ and/or Zₚ is cleaved from the epitope when the polypeptide is processed by the APC.

In some embodiments, the epitope is presented by a class II MHC. In some embodiments, m is an integer from 9 to 25. In some embodiments, t is 1, 2, 3, 4, or 5 or more and r is 0. In some embodiments, u is 1, 2, 3, 4, or 5 or more and s is 0. In some embodiments, t is 1 or more, r is 0, and n is from 1-1000. In some embodiments, u is 1 or more, s is 0, and p is from 1-1000. In some embodiments, t is 0. In some embodiments, u is 0. In some embodiments, t is at least 1 and Bₜ comprises a lysine. In some embodiments, u is at least 1 and Cᵤ comprises a lysine. In some embodiments, Bₜ is cleaved from the epitope when the polypeptide is processed by the APC. In some embodiments, Cᵤ is cleaved from the epitope when the polypeptide is processed by the APC. In some embodiments, n is an integer from 1 to 5 or 7-1000. In some embodiments, p is an integer from 1 to 4 or 6-1000.

In some embodiments, the polypeptide does not consist of four different epitopes presented by a class I MHC. In some embodiments, the polypeptide does not comprise four different epitopes presented by a class I MHC. In some embodiments, the polypeptide comprises at least two different polypeptide molecules. In some embodiments, the epitope comprises at least one mutant amino acid. In some embodiments, the at least one mutant amino acid is encoded by an insertion, a deletion, a frameshift, a neoORF, or a point mutation in the nucleic acid sequence in the genome of the subject. In some embodiments, Yₙ and/or Zₚ is cleaved from the epitope when the polypeptide is processed by the APC. In some embodiments, m of Xₘ is at least 8 and Xₘ is AA₁AA₂AA₃AA₄AA₅AA₆AA₇AA₈AA₉AA₁₀AA₁₁AA₁₂AA₁₃AA₁₄AA₁₅AA₁₆AA₁₇AA₁₈AA₁₉AA₂₀AA₂₁AA₂₂AA₂₃AA₂₄AA₂₅, wherein each AA is an amino acid, and wherein one or more of AA₉, AA₁₀, AA₁₁, AA₁₂, AA₁₃, AA₁₄, AA₁₅, AA₁₆, AA₁₇, AA₁₈, AA₁₉, AA₂₀, AA₂₁, AA₂₂, AA₂₃, AA₂₄, and AA₂₅ are optionally present, and further wherein at least one AA is a mutant amino acid. In some embodiments, r is 1. In some embodiments, s is 1. In some embodiments, r is 1 and s is 1. In some embodiments, r is 0. In some embodiments, s is 0. In some embodiments, r is 0 and s is 0.

In some embodiments, Aᵣ and/or Aₛ is a non-polypeptide linker. In some embodiments, Aᵣ and/or Aₛ is chemical linker. In some embodiments, Aᵣ and/or Aₛ comprises a non-natural amino acid. In some embodiments, Aᵣ and/or Aₛ does not comprise an amino acid. In some embodiments, Aᵣ and/or Aₛ does not comprise a natural amino acid. In some embodiments, Aᵣ and/or Aₛ comprises a bond other than a peptide bond. In some embodiments, Aᵣ and/or Aₛ comprises a disulfide bond. In some embodiments, Aᵣ and Aₛ are different. In some embodiments, Aᵣ and Aₛ are the same.

In some embodiments, the polypeptide comprises a hydrophilic tail. In some embodiments, Yₙ-Bₜ-Aᵣ and/or Aₛ-Cᵤ-Zₚ enhances solubility of the polypeptide compared to a corresponding peptide that does not contain Yₙ-Bₜ-Aᵣ and/or Aₛ-Zₚ. In some embodiments, each X of Xₘ is a natural amino acid.

In some embodiments, the epitope is released from Yₙ-Bₜ-Aᵣ and/or Aₛ-Cᵤ-Zₚ when the polypeptide is processed by the APC. In some embodiments, the polypeptide is cleaved at Aᵣ and/or Aₛ. In some embodiments, the polypeptide is cleaved at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ; and/or the polypeptide is cleaved at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, the polypeptide is cleaved at a higher rate when n is an integer from 1 to 1000 compared to cleavage of a corresponding polypeptide of the same length that comprises Bₜ-Xₘ wherein t is at least one and r of variable Aᵣ in Formula (I) is 0; and/or wherein the polypeptide is cleaved at a higher rate when p is an integer from 1 to 1000 compared to cleavage of a corresponding polypeptide of the same length that comprises Xₘ-Cᵤ wherein u is at least one and s of variable Aₛ in Formula (I) is 0.

In some embodiments, the polypeptide is cleaved at Aᵣ at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ; and/or the polypeptide is cleaved at Aₛ at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, epitope presentation by the APC is enhanced when n is an integer from 1 to 1000 compared to epitope presentation of a corresponding polypeptide of the same length that comprises Xₘ and at least one additional amino acid encoded by a nucleic acid sequence immediately upstream of the nucleic acid sequence in the genome of the subject that encodes Xₘ; and/or epitope presentation by the APC is enhanced when p is an integer from 1 to 1000 compared to epitope presentation of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, epitope presentation by the APC is enhanced when n is an integer from 1 to 1000 compared to epitope presentation of a corresponding polypeptide of the same length that comprises Bₜ-Xₘ wherein t is at least one and r of variable Aᵣ in Formula (I) is 0; and/or wherein epitope presentation by the APC is enhanced when p is an integer from 1 to 1000 compared to epitope presentation of a corresponding polypeptide of the same length that comprises Xₘ-Cᵤ wherein u is at least one and s of variable Aₛ in Formula (I) is 0.

In some embodiments, the APC presents the epitope to an immune cell. In some embodiments, the APC presents the epitope to a phagocytic cell. In some embodiments, the APC presents the epitope to a dendritic cell, a macrophage, a mast cell, a neutrophil, or a monocyte. In some embodiments, the APC presents the epitope preferentially or specifically to the immune cell, the phagocytic cell, the dendritic cell, the macrophage, the mast cell, the neutrophil, or the monocyte.

In some embodiments, immunogenicity is enhanced when n is an integer from 1 to 1000 compared to immunogenicity of a corresponding polypeptide of the same length that comprises Xₘ and at least one additional amino acid encoded by a nucleic acid sequence immediately upstream of the nucleic acid sequence in the genome of the subject that encodes Xₘ; and/or immunogenicity is enhanced when p is an integer from 1 to 1000 compared to immunogenicity of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, immunogenicity is enhanced when n is an integer from 1 to 1000 compared to immunogenicity of a corresponding polypeptide of the same length that comprises Bₜ-Xₘ wherein t is at least one and r of variable Aᵣ in Formula (I) is 0; and/or wherein immunogenicity is enhanced when p is an integer from 1 to 1000 compared to immunogenicity of a corresponding polypeptide of the same length that comprises Xₘ-Cᵤ wherein u is at least one and s of variable Aₛ in Formula (I) is 0.

In some embodiments, anti-tumor activity is enhanced when n is an integer from 1 to 1000 compared to anti-tumor activity of a corresponding polypeptide of the same length that comprises Xₘ and at least one additional amino acid encoded by a nucleic acid sequence immediately upstream of the nucleic acid sequence in the genome of the subject that encodes Xₘ; and/or anti-tumor activity is enhanced when p is an integer from 1 to 1000 compared to anti-tumor activity of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, anti-tumor activity is enhanced when n is an integer from 1 to 1000 compared to anti-tumor activity of a corresponding polypeptide of the same length that comprises Bₜ-Xₘ wherein t is at least one and r of variable Aᵣ in Formula (I) is 0; and/or wherein anti-tumor activity is enhanced when p is an integer from 1 to 1000 compared to anti-tumor activity of a corresponding polypeptide of the same length that comprises Xₘ-Cᵤ wherein u is at least one and s of variable Aₛ in Formula (I) is 0.

In some embodiments, Yₙ and/or Zₚ comprises a sequence selected from the group consisting of poly-Lys (polyK) and poly-Arg (polyR). In some embodiments, Yₙ and/or Zₚ comprises a sequence selected from the group consisting of polyK-AA-AA and polyR-AA-AA, wherein each AA is an amino acid or analogue or derivative thereof. In some embodiments, polyK comprises poly-L-Lys. In some embodiments, polyR comprises poly-L-Arg. In some embodiments, polyK or polyR comprises at least three or four contiguous lysine or arginine residues, respectively. In some embodiments, Aᵣ and/or Aₛ is selected from the group consisting of a disulfide; p-aminobenzyloxycarbonyl (PABC); and AA-AA-PABC, wherein each AA is an amino acid or analogue or derivative thereof. In some embodiments, AA-AA-PABC is selected from the group consisting of Ala-Lys-PABC, Val-Cit-PABC, and Phe-Lys-PABC.

In some embodiments, Aᵣ and/or Aₛ is

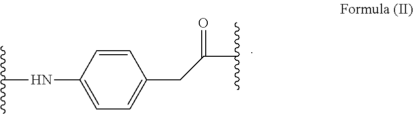 [SMILES: *Nc1ccc(CC(*)=O)cc1]

In some embodiments, Aᵣ and/or Aₛ is

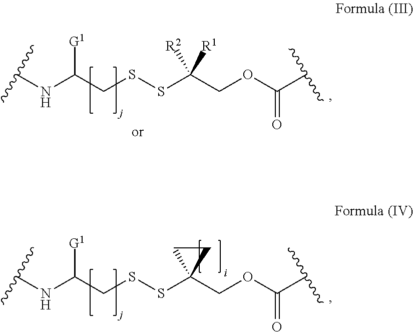

wherein,
R¹ and R² is independently H or an (C₁-C₆) alkyl; j is 1 or 2; G¹ is H or COOH; and i is 1, 2, 3, 4, or 5.

In some embodiments, the polypeptide is ubiquitinated. In some embodiments, the polypeptide is ubiquitinated prior to cleavage. In some embodiments, the polypeptide is ubiquitinated on a lysine residue. In some embodiments, the polypeptide is not cleaved before processing by an APC before internalization by an APC in a subject. In some embodiments, the polypeptide is not cleaved in blood in a subject before processing by an APC or before internalization by an APC. In some embodiments, the polypeptide is not cleaved by a protease in blood. In some embodiments, the polypeptide is not cleaved by plasmin, plasma kallikrein, tissue kallikrein, thrombin, or a coagulation factor. In some embodiments, the polypeptide is stable in human plasma. In some embodiments, the polypeptide has a half-life of from 1 hour to 5 days in human plasma. In some embodiments, the polypeptide is cleaved in a lysosome, an endolysosome, an endosome, or an endoplasmic reticulum (ER). In some embodiments, the polypeptide is cleaved by an aminopeptidase. In some embodiments, the aminopeptidase is an insulin-regulated aminopeptidase IRAP) or an endoplasmic reticulum aminopeptidase (ERAP). In some embodiments, the polypeptide is processed by a trypsin-like domain of a proteasome and/or an immunoproteasome. In some embodiments, the trypsin-like domain comprises trypsin-like activity, chymotrypsin-like activity, or peptidylglutamyl-peptide hydrolase (PGPH) activity. In some embodiments, the polypeptide is cleaved by a protease. In some embodiments, the protease is a trypsin-like protease, a chymotrypsin-like protease, or a peptidylglutamyl-peptide hydrolase (PGPH). In some embodiments, the protease is selected from the group consisting of asparagine peptide lyase, aspartic protease, cysteine protease, glutamic protease, metalloprotease, serine protease, and threonine protease. In some embodiments, the protease is a cysteine protease selected from the group consisting of a Calpain, a Caspase, Cathepsin B, Cathepsin C, Cathepsin F, Cathepsin H, Cathepsin K, Cathepsin L1, Cathepsin L2, Cathepsin O, Cathepsin S, Cathepsin W, and Cathepsin Z.

In some embodiments, the subject is a mammal. In some embodiments, the subject is a human.

In some embodiments, the epitope binds to a MHC class I HLA. In some embodiments, the epitope binds to the MHC class I HLA with a stability of 10 minutes to 24 hours. In some embodiments, the epitope binds to the MHC class I HLA with an affinity of 0.1 nM to 2000 nM. In some embodiments, the epitope binds to MHC class II HLA. In some embodiments, the epitope binds to the MHC class II HLA with a stability of 10 minutes to 24 hours. In some embodiments, the epitope binds to the MHC class II HLA with an affinity of 0.1 nM to 2000 nM, 1 nM to 1000 nM, 10 nM to 500 nM, or less than 1000 nM. In some embodiments, n is an integer from 1 to 20 or 5 to 12. In some embodiments, p is an integer from 1 to 20 or 5 to 12. In some embodiments, the epitope comprises a tumor-specific epitope.

In some embodiments, the polypeptide comprises at least two polypeptides, wherein two or more of the at least two polypeptides have the same formula Yₙ-Bₜ-Aᵣ-Xₘ-Aₛ-Cᵤ-Zₚ. In some embodiments, the polypeptide comprises at least at two polypeptide molecules. In some embodiments, Xₘ of two or more of the at least two polypeptides or polypeptide molecules are the same. In some embodiments, Yₙ of two or more of the at least two polypeptides or polypeptide molecules are the same. In some embodiments, Zₚ of two or more of the at least two polypeptides or polypeptide molecules are the same. In some embodiments, Aᵣ and/or Aₛ of two or more of the at least two polypeptides or polypeptide molecules are different. In some embodiments, r=0 for a first of the at least two polypeptides or polypeptide molecules and r=1 for a second of the at least two polypeptides or polypeptide molecules. In some embodiments, s=0 for a first of the at least two polypeptides or polypeptide molecules and s=1 for a second of the at least two polypeptides or polypeptide molecules. In some embodiments, the polypeptide comprises at least 3, 4, 5, 6, 7, 8, 9, 10, or more polypeptides or polypeptide molecules.

In some embodiments, the epitope is a RAS epitope. In some embodiments, the epitope comprises a mutant RAS peptide sequence that comprises at least 8 contiguous amino acids of a mutant RAS protein comprising a mutation at G12, G13, or Q61 and the mutation at G12, G13, or Q61. In some embodiments, the at least 8 contiguous amino acids of the mutant RAS protein comprising the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, Yₙ and/or Zₚ comprises an amino acid sequence of a protein of cytomegalovirus (CMV), such as pp65, human immunodeficiency virus (HIV), or MART-1. In some embodiments, n and/or p is 1, 2, 3, or an integer greater than 3. In some embodiments, Yₙ and/or Zₚ comprises a lysine or a poly-lysine. In some embodiments, Yₙ and/or Zₚ comprises K, KK, KKK, KKKK or KKKKK.

In some embodiments, the epitope binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM. In some embodiments, the epitope binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes. In some embodiments, the HLA allele is selected from the group consisting of an HLA-A02:01 allele, an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, an HLA-A74:01 allele, and/or an HLA-C08:02 allele and any combination thereof.

In some embodiments, the epitope comprises an amino acid sequence of GADGVGKSAL, GACGVGKSAL, GAVGVGKSAL, GADGVGKSA, GACGVGKSA, GAVGVGKSA, KLVVVGACGV, FLVVVGACGL, FMVVVGACGI, FLVVVGACGI, FMVVVGACGV, FLVVVGACGV, MLVVVGACGV, FMVVVGACGL, YLVVVGACGV, KMVVVGACGV, YMVVVGACGV, MMVVVGACGV, DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, or DILDTAGKE.

In some embodiments, Yₙ comprises an amino acid sequence of IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, TEYKLV, WQAGILAR, HSYTTAE, PLTEEKIK, GALHFKPGSR, RRANKDATAE, KAFISHEEKR, TDLSSRFSKS, FDLGGGTFDV, CLLLHYSVSK, KKKKIIMKIRNA, or MTEYKLVVV. In some embodiments, Zₚ comprises an amino acid sequence of KKNKKDDI, KKNKKDDIKD, AGNDDDDDDDDDDDDDDDDDKKDKDDDDDD, AGNKKKKKKKNNNNNNNNNNNNNNNNNNNN, AGRDDDDDDDDDDDDDDDDDDDDDDDDDDD, SALTI, SALTIQL, GKSALTIQL, GKSALTI, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

In some embodiments, the epitope is not a RAS epitope. In some embodiments, the polypeptide is not KKKKKPKRDGYMFLKAESKIMFAT, KKKKYMFLKAESKIMFATLQRSS, KKKKKAESKIMFATLQRSSLWCL, KKKKKIMFATLQRSSLWCLCSNH, or KKKKMFATLQRSSLWCLCSNH.

In some embodiments, the epitope is a GATA3 epitope. In some embodiments, the GATA3 epitope comprises an amino acid sequence of MLTGPPARV, SMLTGPPARV, VLPEPHLAL, KPKRDGYMF, KPKRDGYMFL, ESKIMFATL, KRDGYMFL, PAVPFDLHF, AESKIMFATL, FATLQRSSL, ARVPAVPFD, IMKPKRDGY, DGYMFLKA, MFLKAESKIMF, LTGPPARV, ARVPAVPF, SMLTGPPAR, RVPAVPFDL, or LTGPPARVP.

In some aspects, provided herein is a cell comprising the polypeptide described herein. In some embodiments, the cell is an antigen presenting cell. In some embodiments, the cell is a dendritic cell. In some embodiments, the cell is a mature antigen presenting cell.

In some aspects, provided herein is a method of cleaving a polypeptide comprising contacting the polypeptide described herein to an antigen presenting cell (APC). In some embodiments, the method is performed in vivo. In some embodiments, the method is performed ex vivo.

In some aspects, provided herein is a method of manufacturing a polypeptide comprising linking Yₙ-Aᵣ and/or Aₛ-Zₚ to a sequence comprising an epitope sequence, wherein the epitope sequence is presented by a class I MHC or a class II MHC of an antigen presenting cell (APC); wherein (i) each Y is independently an amino acid, analog, or derivative thereof of and wherein Yₙ is not encoded by a nucleic acid sequence immediately upstream of a nucleic acid sequence in a genome of a subject that encodes the epitope and n is an integer from 0 to 1000; (ii) each Z is independently an amino acid, analog, or derivative thereof of and wherein Zₚ is not encoded by a nucleic acid sequence immediately downstream of the nucleic acid sequence in the genome of the subject that encodes the epitope and p is an integer from 0 to 1000; and (iii) Aᵣ is a linker and Aₛ is a linker, wherein at least one of r and s is 1; and further wherein (a) the polypeptide does not consist of four different epitopes presented by a class I MHC; (b) the polypeptide comprises at least two different polypeptide molecules; (c) the epitope comprises at least one mutant amino acid; and/or (d) Yₙ and/or Zₚ is cleaved from the epitope when the polypeptide is processed by the APC.

In some aspects, provided herein is a method of manufacturing a polypeptide comprising linking Yₙ to Bₜ-Xₘ and/or Zₚ to Xₘ-Cᵤ, wherein Xₘ is an epitope sequence presented by a class I MHC or a class II MHC of an antigen presenting cell (APC); and wherein (i) each B independently represents an amino acid encoded by a nucleic acid sequence in a genome of a subject that is immediately upstream of a nucleic acid sequence in the genome of the subject that encodes Xₘ, and wherein t is an integer from 0 to 1000; (ii) wherein each C independently represents an amino acid encoded by a nucleic acid sequence in the genome of the subject that is immediately downstream of the nucleic acid sequence in the genome of the subject that encodes Xₘ, and wherein, u is an integer from 0 to 1000; (iii) each Y is independently an amino acid, analog, or derivative thereof of and wherein Yₙ is not encoded by a nucleic acid sequence immediately upstream of a nucleic acid sequence in the genome of the subject that encodes Bₜ-Xₘ, and wherein, n is an integer from 0 to 1000; and (iv) each Z is independently an amino acid, analog, or derivative thereof of and wherein Zₚ is not encoded by a nucleic acid sequence immediately downstream of a nucleic acid sequence in the genome of the subject that encodes Xₘ-Cᵤ, and wherein, p is an integer from 0 to 1000; and further wherein (a) the polypeptide does not consist of four different epitopes presented by a class I MHC; (b) the polypeptide comprises at least two different polypeptide molecules; (c) the epitope comprises at least one mutant amino acid; and/or (d) Yₙ-Bₜ and/or Cᵤ-Zₚ is cleaved from the epitope when the polypeptide is processed by the APC.

In some embodiments, when n is 0, p is an integer from 1 to 1000 and when p is 0, n is an integer from 1 to 1000. In some embodiments, each X independently represents an amino acid of a peptide sequence comprising any contiguous amino acid sequence encoded by a nucleic acid sequence in a genome of a subject, and wherein (a) the MHC is a class I MHC and m is an integer from 8 to 12 or (b) the MHC is a class II MHC and m is an integer from 9 to 25.

In some aspects, provided herein is a pharmaceutical composition comprising the polypeptide described herein and a pharmaceutically acceptable excipient. In some embodiments, the pharmaceutical composition further comprises an immunomodulatory agent or an adjuvant. In some embodiments, the immunomodulatory agent or an adjuvant is selected from the group consisting of poly-ICLC, 1018 ISS, aluminum salts, Amplivax, AS15, BCG, CP-870,893, CpG7909, CyaA, ARNAX, STING agonists, dSLIM, GM-CSF, IC30, IC31, Imiquimod, ImuFact IMP321, IS Patch, ISS, ISCOMATRIX, Juvlmmune, LipoVac, MF59, monophosphoryllipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam2Cys, Pam3Cys, Pam3CSK4, and Aquila's QS21 stimulon. In some embodiments, the immunomodulatory agent or adjuvant comprises poly-ICLC. In some embodiments, the pharmaceutical composition is a vaccine composition. In some embodiments, the pharmaceutical composition is aqueous or a liquid.

In some embodiments, the epitope is present in the pharmaceutical composition at an amount of from 1 ng to 10 mg or 5 μg to 1.5 mg. In some embodiments, the pharmaceutical composition further comprises DMSO. In some embodiments, the pharmaceutically acceptable excipient comprises water. In some embodiments, the pharmaceutical composition comprises a pH modifier present at a concentration of less than 1 mM or greater than 1 mM. In some embodiments, the pH modifier is a dicarboxylate salt or a tricarboxylate salt. In some embodiments, the pH modifier is a dicarboxylate salt of succinic acid, or a disuccinate salt. In some embodiments, the pH modifier is a tricarboxylate salt of citric acid, or a tricitrate salt. In some embodiments, the pH modifier is disodium succinate. In some embodiments, the dicarboxylate salt of succinic acid, or the disuccinate salt, is present in the pharmaceutical composition at a concentration of 0.1 mM-1 mM. In some embodiments, the dicarboxylate salt of succinic acid, or the disuccinate salt, is present in the pharmaceutical composition at a concentration of 1 mM-5 mM. In some embodiments, an immune response to the epitope is increased when administered to a subject.

In some aspects, provided herein is a method of treating a disease or a condition comprising administering a therapeutically effective amount of the pharmaceutical composition described herein to a subject in need thereof. In some embodiments, the disease or condition is a cancer. In some embodiments, the cancer is selected from the group consisting of lung cancer, non-small cell lung cancer, pancreatic cancer, colorectal cancer, uterine cancer, and liver cancer. In some embodiments, administering comprises intradermal injection, intranasal spray application, intramuscular injection, intraperitoneal injection, intravenous injection, oral administration, or subcutaneous injection.

In some aspects, provided herein is a method of prophylaxis of a subject comprising contacting a cell of the subject with the polypeptide, cell, or pharmaceutical composition described herein.

In some aspects, provided herein is a method comprising identifying an epitope expressed by a subject's tumor cells and producing a polypeptide comprising the epitope, wherein the polypeptide has a structure of Formula (I),

Yₙ-Bₜ-Aᵣ-Xₘ-Aₛ-Cᵤ-Zₚ Formula (I),

or a pharmaceutically acceptable salt thereof,

(i) wherein Xₘ is the epitope, wherein each X independently represents an amino acid of a contiguous amino acid sequence encoded by a nucleic acid sequence in a genome of a subject,

and wherein, (a) the MHC is a class I MHC and m is an integer from 8 to 12, or
(b) the MHC is a class II MHC and m is an integer from 9 to 25;

(ii) wherein each Y is independently an amino acid, analog, or derivative thereof, and wherein:
(A) when variable r of Aᵣ in Formula (I) is 0, Yₙ is not encoded by a nucleic acid sequence immediately upstream of the nucleic acid sequence in the genome of the subject that encodes Bₜ-Aᵣ-Xₘ,
(B) when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or
(C) when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and further wherein n is an integer from 0 to 1000;

(iii) wherein each Z is independently an amino acid, analog, or derivative thereof, and wherein:
(A) when variable s of Aₛ in Formula (I) is 0, Zₚ is not encoded by a nucleic acid sequence immediately downstream of the nucleic acid sequence in the genome of the subject that encodes Xₘ-Aₛ-Cᵤ,
(B) when variable s of Aₛ in Formula (I) is 1 and variable u of Cᵤ in Formula (I) is 0, Zₚ is not encoded by a nucleic acid sequence immediately downstream of the nucleic acid sequence in the genome of the subject that encodes Xₘ, or
(C) when variable s of Aₛ in Formula (I) is 1 and variable u of C in Formula (I) is 1 or more, Zₚ is not encoded by a nucleic acid sequence immediately downstream of the nucleic acid sequence in the genome of the subject that encodes Cᵤ; and

further wherein p is an integer from 0 to 1000;

and further wherein,

when n is 0, p is an integer from 1 to 1000; and

when p is 0, n is an integer from 1 to 1000;

(iv) wherein Aᵣ is a linker, and r is 0 or 1;

(v) wherein Aₛ is a linker, and s is 0 or 1;

(vi) wherein each B independently represents an amino acid encoded by a nucleic acid sequence in the genome of the subject that is immediately upstream of the nucleic acid sequence in the genome of the subject that encodes Xₘ,

and wherein t is an integer from 0 to 1000; and

(vii) wherein each C independently represents an amino acid encoded by a nucleic acid sequence in the genome of the subject that is immediately downstream of the nucleic acid sequence in the genome of the subject that encodes Xₘ, and wherein, u is an integer from 0 to 1000;

and further wherein,
(a) the polypeptide does not consist of four different epitopes presented by a class I MHC;
(b) the polypeptide comprises at least two different polypeptide molecules;
(c) the epitope comprises at least one mutant amino acid; and/or
(d) Yₙ and/or Zₚ is cleaved from the epitope when the polypeptide is processed by the APC.

In some embodiments, identifying comprises selecting a plurality of nucleic acid sequences from a pool of nucleic acid sequences sequenced from the subject's tumor cells that encode a plurality of candidate peptide sequences comprising one or more different mutations not present in a pool of nucleic acid sequences sequenced from the subject's non-tumor cells, wherein the pool of nucleic acid sequences sequenced from the subject's tumor cells and the pool of nucleic acid sequences sequenced from the subject's non-tumor cells are sequenced by whole genome sequencing or whole exome sequencing. In some embodiments, identifying further comprises predicting or measuring which candidate peptide sequences of the plurality of candidate peptide sequences form a complex with a protein encoded by an HLA allele of the same subject by an HLA peptide binding analysis. In some embodiments, identifying further comprises selecting the plurality of selected tumor-specific peptides or one or more polynucleotides encoding the plurality of selected tumor-specific peptides from the candidate peptide sequences based on the HLA peptide binding analysis.

In some embodiments, the method further comprises administering the polypeptide to the subject. In some embodiments, administering comprises intradermal injection, intranasal spray application, intramuscular injection, intraperitoneal injection, intravenous injection, oral administration, or subcutaneous injection. In some embodiments, an immune response is elicited in the subject. In some embodiments, the epitope expressed by the subject's tumor cells is a neoantigen, a tumor associated antigen, a mutated tumor associated antigen, and/or wherein expression of the epitope is higher in the subject's tumor cells compared to expression of the epitope in a normal cell of the subject.

Provided herein is polypeptide comprising an epitope presented by a class I MHC or a class II MHC of an antigen presenting cell (APC), the polypeptide having a structure of Formula (I):

Yₙ-Bₜ-Aᵣ-Xₘ-Aₛ-Cᵤ-Zₚ Formula (I),

or a pharmaceutically acceptable salt thereof, wherein Xₘ is the epitope, wherein each X independently represents an amino acid of a contiguous amino acid sequence encoded by a nucleic acid sequence in a genome of a subject, and wherein, (a) the MHC is a class I MHC and m is an integer from 8 to 12, or (b) the MHC is a class II MHC and m is an integer from 9 to 25; wherein each Y is independently an amino acid, analog, or derivative thereof, and wherein: when variable r of Aᵣ in Formula (I) is 0, Yₙ is not encoded by a nucleic acid sequence immediately upstream of the nucleic acid sequence in the genome of the subject that encodes Bₜ-Aᵣ-Xₘ, when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and further wherein, n is an integer from 0 to 1000; wherein each Z is independently an amino acid, analog, or derivative thereof, and wherein: when variable s of Aₛ in Formula (I) is 0, Zₚ is not encoded by a nucleic acid sequence immediately downstream of the nucleic acid sequence in the genome of the subject that encodes Xₘ-Aₛ-Cᵤ, when variable s of Aₛ in Formula (I) is 1 and variable u of Cᵤ in Formula (I) is 0, Zₚ is not encoded by a nucleic acid sequence immediately downstream of the nucleic acid sequence in the genome of the subject that encodes Xₘ, or when variable s of Aₛ in Formula (I) is 1 and variable u of Cᵤ in Formula (I) is 1 or more, Zₚ is not encoded by a nucleic acid sequence immediately downstream of the nucleic acid sequence in the genome of the subject that encodes Cᵤ; and further wherein, p is an integer from 0 to 1000; and further wherein, when n is 0, p is an integer from 1 to 1000; and when p is 0, n is an integer from 1 to 1000; wherein Aᵣ is a linker, and r is 0 or 1; wherein Aₛ is a linker, and s is 0 or 1; wherein each B independently represents an amino acid encoded by a nucleic acid sequence in the genome of the subject that is immediately upstream of the nucleic acid sequence in the genome of the subject that encodes Xₘ, and wherein t is an integer from 0 to 1000; and wherein each C independently represents an amino acid encoded by a nucleic acid sequence in the genome of the subject that is immediately downstream of the nucleic acid sequence in the genome of the subject that encodes Xₘ, and wherein, u is an integer from 0 to 1000; and further wherein, the polypeptide does not consist of four different epitopes presented by a class I MHC; the polypeptide comprises at least two different polypeptide molecules; the epitope comprises at least one mutant amino acid; and/or Yₙ and/or Zₚ is cleaved from the epitope when the polypeptide is processed by the APC.

In some embodiments, the epitope is presented by a class II MHC and m is an integer from 9 to 25.

In some embodiments, Yₙ-Bₜ-Aᵣ and/or Aₛ-Cᵤ-Zₚ enhances solubility of the polypeptide compared to a corresponding peptide that does not contain Yₙ-Bₜ-Aᵣ and/or Aₛ-Cᵤ-Zₚ. In some embodiments, the epitope is released from Yₙ-Bₜ-Aᵣ and/or Aₛ-Cᵤ-Zₚ when the polypeptide is processed by the APC. In some embodiments, the polypeptide is cleaved at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ; and/or wherein the polypeptide is cleaved at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ. In some embodiments, epitope presentation by the APC is enhanced when n is an integer from 1 to 1000 compared to epitope presentation of a corresponding polypeptide of the same length that comprises Xₘ and at least one additional amino acid encoded by a nucleic acid sequence immediately upstream of the nucleic acid sequence in the genome of the subject that encodes Xₘ; and/or wherein epitope presentation by the APC is enhanced when p is an integer from 1 to 1000 compared to epitope presentation of a corresponding polypeptide of the same length that comprises Xₘ and at least one additional amino acid encoded by a nucleic acid sequence immediately downstream of the nucleic acid sequence in the genome of the subject that encodes Xₘ. In some embodiments, the APC presents the epitope to an immune cell.

In some embodiments, immunogenicity is enhanced when n is an integer from 1 to 1000 compared to immunogenicity of a corresponding polypeptide of the same length that comprises Xₘ and at least one additional amino acid encoded by a nucleic acid sequence immediately upstream of the nucleic acid sequence in the genome of the subject that encodes Xₘ; and/or wherein immunogenicity is enhanced when p is an integer from 1 to 1000 compared to immunogenicity of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, anti-tumor activity is enhanced when n is an integer from 1 to 1000 compared to anti-tumor activity of a corresponding polypeptide of the same length that comprises Xₘ and at least one additional amino acid encoded by a nucleic acid sequence immediately upstream of the nucleic acid sequence in the genome of the subject that encodes Xₘ; and/or wherein anti-tumor activity is enhanced when p is an integer from 1 to 1000 compared to anti-tumor activity of a corresponding polypeptide of the same length that comprises Xₘ and at least one additional amino acid encoded by a nucleic acid sequence immediately downstream of the nucleic acid sequence in the genome of the subject that encodes Xₘ.

In some embodiments, Yₙ and/or Zₚ comprises a sequence selected from the group consisting of lysine (Lys), poly-Lys (polyK) and poly-Arg (polyR). In some embodiments, the polyK comprises poly-L-Lys. In some embodiments, the polyR comprises poly-L-Arg. In some embodiments, the polyK or polyR comprises at least two, three or four contiguous lysine or arginine residues, respectively.

In some embodiments, the epitope binds to MHC II class HLA. In some embodiments, the epitope binds to the MHC II class HLA with a stability of 10 minutes to 24 hours. In some embodiments, the epitope binds to the MHC II class HLA with an affinity of 0.1 nM to 2000 nM, 1 nM to 1000 nM, 10 nM to 500 nM, or less than 1000 nM.

In some embodiments, the polypeptide is not cleaved before processing by an APC or before internalization by an APC in a subject. In some embodiments, the polypeptide is stable in human plasma. In some embodiments, the polypeptide has a half-life of from 1 hour to 5 days in human plasma. In some embodiments, the subject is a human.

In some embodiments, the epitope binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM. In some embodiments, the epitope binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes. In some embodiments, the HLA allele is selected from the group consisting of HLA-A02:01 allele, an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, an HLA-A74:01 allele, and/or an HLA-C08:02 allele and any combination thereof. In some embodiments, the epitope comprises a tumor-specific epitope. In some embodiments, the epitope comprises at least one mutant amino acid In some embodiments, the at least one mutant amino acid is encoded by an insertion, a deletion, a frameshift, a neoORF, or a point mutation in the nucleic acid sequence in the genome of the subject.

In some embodiments, the epitope is a RAS epitope. In some embodiments, the epitope comprises a mutant RAS peptide sequence that comprises at least 8 contiguous amino acids of a mutant RAS protein comprising a mutation at G12, G13, or Q61 and the mutation at G12, G13, or Q61. In some embodiments, the at least 8 contiguous amino acids of a mutant RAS protein comprising a mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, the RAS epitope comprises an amino acid sequence of VVVGAAGVGK, VVVGAAGVG, VVVGAAGV, VVGAAGVGK, VVGAAGVG, VGAAGVGK, VVVGACGVGK, VVVGACGVG, VVVGACGV, VVGACGVGK, VVGACGVG, VGACGVGK, VVVGADGVGK, VVVGADGVG, VVVGADGV, VVGADGVGK, VVGADGVG, VGADGVGK, VVVGARGVGK, VVVGARGVG, VVVGARGV, VVGARGVGK, VVGARGVG, VGARGVGK, VVVGASGVGK, VVVGASGVG, VVVGASGV, VVGASGVGK, VVGASGVG, VGASGVGK, VVVGAVGVGK, VVVGAVGVG, VVVGAVGV, VVGAVGVGK, VVGAVGVG, or VGAVGVGK. In some embodiments, Yₙ comprises an amino acid sequence of K, KK, KKK, KKKK, KKKKK, KKKKKKK, KKKKKKKK, KTEY, KTEYK, KTEYKL, KTEYKLV, KTEYKLVV, KTEYKLVVV, KKTEY, KKTEYK, KKTEYKL, KKTEYKLV, KKTEYKLVV, KKTEYKLVVV, KKKTEY, KKKTEYK, KKKTEYKL, KKKTEYKLV, KKKTEYKLVV, KKKTEYKLVVV, KKKKTEY, KKKKTEYK, KKKKTEYKL, KKKKTEYKLV, KKKKTEYKLVV, KKKKTEYKLVVV, IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, TEYK, TEYKL, TEYKLV, TEYKLVV, TEYKLVVV, WQAGILAR, HSYTTAE, PLTEEKIK, GALHFKPGSR, RRANKDATAE, KAFISHEEKR, TDLSSRFSKS, FDLGGGTFDV, CLLLHYSVSK, KKKKIIMKIRNA, or MTEYKLVVV. In some embodiments, Zₚ comprises an amino acid sequence of K, KK, KKK, KKKK, KKKKK, KKKKKKK, KKKKKKKK, KKNKKDDI, KKNKKDDIKD, AGNDDDDDDDDDDDDDDDDDKKDKDDDDDD, AGNKKKKKKKNNNNNNNNNNNNNNNNNNNN, AGRDDDDDDDDDDDDDDDDDDDDDDDDDDD, SALTI, SALTIQL, GKSALTIQL, GKSALTI, SALTIK, SALTIQLK, GKSALTIQLK, GKSALTIK, SALTIKK, SALTIQLKK, GKSALTIQLKK, GKSALTIKK, SALTIKKK, SALTIQLKKK, GKSALTIQLKKK, GKSALTIKKK, SALTIKKKK, SALTIQLKKKK, GKSALTIQLKKKK, GKSALTI, KKKK, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE. In some embodiments, the polypeptide comprises an amino acid sequence of KTEYKLVVVGAVGVGKSALTIQL, KTEYKLVVVGADGVGKSALTIQL, KTEYKLVVVGARGVGKSALTIQL, KTEYKLVVVGACGVGKSALTIQL, KKTEYKLVVVGAVGVGKSALTIQL, KKTEYKLVVVGADGVGKSALTIQL, KKTEYKLVVVGARGVGKSALTIQL, KKTEYKLVVVGACGVGKSALTIQL, KKKTEYKLVVVGAVGVGKSALTIQL, KKKTEYKLVVVGADGVGKSALTIQL, KKKTEYKLVVVGARGVGKSALTIQL, KKKTEYKLVVVGACGVGKSALTIQL, KKKKTEYKLVVVGAVGVGKSALTIQL, KKKKTEYKLVVVGADGVGKSALTIQL, KKKKTEYKLVVVGARGVGKSALTIQL, KKKKTEYKLVVVGACGVGKSALTIQL, KKTEYKLVVVGAVGVGKSALTIQLKK, KKTEYKLVVVGADGVGKSALTIQLKK, KKTEYKLVVVGARGVGKSALTIQLKK, KKTEYKLVVVGACGVGKSALTIQLKK, TEYKLVVVGAVGVGKSALTIQLK, TEYKLVVVGADGVGKSALTIQLK, TEYKLVVVGARGVGKSALTIQLK, TEYKLVVVGACGVGKSALTIQLK, TEYKLVVVGAVGVGKSALTIQLKK, TEYKLVVVGADGVGKSALTIQLKK, TEYKLVVVGARGVGKSALTIQLKK, TEYKLVVVGACGVGKSALTIQLKK, TEYKLVVVGAVGVGKSALTIQLKKK, TEYKLVVVGADGVGKSALTIQLKKK, TEYKLVVVGARGVGKSALTIQLKKK, TEYKLVVVGACGVGKSALTIQLKKK, TEYKLVVVGAVGVGKSALTIQLKKKK, TEYKLVVVGADGVGKSALTIQLKKKK, TEYKLVVVGARGVGKSALTIQLKKKK, or TEYKLVVVGACGVGKSALTIQLKKKK. In some embodiments, the epitope is not a RAS epitope. In some embodiments, the polypeptide is not KKKKKPKRDGYMFLKAESKIMFAT, KKKKYMFLKAESKIMFATLQRSS, KKKKKAESKIMFATLQRSSLWCL, KKKKKIMFATLQRSSLWCLCSNH, or KKKKMFATLQRSSLWCLCSNH.

In some embodiments, Yₙ and/or Zₚ comprises an amino acid sequence of a protein different from the protein from which the epitope is derived. In some embodiments, Yₙ and/or Zₚ comprises an amino acid sequence of a protein of CMV such as pp65, HIV, or MART-1. In some embodiments, n is 1, 2, 3, 4, 5, 6, 7, 8, 9, 10, 11, 12, 13, 14, 15, 16, 17, 18, 19, 20 or an integer greater than 20. In some embodiments, p is 1, 2, 3, 4, 5, 6, 7, 8, 9, 10, 11, 12, 13, 14, 15, 16, 17, 18, 19, 20 or an integer greater than 20.

In some embodiments, the epitope is a TMPRSS2:ERG epitope. In some embodiments, the TMPRSS2:ERG epitope comprises an amino acid sequence of ALNSEALSV.

Also provided herein is a polynucleotide comprising a sequence encoding a polypeptide described herein. In some embodiments, the polynucleotide is an mRNA.

Also provided herein is a pharmaceutical composition comprising a polypeptide described herein or a polynucleotide described herein; and a pharmaceutically acceptable excipient.

Also provided herein is a method of treating a disease or a condition comprising administering a therapeutically effective amount of a pharmaceutical composition described herein to a subject in need thereof. In some embodiments, the disease or the condition is a cancer selected from the group consisting of lung cancer, non-small cell lung cancer, pancreatic cancer, colorectal cancer, uterine cancer, prostate cancer, liver cancer, a biliary tract malignancy, endometrial cancer, cervical cancer, bladder cancer, liver cancer, myeloid leukemia and breast cancer. In some embodiments, administering comprises intradermal injection, intranasal spray application, intramuscular injection, intraperitoneal injection, intravenous injection, oral administration, or subcutaneous injection.

Also provided herein is a method of preparing antigen-specific T cells comprising stimulating T cells with antigen presenting cells comprising a polypeptide described herein or a polynucleotide encoding the polypeptide described herein. In some embodiments, the method is performed ex vivo.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 depicts a simplified exemplary epitope processing and presentation of epitope X on HLA allele X by antigen presenting cells (APCs). In natural context, the peptide comprises an amino acid or an amino acid sequence that is naturally flanking the epitope sequence. In rational context, the peptide comprises, to the N- and/or C-terminus of the epitope sequence, an amino acid or an amino acid sequence that is not encoded by the genome that encodes the epitope sequence, and/or a linker.

FIG. 2 illustrates an exemplary Cathepsin B cleavage of a polypeptide containing Cathepsin B-cleavable linker.

FIG. 3 depicts a diagram of the experimental design to screen polypeptides in vitro for epitope processing and presentation using T cell receptor (TCR)-transduced cells (results shown in FIGS. 4 and 5).

FIG. 4 depicts a graph demonstrating the level of IL-2 (pg/mL) secreted by KRAS specific Jurkat cells after a 48 hour co-culture with an equal amount of peripheral blood mononuclear cells (PBMCs) loaded with either a peptide containing the KRAS-G12V epitope only or a peptide containing the KRAS-G12V epitope and additional amino acid sequences naturally flanking KRAS-G12V epitope on the N- and C-terminus.

FIG. 5 depicts a graph demonstrating the level of IL-2 (pg/mL) secreted by KRAS specific Jurkat cells after a 48 hour co-culture with an equal amount of peripheral blood mononuclear cells (PBMCs) loaded with either a peptide containing the KRAS-G12V epitope only, a peptide containing the KRAS-G12V epitope and additional amino acid sequences naturally flanking KRAS-G12V epitope on the N- and C-terminus, or a peptide containing the KRAS-G12V epitope and additional amino acid sequences rationally designed not naturally flanking KRAS-G12V epitope (rational context) on the N- and/or C-terminus.

FIG. 6 depicts a diagram of the experimental design of an immunogenicity study. Mice were immunized on days 0, 7, and 14 with various polypeptide designs, and bled on days 7, 14, and 21 to evaluate antigen-specific CD8+ T cell responses (results shown in FIGS. 7-9).

FIG. 7 depicts graphs demonstrating total immune responses (7A: H-2K^{b}, 7B: H-2D^{b}, 7C: total).

FIG. 8 depicts graphs demonstrating that immunization with K4-epitopes enhance immune responses to H-2K^{b}-presented epitopes (8A: Alg8, 8B: Lama4).

FIG. 9 depicts graphs demonstrating that immunization with K4-epitope increases immune responses to H-2D^{b}-presented epitopes (9A: Reps1, 9B: Adpgk, 9C: Irgq, 9D: Obsl1).

FIG. 10 depicts a graph demonstrating that the level of IL-2 (pg/mL) secreted by Jurkat cells after a 24 hour co-culture with 293T cells (5:1 ratio of Jurkats to 293T cells) loaded with a peptide containing the TMPRSS2::ERG epitope only or transduced with a plasmid encoding a peptide containing the TMPRSS2:: ERG epitope in natural context (i.e., the peptide additionally comprises an amino acid or an amino acid sequence that is naturally flanking the epitope sequence on the N- and/or C-terminus), a plasmid encoding a peptide containing the TMPRSS2::ERG epitope in non-natural context (i.e., the peptide additionally comprises an amino acid or an amino acid sequence that is not naturally flanking the epitope sequence), or a plasmid encoding an irrelevant epitope in non-natural context (as a control).

FIG. 11 depicts a graph of IL-2 concentration (pg/mL) vs peptide concentration (nM) in FLT3L-treated PBMCs contacted with increasing amounts of the indicated RAS-G12V mutant peptides after being co-cultured with Jurkat cells transduced with a TCR that binds to the underlined RAS-G12V epitope bound to an MHC encoded by the HLA-A11:01 allele.

FIG. 12 depicts data illustrating the immunogenicity of the indicated RAS-G12V mutant peptides from FIG. 11 both in vitro using PBMCs from healthy donors (top) and in vivo using HLA-A11:01 transgenic mice immunized with the peptides (bottom).

FIG. 13A depicts exemplary schematics of mRNA constructs using shortmers (9-10 amino acids, top) and longmers (25 amino acids, bottom) used for expression in cells.

FIG. 13B depicts an exemplary graph of multimer specific CD8+ cells as the percentage of total CD8+ cells. The antigens used for the multimer assay are shown.

FIG. 13C depicts exemplary flow cytometry analyses of detection of multimer positive CD8+ T cells, comparing shortmer (9-10 amino acids) and longmer (25 amino acids) peptide stimulated APCs and APCs containing RNAs encoding the same shortmer (9-10 amino acids) and longmer (25 amino acids) peptides.

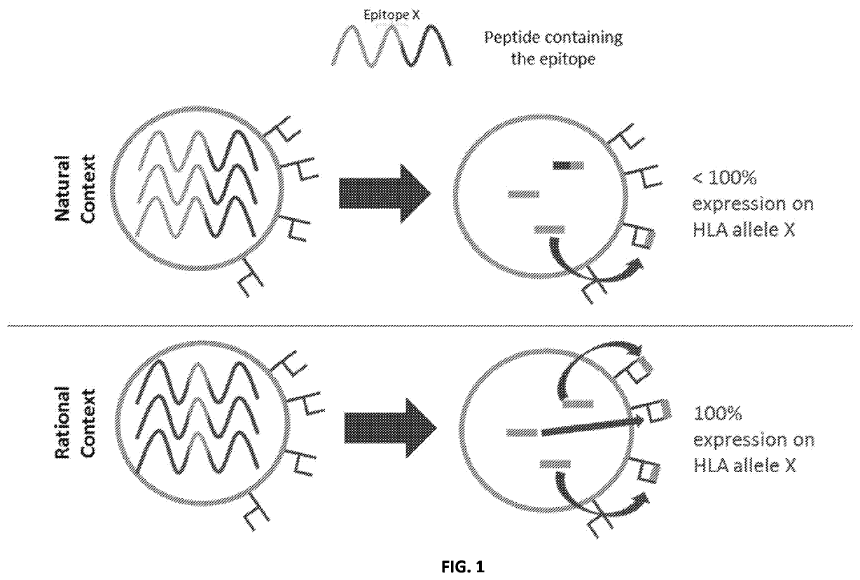

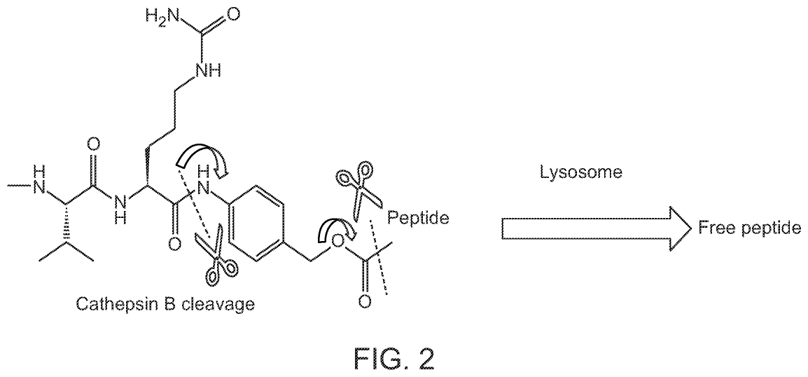

### DETAILED DESCRIPTION

Described herein are new immunotherapeutic compositions comprising an individual's tumor-specific antigen or neoepitope and uses thereof based on the discovery of methods for enhancing epitope processing and presentation to stimulate an immune response. Accordingly, the present disclosure described herein provides peptides that can be used, for example, to stimulate an immune response to a tumor associated antigen or neoepitope, to create an immunogenic composition or cancer vaccine for use in treating a cancer, disease, or condition.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### 1. Definitions

The terminology used herein is for the purpose of describing particular cases only and is not intended to be limiting. In this application, the use of the singular includes the plural unless specifically stated otherwise. As used herein, the singular forms “a,” “an,” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part. In the formula representing selected specific embodiments of the present disclosure, the amino- and carboxyl-terminal groups, although not specifically shown, are in the form they would assume at physiologic pH values, unless otherwise specified. In the amino acid structure formula, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G.” The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine).

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

“Polypeptide,” “peptide,” and their grammatical equivalents as used herein refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, famesylation, geranylation, glypiation, lipoylation and iodination.

The terms “peptide” refers to a series of amino acid residues connected one to the other, typically by peptide bonds between the α-amino and carboxyl groups of adjacent amino acid residues.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

An “epitope” is the collective features of a molecule, such as primary, secondary, and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. A “T cell epitope” is to be understood as meaning a peptide sequence which can be bound by the MHC molecules of class I or II in the form of a peptide-presenting MHC molecule or MHC complex and then, in this form, be recognized and bound by T cells, such as T-lymphocytes or T-helper cells. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes. It is to be appreciated that proteins or peptides that comprise an epitope or an analog described herein as well as additional amino acid(s) are still within the bounds of the present disclosure. In certain embodiments, the peptide comprises a fragment of an antigen. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acid residues.

The term “derived” and its grammatical equivalents when used to discuss an epitope is a synonym for “prepared” and its grammatical equivalents. A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein are capable of binding to an appropriate HLA molecule and thereafter inducing a CTL (cytotoxic) response, or a HTL (and humoral) response, to the peptide.

“Neoantigen” means a class of tumor antigens which arise from tumor-specific changes in proteins. Neoantigens encompass, but are not limited to, tumor antigens which arise from, for example, substitution in the protein sequence, frame shift mutation, fusion polypeptide, in-frame deletion, insertion, expression of endogenous retroviral polypeptides, and tumor-specific overexpression of polypeptides.

The term “mutant peptide,” “tumor-specific peptide,” “neoantigen peptide,” and “neoantigenic peptide,” used interchangeably with “peptide” in the present specification, refers to a series of residues, typically L-amino acids, connected one to the other, typically by peptide bonds between the α-amino and carboxyl groups of adjacent amino acids. Similarly, the term “polypeptide” is used interchangeably with “mutant polypeptide,” “neoantigen polypeptide,” and “neoantigenic polypeptide” in the present specification to designate a series of residues, e.g., L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. A peptide or polypeptide as used herein comprises at least one flanking sequence. The term “flanking sequence” as used herein refers to a fragment or region of the neoantigen peptide that is not a part of the neoepitope.

A “neoepitope,” “tumor-specific neoepitope,” “tumor-specific epitope,” or “tumor antigen” refers to an epitope or antigenic determinant region that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” as used herein refers to an antigenic determinant region within the peptide or neoantigenic peptide. A neoepitope may comprise at least one “anchor residue” and at least one “anchor residue flanking region.” A neoepitope may further comprise a “separation region.” The term “anchor residue” refers to an amino acid residue that binds to specific pockets on HLAs, resulting in specificity of interactions with HLAs. In some cases, an anchor residue may be at a canonical anchor position. In other cases, an anchor residue may be at a non-canonical anchor position. Neoepitopes may bind to HLA molecules through primary and secondary anchor residues protruding into the pockets in the peptide-binding grooves. In the peptide-binding grooves, specific amino acids compose pockets that accommodate the corresponding side chains of the anchor residues of the presented neoepitopes. Peptide-binding preferences exist among different alleles of both of HLA I and HLA II molecules. HLA class I molecules bind short neoepitopes, whose N- and C-terminal ends are anchored into the pockets located at the ends of the neoepitope binding groove. While the majority of the HLA class I binding neoepitopes are of about 9 amino acids, longer neoepitopes can be accommodated by the bulging of their central portion, resulting in binding neoepitopes of about 8 to 12 amino acids. Neoepitopes binding to HLA class II proteins are not constrained in size and can vary from about 16 to 25 amino acids. The neoepitope binding groove in the HLA class II molecules is open at both ends, which enables binding of peptides with relatively longer length. Though the core 9 amino acid residues long segment contributes the most to the recognition of the neoepitope, the anchor residue flanking regions are also important for the specificity of the peptide to the HLA class II allele. In some cases, the anchor residue flanking region is N-terminus residues. In another case, the anchor residue flanking region is C-terminus residues. In yet another case, the anchor residue flanking region is both N-terminus residues and C-terminus residues. In some cases, the anchor residue flanking region is flanked by at least two anchor residues. An anchor residue flanking region flanked by anchor residues is a “separation region.”

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

“Human Leukocyte Antigen” or “HLA” is a human class I or class II Major Histocompatibility Complex (MHC) protein (see, e.g., Stites, et al., Immunology, 8^{th} Ed., Lange Publishing, Los Altos, Calif. (1994).

“Peptide-MHC (pMHC) stability” refers to the length of time it takes for half of the amount of a specific peptide to dissociate from the cognate HLA in a biochemical assay.

“Antigen presenting cells” (APC) are cells which present peptide fragments of protein antigens in association with MHC molecules on their cell surface. Some APCs may activate antigen specific T cells. Mature professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen presenting cells, macrophages, B-cells, and certain activated epithelial cells. “Dendritic cells (DCs)” are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc receptor (FcR) and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

The terms “polynucleotide,” “nucleotide,” “nucleic acid,” “polynucleic acid,” or “oligonucleotide” and their grammatical equivalents are used interchangeably herein and refer to polymers of nucleotides of any length, and include DNA and RNA, for example, mRNA. Thus, these terms includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein may be introduced into a cell by, for example, transfection, transformation, or transduction. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the present disclosure is mRNA.

A “reference” can be used to correlate and compare the results obtained in the methods of the present disclosure from a tumor specimen. Typically the “reference” may be obtained on the basis of one or more normal specimens, in particular, specimens which are not affected by a cancer disease, either obtained from a patient or one or more different individuals, for example, healthy individuals, in particular, individuals of the same species. A “reference” can be determined empirically by testing a sufficiently large number of normal specimens.

The term “mutation” or “mutant” refers to a change of or difference in the nucleic acid sequence (nucleotide substitution, addition, insertion, or deletion) compared to a reference. A “somatic mutation” can occur in any of the cells of the body except the germ cells (sperm and egg) and therefore are not passed on to children. These alterations can (but do not always) cause cancer or other diseases. In some embodiments, a mutation is a non-synonymous mutation. The term “non-synonymous mutation” refers to a mutation, for example, a nucleotide substitution, which does result in an amino acid change such as an amino acid substitution in the translation product. A “frameshift” occurs when a mutation disrupts the normal phase of a gene's codon periodicity (also known as “reading frame”), resulting in the translation of a non-native protein sequence. It is possible for different mutations in a gene to achieve the same altered reading frame. A “neoORF” can be created when an open reading frame (ORF) is altered through various mutational events in the genome, such as missense mutations, fusion transcripts, frameshifts, and/or stop codon losses. A neoORF can encode novel amino acid sequences that are not present in the normal genome.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

A “native” or a “wild-type” sequence refers to a sequence found in nature. Such a sequence can comprise a longer sequence in nature.

As used herein, the term “affinity” refers to a measure of the strength of binding between two members of a binding pair, for example, an HLA binding peptide and a class I or II HLA. K_{D} is the dissociation constant and has units of molarity. The affinity constant is the inverse of the dissociation constant. An affinity constant is sometimes used as a generic term to describe this chemical entity. It is a direct measure of the energy of binding. Affinity may be determined experimentally, for example by surface plasmon resonance (SPR) using commercially available Biacore SPR units. Affinity may also be expressed as the inhibitory concentration 50 (IC₅₀), that concentration at which 50% of the peptide is displaced. Likewise, ln(IC₅₀) refers to the natural log of the IC₅₀. K_{off} refers to the off-rate constant, for example, for dissociation of an HLA binding peptide and a class I or II HLA. Throughout this disclosure, “binding data” or “binding analysis” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)). “Cross-reactive binding” indicates that a peptide is bound by more than one HLA molecule; a synonym is degenerate binding.

The term “naturally occurring” and its grammatical equivalents as used herein refer to the fact that an object can be found in nature. For example, a peptide or nucleic acid that is present in an organism (including viruses) and can be isolated from a source in nature and which has not been intentionally modified by man in the laboratory is naturally occurring.

“Antigen processing” or “processing” and its grammatical equivalents refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

A “cell” and their grammatical equivalents refers to a cell of human or non-human animal origin.

A “T cell” includes CD4+ T cells and CD8+ T cells. The term T cell also includes both T helper 1 type T cells and T helper 2 type T cells.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder; and (2) prophylactic or preventative measures that prevent or slow the development of a targeted pathologic condition or disorder. Thus those in need of treatment include those already with the disorder; those prone to have the disorder; and those in whom the disorder is to be prevented.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

An “immunomodulatory agent” or its grammatical equivalent as used herein can refer to a substance that can stimulate or suppress the immune system and may help an individual's body to fight a disease, for example, infection, cancer, etc. Examples of specific immunomodulatory agent that affects specific parts of the immune system include, but are not limited to, monoclonal antibodies, cytokines, and vaccines. Nonspecific immunomodulatory agents affect the immune system in a general way and non-limiting examples include Bacillus Calmette-Guerin (BCG) and levamisole.

The term “cancer” and its grammatical equivalents as used herein can refer to a hyperproliferation of cells whose unique trait—loss of normal controls—results in unregulated growth, lack of differentiation, local tissue invation, and metastasis. With respect to the inventive compositions and methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “exome” refers to the part of genome that encodes for functional proteins, or the sequence encompassing all exons, or coding regions, of protein coding genes in the genome. It is about 1-2% of the whole genome depending on species.

A “diluent” includes sterile liquids, such as water and oils, including those of petroleum, animal, vegetable or synthetic origin, such as peanut oil, soybean oil, mineral oil, sesame oil and the like. Water is also a diluent for pharmaceutical compositions. Saline solutions and aqueous dextrose and glycerol solutions can also be employed as diluents, for example, in injectable solutions.

A “receptor” is to be understood as meaning a biological molecule or a molecule grouping capable of binding a ligand. A receptor may serve, to transmit information in a cell, a cell formation, or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex,” including a peptide- or peptide fragment-presenting MHC molecule of class I or of class II.

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99%, or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98%, and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. In some embodiments, an “isolated polynucleotide” encompasses a PCR or quantitative PCR reaction comprising the polynucleotide amplified in the PCR or quantitative PCR reaction.

The term “isolated,” “biologically pure,” or their grammatical equivalents refers to material which is substantially or essentially free from components which normally accompany the material as it is found in its native state. Thus, isolated peptides described herein do not contain some or all of the materials normally associated with the peptides in their in situ environment. An “isolated” epitope refers to an epitope that does not include the whole sequence of the antigen from which the epitope was derived. Typically the “isolated” epitope does not have attached thereto additional amino acid residues that result in a sequence that has 100% identity over the entire length of a native sequence. The native sequence can be a sequence such as a tumor-associated antigen from which the epitope is derived. Thus, the term “isolated” means that the material is removed from its original environment (e.g., the natural environment if it is naturally occurring). An “isolated” nucleic acid is a nucleic acid removed from its natural environment. For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

The term “substantially pure” as used herein refers to material which is at least 50% pure (i.e., free from contaminants), at least 90% pure, at least 95% pure, at least 98% pure, or at least 99% pure.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available. 2. Enhanced cleavage and Uses Thereof

One of the critical barriers to developing curative and tumor-specific immunotherapy is insufficient processing and release of minimal epitopes for antigen presentation to generate adequate immune responses. Antigen processing and presentation refer to the processes that occur within a cell that result in fragmentation, or proteolysis, of proteins, association of the protein fragments, or peptides, with major histocompatibility complex (MHC) molecules, and the expression of the peptide-MHC (pMHC) molecules on the cell surface for recognition by T cell receptor (TCR) on a T cell. Antigen presentation is mediated by MHC class I molecules and MHC class II molecules found on the surface of antigen-presenting cells (APCs) and certain other cells. MHC class I and MHC class II molecules deliver short peptides to the cell surface allowing these peptides to be recognized by cytotoxic (CD8+) and helper (CD4+) T cells, respectively. The TCR can recognize antigen only in the form of a peptide bound to an MHC molecule on a cell surface and the antigens recognized by T cells are peptides that arise from the breakdown of macromolecular structures, the unfolding of individual proteins, and their cleavage into short fragments through antigen processing.

Antigen presentation on the cell surface requires correct processing of peptides to release minimal epitopes by the proteasome, cytosolic and endoplasmic reticulum (ER) aminopeptidases, efficient transporter associated with antigen processing (TAP) transport, and sufficient binding to MHC class I molecules. The efficiency of the epitope generation depends not only on the epitope itself but also on its flanking regions or the amino acid sequence flanking the amino acid sequence of the epitope. The efficiency of processing minimal epitope from the peptide comprising the epitope sequence and amino acid sequence flanking the epitope sequence is not completely understood but is known to be affected by multiple factors including the specific amino acid residues on both sides of the cleavage site in the peptide and other competing cleavage sites nearby.

One way to address insufficient processing and release of minimal epitope problem is to study and design the specific amino acid residues or sequences that can be added to N- and/or C-terminus of the epitope sequence to enhance cleavage and processing of peptides and presentation of epitopes. For example, amino acid residues or sequences from other epitopes that are known to be processed efficiently can be added to an epitope sequence. Another example is to use amino acid residues that are known to be commonly observed around epitopes (Abelin, et al., 2017, Immunity 46, 315-326). This approach can confer additional benefits including facilitating the manufacture (e.g., synthesis, purification, and/or formulation) or easier downstream modification (e.g., conjugation to other molecules) of peptides.

Another way to address the current barriers to efficient processing and release of minimal epitopes is to use a protease-cleavable linker to target an epitope-containing peptide for site-specific protease processing for the release of the epitope. For example, specific linkers that can be readily cleaved inside dendritic cells (DCs) to release minimal epitope sequences can used to enhance CD8-dependent immune responses after vaccination. These peptides, additionally, will not have non-selective binding to MHC class I molecules on the surfaces of non-professional APCs, and instead will go through specific (e.g., endocytosis) pathways to be properly processed and presented to T cells. Yet, another example to promote sufficient epitope processing and presentation is to combine the two strategies, i.e., the specific amino acid residues and specific linkers.

Provided herein is a polypeptide comprising an epitope sequence encoded by a genome of a subject, an amino acid or an amino acid sequence that may or may not be encoded by a nucleic acid sequence immediately upstream or downstream of the nucleic acid sequence encoding the epitope sequence in the genome of the subject, an amino acid or an amino acid sequence, and/or a linker. The addition of an amino acid, an amino acid sequence, and/or a linker to the epitope sequence can enhance epitope processing and presentation by APCs for generation of an immune response. In one aspect, the amino acid or the amino acid sequence is of an amino acid sequence or a peptide sequence. In one embodiment, the amino acid sequence or the peptide sequence is not encoded by a nucleic acid sequence immediately upstream or downstream of the nucleic acid sequence in the genome of the subject that encodes the epitope sequence. In another embodiment, the amino acid or the amino acid sequence is contiguous with the epitope sequence and is encoded by the genome of the subject that encodes the epitope sequence. For example, the amino acid or the amino acid sequence contiguous with the epitope sequence may comprise one or more amino acid residues that enhances cleavage of the polypeptide (e.g., lysine). In such embodiment, the polypeptide may comprise the amino acid or the amino acid sequence contiguous with the epitope sequence and may further comprise the amino acid or the amino acid sequence that is not encoded by the nucleic acid sequence immediately upstream or downstream of the nucleic acid sequence encoding the epitope sequence in the genome of the subject.

In some embodiments, the epitope is presented by a class I MHC of an APC. In some embodiments, the epitope is presented by a class II MHC of an APC. In some embodiments, each amino acid of the epitope represents an amino acid of a peptide sequence comprising any contiguous amino acid sequence encoded by a nucleic acid sequence in a genome of a subject. In some embodiments, the epitope comprises 8 to 12 contiguous amino acid residues and is presented by a class I MHC of an APC. In some embodiments, the epitope comprises 8, 9, 10, 11, or 12 contiguous amino residues and is presented by a class I MHC of an APC. In some embodiments, the epitope comprises 9 to 25 contiguous amino acid residues and is presented by a class II MHC of an APC. In some embodiments, the epitope comprises 9, 10, 11, 12, 13, 14, 15, 16, 17, 18, 19, 20, 21, 22, 23, 24, or 25 contiguous amino acid residues and is presented by a class II MHC of an APC. In some embodiments, the epitope sequence comprises 8, 9, 10, 11, 12, 13, 14, 15, 16, 17, 18, 19, 20, 21, 22, 23, 24, or 25 contiguous amino acid residues, of which one of more of 13^{th} to 25^{th} amino acids are optionally present and at least one amino acid is a mutant amino acid. In some embodiments, the epitope sequence comprises AA₁AA₂AA₃AA₄AA₅AA₆AA₇AA₈AA₉AA₁₀AA₁₁AA₁₂AA₁₃AA₁₄AA₁₅AA₁₆AA₁₇AA₁₈AA₁₉AA₂₀AA₂₁AA₂₂AA₂₃AA₂₄AA₂₅, wherein each AA is an amino acid, and one or more of AA₉, AA₁₀, AA₁₁, AA₁₂, AA₁₃, AA₁₄, AA₁₅, AA₁₆, AA₁₇, AA₁₈, AA₁₉, AA₂₀, AA₂1, AA₂₂, AA₂₃, AA₂₄, and AA₂₅ are optionally present, and at least one AA is a mutant amino acid.

In some embodiments, the polypeptide comprising an epitope sequence and an amino acid or an amino acid sequence that is contiguous with the epitope sequence and is encoded by a nucleic acid sequence immediately upstream or downstream of the nucleic acid sequence encoding the epitope in the genome of the subject may not comprise a linker. In some embodiments, the polypeptide comprising an epitope sequence and an amino acid or an amino acid sequence that is contiguous with the epitope sequence and is encoded by a nucleic acid sequence immediately upstream or downstream of the nucleic acid sequence encoding the epitope in the genome of the subject may comprise a linker. In some embodiments, the polypeptide comprising the epitope sequence and an amino acid or an amino acid sequence that is not encoded by the nucleic acid sequence immediately upstream or downstream of the nucleic acid sequence in the genome of the subject that encodes the epitope sequence may further comprise a linker. In some embodiments, the polypeptide comprising an epitope sequence and an amino acid or an amino acid sequence that is not encoded by the nucleic acid sequence immediately upstream or downstream of the nucleic acid sequence in the genome of the subject that encodes the epitope sequence may not comprise a linker.

In some embodiments, the amino acid or the amino acid sequence comprises 0 to 1000 amino acid residues in length. In some embodiments, the amino acid or the amino acid sequence that is encoded by a nucleic acid sequence immediately upstream of the nucleic acid sequence in the genome of the subject that encodes the epitope comprises 0 to 1000 amino acid residues in length. In some embodiments, the amino acid or the amino acid sequence that is encoded by a nucleic acid sequence immediately downstream of the nucleic acid sequence in the genome of the subject that encodes the epitope comprises 0 to 1000 amino acid residues in length. In some embodiments, the amino acid or the amino acid sequence comprises more than 0, more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 55, more than 60, more than 65, more than 70, more than 75, more than 80, more than 85, more than 90, more than 95, more than 100, more than 150, more than 200, more than 250, more than 300, more than 350, more than 400, more than 450, more than 500, more than 550, more than 600, more than 650, more than 700, more than 750, more than 800, more than 850, more than 900, or more than 950 amino acid residues in length. In some embodiments, the amino acid or the amino acid sequence that is encoded by a nucleic acid sequence immediately upstream of the nucleic acid sequence in the genome of the subject that that encodes the epitope comprises more than 0, more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 55, more than 60, more than 65, more than 70, more than 75, more than 80, more than 85, more than 90, more than 95, more than 100, more than 150, more than 200, more than 250, more than 300, more than 350, more than 400, more than 450, more than 500, more than 550, more than 600, more than 650, more than 700, more than 750, more than 800, more than 850, more than 900, or more than 950 amino acid residues in length. In some embodiments, the amino acid or the amino acid sequence that is encoded by a nucleic acid sequence immediately downstream of the nucleic acid sequence in the genome of the subject that that encodes the epitope comprises more than 0, more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 55, more than 60, more than 65, more than 70, more than 75, more than 80, more than 85, more than 90, more than 95, more than 100, more than 150, more than 200, more than 250, more than 300, more than 350, more than 400, more than 450, more than 500, more than 550, more than 600, more than 650, more than 700, more than 750, more than 800, more than 850, more than 900, or more than 950 amino acid residues in length.

In some embodiments, the amino acid or the amino acid sequence comprises 1-5 or 7-1000 amino acid residues in length. In some embodiments, the amino acid or the amino acid sequence does not comprise 6 amino acid residues in length. In some embodiments, the amino acid or the amino acid sequence of a peptide sequence that is encoded by a nucleic acid sequence immediately upstream of the nucleic acid sequence in the genome of the subject that encodes the epitope comprises 1-5 or 7-1000 amino acid residues in length. In some embodiments, the amino acid or the amino acid sequence of a peptide sequence that is encoded by a nucleic acid sequence immediately upstream of the nucleic acid sequence in the genome of the subject that encodes the epitope does not comprise 6 amino acid residues in length. In some embodiments, the amino acid or the amino acid sequence of a peptide sequence that is encoded by a nucleic acid sequence immediately downstream of the nucleic acid sequence in the genome of the subject that encodes the epitope comprises 1-4 or 6-1000 amino acid residues in length. In some embodiments, the amino acid or the amino acid sequence of a peptide sequence that is encoded by a nucleic acid sequence immediately downstream of the nucleic acid sequence in the genome of the subject that encodes the epitope does not comprise 5 amino acid residues in length.

In some embodiments, the polypeptide further comprises a linker. In some embodiments, the polypeptide does not consist of four different epitopes presented by a class I MHC. In some embodiments, the polypeptide does not comprise four different epitopes presented by a class I MHC. In some embodiments, the polypeptide comprises at least two different epitopes presented by a class I MHC. In some embodiments, the polypeptide comprises at least three, at least five, or at least six different epitopes presented by a class I MHC. In some embodiments, the epitope comprises at least one mutant amino acid. In some embodiments, the at least one mutant amino acid is encoded by an insertion, a deletion, a frameshift, a neoORF, or a point mutation in the nucleic acid sequence in the genome of the subject. In some embodiments, the amino acid or an amino acid sequence of a peptide sequence that is not encoded by a nucleic acid sequence immediately downstream or upstream of the nucleic acid sequence in the genome of the subject that encodes the epitope is cleaved from the epitope when the polypeptide is processed by the APC. In some embodiments, the polypeptide comprises at least two different polypeptide molecules. In some embodiments, the polypeptide comprises at least three, at least four, or at least five different polypeptide molecules.

In some embodiments, the present disclosure includes a polypeptide comprising an amino acid or an amino acid sequence of a peptide sequence that is not encoded by a nucleic acid sequence immediately downstream or upstream of the nucleic acid sequence in the genome of the subject that encodes the epitope, and/or a linker. The amino acid or the amino acid sequence and/or the linker can provide the polypeptide desired properties such as increased solubility, stability, immunogenecity, antigen processing, or antigen presentation. In some embodiments, a polypeptide may comprise an amino acid or an amino acid sequence that enhances processing and presentation of epitopes by APCs, for example, for generation of an immune response. In some embodiments, the polypeptide may include an amino acid or an amino acid sequence either on the N- and/or C-terminus of the epitope sequence. In some embodiments, the amino acid or the amino acid sequence can comprise poly-lysine (poly-Lys or polyK) or poly-arginine (poly-Arg or polyR). In some embodiments, the amino acid or the amino acid sequence can be of a polypeptide sequence of a protein not expressed in a subject expressing the epitope (e.g., not encoded by the genome of the subject encoding the epitope sequence). In another embodiment, the polypeptide may comprise a linker that is cleavable by a protease. In some embodiments, the polypeptide can comprise both the protease-cleavable linker and the amino acid or the amino acid sequence. In some embodiments, provided herein is a polypeptide of formula (I), (II), (III), and/or (IV), or a pharmaceutically acceptable salt of a polypeptide of formula (I), (II), (III), and/or (IV), wherein the stereochemistry is undefined, e.g., a racemate or a mixture of diastereomers or individual diastereomers. The skilled person in the art will recognize that at any stage of the preparation of the compounds of formula (I), (II), (III), and/or (IV), mixtures of isomers (e.g., racemates) of compounds corresponding to any of formula (I), (II), (III), and/or may be utilized. At any stage of the preparation, a single stereoisomer may be obtained by isolating it from a mixture of isomers (e.g., a racemate) using, for example, chiral chromatographic separation.

In some embodiments, the linker comprises a non-polypeptide linker. In some embodiments, the linker comprises a chemical linker. In some embodiments, the linker comprises a non-natural amino acid. In some embodiments, the non-natural amino acid comprises β-γ-δ-amino acids. In some embodiments, the non-natural amino acid comprises derivatives of L-α-amino acids. In some embodiments, the linker does not comprise an amino acid. In some embodiments, the linker does not comprise a natural amino acid. In some embodiments, the linker comprises a bond other than a peptide bond. In some embodiments, the linker comprises a disulfide bond. In some embodiments, the polypeptide described herein comprises more than one linker. In some embodiments, the polypeptide described herein comprises a first linker and a second linker wherein the first linker is at the N-terminus of the epitope and the second linker is at the C-terminus of the epitope. In some embodiments, the first linker and the second linker are different. In some embodiments, the first linker and the second linker are the same.

In some embodiments, the polypeptide comprises a hydrophilic tail. In some embodiments, the polypeptide comprising an epitope sequence, an amino acid or an amino acid sequence of a peptide sequence that is not encoded by the nucleic acid sequence immediately downstream or upstream of the nucleic acid sequence in the genome of the subject that encodes the epitope and/or a linker has enhanced solubility compared to a polypeptide comprising the same epitope sequence without the amino acid or the amino acid sequence and/or the linker. In some embodiments, the polypeptide comprising an epitope sequence and an amino acid or an amino acid sequence contiguous with the epitope sequence encoded by the nucleic acid sequence in the genome of a subject has enhanced solubility compared to a polypeptide comprising the same epitope sequence without the amino acid or the amino acid sequence. For example, the amino acid or the amino acid sequence contiguous with the epitope sequence may comprise one or more amino acid residues that enhances solubility of the polypeptide (e.g., lysine). In such embodiment, the polypeptide may comprise the amino acid or the amino acid sequence contiguous with the epitope sequence and may further comprise an amino acid or an amino acid sequence of a peptide sequence that is not encoded by the nucleic acid sequence immediately downstream or upstream of the nucleic acid sequence in the genome of the subject that encodes the epitope.

In some embodiments, the epitope is released from the polypeptide comprising the epitope sequence when the polypeptide is processed by an APC. In some embodiments, the epitope is released at a higher rate when the polypeptide further comprises an amino acid or an amino acid sequence that does not comprise at least one additional amino acid encoded by a nucleic acid sequence immediately upstream of the nucleic acid sequence in the genome of the subject that encodes the epitope, and/or a linker, compared to a polypeptide that comprises the same epitope but does not comprise the amino acid or the amino acid sequence that does not comprise at least one additional amino acid encoded by the nucleic acid sequence immediately upstream of the nucleic acid sequence in the genome of the subject that encodes the epitope, and/or a linker. In some embodiments, the epitope is released at a higher rate when the polypeptide further comprises an amino acid or an amino acid sequence that does not comprise at least one additional amino acid encoded by a nucleic acid sequence immediately downstream of the nucleic acid sequence in the genome of the subject that encodes the epitope, and/or a linker, compared to a polypeptide that comprises the same epitope but does not comprise the amino acid or the amino acid sequence that does not comprise at least one additional amino acid encoded by the nucleic acid sequence immediately downstream of the nucleic acid sequence in the genome of the subject that encodes the epitope, and/or a linker. In some embodiments, the epitope is released at a higher rate when the amino acid or the amino acid sequence is not of a peptide sequence of a protein expressed in the subject. In some embodiments, the epitope is released at a higher rate when the polypeptide comprises a linker compared to a polypeptide that comprises the same epitope but does not comprise a linker. In some embodiments, the epitope is released at a higher rate when the polypeptide comprises a linker that is cleavable by a protease compared to a polypeptide that comprises the same epitope but does not comprise a linker that is cleavable by a protease.

In some embodiments, the epitope is released at a higher rate when the polypeptide comprising an epitope and an amino acid or an amino acid sequence that comprises at least one additional amino acid that is encoded by the nucleic acid sequence immediately upstream or downstream of the nucleic acid sequence in the genome of the subject that encodes the epitope further comprises an amino acid or an amino acid sequence that is not encoded by a nucleic acid sequence immediately upstream or downstream of the nucleic acid sequence in the genome of the subject that encodes the epitope, and/or a linker, compared to a corresponding polypeptide that comprises the same epitope and an amino acid or an amino acid sequence that comprises at least one additional amino acid that is encoded by the nucleic acid sequence immediately upstream or downstream of the nucleic acid sequence in the genome of the subject that encodes the epitope, but does not comprise the amino acid or the amino acid sequence that is not encoded by the nucleic acid sequence immediately upstream or downstream of the nucleic acid sequence in the genome of the subject that encodes the epitope, and/or a linker.

In some embodiments, the polypeptide is cleaved at a higher rate when the polypeptide comprises an amino acid or an amino acid sequence that does not comprise at least one additional amino acid encoded by a nucleic acid sequence immediately upstream of the nucleic acid sequence in the genome of the subject that encodes the epitope, and/or a linker, compared to a corresponding polypeptide of the same length and epitope and the amino acid or the amino acid sequence is encoded by a nucleic acid sequence immediately upstream of the nucleic acid sequence in the genome of the subject that encodes the epitope. In some embodiments, the polypeptide is cleaved at a higher rate when the polypeptide comprises an amino acid or an amino acid sequence that does not comprise at least one additional amino acid encoded by a nucleic acid sequence immediately downstream of the nucleic acid sequence in the genome of the subject that encodes the epitope, and/or a linker, compared to a corresponding polypeptide of the same length and epitope and the amino acid or the amino acid sequence is encoded by a nucleic acid sequence immediately downstream of the nucleic acid sequence in the genome of the subject that encodes the epitope. In some embodiments, the polypeptide is cleaved at a higher rate when the amino acid or the amino acid sequence is not of a peptide sequence of a protein expressed in the subject. In some embodiments, the polypeptide is cleaved at a higher rate when the polypeptide comprises a linker compared to a polypeptide that comprises the same epitope but does not comprise a linker. In some embodiments, the polypeptide is cleaved at a higher rate when the polypeptide comprises a linker that is cleavable by a protease compared to a polypeptide that comprises the same epitope but does not comprise a linker that is cleavable by a protease.

In some embodiments, the polypeptide is cleaved at a higher rate when the polypeptide further comprises an amino acid or an amino acid sequence that does not comprise at least one additional amino acid encoded by a nucleic acid sequence immediately upstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker. In some embodiments, the polypeptide is cleaved at a higher rate when the polypeptide further comprises an amino acid or an amino acid sequence that does not comprise at least one additional amino acid encoded by a nucleic acid sequence immediately downstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker.

In some embodiments, the polypeptide is cleaved at a higher rate when the polypeptide comprises (i) an amino acid or an amino acid sequence that is encoded by a nucleic acid sequence immediately upstream or downstream of the nucleic acid sequence in the genome of the subject that encodes the epitope, and (ii) an amino acid or an amino acid sequence that is not encoded by a nucleic acid sequence immediately upstream or downstream of the nucleic acid sequence in the genome of the subject that encodes the epitope, and/or (iii) a linker, compared to a corresponding polypeptide of the same length and epitope and the amino acid or the amino acid sequence that is encoded by a nucleic acid sequence immediately upstream or downstream of the nucleic acid sequence in the genome of the subject that encodes the epitope.

In some embodiments, the polypeptide is cleaved at the linker region when the polypeptide is processed by an APC. In some embodiments, the polypeptide is cleaved at the linker region at a higher rate when the polypeptide further comprises an amino acid or an amino acid sequence that does not comprise at least one additional amino acid encoded by a nucleic acid sequence immediately upstream of the nucleic acid sequence in the genome of the subject that encodes the epitope, and a linker, compared to a corresponding polypeptide of the same length and epitope and an amino acid or an amino acid sequence is encoded by a nucleic acid sequence immediately upstream of the nucleic acid sequence in the genome of the subject that encodes the epitope. In some embodiments, the polypeptide is cleaved at the linker region at a higher rate when the polypeptide further comprises an amino acid or an amino acid sequence that does not comprise at least one additional amino acid encoded by a nucleic acid sequence immediately downstream of the nucleic acid sequence in the genome of the subject that encodes the epitope, and a linker, compared to a corresponding polypeptide of the same length and epitope and an amino acid or an amino acid sequence is encoded by a nucleic acid sequence immediately downstream of the nucleic acid sequence in the genome of the subject that encodes the epitope. In some embodiments, the polypeptide is cleaved at the linker region at a higher rate when the amino acid or the amino acid sequence is not of a peptide sequence of a protein expressed in the subject.

In some embodiments, epitope presentation by the APC is enhanced when the polypeptide is processed by an APC. In some embodiments, epitope presentation by the APC is enhanced when the polypeptide comprising the epitope further comprises an amino acid or an amino acid sequence that does not comprise at least one additional amino acid encoded by a nucleic acid sequence immediately upstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately upstream of the nucleic acid sequence in the genome of the subject that encodes the epitope. In some embodiments, epitope presentation by the APC is enhanced when the polypeptide comprising the epitope further comprises an amino acid or an amino acid sequence that does not comprise at least one additional amino acid encoded by a nucleic acid sequence immediately downstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately downstream of the nucleic acid sequence in the genome of the subject that encodes the epitope. In some embodiments, epitope presentation by the APC is enhanced when the amino acid or the amino acid sequence is not of a peptide sequence of a protein expressed in the subject. In some embodiments, epitope presentation the APC is enhanced when the polypeptide comprises a linker compared to a polypeptide that comprises the same epitope but does not comprise a linker. In some embodiments, epitope presentation by the APC is enhanced when the polypeptide comprises a linker that is cleavable by a protease compared to a polypeptide that comprises the same epitope but does not comprise a linker that is cleavable by a protease.

In some embodiments, epitope presentation by the APC is enhanced when the polypeptide further comprises an amino acid or an amino acid sequence that does not comprise at least one additional amino acid encoded by a nucleic acid sequence immediately upstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker. In some embodiments, epitope presentation by the APC is enhanced when the polypeptide further comprises an amino acid or an amino acid sequence that does not comprise at least one additional amino acid encoded by a nucleic acid sequence immediately downstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker.

In some embodiments, epitope presentation by the APC is enhanced when the polypeptide comprises (i) an amino acid or an amino acid sequence that is encoded by a nucleic acid sequence immediately upstream or downstream of the nucleic acid sequence in the genome of the subject that encodes the epitope, and (ii) an amino acid or an amino acid sequence that is not encoded by a nucleic acid sequence immediately upstream or downstream of the nucleic acid sequence in the genome of the subject that encodes the epitope, and/or (iii) a linker, compared to a corresponding polypeptide of the same length and epitope and the amino acid or the amino acid sequence that is encoded by a nucleic acid sequence immediately upstream or downstream of the nucleic acid sequence in the genome of the subject that encodes the epitope.

In some embodiments, immunogenicity is enhanced when the polypeptide is processed by an APC. In some embodiments, immunogenicity is enhanced when the polypeptide comprising the epitope further comprises an amino acid or an amino acid sequence that does not comprise at least one additional amino acid encoded by a nucleic acid sequence immediately upstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately upstream of the nucleic acid sequence in the genome of the subject that encodes the epitope. In some embodiments, immunogenicity is enhanced when the polypeptide comprising the epitope further comprises an amino acid or an amino acid sequence that does not comprise at least one additional amino acid encoded by a nucleic acid sequence immediately downstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately downstream of the nucleic acid sequence in the genome of the subject that encodes the epitope. In some embodiments, immunogenicity is enhanced when the amino acid or the amino acid sequence is not of a peptide sequence of a protein expressed in the subject. In some embodiments, immunogenicity is enhanced when the polypeptide comprises a linker compared to a polypeptide that comprises the same epitope but does not comprise a linker. In some embodiments, immunogenicity is enhanced when the polypeptide comprises a linker that is cleavable by a protease compared to a polypeptide that comprises the same epitope but does not comprise a linker that is cleavable by a protease.

In some embodiments, immunogenicity is enhanced when the polypeptide further comprises an amino acid or an amino acid sequence that does not comprise at least one additional amino acid encoded by a nucleic acid sequence immediately upstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker. In some embodiments, immunogenicity is enhanced when the polypeptide further comprises an amino acid or an amino acid sequence that does not comprise at least one additional amino acid encoded by a nucleic acid sequence immediately downstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker.

In some embodiments, immunogenicity is enhanced when the polypeptide comprises (i) an amino acid or an amino acid sequence that is encoded by a nucleic acid sequence immediately upstream or downstream of the nucleic acid sequence in the genome of the subject that encodes the epitope, and (ii) an amino acid or an amino acid sequence that is not encoded by a nucleic acid sequence immediately upstream or downstream of the nucleic acid sequence in the genome of the subject that encodes the epitope, and/or (iii) a linker, compared to a corresponding polypeptide of the same length and epitope and the amino acid or the amino acid sequence that is encoded by a nucleic acid sequence immediately upstream or downstream of the nucleic acid sequence in the genome of the subject that encodes the epitope.

In some embodiments, anti-tumor activity is enhanced when the polypeptide is processed by an APC. In some embodiments, anti-tumor activity by the APC is enhanced when the polypeptide comprising the epitope further comprises an amino acid or an amino acid sequence that does not comprise at least one additional amino acid encoded by a nucleic acid sequence immediately upstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately upstream of the nucleic acid sequence in the genome of the subject that encodes the epitope. In some embodiments, anti-tumor activity is enhanced when the polypeptide comprising the epitope further comprises an amino acid or an amino acid sequence that does not comprise at least one additional amino acid encoded by a nucleic acid sequence immediately downstream of the nucleic acid sequence in the genome of the subject that encodes the epitope, and/or a linker, compared to the corresponding polypeptide of the same length and epitope and an amino acid or an amino acid sequence is encoded by a nucleic acid sequence immediately downstream of the nucleic acid sequence in the genome of the subject that encodes the epitope. In some embodiments, anti-tumor activity is enhanced when the amino acid or the amino acid sequence is not of a peptide sequence of a protein expressed in the subject. In some embodiments, anti-tumor activity is enhanced when the polypeptide comprises a linker compared to a polypeptide that comprises the same epitope but does not comprise a linker. In some embodiments, anti-tumor activity is enhanced when the polypeptide comprises a linker that is cleavable by a protease compared to a polypeptide that comprises the same epitope but does not comprise a linker that is cleavable by a protease.

In some embodiments, anti-tumor activity is enhanced when the polypeptide further comprises an amino acid or an amino acid sequence that does not comprise at least one additional amino acid encoded by a nucleic acid sequence immediately upstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker. In some embodiments, anti-tumor activity is enhanced when the polypeptide further comprises an amino acid or an amino acid sequence that does not comprise at least one additional amino acid encoded by a nucleic acid sequence immediately downstream of the nucleic acid sequence in the genome of the subject that encodes the epitope compared to cleavage of a corresponding polypeptide of the same length that comprises an epitope sequence and an amino acid or an amino acid sequence contiguous with the epitope sequence that is encoded by a nucleic acid sequence and does not comprise a linker.

In some embodiments, anti-tumor activity is enhanced when the polypeptide comprises (i) an amino acid or an amino acid sequence that is encoded by a nucleic acid sequence immediately upstream or downstream of the nucleic acid sequence in the genome of the subject that encodes the epitope, and (ii) an amino acid or an amino acid sequence that is not encoded by a nucleic acid sequence immediately upstream or downstream of the nucleic acid sequence in the genome of the subject that encodes the epitope, and/or (iii) a linker, compared to a corresponding polypeptide of the same length and epitope and the amino acid or the amino acid sequence that is encoded by a nucleic acid sequence immediately upstream or downstream of the nucleic acid sequence in the genome of the subject that encodes the epitope.

In some embodiments, the APC presents the epitope to an immune cell when the polypeptide is processed by the APC. In some embodiments, the APC presents the epitope preferentially or specifically to the immune cell when the polypeptide is processed by the APC. In some embodiments, the APC presents the epitope to a phagocytic cell when the polypeptide is processed by the APC. In some embodiments, the APC presents the epitope preferentially or specifically to the phagocytic cell when the polypeptide is processed by the APC. In some embodiments, the APC presents the epitope to a dendritic cell, a macrophage, a mast cell, a neutrophil, or a monocyte when the polypeptide is processed by the APC. In some embodiments, the APC presents the epitope preferentially or specifically the dendritic cell, the macrophage, the mast cell, the neutrophil, or the monocyte.

In some embodiments, the polypeptide comprises an amino acid sequence selected from the group consisting of poly-Lys (polyK) and poly-Arg (polyR). In a preferred embodiment, the polypeptide comprises polyK sequence. In some embodiments, the polypeptide comprises a sequence selected from the group consisting of polyK-AA-AA and polyR-AA-AA, wherein each AA is an amino acid or analogue or derivative thereof. In a preferred embodiment, the polypeptide comprises polyK-AA-AA. In some embodiments, polyK comprises poly-L-Lys. In some embodiments, polyK comprises at least two contiguous lysine residues. In some embodiments, polyK comprises at least three contiguous lysine residues, for example, Lys-Lys-Lys. In a preferred embodiment, polyK comprises at least four contiguous lysine residues, for example, Lys-Lys-Lys-Lys, also known as K4. In some embodiments, polyK comprises at least five, at least six, at least seven, at least eight, at least nine, or at least 10 contiguous lysine residues. In some embodiments, polyR comprises poly-L-Arg. In some embodiments, polyR comprises at least two contiguous arginine residues. In some embodiments, polyR comprises at least three contiguous arginine residues, for example, Arg-Arg-Arg. In some embodiments, polyR comprises at least four, at least five, at least six, or at least seven contiguous arginine residues. In some embodiments, polyR comprises at least eight contiguous arginine residues, for example, Arg-Arg-Arg-Arg-Arg-Arg-Arg-Arg, also known as R8. In some embodiments, polyR comprises at least five, at least six, at least seven, at least eight, at least nine, or at least 10 contiguous arginine residues. In some embodiments, the lysine units in polyK and/or the arginine units in polyR each may have an (L) stereochemical configuration, a (D) stereochemical configuration, or any mixture of (L) and (D) stereochemical configuration.

In some embodiments, the polypeptide comprises a linker selected from the group consisting of a disulfide, p-aminobenzyloxycarbonyl (PABC), and AA-AA-PABC, wherein AA is an amino acid or analogue or derivative thereof. In some embodiments, AA-AA-PABC is selected from the group consisting of alanine-lysine-PABC (Ala-Lys-PABC), valine-citrulline-PABC (Val-Cit-PABC), and phenylalanine-lysine-PABC (Phe-Lys-PABC). In some embodiments, AA-AA-PABC is Ala-Lys-PABC. In some embodiments, AA-AA-PABC is Val-Cit-PABC. In some embodiments, AA-AA-PABC is Phe-Lys-PABC. In some embodiments, the valine and citrulline units in Val-Cit-PABC each have an (L) stereochemical configuration. In some embodiments, the phenyalanine and lysine units in Phe-Lys-PABC each have an (L) stereochemical configuration. In some embodiments, the valine and citrulline units in Val-Cit-PABC each have an (D) stereochemical configuration. In some embodiments, the phenyalanine and lysine units in Phe-Lys-PABC each have an (D) stereochemical configuration. In some embodiments, the valine and citrulline units in Val-Cit-PABC have a mixture of (L) and (D) stereochemical configuration. In some embodiments, the phenyalanine and lysine units in Phe-Lys-PABC have a mixture of (L) and (D) stereochemical configuration.

In some embodiments, the polypeptide comprises a linker that has the following structure:

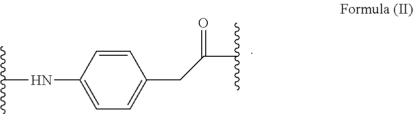 [SMILES: *Nc1ccc(CC(*)=O)cc1]

In some embodiments, the polypeptide comprises a linker that is

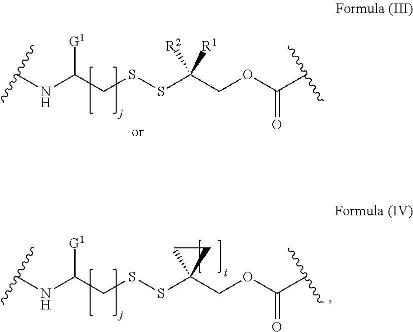

Wherein R₁ and R₂ is independently H or an (C₁-C₆) alkyl; j is 1 or 2; G₁ is H or COOH; and i is 1, 2, 3, 4, or 5.

In some embodiments, Aᵣ and/or Aₛ is Formula (III) or Formula (IV) wherein, R¹ and R² is independently H or an (C₁-C₆) alkyl; j is 1 or 2; G¹ is H or COOH; and i is 1, 2, 3, 4, or 5.

In some embodiments, the polypeptide comprises a linker that is Formula (III) or Formula (IV).

Disulfide linkers of Formula (IV) can be synthesized according to Zhang, Donglu, et al., ACS Med. Chem. Lett. 2016, 7, 988-993; and Pillow, Thomas H., et al., Chem. Sci., 2017, 8, 366-370. PABC-containing peptides can be synthesized according to Laurent Ducry (ed.), Antibody-Drug Conju gates, Methods in Molecular Biology, vol. 1045, DOI 10.1007/978-1-62703-541-5_5, Springer Science+Business Media, LLC 2013. In some embodiments, any resins made for solid phase peptide synthesis can be used.

### Antigen Processing Pathways

The polypeptide described herein can be processed by different pathways to release the epitope for epitope presentation. To generate optimal peptide antigens, two key processing events exist within the antigen processing and presentation pathway. Cytosolic proteins are primarily processed by proteasomes. The short peptides are then transported into the endoplasmic reticulum (ER) by transporter associated with antigen processing (TAP) for subsequent assembly with MHC class I molecules. Exogenous proteins are primarily presented by MHC class II molecules. Antigens are internalized by several pathways, including phagocytosis, macropinocytosis, and endocytosis, and eventually traffic to a mature or late endosomal compartment where they are processed and loaded on to MHC class II molecules. Cytoplasmic/nuclear antigens can also be trafficked into the endosomal network via autophagy for subsequent processing and presentation with MHC class II molecules.

The initial peptide proteolysis occurs within the cytosol of the cell and degrades larger protein fragments into smaller peptides by the proteasome or immunoproteasome. This processing event is often responsible for generating the final C-terminal residue of peptides that bind to class I MHC. The proteasome is a large proteolytic complex that contains multiple subunits, including two subunits, large multifunctional protease (LMP) 2 and LMP7. Proteins bound for degradation are targeted to the proteasome by covalent linkage with ubiquitin. LMP2 and LMP7 induce the proteolytic complex to generate peptides that bind to class I MHC I. The peptides generated in the cytosol are then transported through TAP into ER. As TAP preferentially transports peptides of 11-14 amino acids, peptides are often too long for stable class I MHC binding and require further processing upon entering the ER. This processing includes trimming of the N-terminal region of the antigenic peptides by endoplasmic reticulum aminopeptidase (ERAP) 1 and ERAP2. This process creates a pool of peptides that have with high affinity for class I MHC association.

In normal cellular environments, classical class II MHC molecules are only expressed on professional APCs such as dendritic cells (DCs) or macrophages. Exogenous or extracellular antigens that are internalized by phagocytosis, endocytosis, or pinocytosis are primarily presented on class II MHC to CD4+ T cells. A small subset of cytosolic antigens, however, is also expressed on class II MHC as a result of autophagy. In brief, endocytosed antigens are processed in a vesicular pathway consisting of progressively more acidic and proteolytically active compartments classically described as early endosomes (pH 6.0-pH 6.5), late endosomes or endolysosomes (pH 5.0-pH 6.0), and lysosomes (pH 4.5-pH 5.0). Antigens internalized by phagocytosis follow a similar path, terminating in phagolysosomes formed by the fusion of phagosomes and lysosomes. Lysosomes and phagolysosomes (pH 4.0-pH 4.5) contain a number of acid pH-optimum proteases generically called cathepsins. In highly degradative cells such as macrophages, successive cleavages by these enzymes result in very short peptides and free amino acids that are translocated into the cytosol to replenish tRNAs for new protein synthesis. In APCs which are less proteolytically active, larger intermediates form the dominant source of peptides for class II MHC binding and these peptides are usually consisting of 13-18 amino acids.

Both class I and class II MHC can access peptides processed from endogenous and exogenous antigens. For example, class II MHC bind peptides derived from endogenous membrane proteins that are degraded in the lysosome. Likewise, class I MHC can bind peptides derived from exogenous proteins internalized by endocytosis or phagocytosis, a phenomenon called cross-presentation. Specific subsets of DCs are particularly adept at mediating this process, which is critically important for the initiation of a primary response by naive CD8+ T cells.

In one aspect, provided herein is a method of cleaving a polypeptide, comprising contacting the polypeptide described herein to an APC. In some embodiments, the method can be performed in vivo. In some embodiments, the method can be performed in vitro.

In some embodiments, the polypeptide is ubiquitinated. In some embodiments, the polypeptide is ubiquitinated prior to cleavage. In some embodiments, the polypeptide is ubiquitinated prior to proteasome and/or immunoproteasome processing. In some embodiments, the polypeptide is ubiquitinated on a lysine residue. In some embodiments, the polypeptide is ubiquitinated on a lysine residue that is not on the epitope sequence. In some embodiments, the polypeptide is ubiquitinated on a lysine residue on polyK. In some embodiments, the polypeptide is ubiquitinated on the first lysine on polyK. In some embodiments, the polypeptide is ubiquitinated on the second lysine on polyK. In some embodiments, the polypeptide is ubiquitinated on the third lysine on polyK. In some embodiments, the polypeptide is ubiquitinated on the fourth lysine on polyK. In some embodiment, the polypeptide is ubiquitinated on the fifth, sixth, seventh, eighth, ninth, or tenth lysine on the polyK. In some embodiments, the polypeptide is ubiquitinated on at least one lysine residues. In some embodiments, the polypeptide is ubiquitinated on more than one lysine residues. In some embodiments, the polypeptide is ubiquitinated on more than one lysine residues on polyK. In some embodiments, the polypeptide is ubiquitinated on each lysine residue. In some embodiments, the polypeptide is ubiquitinated on each lysine residue on polyK. In some embodiments, the polypeptide is ubiquitinated on two lysine residues on polyK. In some embodiments, the polypeptide is ubiquitinated on three lysine residues on polyK. In some embodiments, the polypeptide is ubiquitinated on four lysine residues on polyK. In some embodiments, the polypeptide is ubiquitinated on five, six, seven, eight, nine, or ten lysine residues on polyK. In some embodiments, the polypeptide is sequentially ubiquitinated on each lysine residues on polyK. In some embodiments, the polypeptide is not sequentially ubiquitinated on each lysine residues on polyK.

In some embodiments, the polypeptide is ubiquitinated on a lysine residue on Ala-Lys-PABC. In some embodiments, the polypeptide is ubiquitinated on a lysine residue on Phe-Lys-PABC. In some embodiments, the polypeptide comprises polyK and AA-AA-PABC wherein each AA is an amino acid or analogue or derivative thereof. In some embodiments, the polypeptide is ubiquitinated on at least one lysine residue on polyK and AA-AA-PABC. In some embodiments, the polypeptide is ubiquitinated on one or more lysine residue on polyK and AA-AA-PABC. In some embodiments, the polypeptide is ubiquitinated on one or more lysine residue on polyK and Ala-Lys-PABC. In some embodiments, the polypeptide is ubiquitinated on one or more lysine residue on polyK and Phe-Lys-PABC.

In some embodiments, the polypeptide is internalized by an APC. In some embodiments, the polypeptide is internalized by an APC via endocytosis. In some embodiments, the polypeptide is internalized by an APC via phagocytosis. In some embodiments, the polypeptide is internalized by an APC via pinocytosis. In some embodiments, the polypeptide is cleaved in cytoplasm. In some embodiments, the polypeptide is cleaved in an endosome. In some embodiments, the polypeptide is cleaved in an endolysosome. In some embodiments, the polypeptide is cleaved in a lysosome. In some embodiments, the polypeptide is cleaved in an ER. In some embodiments, the polypeptide is cleaved by an aminopeptidase. In some embodiments, the aminopeptidase is an insulin-regulated aminopeptidase (IRAP). In some embodiments, the aminopeptidase is an endoplasmic reticulum aminopeptidase (ERAP). In some embodiments, the polypeptide is processed by a trypsin-like domain of a proteasome and/or an immunoproteasome. In some embodiments, the trypsin-like domain comprises trypsin-like activity. In some embodiments, the trypsin-like domain comprises chymotrypsin-like activity. In some embodiments, the trypsin-like activity comprises peptidylglutamyl-peptide hydrolase (PGPH) activity. In some embodiments, the polypeptide is cleaved by a protease. In some embodiments, the protease is a trypsin-like protease. In some embodiments, the protease is a chymotrypsin-like protease. In some embodiments, the protease is a peptidylglutamyl-peptide hydrolase (PGPH). In some embodiments, the protease is selected from the group consisting of asparagine peptide lyase, aspartic protease, cysteine protease, glutamic protease, metalloprotease, serine protease, and threonine protease. In a preferred embodiment, the protease is a cysteine protease. In some embodiments, the cysteine protease is selected from the group consisting of a Calpain, a Caspase, Cathepsin B, Cathepsin C, Cathepsin F, Cathepsin H, Cathepsin K, Cathepsin L1, Cathepsin L2, Cathepsin O, Cathepsin S, Cathepsin W, and Cathepsin Z. In some embodiments, the protease is Cathepsin B. In some embodiments, the protease is Cathepsin C. In some embodiments, the protease is Cathepsin F. In some embodiments, the protease is Cathepsin Z.

In some embodiments, the polypeptide is cleaved at a lysine residue. In some embodiments, the polypeptide is cleaved at a lysine residue on polyK. In some embodiments, the polypeptide is cleaved at the first lysine residue on polyK. In some embodiments, the polypeptide is cleaved at the second lysine residue on polyK. In some embodiments, the polypeptide is cleaved at the third lysine residue on polyK. In some embodiments, the polypeptide is cleaved at the fourth lysine residue on polyK. In some embodiments, the polypeptide is cleaved on the fifth, sixth, seventh, eighth, ninth, or tenth lysine residue on polyK. In some embodiments, the polypeptide is leaved at more than one lysine residues on polyK. In some embodiments, the polypeptide is leaved at each lysine residue on polyK. In some embodiments, the polypeptide is sequentially cleaved at each lysine residue on polyK. In some embodiments, the polypeptide is not sequentially cleaved at each lysine residue on polyK.

In some embodiments, the polypeptide is cleaved at AA-AA-PABC, wherein each AA is an amino acid or analogue or derivative thereof. In some embodiments, the polypeptide is cleaved at Ala-Lys-PABC. In some embodiments, the polypeptide is cleaved at the lysine residue in Ala-Lys-PABC. In some embodiments, the polypeptide is cleaved at Phe-Lys-PABC. In some embodiments, the polypeptide is cleaved at the lysine residue in Phe-Lys-PABC. In some embodiments, the polypeptide is cleaved at Val-Cit-PABC. In some embodiments, the polypeptide is cleaved at the citrulline (Cit) residue in Val-Cit-PABC. In some embodiments, the epitope is released when the polypeptide is cleaved.

One major weakness of peptide-based drugs limiting systemic therapeutic applications is proteolytic degradation of peptides. Peptides administered by the injection routes reach the bloodstream that contains proteases functioning in hemostasis, fibrinolysis, and tissue conversion, i.e., important processes in case of injury. Thus, it is important to stabilize the peptide against proteases present in blood, serum, or plasma. In one aspect, the polypeptide described herein is stable in plasma, blood, and/or serum. In some embodiments, the polypeptide is not cleaved before internalization by an APC in a subject. In some embodiments, the polypeptide is not cleaved before processing by an APC in a subject. In some embodiments, the polypeptide is not cleaved in blood in a subject before internalization by an APC. In some embodiments, the polypeptide is not cleaved in blood in a subject before processing by an APC. In some embodiments, the polypeptide is not cleaved by a protease in blood. In some embodiments, the polypeptide is not cleaved by plasmin. In some embodiments, the polypeptide is not cleaved by plasma kallikrein. In some embodiments, the polypeptide is not cleaved by tissue kallikrein. In some embodiments, the polypeptide is not cleaved by thrombin. In some embodiments, the polypeptide is not cleaved by a coagulation factor. In some embodiments, the polypeptide is not cleaved by coagulation factor XII. In some embodiments, the polypeptide is stable in human plasma. In some embodiments, the polypeptide is stable in human blood. In some embodiments, the polypeptide is stable in human serum.

In some embodiments, the polypeptide has a half-life of from 1 hour to 5 days in human plasma. In some embodiments, the polypeptide has a half-life about 1 hour to about 120 hours. In some embodiments, the polypeptide has a half-life about 1 hour to about 5 hours, about 1 hour to about 10 hours, about 1 hour to about 12 hours, about 1 hour to about 24 hours, about 1 hour to about 36 hours, about 1 hour to about 48 hours, about 1 hour to about 60 hours, about 1 hour to about 72 hours, about 1 hour to about 84 hours, about 1 hour to about 96 hours, about 1 hour to about 120 hours, about 5 hours to about 10 hours, about 5 hours to about 12 hours, about 5 hours to about 24 hours, about 5 hours to about 36 hours, about 5 hours to about 48 hours, about 5 hours to about 60 hours, about 5 hours to about 72 hours, about 5 hours to about 84 hours, about 5 hours to about 96 hours, about 5 hours to about 120 hours, about 10 hours to about 12 hours, about 10 hours to about 24 hours, about 10 hours to about 36 hours, about 10 hours to about 48 hours, about 10 hours to about 60 hours, about 10 hours to about 72 hours, about 10 hours to about 84 hours, about 10 hours to about 96 hours, about 10 hours to about 120 hours, about 12 hours to about 24 hours, about 12 hours to about 36 hours, about 12 hours to about 48 hours, about 12 hours to about 60 hours, about 12 hours to about 72 hours, about 12 hours to about 84 hours, about 12 hours to about 96 hours, about 12 hours to about 120 hours, about 24 hours to about 36 hours, about 24 hours to about 48 hours, about 24 hours to about 60 hours, about 24 hours to about 72 hours, about 24 hours to about 84 hours, about 24 hours to about 96 hours, about 24 hours to about 120 hours, about 36 hours to about 48 hours, about 36 hours to about 60 hours, about 36 hours to about 72 hours, about 36 hours to about 84 hours, about 36 hours to about 96 hours, about 36 hours to about 120 hours, about 48 hours to about 60 hours, about 48 hours to about 72 hours, about 48 hours to about 84 hours, about 48 hours to about 96 hours, about 48 hours to about 120 hours, about 60 hours to about 72 hours, about 60 hours to about 84 hours, about 60 hours to about 96 hours, about 60 hours to about 120 hours, about 72 hours to about 84 hours, about 72 hours to about 96 hours, about 72 hours to about 120 hours, about 84 hours to about 96 hours, about 84 hours to about 120 hours, or about 96 hours to about 120 hours. In some embodiments, the polypeptide has a half-life about 1 hour, about 5 hours, about 10 hours, about 12 hours, about 24 hours, about 36 hours, about 48 hours, about 60 hours, about 72 hours, about 84 hours, about 96 hours, or about 120 hours. In some embodiments, the polypeptide has a half-life at least about 1 hour, about 5 hours, about 10 hours, about 12 hours, about 24 hours, about 36 hours, about 48 hours, about 60 hours, about 72 hours, about 84 hours, or about 96 hours. In some embodiments, the polypeptide has a half-life at most about 5 hours, about 10 hours, about 12 hours, about 24 hours, about 36 hours, about 48 hours, about 60 hours, about 72 hours, about 84 hours, about 96 hours, or about 120 hours. 3. Neoantigens and Uses Thereof

One of the critical barriers to developing curative and tumor-specific immunotherapy is the identification and selection of highly specific and restricted tumor antigens to avoid autoimmunity. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized antigens, and producing neoantigens for use in a vaccine or immunogenic composition. These problems may be addressed by: identifying mutations in neoplasias/tumors which are present at the DNA level in tumor but not in matched germline samples from a high proportion of subjects having cancer; analyzing the identified mutations with one or more peptide-MHC binding prediction algorithms to generate a plurality of neoantigen T cell epitopes that are expressed within the neoplasia/tumor and that bind to a high proportion of patient HLA alleles; and synthesizing the plurality of neoantigenic peptides selected from the sets of all neoantigen peptides and predicted binding peptides for use in a cancer vaccine or immunogenic composition suitable for treating a high proportion of subjects having cancer.

For example, translating peptide sequencing information into a therapeutic vaccine may include prediction of mutated peptides that can bind to HLA molecules of a high proportion of individuals. Efficiently choosing which particular mutations to utilize as immunogen requires the ability to predict which mutated peptides would efficiently bind to a high proportion of patient's HLA alleles. Recently, neural network based learning approaches with validated binding and non-binding peptides have advanced the accuracy of prediction algorithms for the major HLA-A and -B alleles. However, even using advanced neural network-based algorithms to encode HLA-peptide binding rules, several factors limit the power to predict peptides presented on HLA alleles.

Another example of translating peptide sequencing information into a therapeutic vaccine may include formulating the drug as a multi-epitope vaccine of long peptides. Targeting as many mutated epitopes as practically possible takes advantage of the enormous capacity of the immune system, prevents the opportunity for immunological escape by down-modulation of an immune targeted gene product, and compensates for the known inaccuracy of epitope prediction approaches. Synthetic peptides provide a useful means to prepare multiple immunogens efficiently and to rapidly translate identification of mutant epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (non-mutated protein or viral vector antigens).

Yet another example of translating peptide sequencing information into a therapeutic vaccine may include a combination with a strong vaccine adjuvant. Effective vaccines may require a strong adjuvant to initiate an immune response. For example, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by dendritic cells (DCs). Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NYESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile.

### Peptides

In some aspects, the present disclosure provides isolated peptides that comprise a tumor-specific mutation. These peptides and polypeptides are referred to herein as “neoantigenic peptides” or “neoantigenic polypeptides.” The term “peptide” is used interchangeably with “mutant peptide”, “neoantigen peptide” and “neoantigenic peptide” in the present specification to designate a series of residues, typically L-amino acids, connected one to the other, typically by peptide bonds between the α-amino and carboxyl groups of adjacent amino acids. Similarly, the term “polypeptide” is used interchangeably with “mutant polypeptide,” “neoantigen polypeptide,” and “neoantigenic polypeptide” in the present specification to designate a series of residues, e.g., L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In some embodiments, genomic or exomic sequencing methods are used to identify tumor-specific mutations. Any suitable sequencing method can be used according to the present disclosure, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present disclosure mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome), or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the present disclosure e.g., those described in detail in WO 2012/159643.

In certain embodiments, the polypeptide described herein can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120, about 150, about 200, about 300, about 350, about 400, about 450, about 500, about 600, about 700, about 800, about 900, about 1,000, about 1,500, about 2,000, about 2,500, about 3,000, about 4,000, about 5,000, about 7,500, about 10,000 amino acids or greater amino acid residues, and any range derivable therein. In specific embodiments, a neoantigenic peptide molecule is equal to or less than 100 amino acids.

In some embodiments, the polypeptide can be from about 8 and about 50 amino acid residues in length, or from about 8 and about 30, from about 8 and about 20, from about 8 and about 18, from about 8 and about 15, or from about 8 and about 12 amino acid residues in length. In some embodiments, the peptides can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In some embodiments, the polypeptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the polypeptide can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or more amino acid residues in length. In some embodiments, the polypeptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the polypeptide can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, the polypeptide has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, at least 500, at least 1000, or at least 1500 amino acids.

In some embodiments, the polypeptide has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, at most 500, at most 1000, or at most 1500 amino acids.

In certain embodiments, the polypeptide described herein can comprise an epitope. In certain embodiments, the epitope can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120, about 150, about 200, about 300, about 350, about 400, about 450, about 500, about 600, about 700, about 800, about 900, about 1,000, about 1,500, about 2,000, about 2,500, about 3,000, about 4,000, about 5,000, about 7,500, about 10,000 amino acids or greater amino acid residues, and any range derivable therein.

In certain embodiments, the epitope can be from about 8 and about 50 amino acid residues in length, or from about 8 and about 30, from about 8 and about 20, from about 8 and about 18, from about 8 and about 15, or from about 8 and about 12 amino acid residues in length. In some embodiments, the peptides can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In certain embodiments, the epitope can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the epitope can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or more amino acid residues in length. In some embodiments, the epitope can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the epitope can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

A longer peptide can be designed in several ways. In some embodiments, when HLA-binding peptides are predicted or known, a longer peptide comprises (1) individual binding peptides with extensions of 2-5 amino acids toward the N- and C-terminus of each corresponding gene product; or (2) a concatenation of some or all of the binding peptides with extended sequences for each. In other embodiments, when sequencing reveals a long (>10 residues) neoepitope sequence present in the tumor (e.g., due to a frameshift, read-through or intron inclusion that leads to a novel peptide sequence), a longer peptide could consist of the entire stretch of novel tumor-specific amino acids as either a single longer peptide or several overlapping longer peptides. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long neoantigenic peptide.

In some embodiments, an immunogenic antigen, a neoantigen peptide, or an epitope thereof for MHC Class I is 12 amino acid residues or less in length and usually consists of between about 8 and about 12 amino acid residues. In some embodiments, an immunogenic antigen, a neoantigen peptide, or an epitope thereof for MHC Class I is about 8, about 9, about 10, about 11, or about 12 amino acid residues. In some embodiments, an immunogenic antigen, a neoantigen peptide, or an epitope thereof for MHC Class II is 25 amino acid residues or less in length and usually consists of between about 9 and about 25 amino acid residues. In some embodiments, an immunogenic antigen, a neoantigen peptide, or an epitope thereof for MHC Class II is about 15, about 16, about 17, about 18, about 19, about 20, about 21, about 22, about 23, about 24, or about 25 amino acid residues.

In some embodiments, an antigen, a neoantigen peptide, or an epitope binds an HLA protein (e.g., MHC class I HLA or MHC class II HLA). In specific embodiments, an antigen, a neoantigen peptide, or an epitope binds an HLA protein with greater affinity than a corresponding wild-type peptide. In specific embodiments, an antigen, a neoantigen peptide, or an epitope has an IC₅₀ or K_{D} of at least less than 5000 nM, at least less than 500 nM, at least less than 100 nM, at least less than 50 nM or less. In some embodiments, an antigen, a neoantigen peptide, or an epitope binds to MHC class I HLA. In some embodiments, an antigen, a neoantigen peptide, or an epitope binds to MHC class I HLA with an affinity of 0.1 nM to 2000 nM. In some embodiments, an antigen, a neoantigen peptide, or an epitope binds to MHC class I HLA with an affinity of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nM. In some embodiments, an antigen, a neoantigen peptide, or an epitope binds to MHC class II HLA. In some embodiments, an antigen, a neoantigen peptide, or an epitope binds to MHC class II HLA with an affinity of 0.1 nM to 2000 nM, 1 nM to 1000 nM, 10 nM to 500 nM, or less than 1000 nM. In some embodiments, an antigen, a neoantigen peptide, or an epitope binds to MHC class II HLA with an affinity of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nM.

In some embodiments, an antigen, a neoantigen peptide, or an epitope binds to MHC class I HLA with a stability of 10 minutes to 24 hours. In some embodiments, an antigen, a neoantigen peptide, or an epitope binds to MHC class I HLA with a stability of 10, 11, 12, 13, 14, 15, 16, 17, 18, 19, 20, 25, 30, 35, 40, 45, 50, 55, or 60 minutes. In some embodiments, an antigen, a neoantigen peptide, or an epitope binds to MHC class I HLA with a stability of 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, or 24 hours. In some embodiments, an antigen, a neoantigen peptide, or an epitope binds to MHC class II HLA with a stability of 10 minutes to 24 hours. In some embodiments, an antigen, a neoantigen peptide, or an epitope binds to MHC class II HLA with a stability of 10, 11, 12, 13, 14, 15, 16, 17, 18, 19, 20, 25, 30, 35, 40, 45, 50, 55, or 60 minutes. In some embodiments, an antigen, a neoantigen peptide, or an epitope binds to MHC class II HLA with a stability of 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, or 24 hours.

In some embodiments, the polypeptide can have a pI value of from about 0.5 to about 12, from about 2 to about 10, or from about 4 to about 8. In some embodiments, the peptides can have a pI value of at least 4.5, 5, 5.5, 6, 6.5, 7, 7.5, or more. In some embodiments, the polypeptide can have a pI value of at most 4.5, 5, 5.5, 6, 6.5, 7, 7.5, or less.

In some embodiments, the polypeptide described herein comprises an amino acid or an amino acid sequence of a peptide sequence that is not encoded by a nucleic acid sequence immediately upstream of the nucleic acid sequence in the genome of the subject that encodes the epitope. In some embodiments, the polypeptide described herein comprises an amino acid or an amino acid sequence of a peptide sequence that is not encoded by a nucleic acid sequence immediately downstream of the nucleic acid sequence in the genome of the subject that encodes the epitope. In some embodiments, the amino acid or the amino acid sequence comprises 0-1000, 1-900, 5-800, 10-700, 20-600, 30-500, 40-400, 50-300, 60-200, or 70-100 amino acid residues. In a preferred embodiment, the amino acid or the amino acid sequence comprises from 1 to 20 amino acid residues. In another preferred embodiments, the amino acid or the amino acid sequence comprises from 5 to 12 amino acid residues. In some embodiments, the amino acid or the amino acid sequence comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, at least 500, at least 1000, or at least 1500 amino acid residues. In some embodiments, the amino acid or the amino acid sequence comprises about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 40, about 50, about 60, about 70, about 80, about 90, about 100, about 150, about 200, about 250, about 300, about 350, about 400, about 450, about 500, about 1000, or about 1500 amino acid residues.

In one aspect, provided herein is a method of manufacturing a polypeptide, comprising linking an amino acid or an amino acid sequence and/or a linker to the N- and/or C-terminus of a sequence comprising an epitope sequence. In some embodiments, the polypeptide described herein can be in solution, lyophilized, or can be in crystal form. In some embodiments, the polypeptide described herein can be prepared synthetically, by recombinant DNA technology or chemical synthesis, or can be isolated from natural sources such as native tumors or pathogenic organisms. Epitopes or neoepitopes can be synthesized individually or joined directly or indirectly in the polypeptide. Although the polypeptide described herein can be substantially free of other naturally occurring host cell proteins and fragments thereof, in some embodiments, the polypeptide can be synthetically conjugated to be joined to native fragments or particles.

In some embodiments, the polypeptide described herein can be prepared in a wide variety of ways. In some embodiments, the polypeptide can be synthesized in solution or on a solid support according to conventional techniques. Various automatic synthesizers are commercially available and can be used according to known protocols. See, for example, Stewart & Young, Solid Phase Peptide Synthesis, 2d. Ed., Pierce Chemical Co., 1984. Further, individual polypeptide can be joined using chemical ligation to produce larger polypeptides that are still within the bounds of the present disclosure.

Alternatively, recombinant DNA technology can be employed wherein a nucleotide sequence which encodes the polypeptide or a part of the polypeptide inserted into an expression vector, transformed, or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1989). Thus, recombinant peptides, which comprise one or more neoantigenic peptides described herein, can be used to present the appropriate T cell epitope.

In some embodiments, the polypeptide comprises at least one mutant amino acid. In some embodiments, the at least one mutant amino acid is encoded by an insertion of one or more nucleotide in the nucleic acid sequence in the genome of the subject. In some embodiments, the at least one mutant amino acid is encoded by a deletion of one or more nucleotide in the nucleic acid sequence in the genome of the subject. In some embodiments, the at least one mutant amino acid is encoded by a frameshift in the nucleic acid sequence in the genome of the subject. A frameshift occurs when a mutation disrupts the normal phase of a gene's codon periodicity (also known as “reading frame”), resulting in the translation of a non-native protein sequence. It is possible for different mutations in a gene to achieve the same altered reading frame. In some embodiments, the at least one mutant amino acid is encoded by a neoORF in the nucleic acid sequence in the genome of the subject. In some embodiments, the at least one mutant amino acid is encoded by a point mutation in the nucleic acid sequence in the genome of the subject. In some embodiments, the at least one mutant amino acid is encoded by a gene with a mutation resulting in fusion polypeptide, in-frame deletion, insertion, expression of endogenous retroviral polypeptides, and tumor-specific overexpression of polypeptides. In some embodiments, the at least one mutant amino acid is encoded by a fusion of a first gene with a second gene in the genome of the subject. In some embodiments, the at least one mutant amino acid is encoded by an in-frame fusion of a first gene with a second gene in the genome of the subject. In some embodiments, the at least one mutant amino acid is encoded by a fusion of a first gene with an exon of a splice variant of the first gene in the genome of the subject. In some embodiments, the at least one mutant amino acid is encoded by a fusion of a first gene with a cryptic exon of the first gene in the genome of the subject.

In some aspects, the present disclosure provides a polypeptide comprising at least two polypeptide molecules. In some embodiments, the two or more of the at least two polypeptides or polypeptide molecules comprise an epitope. In some embodiments, the two or more of the at least two polypeptides or polypeptide molecules comprise the same epitope. In some embodiments, the two or more of the at least two polypeptides or polypeptide molecules comprise the same epitope of the same length. In some embodiments, the two or more of the at least two polypeptides or polypeptide molecules comprise an amino acid or an amino acid sequence that is of a peptide sequence that is not encoded by a nucleic acid sequence immediately upstream or downstream of the nucleic acid sequence in the genome of the subject that encodes the epitope. In some embodiments, the amino acid or amino acid sequence that is of a peptide sequence that is not encoded by a nucleic acid sequence immediately upstream of the nucleic acid sequence in the genome of the subject that encodes the epitope of the two or more of the at least two polypeptides or polypeptide molecules are the same. In some embodiments, the amino acid or amino acid sequence that is of a peptide sequence that is not encoded by a nucleic acid sequence immediately downstream of the nucleic acid sequence in the genome of the subject that encodes the epitope of the two or more of the at least two polypeptides or polypeptide molecules are the same.

In some embodiments, the two or more of the at least two polypeptides or polypeptide molecules comprise a linker. In some embodiments, the two or more of the at least two polypeptides or polypeptide molecules comprise a linker on the N- and/or C-terminus of the epitope. In some embodiments, the two or more of the at least two polypeptides or polypeptide molecules comprise different linkers. In some embodiments, a first polypeptide or polypeptide molecule of the at least two polypeptides or polypeptide molecules does not comprise a linker and a second polypeptide or polypeptide molecule of the at least two polypeptides or polypeptide molecules comprises a linker. In some embodiments, the first polypeptide or polypeptide molecule of the at least two polypeptides or polypeptide molecules does not comprise a linker on the N-terminus of the epitope and the second polypeptide or polypeptide molecule of the at least two polypeptides or polypeptide molecules comprises a linker on the N-terminus of the epitope. In some embodiments, the first polypeptide or polypeptide molecule of the at least two polypeptides or polypeptide molecules does not comprise a linker on the C-terminus of the epitope and the second polypeptide or polypeptide molecule of the at least two polypeptides or polypeptide molecules comprises a linker on the C-terminus of the epitope. In some embodiments, a first polypeptide or polypeptide molecule of the at least two polypeptides or polypeptide molecules comprises a linker and a second polypeptide or polypeptide molecule of the at least two polypeptides or polypeptide molecules does not comprise a linker. In some embodiments, the first polypeptide or polypeptide molecule of the at least two polypeptides or polypeptide molecules comprises a linker on the N-terminus of the epitope and the second polypeptide or polypeptide molecule of the at least two polypeptides or polypeptide molecules does not comprise a linker on the N-terminus of the epitope. In some embodiments, the first polypeptide or polypeptide molecule of the at least two polypeptides or polypeptide molecules comprises a linker on the C-terminus of the epitope and the second polypeptide or polypeptide molecule of the at least two polypeptides or polypeptide molecules does not comprise a linker on the C-terminus of the epitope.

Disulfide linkers can be synthesized using well known methods in the art. For example, disulfide linkers can be synthesized according to Zhang, Donglu, et al., ACS Med. Chem. Lett. 2016, 7, 988-993; and Pillow, Thomas H., et al., Chem. Sci., 2017, 8, 366-370. An example of disulfide linker synthesis and disulfide containing peptide synthesis is shown in Example 3 and 4. PABC-containing peptides can be synthesized using well known methods in the art. For example, PABC-containing peptides can be synthesized according to Laurent Ducry (ed.), Antibody-Drug Conju gates, Methods in Molecular Biology, vol. 1045, DOI 10.1007/978-1-62703-541-5_5, Springer Science+Business Media, LLC 2013. An example of PABC-containing peptide synthesis is shown in Example 5. In some embodiments, any resins made for solid phase peptide synthesis can be used.

In some embodiments, the polypeptide comprises at least 3, at least 4, at least 5, at least 6, at least 7, at least 8, at least 9, at least 10, or more polypeptides or polypeptide molecules. For example, the polypeptide can comprise 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 or more polypeptide or polypeptide molecules.

In some embodiments, a polypeptide comprising an antigen, a neoantigen peptide, or an epitope comprises a RAS epitope. In some embodiments, the peptide can be derived from a protein with a substitution mutation, e.g., the KRAS G12C, G12D, G12V, Q61H, or Q61L mutation, or the NRAS Q61K or Q61R mutation. The substitution may be positioned anywhere along the length of the peptide. For example, it can be located in the N-terminal third of the peptide, the central third of the peptide or the C-terminal third of the peptide. In another embodiment, the substituted residue is located 2-5 residues away from the N-terminal end or 2-5 residues away from the C-terminal end. The peptides can be similarly derived from tumor-specific insertion mutations where the peptide comprises one or more, or all of the inserted residues. In some embodiments, the epitope comprises a mutant RAS sequence that comprises at least 8 continuous amino acids of a mutant RAS protein comprising a mutation at G12, G13, or Q61 and the mutation at G12, G13, or Q61. In some embodiments, the at least 8 contiguous amino acids of the mutant RAS protein comprising the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation.

In some embodiments, the polypeptide comprising the RAS epitope further comprises an amino acid sequence. In some embodiments, the amino acid sequence is of a protein of cytomegalovirus (CMV), such as pp65. In some embodiments, the amino acid sequence is of a protein of human immunodeficiency virus (HIV). In some embodiments, the amino acid sequence is of a protein of MART-1. In some embodiments, the amino acid sequence of the protein of CMV, such as pp65, comprises 1, 2, 3, or more than 3 amino acid residues. In some embodiments, the amino acid sequence of the protein of CMV, such as pp65, comprises 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acid residues. In some embodiments, the amino acid sequence of the protein of HIV comprises 1, 2, 3, or more than 3 amino acid residues. In some embodiments, the amino acid sequence of the protein of HIV comprises 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acid residues. In some embodiments, the amino acid sequence of the protein of MART-1 comprises 1, 2, 3, or more than 3 amino acid residues. In some embodiments, the amino acid sequence of the protein of MART-1 comprises 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 amino acid residues.

In some embodiments, the RAS epitope binds to a protein encoded by an HLA allele. In some embodiments, the RAS epitope binds to a protein encoded by an HLA allele with an affinity of less than 10 μM, less than 9 μM, less than 8 μM, less than 7 μM, less than 6 μM, less than 5 μM, less than 4 μM, less than 3 μM, less than 2 μM, less than 1 μM, less than 950 nM, less than 900 nM, less than 850 nM, less than 800 nM, less than 750 nM, less than 600 nM, less than 550 nM, less than 500 nM, less than 450 nM, less than 400 nM, less than 350 nM, less than 300 nM, less than 250 nM, less than 200 nM, less than 150 nM, less than 100 nM, less than 90 nM, less than 80 nM, less than 70 nM, less than 60 nM, less than 50 nM, less than 40 nM, less than 30 nM, less than 20 nM, or less than 10 nM. In some embodiments, the RAS epitope binds to a protein encoded by an HLA allele with a stability of greater than 24 hours, greater than 23 hours, greater than 22 hours, greater than 21 hours, greater than 20 hours, greater than 19 hours, greater than 18 hours, greater than 17 hours, greater than 16 hours, greater than 15 hours, greater than 14 hours, greater than 13 hours, greater than 12 hours, greater than 11 hours, greater than 10 hours, greater than 9 hours, greater than 8 hours, greater than 7 hours, greater than 6 hours, greater than 5 hours, greater than 4 hours, greater than 3 hours, greater than 2 hours, greater than 1 hour, greater than 55 minutes, greater than 50 minutes, greater than 45 minutes, greater than 40 minutes, greater than 35 minutes, greater than 30 minutes, greater than 25 minutes, greater than 20 minutes, greater than 15 minutes, greater than 10 minutes, greater than 9 minutes, greater than 8 minutes, greater than 7 minutes, greater than 6 minutes, greater than 5 minutes, greater than 4 minutes, greater than 3 minutes, greater than 2 minutes, or greater than 1 minutes.

In some embodiments, the HLA allele is selected from the group consisting of an HLA-A02:01 allele, an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, an HLA-A74:01 allele, and/or an HLA-C08:02 allele and any combination thereof. In some embodiments, the HLA allele is an HLA-A02:01. In some embodiments, the HLA allele is an HLA-A03:01 allele. In some embodiments, the HLA allele is an HLA-A11:01 allele. In some embodiments, the HLA allele is an HLA-A03:02 allele. In some embodiments, the HLA allele is an HLA-A30:01 allele. In some embodiments, the HLA allele is an HLA-A31:01 allele. In some embodiments, the HLA allele is an HLA-A33:01 allele. In some embodiments, the HLA allele is an HLA-A33:03 allele. In some embodiments, the HLA allele is an HLA-A68:01 allele. In some embodiments, the HLA allele is an HLA-A74:01 allele. In some embodiments, the HLA allele is an HLA-C08:02.

In some aspects, the present disclosure provides a composition comprising a single polypeptide comprises the first peptide and the second peptide, or a single polynucleotide encodes the first peptide and the second peptide. In some embodiments, the composition provided herein comprises one or more additional peptides, wherein the one or more additional peptides comprise a third neoepitope. In some embodiments, the first peptide and the second peptide are encoded by a sequence transcribed from the same transcription start site. In some embodiments, the first peptide is encoded by a sequence transcribed from a first transcription start site and the second peptide is encoded by a sequence transcribed from a second transcription start site. In some embodiments, wherein the polypeptide has a length of at least 26, 27, 28, 29, 30, 40, 50, 60, 70, 80, 90, 100, 150, 200, 250, 300, 350, 400, 450, 500, 600, 700, 800, 900, 1,000, 1,500, 2,000, 2,500, 3,000, 4,000, 5,000, 7,500, or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence. In some embodiments, the polypeptide comprises a first sequence of at least 8 or 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence of at least 16 or 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the second peptide is longer than the first peptide. In some embodiments, the first peptide is longer than the second peptide. In some embodiments, the first peptide has a length of at least 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, 1,000, 1,500, 2,000, 2,500, 3,000, 4,000, 5,000, 7,500, or 10,000 amino acids. In some embodiments, the second peptide has a length of at least 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, 1,000, 1,500, 2,000, 2,500, 3,000, 4,000, 5,000, 7,500, or 10,000 amino acids. In some embodiments, the first peptide comprises a sequence of at least 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second peptide comprises a sequence of at least 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the first peptide, the second peptide, or both comprise at least one flanking sequence, wherein the at least one flanking sequence is upstream or downstream of the neoepitope. In some embodiments, the at least one flanking sequence has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, the at least one flanking sequence comprises a non-wild-type sequence. In some embodiments, the at least one flanking sequence is a N-terminus flanking sequence. In some embodiments, the at least one flanking sequence is a C-terminus flanking sequence. In some embodiments, the at least one flanking sequence of the first peptide has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the at least one flanking sequence of the second peptide. In some embodiments, the at least one flanking region of the first peptide is different from the at least one flanking region of the second peptide. In some embodiments, the at least one flanking residue comprises the mutation.

In some embodiments, a peptide comprises a neoepitope sequence comprising at least one mutant amino acid. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid. In some embodiments, a peptide comprises a neoepitope sequence derived from a protein comprising at least one mutant amino acid; at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid; and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid.

In some embodiments, a peptide comprises a neoepitope sequence derived from a protein comprising at least one mutant amino acid and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, a peptide comprises a neoepitope sequence derived from a protein comprising at least one mutant amino acid and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, a peptide comprises a neoepitope sequence derived from a protein comprising at least one mutant amino acid, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence.

In some embodiments, a peptide comprises a neoepitope sequence derived from a protein comprising at least one mutant amino acid and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, a peptide comprises a neoepitope sequence derived from a protein comprising at least one mutant amino acid and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, a peptide comprises a neoepitope sequence derived from a protein comprising at least one mutant amino acid, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence.

In some embodiments, the epitope is a TMPRSS2:ERG epitope. In some embodiments, the TMPRSS2:ERG epitope comprises an amino acid sequence of ALNSEALSV. In some embodiments, a polypeptide comprising RAS epitope comprises an amino acid sequence of GADGVGKSAL, GACGVGKSAL, GAVGVGKSAL, GADGVGKSA, GACGVGKSA, GAVGVGKSA, KLVVVGACGV, FLVVVGACGL, FMVVVGACGI, FLVVVGACGI, FMVVVGACGV, FLVVVGACGV, MLVVVGACGV, FMVVVGACGL, YLVVVGACGV, KMVVVGACGV, YMVVVGACGV, MMVVVGACGV, DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, or DILDTAGKE.

In some embodiments, a polypeptide comprising RAS epitope further comprises, such as on the N-terminus, an amino acid sequence of K, KK, KKK, KKKK, KKKKK, KKKKKKK, KKKKKKKK, KTEY, KTEYK, KTEYKL, KTEYKLV, KTEYKLVV, KTEYKLVVV, KKTEY, KKTEYK, KKTEYKL, KKTEYKLV, KKTEYKLVV, KKTEYKLVVV, KKKTEY, KKKTEYK, KKKTEYKL, KKKTEYKLV, KKKTEYKLVV, KKKTEYKLVVV, KKKKTEY, KKKKTEYK, KKKKTEYKL, KKKKTEYKLV, KKKKTEYKLVV, KKKKTEYKLVVV, IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, TEYK, TEYKL, TEYKLV, TEYKLVV, TEYKLVVV, WQAGILAR, HSYTTAE, PLTEEKIK, GALHFKPGSR, RRANKDATAE, KAFISHEEKR, TDLSSRFSKS, FDLGGGTFDV, CLLLHYSVSK, KKKKIIMKIRNA, or MTEYKLVVV.

In some embodiments, a polypeptide comprising RAS epitope further comprises, such as on the C-terminus, an amino acid sequence of K, KK, KKK, KKKK, KKKKK, KKKKKKK, KKKKKKKK, KKNKKDDI, KKNKKDDIKD, AGNDDDDDDDDDDDDDDDDDKKDKDDDDDD, AGNKKKKKKKNNNNNNNNNNNNNNNNNNNN, AGRDDDDDDDDDDDDDDDDDDDDDDDDDDD, SALTI, SALTIQL, GKSALTIQL, GKSALTI, SALTIK, SALTIQLK, GKSALTIQLK, GKSALTIK, SALTIKK, SALTIQLKK, GKSALTIQLKK, GKSALTIKK, SALTIKKK, SALTIQLKKK, GKSALTIQLKKK, GKSALTIKKK, SALTIKKKK, SALTIQLKKKK, GKSALTIQLKKKK, GKSALTI, KKKK, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

In some embodiments, a polypeptide comprising RAS epitope is selected from the group consisting of KTEYKLVVVGAVGVGKSALTIQL, KTEYKLVVVGADGVGKSALTIQL, KTEYKLVVVGARGVGKSALTIQL, KTEYKLVVVGACGVGKSALTIQL, KKTEYKLVVVGAVGVGKSALTIQL, KKTEYKLVVVGADGVGKSALTIQL, KKTEYKLVVVGARGVGKSALTIQL, KKTEYKLVVVGACGVGKSALTIQL, KKKTEYKLVVVGAVGVGKSALTIQL, KKKTEYKLVVVGADGVGKSALTIQL, KKKTEYKLVVVGARGVGKSALTIQL, KKKTEYKLVVVGACGVGKSALTIQL, KKKKTEYKLVVVGAVGVGKSALTIQL, KKKKTEYKLVVVGADGVGKSALTIQL, KKKKTEYKLVVVGARGVGKSALTIQL, KKKKTEYKLVVVGACGVGKSALTIQL, KKTEYKLVVVGAVGVGKSALTIQLKK, KKTEYKLVVVGADGVGKSALTIQLKK, KKTEYKLVVVGARGVGKSALTIQLKK, KKTEYKLVVVGACGVGKSALTIQLKK, TEYKLVVVGAVGVGKSALTIQLK, TEYKLVVVGADGVGKSALTIQLK, TEYKLVVVGARGVGKSALTIQLK, TEYKLVVVGACGVGKSALTIQLK, TEYKLVVVGAVGVGKSALTIQLKK, TEYKLVVVGADGVGKSALTIQLKK, TEYKLVVVGARGVGKSALTIQLKK, TEYKLVVVGACGVGKSALTIQLKK, TEYKLVVVGAVGVGKSALTIQLKKK, TEYKLVVVGADGVGKSALTIQLKKK, TEYKLVVVGARGVGKSALTIQLKKK, TEYKLVVVGACGVGKSALTIQLKKK, TEYKLVVVGAVGVGKSALTIQLKKKK, TEYKLVVVGADGVGKSALTIQLKKKK, and TEYKLVVVGARGVGKSALTIQLKKKK, TEYKLVVVGACGVGKSALTIQLKKKK. In some embodiments, a polypeptide comprising RAS epitope is selected from the group consisting of KKKTEYKLVVVGADGVGKSALTIQL, KKKTEYKLVVVGARGVGKSALTIQL, KKKKTEYKLVVVGAVGVGKSALTIQL, and KKKKTEYKLVVVGACGVGKSALTIQL. In some embodiments, a polypeptide comprising RAS epitope is KKKTEYKLVVVGADGVGKSALTIQL. In some embodiments, a polypeptide comprising RAS epitope is KKKTEYKLVVVGARGVGKSALTIQL. In some embodiments, a polypeptide comprising RAS epitope is KKKKTEYKLVVVGAVGVGKSALTIQL. In some embodiments, a polypeptide comprising RAS epitope is KKKKTEYKLVVVGACGVGKSALTIQL.

In some embodiments, a peptide comprising a KRAS G12C mutation comprises a sequence of MTEYKLVVVGACGVGKSALTIQLIQNHFVDEYDPTIEDSYRKQVVIDGETC LLDILDTAGQE. In some embodiments, a peptide comprising a KRAS G12 C mutation comprises a neoepitope sequence of KLVVVGACGV. In some embodiments, a peptide comprising a KRAS G12 C mutation comprises a neoepitope sequence of LVVVGACGV. In some embodiments, a peptide comprising a KRAS G12 C mutation comprises a neoepitope sequence of VVGACGVGK. In some embodiments, a peptide comprising a KRAS G12 C mutation comprises a neoepitope sequence of VVVGACGVGK.

In some embodiments, a peptide comprising a KRAS G12D mutation comprises a sequence of MTEYKLVVVGADGVGKSALTIQLIQNHFVDEYDPTIEDSYRKQVVIDGETCLLDILDTAGQE. In some embodiments, a peptide comprising a KRAS G12D mutation comprises a neoepitope sequence of VVGADGVGK. In some embodiments, a peptide comprising a KRAS G12D mutation comprises a neoepitope sequence of VVVGADGVGK. In some embodiments, a peptide comprising a KRAS G12D mutation comprises a neoepitope sequence of KLVVVGADGV. In some embodiments, a peptide comprising a KRAS G12D mutation comprises a neoepitope sequence of LVVVGADGV.

In some embodiments, a peptide comprising a KRAS G12V mutation comprises a sequence of MTEYKLVVVGAVGVGKSALTIQLIQNHFVDEYDPTIEDSYRKQVVIDGETCLLDILDTAGQE. In some embodiments, a peptide comprising a KRAS G12V mutation comprises a neoepitope sequence of KLVVVGAVGV. In some embodiments, a peptide comprising a KRAS G12V mutation comprises a neoepitope sequence of LVVVGAVGV. In some embodiments, a peptide comprising a KRAS G12V mutation comprises a neoepitope sequence of VVGAVGVGK. In some embodiments, a peptide comprising a KRAS G12V mutation comprises a neoepitope sequence of VVVGAVGVGK.

In some embodiments, a peptide comprising a KRAS Q61H mutation comprises a sequence of AGGVGKSALTIQLIQNHFVDEYDPTIEDSYRKQVVIDGETCLLDILDTAGHEEYSAMRDQYMRTGEG FLCVFAINNTKSFEDIHHYREQIKRVKDSEDVPM. In some embodiments, a peptide comprising a KRAS Q61H mutation comprises a neoepitope sequence of ILDTAGHEEY.

In some embodiments, a peptide comprising a KRAS Q61L mutation comprises a sequence of AGGVGKSALTIQLIQNHFVDEYDPTIEDSYRKQVVIDGETCLLDILDTAGLEEYSAMRDQYMRTGEG FLCVFAINNTKSFEDIHHYREQIKRVKDSEDVPM. In some embodiments, a peptide comprising a KRAS Q61L mutation comprises a neoepitope sequence of ILDTAGLEEY. In some embodiments, a peptide comprising a KRAS Q61L mutation comprises a neoepitope sequence of LLDILDTAGL.

In some embodiments, a peptide comprising a NRAS Q61K mutation comprises a sequence of AGGVGKSALTIQLIQNHFVDEYDPTIEDSYRKQVVIDGETCLLDILDTAGKEEYSAMRDQYMRTGEG FLCVFAINNSKSFADINLYREQIKRVKDSDDVPM. In some embodiments, a peptide comprising a NRAS Q61K mutation comprises a neoepitope sequence of ILDTAGKEEY.

In some embodiments, a peptide comprising a NRAS Q61R mutation comprises a sequence of AGGVGKSALTIQLIQNHFVDEYDPTIEDSYRKQVVIDGETCLLDILDTAGREEYSAMRDQYMRTGEG FLCVFAINNSKSFADINLYREQIKRVKDSDDVPM. In some embodiments, a peptide comprising a NRAS Q61R mutation comprises a neoepitope sequence of ILDTAGREEY.

In some embodiments, a peptide comprising a RAS Q61H mutation comprises a sequence of TCLLDILDTAGHEEYSAMRDQYM. In some embodiments, a peptide comprising a RAS Q61H mutation comprises a sequence provided in Table 1. In some embodiments, a peptide sequence provided in Table 1 binds to or is predicted to bind to a protein encoded by an HLA allele, which allele is provided in a corresponding column in Table 1 next to the peptide sequence.

TABLE 1
Peptide Sequences Comprising RAS Q61H
Mutation, Corresponding HLA Allele,
and Rank of Binding Potential

| | | Rank of |
|---|---|---|
| | | Binding |
| Peptide | Allele | Potential |
| ILDTAGHEEY | HLA-A36:01 | 1 |
| ILDTAGHEEY | HLA-A01:01 | 2 |
| DTAGHEEYSAM | HLA-A26:01 | 3 |
| DTAGHEEYSAM | HLA-A25:01 | 4 |
| GHEEYSAM | HLA-B15:09 | 4 |
| DTAGHEEY | HLA-A26:01 | 5 |
| ILDTAGHEE | HLA-C08:02 | 5 |
| AGHEEYSAM | HLA-C01:02 | 6 |
| AGHEEYSAM | HLA-B46:01 | 6 |
| DTAGHEEY | HLA-A25:01 | 6 |
| DTAGHEEY | HLA-A01:01 | 6 |
| DTAGHEEY | HLA-B18:01 | 7 |
| DTAGHEEY | HLA-A36:01 | 7 |
| ILDTAGHEE | HLA-C05:01 | 7 |
| ILDTAGHEE | HLA-A02:07 | 7 |
| ILDTAGHEEY | HLA-A29:02 | 7 |
| ILDTAGHEEY | HLA-C08:02 | 7 |
| HEEYSAMRD | HLA-B49:01 | 8 |
| TAGHEEYSA | HLA-B35:03 | 8 |
| DTAGHEEYS | HLA-A68:02 | 9 |
| DTAGHEEYSAMR | HLA-A68:01 | 9 |
| GHEEYSAM | HLA-B39:01 | 9 |
| ILDTAGHEE | HLA-A01:01 | 9 |
| LDTAGHEEY | HLA-B53:01 | 9 |
| HEEYSAMRD | HLA-B41:01 | 10 |
| ILDTAGHEE | HLA-A36:01 | 10 |
| DTAGHEEY | HLA-B58:01 | 11 |
| LLDILDTAGH | HLA-A01:01 | 12 |
| TAGHEEYSAM | HLA-B35:03 | 12 |
| LDTAGHEEY | HLA-B35:01 | 13 |
| DILDTAGHE | HLA-A26:01 | 14 |
| DTAGHEEY | HLA-C12:03 | 14 |
| ILDTAGHEEY | HLA-C05:01 | 14 |
| AGHEEYSAM | HLA-A30:02 | 15 |
| DILDTAGHEEY | HLA-A25:01 | 15 |
| DTAGHEEY | HLA-C02:02 | 15 |
| ILDTAGHEE | HLA-C04:01 | 15 |
| DILDTAGH | HLA-A26:01 | 16 |
| ILDTAGHEE | HLA-A02:01 | 16 |
| LDTAGHEEY | HLA-A29:02 | 16 |
| ILDTAGHE | HLA-A01:01 | 17 |
| LDTAGHEEY | HLA-B18:01 | 17 |
| AGHEEYSAM | HLA-C14:03 | 18 |
| DILDTAGHEEY | HLA-A29:02 | 18 |
| DTAGHEEYS | HLA-A26:01 | 18 |
| ILDTAGHEEY | HLA-B15:01 | 18 |
| DTAGHEEYSA | HLA-A68:02 | 19 |
| ILDTAGHE | HLA-CO5:Ol | 19 |
| ILDTAGHEEY | HLA-A02:07 | 19 |
| ILDTAGHEEY | HLA-A30:02 | 19 |
| LDTAGHEEY | HLA-A36:01 | 19 |
| AGHEEYSAM | HLA-C14:02 | 20 |
| AGHEEYSAM | HLA-B15:03 | 20 |
| LLDILDTAGH | HLA-A02:07 | 20 |

In some embodiments, a peptide comprising a RAS Q61R mutation comprises a sequence of TCLLDILDTAGREEYSAMRDQYM. In some embodiments, a peptide comprising a RAS Q61R mutation comprises a sequence provided in Table 2. In some embodiments, a peptide sequence provided in Table 2 binds to or is predicted to bind to a protein encoded by an HLA allele, which allele is provided in a corresponding column in Table 2 next to the peptide sequence.

TABLE 2
Peptide Sequences Comprising RAS Q61R Mutation, Corresponding HLA Allele, and Rank of
Binding Potential

| | | Rank of |
|---|---|---|
| | | Binding |
| Peptide | Allele | Potential |
| ILDTAGREEY | HLA-A36:01 | 1 |
| ILDTAGREEY | HLA-A01:01 | 2 |
| DTAGREEYSAM | HLA-A26:01 | 3 |
| DILDTAGR | HLA-A33:03 | 4 |
| DILDTAGR | HLA-A68:01 | 5 |
| DTAGREEY | HLA-A26:01 | 6 |
| DTAGREEYSAM | HLA-A25:01 | 6 |
| CLLDILDTAGR | HLA-A74:01 | 7 |
| DTAGREEY | HLA-A01:01 | 7 |
| REEYSAMRD | HLA-B41:01 | 7 |
| GREEYSAMR | HLA-B27:05 | 8 |
| ILDTAGREE | HLA-C08:02 | 8 |
| ILDTAGREEY | HLA-A29:02 | 8 |
| REEYSAMRD | HLA-B49:01 | 8 |
| AGREEYSAM | HLA-B46:01 | 9 |
| DTAGREEY | HLA-B18:01 | 9 |
| DTAGREEY | HLA-A25:01 | 9 |
| DTAGREEY | HLA-A36:01 | 9 |
| DILDTAGR | HLA-A74:01 | 10 |
| DILDTAGRE | HLA-A26:01 | 10 |
| ILDTAGREE | HLA-C05:01 | 10 |
| DILDTAGR | HLA-A26:01 | 11 |
| GREEYSAM | HLA-B39:01 | 11 |
| AGREEYSAM | HLA-B15:03 | 12 |
| GREEYSAM | HLA-C07:02 | 12 |
| ILDTAGREE | HLA-A01:01 | 12 |
| TAGREEYSA | HLA-B35:03 | 12 |
| ILDTAGREEY | HLA-A30:02 | 13 |
| DTAGREEYS | HLA-A68:02 | 14 |
| ILDTAGRE | HLA-A01:01 | 14 |
| CLLDILDTAGR | HLA-A31:01 | 15 |
| DTAGREEYSAMR | HLA-A68:01 | 15 |
| LLDILDTAGR | HLA-A01:01 | 15 |
| DTAGREEY | HLA-B58:01 | 16 |
| ILDTAGREEY | HLA-C08:02 | 16 |
| DILDTAGR | HLA-A31:01 | 17 |
| ILDTAGREE | HLA-C04:01 | 17 |
| ILDTAGREEY | HLA-A32:01 | 17 |
| LLDILDTAGR | HLA-A74:01 | 17 |
| TAGREEYSAM | HLA-B35:03 | 17 |
| DILDTAGREEY | HLA-A32:01 | 18 |
| ILDTAGRE | HLA-C05:01 | 18 |
| ILDTAGREE | HLA-A02:07 | 18 |
| REEYSAMRD | HLA-B40:01 | 18 |
| AGREEYSAM | HLA-B15:01 | 19 |
| AGREEYSAMR | HLA-A31:01 | 19 |
| ILDTAGRE | HLA-A36:01 | 19 |
| LDILDTAGR | HLA-A68:01 | 19 |
| LDTAGREEY | HLA-A29:02 | 19 |
| LDTAGREEY | HLA-B35:01 | 19 |
| REEYSAMRD | HLA-B45:01 | 19 |
| REEYSAMRDQY | HLA-A36:01 | 19 |
| DTAGREEY | HLA-C02:02 | 20 |

In some embodiments, a peptide comprising a RAS Q61K mutation comprises a sequence of TCLLDILDTAGKEEYSAMRDQYM. In some embodiments, a peptide comprising a RAS Q61K mutation comprises a sequence provided in Table 3. In some embodiments, a peptide sequence provided in Table 3 binds to or is predicted to bind to a protein encoded by an HLA allele, which allele is provided in a corresponding column in Table 3 next to the peptide sequence.

TABLE 3
Peptide Sequences Comprising RAS Q61K Mutation,
Corresponding HLA Allele, and Rank of
Binding Potential

| | | Rank of |
|---|---|---|
| | | Binding |
| Peptide | Allele | Potential |
| ILDTAGKEEY | HLA-A36:01 | 1 |
| ILDTAGKEEY | HLA-A01:01 | 2 |
| DTAGKEEYSAM | HLA-A26:01 | 3 |
| CLLDILDTAGK | HLA-A03:01 | 4 |
| DTAGKEEY | HLA-A01:01 | 5 |
| DTAGKEEY | HLA-A26:01 | 5 |
| DTAGKEEYSAM | HLA-A25:01 | 5 |
| AGKEEYSAM | HLA-B46:01 | 6 |
| DILDTAGKE | HLA-A26:01 | 7 |
| KEEYSAMRD | HLA-B41:01 | 7 |
| DTAGKEEY | HLA-B18:01 | 8 |
| GKEEYSAM | HLA-B15:03 | 8 |
| ILDTAGKEE | HLA-C08:02 | 8 |
| ILDTAGKEEY | HLA-A29:02 | 8 |
| DTAGKEEYS | HLA-A68:02 | 9 |
| LDTAGKEEY | HLA-B53:01 | 9 |
| TAGKEEYSA | HLA-B35:03 | 9 |
| DILDTAGK | HLA-A68:01 | 10 |
| DTAGKEEY | HLA-A36:01 | 10 |
| KEEYSAMRD | HLA-B49:01 | 10 |
| LDTAGKEEY | HLA-C07:01 | 10 |
| DTAGKEEYSAMR | HLA-A68:01 | 11 |
| ILDTAGKEE | HLA-C05:01 | 11 |
| ILDTAGKEEY | HLA-C08:02 | 11 |
| LLDILDTAGK | HLA-A01:01 | 12 |
| AGKEEYSAM | HLA-A30:02 | 13 |
| DTAGKEEY | HLA-A25:01 | 13 |
| DTAGKEEYS | HLA-A26:01 | 13 |
| ILDTAGKE | HLA-C05:01 | 13 |
| LDTAGKEEY | HLA-B35:01 | 13 |
| AGKEEYSAMR | HLA-A31:01 | 14 |
| DILDTAGK | HLA-A33:03 | 14 |
| ILDTAGKE | HLA-A01:01 | 14 |
| ILDTAGKEE | HLA-A01:01 | 14 |
| ILDTAGKEE | HLA-A02:07 | 14 |
| TAGKEEYSAM | HLA-B35:03 | 14 |
| AGKEEYSAM | HLA-B15:01 | 15 |
| ILDTAGKEEY | HLA-A30:02 | 15 |
| LDTAGKEEY | HLA-B46:01 | 15 |
| DTAGKEEY | HLA-B58:01 | 16 |
| ILDTAGKEEY | HLA-C05:0l | 17 |
| AGKEEYSAM | HLA-A30:01 | 18 |
| AGKEEYSAM | HLA-B15:03 | 18 |
| DTAGKEEY | HLA-C02:02 | 18 |
| LDTAGKEEY | HLA-A29:02 | 18 |

In some embodiments, a peptide comprising a RAS Q61L mutation comprises a sequence of TCLLDILDTAGLEEYSAMRDQYM. In some embodiments, a peptide comprising a RAS Q61L mutation comprises a sequence provided in Table 4. In some embodiments, a peptide sequence provided in Table 4 binds to or is predicted to bind to a protein encoded by an HLA allele, which allele is provided in a corresponding column in Table 4 next to the peptide sequence.

TABLE 4
Peptide Sequences Comprising RAS Q61L
Mutation, Corresponding HLA Allele, and
Rank of Binding Potential

| | | Rank of |
|---|---|---|
| | | Binding |
| Peptide | Allele | Potential |
| ILDTAGLEEY | HLA-A36:01 | 1 |
| ILDTAGLEEY | HLA-A01:01 | 2 |
| LLDILDTAGL | HLA-A02:07 | 3 |
| GLEEYSAMRDQY | HLA-A36:01 | 4 |
| DTAGLEEY | HLA-A25:01 | 5 |
| DTAGLEEY | HLA-A26:01 | 5 |
| DTAGLEEYSAM | HLA-A26:01 | 5 |
| DTAGLEEY | HLA-A01:01 | 6 |
| ILDTAGLEE | HLA-C08:02 | 6 |
| ILDTAGLEE | HLA-A01:01 | 6 |
| CLLDILDTAGL | HLA-A02:04 | 7 |
| ILDTAGLEE | HLA-A36:01 | 7 |
| LLDILDTAGL | HLA-A01:01 | 7 |
| DILDTAGL | HLA-B14:02 | 8 |
| DILDTAGLEEY | HLA-A25:01 | 8 |
| DTAGLEEYS | HLA-A68:02 | 8 |
| DTAGLEEYSAM | HLA-A25:01 | 8 |
| GLEEYSAMR | HLA-A74:01 | 8 |
| ILDTAGLE | HLA-A01:01 | 8 |
| DILDTAGLEEY | HLA-A26:01 | 9 |
| DTAGLEEY | HLA-A36:01 | 9 |
| ILDTAGLEEY | HLA-A29:02 | 9 |
| DILDTAGL | HLA-B08:01 | 10 |
| DTAGLEEY | HLA-B18:01 | 10 |
| ILDTAGLEE | HLA-A02:07 | 10 |
| LDTAGLEEY | HLA-B35:01 | 10 |
| CLLDILDTAGL | HLA-A02:01 | 11 |
| DTAGLEEY | HLA-C02:02 | 11 |
| ILDTAGLEE | HLA-C05:01 | 11 |
| ILDTAGLEEY | HLA-C08:02 | 11 |
| ILDTAGLEEY | HLA-A02:07 | 11 |
| LLDILDTAGL | HLA-C08:02 | 11 |
| DILDTAGL | HLA-A26:01 | 12 |
| LDTAGLEEY | HLA-B53:01 | 12 |
| DTAGLEEY | HLA-C03:02 | 13 |
| DTAGLEEY | HLA-B58:01 | 13 |
| ILDTAGLEEY | HLA-A30:02 | 13 |
| LLDILDTAGL | HLA-C05:01 | 13 |
| LLDILDTAGL | HLA-C04:01 | 13 |
| DTAGLEEYSAMR | HLA-A68:01 | 14 |
| ILDTAGLE | HLA-A36:01 | 15 |
| LLDILDTAGL | HLA-A02:01 | 15 |
| AGLEEYSAM | HLA-B15:03 | 16 |
| DTAGLEEYSA | HLA-A68:02 | 16 |
| GLEEYSAMRDQY | HLA-A01:01 | 16 |
| ILDTAGLE | HLA-C04:01 | 16 |
| ILDTAGLEEY | HLA-B15:01 | 16 |
| LDILDTAGL | HLA-B37:01 | 16 |
| AGLEEYSAM | HLA-A30:02 | 17 |
| AGLEEYSAM | HLA-B48:01 | 17 |
| AGLEEYSAMR | HLA-A31:01 | 17 |
| ILDTAGLEE | HLA-C04:01 | 17 |
| LDTAGLEEY | HLA-C03:02 | 17 |
| AGLEEYSAM | HLA-C14:02 | 18 |
| GLEEYSAMR | HLA-A31:01 | 18 |
| LEEYSAMRD | HLA-B4L01 | 18 |
| LLDILDTAGLE | HLA-A01:01 | 18 |
| AGLEEYSAM | HLA-C14:03 | 19 |
| LDILDTAGL | HLA-B40:02 | 19 |
| LDTAGLEEY | HLA-A29:02 | 19 |
| DILDTAGLE | HLA-A26:01 | 20 |
| DTAGLEEY | HLA-B15:01 | 20 |
| ILDTAGLEEY | HLA-A02:01 | 20 |
| LDTAGLEEY | HLA-A36:01 | 20 |
| LDTAGLEEY | HLA-B46:01 | 20 |
| DTAGLEEY | HLA-A68:02 | 21 |
| DTAGLEEY | HLA-C12:03 | 21 |
| ILDTAGLE | HLA-C05:01 | 21 |
| LDTAGLEEY | HLA-B18:01 | 21 |
| LEEYSAMRD | HLA-B49:01 | 21 |
| TAGLEEYSA | HLA-B54:01 | 21 |
| DILDTAGLEEY | HLA-A29:02 | 22 |
| GLEEYSAM | HLA-C05:01 | 22 |

In some embodiments, a peptide comprising a RAS G12A mutation comprises a sequence of MTEYKLVVVGAAGVGKSALTIQL. In some embodiments, a peptide comprising a RAS G12A mutation comprises a sequence provided in Table 5. In some embodiments, a peptide sequence provided in Table 5 binds to or is predicted to bind to a protein encoded by an HLA allele, which allele is provided in a corresponding column in Table 5 next to the peptide sequence.

TABLE 5
Peptide Sequences Comprising RAS G12A Mutation,
Corresponding HLA Allele, and Rank of
Binding Potential

| | | Rank of |
|---|---|---|
| | | Binding |
| Peptide | Allele | Potential |
| AAGVGKSAL | HLA-C03:04 | 1 |
| VVVGAAGVGK | HLA-A11:01 | 1 |
| VVGAAGVGK | HLA-A11:01 | 2 |
| TEYKLVVVGAA | HLA-B50:01 | 3 |
| VVGAAGVGK | HLA-A03:01 | 3 |
| VVVGAAGVGK | HLA-A68:01 | 3 |
| AAGVGKSAL | HLA-C08:02 | 4 |
| AAGVGKSAL | HLA-C08:01 | 4 |
| AAGVGKSAL | HLA-B46:01 | 4 |
| AAGVGKSAL | HLA-B81:01 | 5 |
| GAAGVGKSAL | HLA-B48:01 | 5 |
| LVVVGAAGV | HLA-A68:02 | 5 |
| AAGVGKSAL | HLA-C03:04 | 1 |
| VVVGAAGVGK | HLA-A11:0l | 1 |
| VVGAAGVGK | HLA-A11:01 | 2 |
| TEYKLVVVGAA | HLA-B50:01 | 3 |
| VVGAAGVGK | HLA-A03:01 | 3 |
| VVVGAAGVGK | HLA-A68:01 | 3 |
| AAGVGKSAL | HLA-C08:02 | 4 |
| AAGVGKSAL | HLA-C08:01 | 4 |
| AAGVGKSAL | HLA-B46:01 | 4 |
| AAGVGKSAL | HLA-B81:01 | 5 |
| AAGVGKSAL | HLA-C03:02 | 5 |
| AAGVGKSAL | HLA-C01:02 | 5 |
| GAAGVGKSAL | HLA-B48:01 | 5 |
| LVVVGAAGV | HLA-A68:02 | 5 |
| AAGVGKSAL | HLA-C03:03 | 6 |
| VVGAAGVGK | HLA-A68:01 | 6 |
| GAAGVGKSAL | HLA-B81:01 | 7 |
| VVVGAAGVGK | HLA-A03:01 | 7 |
| AAGVGKSAL | HLA-C05:0l | 8 |
| AAGVGKSAL | HLA-C12:03 | 8 |
| GAAGVGKSA | HLA-B46:01 | 8 |
| VVGAAGVGK | HLA-A30:01 | 8 |
| GAAGVGKSA | HLA-B55:01 | 9 |
| KLVVVGAAGV | HLA-A02:01 | 9 |
| AGVGKSAL | HLA-B08:01 | 10 |
| GAAGVGKSAL | HLA-C03:04 | 10 |
| AAGVGKSAL | HLA-C17:01 | 11 |
| GAAGVGKSAL | HLA-C03:03 | 11 |
| VVVGAAGV | HLA-A68:02 | 11 |
| YKLVVVGAA | HLA-B54:01 | 11 |
| AAGVGKSAL | HLA-B48:01 | 12 |
| AGVGKSAL | HLA-C03:04 | 12 |
| AGVGKSAL | HLA-C07:0l | 12 |
| VVVGAAGVGK | HLA-A30:01 | 12 |
| AAGVGKSA | HLA-B46:01 | 13 |
| KLVVVGAAGV | HLA-A02:07 | 13 |
| YKLVVVGAA | HLA-B50:01 | 13 |
| AAGVGKSAL | HLA-B07:02 | 14 |
| GAAGVGKSAL | HLA-A68:02 | 14 |
| VVGAAGVGK | HLA-A74:01 | 14 |
| AGVGKSAL | HLA-C08:0l | 15 |
| GAAGVGKSAL | HLA-C17:01 | 15 |
| GAAGVGKSAL | HLA-C08:0l | 16 |
| GAAGVGKSAL | HLA-B35:03 | 16 |
| AAGVGKSAL | HLA-C02:02 | 17 |
| AAGVGKSAL | HLA-B35:03 | 17 |
| AAGVGKSAL | HLA-C12:02 | 17 |
| AAGVGKSAL | HLA-C14:03 | 17 |
| GAAGVGKSA | HLA-B50:01 | 17 |
| AGVGKSAL | HLA-C03:02 | 18 |
| GAAGVGKSA | HLA-C03:04 | 18 |
| LVVVGAAGV | HLA-B55:01 | 18 |
| TEYKLVVVGAA | HLA-B4L01 | 18 |
| AGVGKSAL | HLA-C0L02 | 19 |
| GAAGVGKSA | HLA-B54:01 | 19 |
| GAAGVGKSAL | HLA-B07:02 | 19 |
| VGAAGVGKSA | HLA-B55:01 | 19 |
| AGVGKSAL | HLA-B48:01 | 20 |
| AGVGKSALTI | HLA-B49:01 | 20 |
| VVVGAAGV | HLA-B55:01 | 20 |

In some embodiments, a peptide comprising a RAS G12C mutation comprises a sequence of MTEYKLVVVGACGVGKSALTIQL. In some embodiments, a peptide comprising a RAS G12C mutation comprises a sequence provided in Table 6. In some embodiments, a peptide sequence provided in Table 6 binds to or is predicted to bind to a protein encoded by an HLA allele, which allele is provided in a corresponding column in Table 6 next to the peptide sequence.

TABLE 6
Peptide Sequences Comprising RAS G12C
Mutation, Corresponding HLA Allele,
and Rank of Binding Potential

| | | Rank of |
|---|---|---|
| | | Binding |
| Peptide | Allele | Potential |
| VVVGACGVGK | HLA-A11:01 | 1 |
| VVGACGVGK | HLA-A03:01 | 2 |
| VVGACGVGK | HLA-A11:01 | 3 |
| VVVGACGVGK | HLA-A68:01 | 4 |
| VVGACGVGK | HLA-A68:01 | 5 |
| VVVGACGVGK | HLA-A03:01 | 5 |
| VVGACGVGK | HLA-A30:01 | 6 |
| ACGVGKSAL | HLA-B81:01 | 7 |
| ACGVGKSAL | HLA-C01:02 | 7 |
| ACGVGKSAL | HLA-C14:03 | 8 |
| ACGVGKSAL | HLA-C03:04 | 9 |
| VVVGACGVGK | HLA-A30:01 | 9 |
| ACGVGKSAL | HLA-C14:02 | 10 |
| CGVGKSAL | HLA-B08:01 | 10 |
| KLVVVGACGV | HLA-A02:01 | 10 |
| ACGVGKSAL | HLA-B07:02 | 11 |
| GACGVGKSAL | HLA-B48:01 | 12 |
| GACGVGKSAL | HLA-C03:03 | 13 |
| ACGVGKSAL | HLA-B48:01 | 14 |
| ACGVGKSAL | HLA-B40:01 | 14 |
| YKLVVVGAC | HLA-B48:01 | 14 |
| YKLVVVGAC | HLA-B15:03 | 14 |
| GACGVGKSA | HLA-B46:01 | 15 |
| GACGVGKSAL | HLA-C03:04 | 15 |
| GACGVGKSAL | HLA-C01:02 | 15 |
| LVVVGACGV | HLA-A68:02 | 15 |
| CGVGKSAL | HLA-C03:04 | 16 |
| GACGVGKSAL | HLA-C08:02 | 16 |
| VVGACGVGK | HLA-A74:01 | 16 |

In some embodiments, a peptide comprising a RAS G12D mutation comprises a sequence of MTEYKLVVVGADGVGKSALTIQL. In some embodiments, a peptide comprising a RAS G12D mutation comprises a sequence provided in Table 7. In some embodiments, a peptide sequence provided in Table 7 binds to or is predicted to bind to a protein encoded by an HLA allele, which allele is provided in a corresponding column in Table 7 next to the peptide sequence

TABLE 7
Peptide Sequences Comprising RAS G12D
Mutation, Corresponding HLA Allele,
and Rank of Binding Potential

| | | Rank of |
|---|---|---|
| | | Binding |
| Peptide | Allele | Potential |
| GADGVGKSAL | HLA-C08:02 | 1 |
| GADGVGKSAL | HLA-C05:01 | 2 |
| VVVGADGVGK | HLA-A11:01 | 3 |
| DGVGKSAL | HLA-B14:02 | 4 |
| VVGADGVGK | HLA-A11:01 | 4 |
| VVGADGVGK | HLA-A03:01 | 5 |
| DGVGKSAL | HLA-B08:01 | 6 |
| VVVGADGVGK | HLA-A68:01 | 6 |
| GADGVGKSAL | HLA-C03:03 | 7 |
| VVGADGVGK | HLA-A30:01 | 7 |
| ADGVGKSAL | HLA-B37:01 | 8 |
| GADGVGKSAL | HLA-C08:01 | 8 |
| VVGADGVGK | HLA-A68:01 | 8 |
| GADGVGKSA | HLA-C08:02 | 9 |
| GADGVGKSAL | HLA-B35:03 | 9 |
| GADGVGKS | HLA-C05:01 | 10 |
| GADGVGKSA | HLA-C05:01 | 10 |
| ADGVGKSAL | HLA-C07:01 | 11 |
| VVVGADGVGK | HLA-A03:01 | 11 |
| ADGVGKSAL | HLA-B40:02 | 12 |
| ADGVGKSAL | HLA-B46:01 | 13 |
| GADGVGKSAL | HLA-C03:04 | 13 |
| ADGVGKSAL | HLA-B81:01 | 14 |
| GADGVGKSAL | HLA-C17:01 | 14 |
| VVVGADGVGK | HLA-A30:01 | 14 |
| GADGVGKSA | HLA-B35:03 | 15 |
| GADGVGKSA | HLA-B46:01 | 15 |
| GADGVGKSAL | HLA-B48:01 | 15 |
| KLVVVGADGV | HLA-A02:01 | 15 |
| LVVVGADGV | HLA-A68:02 | 15 |
| VGADGVGKSA | HLA-B55:01 | 15 |
| VVGADGVGK | HLA-A74:01 | 16 |
| GADGVGKSA | HLA-B53:01 | 17 |
| KLVVVGADGV | HLA-A02:07 | 17 |
| VGADGVGK | HLA-A68:01 | 17 |
| YKLVVVGAD | HLA-B48:01 | 17 |
| ADGVGKSAL | HLA-C14:03 | 18 |
| DGVGKSALTI | HLA-B51:01 | 18 |
| VGADGVGK | HLA-A11:01 | 18 |

In some embodiments, a peptide comprising a RAS G12R mutation comprises a sequence of MTEYKLVVVGARGVGKSALTIQL. In some embodiments, a peptide comprising a RAS G12R mutation comprises a sequence provided in Table 8. In some embodiments, a peptide sequence provided in Table 8 binds to or is predicted to bind to a protein encoded by an HLA allele, which allele is provided in a corresponding column in Table 8 next to the peptide sequence.

TABLE 8
Peptide Sequences Comprising RAS G12R Mutation,
Corresponding HLA Allele, and Rank of
Binding Potential

| | | Rank of |
|---|---|---|
| | | Binding |
| Peptide | Allele | Potential |
| VVGARGVGK | HLA-A11:01 | 1 |
| VVVGARGVGK | HLA-A68:01 | 1 |
| GARGVGKSA | HLA-B46:01 | 2 |
| ARGVGKSAL | HLA-B27:05 | 3 |
| GARGVGKSA | HLA-B55:0I | 3 |
| RGVGKSAL | HLA-C07:0I | 4 |
| VVGARGVGK | HLA-A30:01 | 5 |
| ARGVGKSAL | HLA-B38:01 | 6 |
| ARGVGKSAL | HLA-B14:02 | 6 |
| VVGARGVGK | HLA-A68:01 | 6 |
| VVVGARGVGK | HLA-A03:01 | 7 |
| GARGVGKSAL | HLA-B48:01 | 8 |
| RGVGKSAL | HLA-B48:01 | 8 |
| RGVGKSALT1 | HLA-A23:01 | 8 |
| ARGVGKSAL | HLA-C06:02 | 9 |
| GARGVGKSA | HLA-A30:01 | 9 |
| GARGVGKSAL | HLA-B81:01 | 9 |
| VVVGARGVGK | HLA-A30:01 | 9 |
| GARGVGKSAL | HLA-B07:02 | 10 |
| LVVVGARGV | HLA-C06:02 | 10 |
| RGVGKSAL | HLA-B81:01 | 10 |
| VVGARGVGK | HLA-A74:01 | 11 |
| KLVVVGARGV | HLA-A02:01 | 12 |
| LVVVGARGV | HLA-B35:01 | 12 |
| YKLVVVGAR | HLA-A33:03 | 12 |
| KLVVVGAR | HLA-A74:01 | 13 |
| KLVVVGARGV | HLA-B13:02 | 13 |
| RGVGKSAL | HLA-C01:02 | 13 |
| LVVVGARGV | HLA-A68:02 | 14 |
| VVVGARGV | HLA-B55:01 | 14 |
| ARGVGKSAL | HLA-B13:09 | 15 |
| ARGVGKSAL | HLA-C14:03 | 16 |
| GARGVGKSA | HLA-B34:01 | 16 |
| VVVGARGV | HLA-B52:01 | 16 |

In some embodiments, a peptide comprising a RAS G12S mutation comprises a sequence of MTEYKLVVVGASGVGKSALTIQL. In some embodiments, a peptide comprising a RAS G12S mutation comprises a sequence provided in Table 9. In some embodiments, a peptide sequence provided in Table 9 binds to or is predicted to bind to a protein encoded by an HLA allele, which allele is provided in a corresponding column in Table 9 next to the peptide sequence.

TABLE 9
Peptide Sequences Comprising RAS G12S
Mutation, Corresponding HLA Allele,
and Rank of Binding Potential

| | | Rank of |
|---|---|---|
| | | Binding |
| Peptide | Allele | Potential |
| VVVGASGVGK | HLA-A11:01 | 1 |
| VVGASGVGK | HLA-A11:01 | 2 |
| VVGASGVGK | HLA-A03:01 | 3 |
| VVVGASGVGK | HLA-A68:01 | 4 |
| ASGVGKSAL | HLA-C03:04 | 5 |
| ASGVGKSAL | HLA-B46:01 | 5 |
| VVGASGVGK | HLA-A68:01 | 6 |
| VVVGASGVGK | HLA-A03:01 | 6 |
| ASGVGKSAL | HLA-C01:02 | 7 |
| GASGVGKSAL | HLA-B48:01 | 7 |
| ASGVGKSAL | HLA-C07:01 | 8 |
| ASGVGKSAL | HLA-C08:02 | 9 |
| GASGVGKSAL | HLA-B81:01 | 9 |
| SGVGKSAL | HLA-B08:01 | 9 |
| ASGVGKSAL | HLA-C03:03 | 10 |
| ASGVGKSAL | HLA-C03:02 | 10 |
| SGVGKSAL | HLA-B14:02 | 10 |
| VVGASGVGK | HLA-A30:01 | 10 |
| ASGVGKSAL | HLA-C08:01 | 11 |
| VVVGASGVGK | HLA-A30:01 | 11 |
| GASGVGKSAL | HLA-B35:03 | 12 |
| SGVGKSAL | HLA-C07:01 | 12 |
| ASGVGKSAL | HLA-B81:01 | 13 |
| GASGVGKSA | HLA-B55:01 | 13 |
| GASGVGKSAL | HLA-C03:03 | 13 |
| KLVVVGASGV | HLA-A02:01 | 13 |
| LVVVGASGV | HLA-A68:02 | 13 |
| SGVGKSAL | HLA-C01:02 | 13 |
| ASGVGKSA | HLA-B46:01 | 14 |
| ASGVGKSAL | HLA-C15:02 | 14 |
| GASGVGKSAL | HLA-C08:01 | 15 |
| SGVGKSAL | HLA-C03:04 | 15 |
| ASGVGKSAL | HLA-C05:01 | 16 |
| GASGVGKSAL | HLA-C03:04 | 16 |
| VVGASGVGK | HLA-A74:01 | 16 |
| ASGVGKSAL | HLA-B48:01 | 17 |
| GASGVGKSAL | HLA-C01:02 | 17 |
| SGVGKSAL | HLA-C03:02 | 17 |
| SGVGKSALTI | HLA-A23:01 | 17 |
| VGASGVGKSA | HLA-B55:01 | 18 |
| ASGVGKSAL | HLA-C12:03 | 19 |
| ASGVGKSAL | HLA-B57:03 | 19 |
| KLVVVGASGV | HLA-A02:07 | 19 |
| SGVGKSAL | HLA-B81:01 | 19 |
| ASGVGKSAL | HLA-C17:01 | 20 |
| KLVVVGASG | HLA-A32:01 | 20 |

In some embodiments, a peptide comprising a RAS G12V mutation comprises a sequence of MTEYKLVVVGAVGVGKSALTIQL. In some embodiments, a peptide comprising a RAS G12V mutation comprises a sequence provided in Table 10. In some embodiments, a peptide sequence provided in Table 10 binds to or is predicted to bind to a protein encoded by an HLA allele, which allele is provided in a corresponding column in Table 10 next to the peptide sequence.

TABLE 10
Peptide Sequences Comprising RAS G12V
Mutation, Corresponding HLA Allele,
and Rank Binding Potential

| | | Rank of |
|---|---|---|
| | | Binding |
| Peptide | Allele | Potential |
| VVGAVGVGK | HLA-A03:01 | 1 |
| VVGAVGVGK | HLA-A11:01 | 2 |
| VVVGAVGVGK | HLA-A11:01 | 2 |
| VVVGAVGVGK | HLA-A68:01 | 3 |
| VVGAVGVGK | HLA-A68:01 | 4 |
| LVVVGAVGV | HLA-A68:02 | 5 |
| VVGAVGVGK | HLA-A30:01 | 5 |
| AVGVGKSAL | HLA-B81:01 | 6 |
| KLVVVGAVGV | HLA-A02:01 | 6 |
| AVGVGKSAL | HLA-B46:01 | 7 |
| GAVGVGKSAL | HLA-C03:03 | 7 |
| GAVGVGKSAL | HLA-B48:01 | 7 |
| VVVGAVGVGK | HLA-A03:01 | 7 |
| AVGVGKSAL | HLA-C03:04 | 8 |
| GAVGVGKSAL | HLA-C03:04 | 8 |
| KLVVVGAVGV | HLA-A02:07 | 9 |
| VGVGKSAL | HLA-B08:01 | 9 |
| VVVGAVGV | HLA-A68:02 | 9 |
| AVGVGKSAL | HLA-C08:02 | 10 |
| AVGVGKSAL | HLA-B07:02 | 10 |
| GAVGVGKSAL | HLA-B35:03 | 10 |
| AVGVGKSAL | HLA-C08:01 | 11 |
| AVGVGKSAL | HLA-C01:02 | 11 |
| GAVGVGKSA | HLA-B55:01 | 11 |
| GAVGVGKSAL | HLA-B81:01 | 11 |
| GAVGVGKSAL | HLA-C08:01 | 11 |
| KLVVVGAVGV | HLA-B13:02 | 11 |
| VGVGKSAL | HLA-C03:04 | 11 |
| AVGVGKSAL | HLA-A32:01 | 12 |
| GAVGVGKSA | HLA-B46:01 | 12 |
| VGVGKSAL | HLA-C03:02 | 12 |
| VGVGKSALTI | HLA-A23:01 | 12 |
| GAVGVGKSA | HLA-B54:01 | 13 |
| VGVGKSAL | HLA-C01:02 | 13 |
| AVGVGKSAL | HLA-B48:01 | 14 |
| AVGVGKSAL | HLA-C03:03 | 14 |
| AVGVGKSAL | HLA-B42:01 | 14 |
| LVVVGAVGV | HLA-B55:01 | 14 |
| VGVGKSAL | HLA-C08:01 | 14 |
| VVGAVGVGK | HLA-A74:01 | 14 |
| AVGVGKSAL | HLA-C05:01 | 15 |
| AVGVGKSAL | HLA-C03:02 | 15 |
| GAVGVGKSA | HLA-C03:04 | 15 |
| KLVVVGAVGV | HLA-A02:04 | 15 |
| LVVVGAVGV | HLA-A02:07 | 15 |
| VGVGKSAL | HLA-B14:02 | 15 |
| VVVGAVGVGK | HLA-A30:01 | 15 |
| VVGAVGVGK | HLA-B81:01 | 16 |
| VVVGAVGV | HLA-B55:01 | 16 |
| AVGVGKSAL | HLA-C14:03 | 17 |
| AVGVGKSAL | HLA-B15:01 | 17 |
| LVVVGAVGV | HLA-B54:01 | 17 |
| AVGVGKSA | HLA-B55:01 | 18 |
| AVGVGKSAL | HLA-C17:01 | 18 |
| GAVGVGKSA | HLA-B50:01 | 19 |
| GAVGVGKSAL | HLA-C17:01 | 19 |
| YKLVVVGAV | HLA-A02:04 | 19 |
| GAVGVGKSAL | HLA-B35:01 | 20 |
| VVGAVGVGK | HLA-A31:01 | 20 |
| YKLVVVGAV | HLA-B51:01 | 20 |

In some embodiments, a peptide comprising a RAS G13C mutation comprises a sequence of MTEYKLVVVGAGCVGKSALTIQL. In some embodiments, a peptide comprising a RAS G13C mutation comprises a sequence provided in Table 11. In some embodiments, a peptide sequence provided in Table 11 binds to or is predicted to bind to a protein encoded by an HLA allele, which allele is provided in a corresponding column in Table 11 next to the peptide sequence.

TABLE 11
Peptide Sequences Comprising RAS G13C
Mutation, Corresponding HLA Allele,
and Rank of Binding Potential

| | | Rank of |
|---|---|---|
| | | Binding |
| Peptide | Allele | Potential |
| VVVGAGCVGK | HLA-A11:01 | 1 |
| VVGAGCVGK | HLA-A11:01 | 2 |
| AGCVGKSAL | HLA-C01:02 | 3 |
| VVGAGCVGK | HLA-A03:01 | 4 |
| VVVGAGCVGK | HLA-A68:01 | 4 |
| CVGKSALTI | HLA-B13:02 | 5 |
| VVGAGCVGK | HLA-A68:01 | 5 |
| VVGAGCVGK | HLA-A30:01 | 6 |
| AGCVGKSAL | HLA-B48:01 | 7 |
| AGCVGKSAL | HLA-C03:04 | 8 |
| GCVGKSALTI | HLA-B49:01 | 8 |
| AGCVGKSAL | HLA-C08:02 | 9 |
| VVVGAGCVGK | HLA-A03:01 | 9 |
| KLVVVGAGC | HLA-A30:02 | 10 |
| GCVGKSAL | HLA-C07:01 | 11 |
| VVGAGCVGK | HLA-A74:01 | 12 |
| AGCVGKSAL | HLA-C14:03 | 13 |
| KLVVVGAGC | HLA-B15:01 | 14 |

In some embodiments, a peptide comprising a RAS G13D mutation comprises a sequence of MTEYKLVVVGAGDVGKSALTIQL. In some embodiments, a peptide comprising a RAS G13D mutation comprises a sequence provided in Table 12. In some embodiments, a peptide sequence provided in Table 12 binds to or is predicted to bind to a protein encoded by an HLA allele, which allele is provided in a corresponding column in Table 12 next to the peptide sequence.

TABLE 12
Peptide Sequences Comprising RAS G13D Mutation, Corresponding HLA Allele,
and Rank of Binding Potential

| | | Rank of |
|---|---|---|
| | | Binding |
| Peptide | Allele | Potential |
| AGDVGKSAL | HLA-C08:02 | 1 |
| AGDVGKSAL | HLA-C05:01 | 2 |
| VVGAGDVGK | HLA-A11:01 | 3 |
| VVVGAGDVGK | HLA-A11:01 | 3 |
| VVVGAGDVGK | HLA-A68:01 | 4 |
| GAGDVGKSA | HLA-B46:01 | 5 |
| GAGDVGKSAL | HLA-B48:01 | 5 |
| VVGAGDVGK | HLA-A68:01 | 5 |
| VVGAGDVGK | HLA-A03:01 | 5 |
| AGDVGKSAL | HLA-C03:04 | 6 |
| AGDVGKSAL | HLA-C04:01 | 6 |
| AGDVGKSAL | HLA-C01:02 | 6 |
| DVGKSALTI | HLA-B13:02 | 6 |
| DVGKSALTI | HLA-A25:01 | 6 |
| GDVGKSAL | HLA-C07:01 | 6 |
| GDVGKSAL | HLA-B40:02 | 7 |
| GDVGKSAL | HLA-B37:01 | 8 |
| AGDVGKSAL | HLA-B48:01 | 9 |
| DVGKSALTI | HLA-B51:01 | 10 |
| VVGAGDVGK | HLA-A30:01 | 10 |
| GAGDVGKSAL | HLA-C08:01 | 11 |
| GAGDVGKSAL | HLA-B81:01 | 11 |
| AGDVGKSAL | HLA-C08:01 | 12 |
| GAGDVGKSAL | HLA-C03:04 | 12 |
| DVGKSALTI | HLA-B53:01 | 13 |
| AGDVGKSAL | HLA-B07:02 | 14 |
| AGDVGKSAL | HLA-B46:01 | 14 |
| DVGKSALTI | HLA-A26:01 | 14 |
| VVGAGDVGK | HLA-A74:01 | 14 |
| GAGDVGKSA | HLA-B54:01 | 15 |
| DVGKSALTI | HLA-B38:01 | 16 |
| GAGDVGKSAL | HLA-C03:03 | 16 |
| VVVGAGDVGK | HLA-A03:01 | 16 |

In some embodiments, the polypeptide described herein does not comprise a RAS epitope. In some embodiments, the epitope is not a RAS epitope. In some embodiments, the polypeptide does not comprise KKKKKPKRDGYMFLKAESKIMFAT, KKKKYMFLKAESKIMFATLQRSS, KKKKKAESKIMFATLQRSSLWCL, KKKKKIMFATLQRSSLWCLCSNH, or KKKKMFATLQRSSLWCLCSNH.

In some embodiments, a polypeptide comprising an antigen, a neoantigen peptide, or an epitope comprises a GATA3 epitope. In some embodiments, the GATA3 epitope comprises an amino acid sequence of MLTGPPARV, SMLTGPPARV, VLPEPHLAL, KPKRDGYMF, KPKRDGYMFL, ESKIMFATL, KRDGYMFL, PAVPFDLHF, AESKIMFATL, FATLQRSSL, ARVPAVPFD, IMKPKRDGY, DGYMFLKA, MFLKAESKIMF, LTGPPARV, ARVPAVPF, SMLTGPPAR, RVPAVPFDL, or LTGPPARVP.

### Peptide Modifications

In some embodiments, the present disclosure includes modified peptides. A modification can include a covalent chemical modification that does not alter the primary amino acid sequence of the antigenic peptide itself. Modifications can produce peptides with desired properties, for example, prolonging the in vivo half-life, increasing the stability, reducing the clearance, altering the immunogenicity or allergenicity, enabling the raising of particular antibodies, cellular targeting, antigen uptake, antigen processing, HLA affinity, HLA stability, or antigen presentation. In some embodiments, a peptide may comprise one or more sequences that enhance processing and presentation of epitopes by APCs, for example, for generation of an immune response.

In some embodiments, the polypeptide may be modified to provide desired attributes. For instance, the ability of the peptides to induce cytotoxic T lymphocyte (CTL) activity can be enhanced by linkage to a sequence which contains at least one epitope that is capable of inducing a T helper cell response. In some embodiments, immunogenic peptides/T helper conjugates are linked by a spacer molecule. In some embodiments, a spacer comprises relatively small, neutral molecules, such as amino acids or amino acid mimetics, which are substantially uncharged under physiological conditions. Spacers can be selected from, e.g., Ala, Gly, or other neutral spacers of nonpolar amino acids or neutral polar amino acids. It will be understood that the optionally present spacer need not be comprised of the same residues and thus may be a hetero- or homo-oligomer. The neoantigenic peptide may be linked to the T helper peptide either directly or via a spacer either at the amino or carboxy terminus of the peptide. The amino terminus of either the neoantigenic peptide or the T helper peptide may be acylated. Examples of T helper peptides include tetanus toxoid residues 830-843, influenza residues 307-319, and malaria circumsporozoite residues 382-398 and residues 378-389.

The peptide sequences of the present disclosure may optionally be altered through changes at the DNA level, particularly by mutating the DNA encoding the peptide at preselected bases such that codons are generated that will translate into the desired amino acids.

In some embodiments, the peptide described herein can contain substitutions to modify a physical property (e.g., stability or solubility) of the resulting peptide. For example, the peptides can be modified by the substitution of a cysteine (C) with α-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and cross-binding capability in certain instances. Substitution of cysteine with α-amino butyric acid can occur at any residue of a neoantigenic peptide, e.g., at either anchor or non-anchor positions of an epitope or analog within a peptide, or at other positions of a peptide.

The peptide may also be modified by extending or decreasing the compound's amino acid sequence, e.g., by the addition or deletion of amino acids. The peptides or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

In some embodiments, the peptide may be modified using a series of peptides with single amino acid substitutions to determine the effect of electrostatic charge, hydrophobicity, etc. on HLA binding. For instance, a series of positively charged (e.g., Lys or Arg) or negatively charged (e.g., Glu) amino acid substitutions may be made along the length of the peptide revealing different patterns of sensitivity towards various HLA molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an HLA molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding. Amino acid substitutions are typically of single residues. Substitutions, deletions, insertions, or any combination thereof may be combined to arrive at a final peptide.

In some embodiments, the peptide described herein can comprise amino acid mimetics or unnatural amino acid residues, e.g., D- or L-naphylalanine; D- or L-phenylglycine; D- or L-2-thieneylalanine; D- or L-1,-2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-ρ-fluorophenylalanine; D- or L-ρ-biphenyl-phenylalanine; D- or L-ρ-methoxybiphenylphenylalanine; D- or L-2-indole(allyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides that have various amino acid mimetics or unnatural amino acid residues may have increased stability in vivo. Such peptides may also have improved shelf-life or manufacturing properties.

In some embodiments, a peptide described herein can be modified by terminal-NH₂ acylation, e.g., by alkanoyl (C₁-C₂₀) orthioglycolyl acetylation, terminal-carboxyl amidation, e.g., ammonia, methylamine, etc. In some embodiments these modifications can provide sites for linking to a support or other molecule. In some embodiments, the peptide described herein can contain modifications such as but not limited to glycosylation, side chain oxidation, biotinylation, phosphorylation, addition of a surface active material, e.g., a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, a peptide described herein can comprise carriers such as those well known in the art, e.g., thyroglobulin, albumins such as human serum albumin, tetanus toxoid, polyamino acid residues such as poly L-lysine and poly L-glutamic acid, influenza virus proteins, hepatitis B virus core protein, and the like.

The peptides can be further modified to contain additional chemical moieties not normally part of a protein. Those derivatized moieties can improve the solubility, the biological half-life, absorption of the protein, or binding affinity. The moieties can also reduce or eliminate any desirable side effects of the peptides and the like. An overview for those moieties can be found in Remington's Pharmaceutical Sciences, 20th ed., Mack Publishing Co., Easton, Pa. (2000). For example, neoantigenic peptides having the desired activity may be modified as necessary to provide certain desired attributes, e.g., improved pharmacological characteristics, while increasing or at least retaining substantially all of the biological activity of the unmodified peptide to bind the desired HLA molecule and activate the appropriate T cell. For instance, the peptide may be subject to various changes, such as substitutions, either conservative or non-conservative, where such changes might provide for certain advantages in their use, such as improved HLA binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

In some embodiments, the peptide described herein may be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Changes to the peptide that may include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

Glycosylation can affect the physical properties of proteins and can also be important in protein stability, secretion, and subcellular localization. Proper glycosylation can be important for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. Addition of glycosylation sites can be accomplished by altering the amino acid sequence. The alteration to the peptide or protein may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. Embodiments of the present disclosure comprise the generation and use of N-glycosylation variants. Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Additional suitable components and molecules for conjugation include, for example, molecules for targeting to the lymphatic system, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen; or any combination of the foregoing.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. In some embodiments, fusion of albumin to the peptide or protein of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Furthermore, albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin (see, e.g., WO2011/051489). Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics.

Additional candidate components and molecules for conjugation include those suitable for isolation or purification. Non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography may be used to separate conjugates by charge difference, which effectively separates conjugates into their various molecular weights. The content of the fractions obtained by cation exchange chromatography may be identified by molecular weight using conventional methods, for example, mass spectroscopy, SDS-PAGE, or other known methods for separating molecular entities by molecular weight.

In some embodiments, the amino- or carboxyl-terminus of the peptide or protein sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamics properties of the biopharmaceutical as compared to traditional Fc-fusion conjugates.

The present disclosure contemplates the use of other modifications, currently known or under development, of the peptides to improve one or more properties. One such method for prolonging the circulation half-life, increasing the stability, reducing the clearance, or altering the immunogenicity or allergenicity of the peptide of the present disclosure involves modification of the peptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides described herein is conveniently determined using a 25% human serum (v/v) assay. The protocol is as follows: pooled human serum (Type AB, non-heat inactivated) is dilapidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

Issues associated with short plasma half-life or susceptibility to protease degradation may be overcome by various modifications, including conjugating or linking the peptide or protein sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide or protein sequence are generally soluble in water at room temperature, and have the general formula R—(O—CH₂—CH₂)ₙ—O—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. The present disclosure also contemplates compositions of conjugates wherein the PEGs have different n values and thus the various different PEGs are present in specific ratios. For example, some compositions comprise a mixture of conjugates where n=1, 2, 3, and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to the peptide or protein of the present disclosure via a terminal reactive group (a “spacer”). The spacer is, for example, a terminal reactive group which mediates a bond between the free amino or carboxyl groups of one or more of the polypeptide sequences and PEG. The PEG having the spacer which may be bound to the free amino group includes N-hydroxysuccinylimide PEG which may be prepared by activating succinic acid ester of PEG with N-hydroxysuccinylimide. Another activated PEG which may be bound to a free amino group is 2,4-bis(O-methoxypolyethyleneglycol)-6-chloro-s-triazine which may be prepared by reacting PEG monomethyl ether with cyanuric chloride. The activated PEG which is bound to the free carboxyl group includes polyoxyethylenediamine.

Conjugation of one or more of the peptide or protein sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to peptide/protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

### Neoepitopes

A neoepitope comprises a neoantigenic determinant part of a neoantigenic peptide or neoantigenic polypeptide that is recognized by immune system. A neoepitope refers to an epitope that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” is used interchangeably with “tumor-specific epitope” or “tumor-specific neoepitope” in the present specification to designate a series of residues, typically L-amino acids, connected one to the other, typically by peptide bonds between the α-amino and carboxyl groups of adjacent amino acids. The neoepitope can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. The present disclosure provides isolated neoepitopes that comprise a tumor-specific mutation from Tables 1 to 12.

In some embodiments, neoepitopes described herein for MHC class I HLA are 12 amino acid residues or less in length and usually consist of between about 8 and about 12 amino acid residues. In some embodiments, neoepitopes described herein for MHC class I HLA is about 8, about 9, about 10, about 11, or about 12 amino acid residues. In some embodiments, neoepitopes described herein for MHC class II HLA are 25 amino acid residues or less in length and usually consist of between about 9 and about 25 amino acid residues. In some embodiments, neoepitopes described herein for MHC class II HLA are about 15, about 16, about 17, about 18, about 19, about 20, about 21, about 22, about 23, about 24, or about 25 amino acid residues.

In some embodiments, the composition described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the composition described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation, and any combination thereof.

In some embodiments, the first neoepitope binds to a class I HLA protein to form a class I HLA-peptide complex. In some embodiments, the second neoepitope binds to a class II HLA a protein to form a class II HLA-peptide complex. In some embodiments, the second neoepitope binds to a class I HLA protein to form a class I HLA-peptide complex. In some embodiments, the first neoepitope binds to a class II HLA protein to form a class II HLA-peptide complex. In some embodiments, the first neoepitope activates CD8⁺ T cells. In some embodiments, the first neoepitope activates CD4⁺ T cells. In some embodiments, the second neoepitope activates CD4⁺ T cells. In some embodiments, the second neoepitope activates CD8⁺ T cells. In some embodiments, a TCR of a CD4⁺ T cell binds to a class II HLA-peptide complex. In some embodiments, a TCR of a CD8⁺ T cell binds to a class II HLA-peptide complex. In some embodiments, a TCR of a CD8⁺ T cell binds to a class I HLA-peptide complex. In some embodiments, a TCR of a CD4⁺ T cell binds to a class I HLA-peptide complex.

In some embodiments, the second neoepitope is longer than the first neoepitope. In some embodiments, the first neoepitope has a length of at least 8 amino acids. In some embodiments, the first neoepitope has a length of from 8 to 12 amino acids. In some embodiments, the first neoepitope comprises a sequence of at least 8 contiguous amino acids, wherein at least 1 of the 8 contiguous amino acids are different at corresponding positions of a wild-type sequence. In some embodiments, the first neoepitope comprises a sequence of at least 8 contiguous amino acids, wherein at least 2 of the 8 contiguous amino acids are different at corresponding positions of a wild-type sequence. In some embodiments, the second neoepitope has a length of at least 16 amino acids. In some embodiments, the second neoepitope has a length of from 16 to 25 amino acids. In some embodiments, the second neoepitope comprises a sequence of at least 16 contiguous amino acids, wherein at least 1 of the 16 contiguous amino acids are different at corresponding positions of a wild-type sequence. In some embodiments, the second neoepitope comprises a sequence of at least 16 contiguous amino acids, wherein at least 2 of the 16 contiguous amino acids are different at corresponding positions of a wild-type sequence.

In some embodiments, the neoepitope comprises at least one anchor residue. In some embodiments, the first neoepitope, the second neoepitope or both comprises at least one anchor residue. In one embodiment, the at least one anchor residue of the first neoepitope is at a canonical anchor position or a non-canonical anchor position. In another embodiment, the at least one anchor residue of the second neoepitope is at a canonical anchor position or a non-canonical anchor position. In yet another embodiment, the at least one anchor residue of the first neoepitope is different from the at least one anchor residue of the second neoepitope.

In some embodiments, the at least one anchor residue is a wild-type residue. In some embodiments, the at least one anchor residue is a substitution. In some embodiments, at least one anchor residue does not comprise the mutation.

In some embodiments, the second neoepitope or both comprise at least one anchor residue flanking region. In some embodiments, the neoepitope comprises at least one anchor residue. In some embodiments, the at least one anchor residues comprises at least two anchor residues. In some embodiments, the at least two anchor residues are separated by a separation region comprising at least 1 amino acid. In some embodiments, the at least one anchor residue flanking region is not within the separation region. In some embodiments, the at least one anchor residue flanking region is (a) upstream of a N-terminal anchor residue of the at least two anchor residues; (b) downstream of a C-terminal anchor residue of the at least two anchor residues; or both (a) and (b).

In some embodiments, the neoepitopes bind an HLA protein (e.g., MHC class I HLA or MHC class II HLA). In some embodiments, the neoepitopes bind an HLA protein with greater affinity than the corresponding wild-type peptide. In some embodiments, the neoepitope has an IC₅₀ of less than 5,000 nM, less than 1,000 nM, less than 500 nM, less than 100 nM, less than 50 nM, or less. In some embodiments, the neoepitope can have an HLA binding affinity of between about 1 μM and about 1 mM, about 100 μM and about 500 μM, about 500 μM and about 10 μM, about 1 nM and about 1 μM, or about 10 nM and about 1 μM. In some embodiments, the neoepitope can have an HLA binding affinity of at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, 1,000, 1,500, or 2,000 nM, or more. In some embodiments, the neoepitope can have an HLA binding affinity of at most 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, 1,000, 1,500, or 2,000 nM.

In some embodiments, the first and/or second neoepitope binds to an HLA protein with a greater affinity than a corresponding wild-type neoepitope. In some embodiments, the first and/or second neoepitope binds to an HLA protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class I protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 2,000 nM, 1,500 nM, 1,000 nM, 900 nM, 800 nM, 700 nM, 600 nM, 500 nM, 250 nM, 150 nM, 100 nM, 50 nM, 25 nM or 10 nM.

In some embodiments, the neoepitope binds to MHC class I HLA. In some embodiments, the neoepitope binds to MHC class I HLA with an affinity of 0.1 nM to 2000 nM. In some embodiments, the neoepitope binds to MHC class I HLA with an affinity of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nM. In some embodiments, the neoepitope binds to MHC class II HLA. In some embodiments, the neoepitope binds to MHC class II HLA with an affinity of 0.1 nM to 2000 nM, 1 nM to 1000 nM, 10 nM to 500 nM, or less than 1000 nM. In some embodiments, the neoepitope binds to MHC class II HLA with an affinity of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 nM.

In some embodiments, the neoepitope binds to MHC class I HLA with a stability of 10 minutes to 24 hours. In some embodiments, the neoepitope binds to MHC class I HLA with a stability of 10, 11, 12, 13, 14, 15, 16, 17, 18, 19, 20, 25, 30, 35, 40, 45, 50, 55, or 60 minutes. In some embodiments, the neoepitope binds to MHC class I HLA with a stability of 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, or 24 hours. In some embodiments, the neoepitope binds to MHC class II HLA with a stability of 10 minutes to 24 hours. In some embodiments, the neoepitope binds to MHC class II HLA with a stability of 10, 11, 12, 13, 14, 15, 16, 17, 18, 19, 20, 25, 30, 35, 40, 45, 50, 55, or 60 minutes. In some embodiments, the neoepitope binds to MHC class II HLA with a stability of 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, or 24 hours.

In an aspect, the first and/or second neoepitope binds to a protein encoded by an HLA allele expressed by a subject. In another aspect, the mutation is not present in non-cancer cells of a subject. In yet another aspect, the first and/or second neoepitope is encoded by a gene or an expressed gene of a subject's cancer cells. In some embodiments, the first neoepitope comprises a mutation as depicted in column 1 of Tables 1 to 12. In some embodiments, the second neoepitope comprises a mutation as depicted in column 1 of Tables 1 to 12. For example, the first neoepitope and the second neoepitope can comprise a sequence ALNSEALSVV. For example, the first neoepitope and the second neoepitope can comprise a sequence MALNSEALSV.

In some embodiments, the first neoepitope and the second neoepitope is derived from a KRAS protein. In some embodiments, the first neoepitope and the second neoepitope is derived from a NRAS protein. In some embodiments, the first neoepitope and the second neoepitope is derived from a KRAS protein comprising a mutation of G12C, G12D, G12V, Q61H, or Q61L substitution. In some embodiments, the first neoepitope and the second neoepitope is derived from a NRAS protein comprising a mutation of Q61K or Q61R substitution. In some embodiments, the neoepitope comprises a substitution mutation, e.g., the KRAS G12C, G12D, G12V, Q61H, or Q61L mutation, or the NRAS Q61K or Q61R mutation. In some embodiments, the first neoepitope and the second neoepitope is derived from a KRAS or NRAS protein sequence of MTEYKLVVVGACGVGKSALTIQLIQNHFVDEYDPTIEDSYRKQVVIDGETCLLDILDTAGQE. For example, the first neoepitope and the second neoepitope can comprise a sequence KLVVVGACGV. For example, the first neoepitope and the second neoepitope can comprise a sequence LVVVGACGV. For example, the first neoepitope and the second neoepitope can comprise a sequence VVGACGVGK. For example, the first neoepitope and the second neoepitope can comprise a sequence VVVGACGVGK. In some embodiments, the first neoepitope and the second neoepitope is derived from a KRAS or NRAS protein sequence of MTEYKLVVVGADGVGKSALTIQLIQNHFVDEYDPTIEDSYRKQVVIDGETCLLDILDTAGQEVVGAD GVGK. For example, the first neoepitope and the second neoepitope can comprise a sequence VVVGADGVGK. For example, the first neoepitope and the second neoepitope can comprise a sequence KLVVVGADGV. For example, the first neoepitope and the second neoepitope can comprise a sequence LVVVGADGV.

In some embodiments, the first neoepitope and the second neoepitope is derived from a KRAS or NRAS protein sequence of MTEYKLVVVGAVGVGKSALTIQLIQNHFVDEYDPTIEDSYRKQVVIDGETCLLDILDTAGQE. For example, the first neoepitope and the second neoepitope can comprise a sequence KLVVVGAVGV. For example, the first neoepitope and the second neoepitope can comprise a sequence LVVVGAVGV. For example, the first neoepitope and the second neoepitope can comprise a sequence VVGAVGVGK. For example, the first neoepitope and the second neoepitope can comprise a sequence VVVGAVGVGK.

In some embodiments, the first neoepitope and the second neoepitope is derived from a KRAS or NRAS protein sequence of AGGVGKSALTIQLIQNHFVDEYDPTIEDSYRKQVVIDGETCLLDILDTAGHEEYSAMRDQYMRTGEG FLCVFAINNTKSFEDIHHYREQIKRVKDSEDVPM. For example, the first neoepitope and the second neoepitope can comprise a sequence ILDTAGHEEY.

In some embodiments, the first neoepitope and the second neoepitope is derived from a KRAS or NRAS protein sequence of AGGVGKSALTIQLIQNHFVDEYDPTIEDSYRKQVVIDGETCLLDILDTAGLEEYSAMRDQYMRTGEG FLCVFAINNTKSFEDIHHYREQIKRVKDSEDVPM. For example, the first neoepitope and the second neoepitope can comprise a sequence ILDTAGLEEY. For example, the first neoepitope and the second neoepitope can comprise a sequence LLDILDTAGL.

In some embodiments, the first neoepitope and the second neoepitope is derived from a KRAS or NRAS protein sequence of AGGVGKSALTIQLIQNHFVDEYDPTIEDSYRKQVVIDGETCLLDILDTAGKEEYSAMRDQYMRTGEG FLCVFAINNSKSFADINLYREQIKRVKDSDDVPM. For example, the first neoepitope and the second neoepitope can comprise a sequence ILDTAGKEEY.

In some embodiments, the first neoepitope and the second neoepitope is derived from a KRAS or NRAS protein sequence of AGGVGKSALTIQLIQNHFVDEYDPTIEDSYRKQVVIDGETCLLDILDTAGREEYSAMRDQYMRTGEG FLCVFAINNSKSFADINLYREQIKRVKDSDDVPM. For example, the first neoepitope and the second neoepitope can comprise a sequence ILDTAGREEY.

In some embodiments, the neoepitope comprises a sequence selected from a group consisting of: DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, and DILDTAGKE.

In some embodiments, the neoepitope comprises a RAS epitope. In some embodiments, the neoepitope comprises a mutant RAS sequence that comprises at least 8 continuous amino acids of a mutant RAS protein comprising a mutation at G12, G13, or Q61 and the mutation at G12, G13, or Q61. In some embodiments, the at least 8 contiguous amino acids of the mutant RAS protein comprising the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation. In some embodiments, the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation.

In some embodiments, the neoepitope comprising a mutant RAS sequence comprises an amino acid sequence of GADGVGKSAL, GACGVGKSAL, GAVGVGKSAL, GADGVGKSA, GACGVGKSA, GAVGVGKSA, KLVVVGACGV, FLVVVGACGL, FMVVVGACGI, FLVVVGACGI, FMVVVGACGV, FLVVVGACGV, MLVVVGACGV, FMVVVGACGL, YLVVVGACGV, KMVVVGACGV, YMVVVGACGV, MMVVVGACGV, DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, or DILDTAGKE.

In some embodiments, the neoepitope comprising a mutant RAS sequence binds to a protein encoded by an HLA allele. In some embodiments, the neoepitope comprising a mutant RAS sequence binds to a protein encoded by an HLA allele with an affinity of less than 10 μM, less than 9 μM, less than 8 μM, less than 7 μM, less than 6 μM, less than 5 μM, less than 4 μM, less than 3 μM, less than 2 μM, less than 1 μM, less than 950 nM, less than 900 nM, less than 850 nM, less than 800 nM, less than 750 nM, less than 600 nM, less than 550 nM, less than 500 nM, less than 450 nM, less than 400 nM, less than 350 nM, less than 300 nM, less than 250 nM, less than 200 nM, less than 150 nM, less than 100 nM, less than 90 nM, less than 80 nM, less than 70 nM, less than 60 nM, less than 50 nM, less than 40 nM, less than 30 nM, less than 20 nM, or less than 10 nM. In some embodiments, the neoepitope comprising a mutant RAS sequence binds to a protein encoded by an HLA allele with a stability of greater than 24 hours, greater than 23 hours, greater than 22 hours, greater than 21 hours, greater than 20 hours, greater than 19 hours, greater than 18 hours, greater than 17 hours, greater than 16 hours, greater than 15 hours, greater than 14 hours, greater than 13 hours, greater than 12 hours, greater than 11 hours, greater than 10 hours, greater than 9 hours, greater than 8 hours, greater than 7 hours, greater than 6 hours, greater than 5 hours, greater than 4 hours, greater than 3 hours, greater than 2 hours, greater than 1 hour, greater than 55 minutes, greater than 50 minutes, greater than 45 minutes, greater than 40 minutes, greater than 35 minutes, greater than 30 minutes, greater than 25 minutes, greater than 20 minutes, greater than 15 minutes, greater than 10 minutes, greater than 9 minutes, greater than 8 minutes, greater than 7 minutes, greater than 6 minutes, greater than 5 minutes, greater than 4 minutes, greater than 3 minutes, greater than 2 minutes, or greater than 1 minutes.

The substitution may be positioned anywhere along the length of the neoepitope. For example, it can be located in the N-terminal third of the peptide, the central third of the peptide or the C-terminal third of the peptide. In another embodiment, the substituted residue is located 2-5 residues away from the N-terminal end or 2-5 residues away from the C-terminal end. The peptides can be similarly derived from tumor-specific insertion mutations where the peptide comprises one or more, or all of the inserted residues.

In some embodiments, the peptide as described herein can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In some embodiments, peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients. In some embodiments, any resins made for solid phase peptide synthesis can be used.

### Polynucleotides

Alternatively, a nucleic acid (e.g., a polynucleotide) encoding the peptide of the present disclosure may be used to produce the neoantigenic peptide in vitro. The polynucleotide may be, e.g., DNA, cDNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In some embodiments in vitro translation is used to produce the peptide.

Provided herein are neoantigenic polynucleotides encoding each of the neoantigenic polypeptides described in the present disclosure. The term “polynucleotide”, “nucleotides” or “nucleic acid” is used interchangeably with “mutant polynucleotide”, “mutant nucleotide”, “mutant nucleic acid”, “neoantigenic polynucleotide”, “neoantigenic nucleotide” or “neoantigenic mutant nucleic acid” in the present disclosure. Various nucleic acid sequences can encode the same peptide due to the redundancy of the genetic code. Each of these nucleic acids falls within the scope of the present disclosure. Nucleic acids encoding peptides can be DNA or RNA, for example, mRNA, or a combination of DNA and RNA. In some embodiments, a nucleic acid encoding a peptide is a self-amplifying mRNA (Brito et al., Adv. Genet. 2015; 89:179-233). Any suitable polynucleotide that encodes a peptide described herein falls within the scope of the present disclosure.

In some embodiments, the coding sequences for two consecutive antigenic peptides are separated by a spacer or linker. In some embodiments, the coding sequences for two consecutive antigenic peptides are adjacent to each other. In some embodiments, the coding sequences for two consecutive antigenic peptides are not separated by a spacer or linker.

In some embodiments, the spacer or linker comprises up to 5000 nucleotide residues. An exemplary spacer sequence is GGCGGCAGCGGCGGCGGCGGCAGCGGCGGC. Another exemplary spacer sequence is GGCGGCAGCCTGGGCGGCGGCGGCAGCGGC. Another exemplary spacer sequence is GGCGTCGGCACC. Another exemplary spacer sequence is CAGCTGGGCCTG. Another exemplary spacer is a sequence that encodes a lysine, such as AAA or AAG. Another exemplary spacer sequence is CAACTGGGATTG.

In some embodiments, the mRNA comprises one or more additional structures to enhance antigen epitope processing and presentation by APCs.

In some embodiments, the linker or spacer region may contain cleavage sites. The cleavage sites ensure cleavage of the protein product comprising strings of epitope sequences into separate epitope sequences for presentation. The preferred cleavage sites are placed adjacent to certain epitopes in order to avoid inadvertent cleavage of the epitopes within the sequences. In some embodiments, the design of epitopes and cleavage regions on the mRNA encoding strings of epitopes are non-random.

The term “RNA” includes and in some embodiments relates to “mRNA.” The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In some embodiments, the mRNA is self-amplifying mRNA. In the context of the present disclosure, mRNA may be generated by in vitro transcription from a DNA template. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448, incorporated herein by reference. In order to increase expression of the RNA used according to the present disclosure, it may be modified within the coding region, i.e., the sequence encoding the expressed peptide or protein, without altering the sequence of the expressed peptide or protein, so as to increase the GC-content to increase mRNA stability and to perform a codon optimization and, thus, enhance translation in cells.

The term “modification” in the context of the RNA used in the present disclosure includes any modification of an RNA which is not naturally present in said RNA. In some embodiments, the RNA does not have uncapped 5′-triphosphates. Removal of such uncapped 5′-triphosphates can be achieved by treating RNA with a phosphatase. In other embodiments, the RNA may have modified ribonucleotides in order to increase its stability and/or decrease cytotoxicity. In some embodiments, 5-methylcytidine can be substituted partially or completely in the RNA, for example, for cytidine. Alternatively, pseudouridine is substituted partially or completely, for example, for uridine.

In some embodiments, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In some embodiments, this guanosine is methylated at the 7-position. The term “conventional 5′-cap” refers to a naturally occurring RNA 5′-cap, to the 7-methylguanosine cap (m G). In the context of the present disclosure, the term “5′-cap” includes a 5′-cap analog that resembles the RNA cap structure and is modified to possess the ability to stabilize RNA and/or enhance translation of RNA if attached thereto, in vivo and/or in a cell.

In certain embodiments, an mRNA encoding a neoantigenic peptide of the present disclosure is administered to a subject in need thereof. In some embodiments, the present disclosure provides RNA, oligoribonucleotide, and polyribonucleotide molecules comprising a modified nucleoside, gene therapy vectors comprising same, gene therapy methods and gene transcription silencing methods comprising same. In some embodiments, the mRNA to be administered comprises at least one modified nucleoside.

The polynucleotides encoding peptides described herein can be synthesized by chemical techniques, for example, the phosphotriester method of Matteucci, et al., J. Am. Chem. Soc. 103:3185 (1981). Polynucleotides encoding peptides comprising or consisting of an analog can be made simply by substituting the appropriate and desired nucleic acid base(s) for those that encode the native epitope.

In some embodiments, the polynucleotides may comprise the coding sequence for the peptide or protein fused in the same reading frame to a polynucleotide which aids, for example, in expression and/or secretion of the peptide or protein from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide.

In some embodiments, the polynucleotides can comprise the coding sequence for the peptide or protein fused in the same reading frame to a marker sequence that allows, for example, for purification of the encoded peptide, which may then be incorporated into a personalized disease vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In some embodiments, the polynucleotides may comprise the coding sequence for one or more of the presently described peptides or proteins fused in the same reading frame to create a single concatamerized neoantigenic peptide construct capable of producing multiple neoantigenic peptides.

In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585. In another embodiment, a DNA sequence encoding the peptide or protein of interest would be constructed by chemical synthesis using an oligonucleotide synthesizer. Such oligonucleotides can be designed based on the amino acid sequence of the desired peptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host. Thus, the present disclosure is also directed to vectors, and expression vectors useful for the production and administration of the neoantigenic polypeptides and neoepitopes described herein, and to host cells comprising such vectors.

In some embodiments, an expression vector capable of expressing the peptide or protein as described herein can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

A large number of vectors and host systems suitable for producing and administering a neoantigenic polypeptide described herein are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, pXT1, pSG (Stratagene) pSVK3, pBPV, pMSG, pSVL (Pharmacia); p75.6 (Valentis); pCEP (Invitrogen); pCEI (Epimmune). However, any other plasmid or vector can be used as long as it is replicable and viable in the host.

Polynucleotides encoding neoantigenic peptides described herein can also comprise a ubiquitination signal sequence, and/or a targeting sequence such as an endoplasmic reticulum (ER) signal sequence to facilitate movement of the resulting peptide into the endoplasmic reticulum.

In some embodiments, the neoantigenic peptide described herein can also be administered and/or expressed by viral or bacterial vectors. Examples of expression vectors include attenuated viral hosts, such as vaccinia or fowlpox. Vaccinia vectors and methods useful in immunization protocols are described in, e.g., U.S. Pat. No. 4,722,848. Another vector is BCG (Bacille Calmette Guerin). BCG vectors are described by Stover et al., Nature 351:456-460 (1991). A wide variety of other vectors useful for therapeutic administration or immunization of the neoantigenic polypeptides described herein, e.g., adeno and adeno-associated virus vectors, retroviral vectors, Salmonella Typhimurium vectors, detoxified anthrax toxin vectors, Sendai virus vectors, poxvirus vectors, canarypox vectors, and the like, will be apparent to those skilled in the art from the description herein. In some embodiments, the vector is Modified Vaccinia Ankara (VA) (e.g. Bavarian Noridic (MVA-BN)).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

Host cells are genetically engineered (transduced or transformed or transfected) with the vectors which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Polynucleotides described herein can be administered and expressed in human cells (e.g., immune cells, including dendritic cells). A human codon usage table can be used to guide the codon choice for each amino acid. Such polynucleotides comprise spacer amino acid residues between epitopes and/or analogs, such as those described above, or can comprise naturally-occurring flanking sequences adjacent to the epitopes and/or analogs (and/or CTL (e.g., CD8+), Th (e.g., CD4+), and B cell epitopes).

Standard regulatory sequences well known to those of skill in the art can be included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. In some embodiments, the promoter is the CMV-IE promoter.

Vectors may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Sharei et al., Ex Vivo Cytosolic Delivery of Functional Macromolecules to Immune Cells, PLOS ONE (2015)).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. Stock solutions of lipids in chloroform or chloroform/methanol can be stored at about −20° C. Chloroform is used as the only solvent since it is more readily evaporated than methanol.

### 4. Antigen Presenting Cells (APCs)

Antigen presenting cells (APC) present peptide fragments of protein antigens in association with MHC molecules on their cell surface. A presented peptide is associated with a MHC molecule as a peptide-MHC complex (pMHC) on the cell surface of the APC. Processing and presentation of peptide-MHC complexes can involve a series of sequential stages comprising: protease-mediated digestion of proteins; peptide transport into the endoplasmic reticulum (ER) mediated by the transporter associated with antigen processing (TAP); formation of peptide-MHC I molecules using newly synthesized MHC molecules; and transport of peptide-MHC molecules to the cell surface.

Some APCs may activate antigen specific T cells. For example, a T cell comprising a T cell receptor (TCR) that interacts with a pMHC may be activated, stimulated, induced, or expanded upon formation of a TCR-pMHC. In some embodiments, an MHC (e.g., a class I MHC or a class II MHC) of an APC can be loaded with a peptide and presented by an APC by introducing into the APC a nucleic acid (e.g., an RNA) encoding an antigen peptide or polypeptide comprising the peptide sequence to be presented.

From a biological perspective, in order for a somatic mutation to generate an immune response several criteria need to be satisfied: the allele containing the mutation should be expressed by the cell, the mutation should be in a protein coding region and nonsynonymous, the translated protein should be cleaved by the proteasome or other cellular protein degradation pathway and an epitope containing the mutation should be presented by the MHC complex, the presented epitope should be recognized by a TCR and, finally, the TCR-pMHC complex should launch a signaling cascade that activates the T cell.

Monocytes can circulate in the bloodstream and then move into tissues where they can differentiate into macrophages and dendritic cells. Classical monocytes are typically characterized by high levels of expression of the CD14 cell surface receptor. Monocytes and B cells can be competent APCs, although their antigen presenting capacities appear to be limited to the re-activation of previously sensitized T cells. These cell types may not be capable of directly activating functionally naïve or unprimed T cell populations. Professional antigen-presenting cells are very efficient at internalizing antigen, either by phagocytosis or by receptor-mediated endocytosis, and then displaying a fragment of the antigen, bound to a MHC molecule, on their membrane. The T cell recognizes and interacts with the antigen-MHC molecule complex on the membrane of the APC. An additional co-stimulatory signal is then produced by the APC, leading to activation of the T cell. The expression of co-stimulatory molecules is a typical feature of professional APCs.

Professional APCs can be very efficient at internalizing antigen, either by phagocytosis or by receptor-mediated endocytosis, and then displaying a fragment of the antigen, bound to a MHC molecule, on their membrane. The T cell can recognize and interact with the antigen-MHC molecule complex on the membrane of the APC. An additional co-stimulatory signal can then be produced by the APC, leading to activation of the T cell. The expression of co-stimulatory molecules can be a defining feature of professional antigen-presenting cells. Examples of professional APCs can include, but are not limited to, dendritic cells (DCs), macrophages, and B-cells. Professional APCs may express high levels of MHC class II, ICAM-1 and B7-2.

One of the main types of professional APCs is DCs, which have the broadest range of antigen presentation. Other main types of professional APCs include macrophages, B-cells, and certain activated epithelial cells. DCs are leukocyte populations that present antigens (e.g., antigens captured in peripheral tissues) to T cells via MHC class II and I antigen presentation pathways. DCs are capable of both activating naïve and previously primed T cells (e.g., memory T cells). DCs can be leukocyte populations that present antigens captured in peripheral tissues to T cells via MHC class I and II antigen presentation pathways. DCs can be potent inducers of immune responses and the activation of these cells can be a critical step for the induction of antitumoral immunity.

DCs can be categorized as “immature” and “mature” cells, which can be used as a simple way to discriminate between two well characterized phenotypes. However, this nomenclature should not be construed to exclude all possible intermediate stages of differentiation. Immature DCs can be characterized as APCs with a high capacity for antigen uptake and processing, which correlates with the high expression of Fcγ receptor and mannose receptor. The mature phenotype can be typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB). Mature DCs may be CD11b⁺, CD11c⁺, HLA-DR⁺, CD80⁺, CD86⁺, CD54⁺, CD3⁻, CD19⁻, CD14⁻, CD141⁺ (BDCA-3), and/or CD1a⁺. DC maturation can be referred to as the status of DC activation at which such antigen presenting DCs lead to T cell priming, while presentation by immature DCs results in tolerance. DC maturation can be caused by biomolecules with microbial features detected by innate receptors (e.g., bacterial DNA, viral RNA, endotoxins, etc.), pro-inflammatory cytokines (e.g., TNFs, interleukins, and interferons), ligation of CD40 on the DC surface by CD40L, and substances released from cells undergoing cell death. Further non-limiting examples of cytokines that can induce DC maturation include IL-4, GM-CSF, TNF-α, IL-1β, PGE1, and IL-6. For example, DCs may be derived by culturing bone marrow cells in vitro with cytokines, such as granulocyte-macrophage colony-stimulating factor (GM-CSF) and tumor necrosis factor alpha (TNF-α). For example, DCs may be derived from CD14′ monocytes isolated from PBMCs. Cytokines or growth factors that can be used for deriving monocytes into DCs include, but are not limited to, GM-CSF, IL-4, FLT3L, TNF-α, IL-1β, PGE1, IL-6, IL-7, IFN-α, R848, LPS, ss-rna40, and polyLC.

Typically, non-professional antigen-presenting cells do not constitutively express MHC class II proteins. MHC class II proteins are typically expressed only upon stimulation of the non-professional APCs by certain cytokines such as IFN-γ.

The source of APC can be typically a tissue source comprising APCs or APC precursors that are capable of expressing and presenting antigen peptides in vitro. In some embodiments, APCs are capable of proliferating and becoming professional APCs when loaded with target RNA and/or treated with the necessary cytokines or factors.

In one aspect, the antigenic polypeptide or protein can be provided as a cell containing such polypeptides, peptides, proteins, or polynucleotides as described herein. In some embodiments, the cell is an antigen presenting cell (APC). In some embodiments, the cell is a dendritic cell (DC). In some embodiments, the cell is a mature antigen presenting cell. In some embodiments, the neoantigenic peptide or protein can be provided as APCs (e.g., dendritic cells) containing such polypeptides, peptides, proteins, or polynucleotides as described herein. In other embodiments, such APCs are used to stimulate T cells for use in patients. Thus, one embodiment of the present disclosure is a composition containing at least one APC (e.g., a dendritic cell) that is pulsed or loaded with one or more neoantigenic peptides or polynucleotides described herein. In some embodiments, such APCs are autologous (e.g., autologous dendritic cells). Alternatively, peripheral blood mononuclear cells (PBMCs) isolated from a patient can be loaded with neoantigenic peptides or polynucleotides ex vivo. In related embodiments, such APCs or PBMCs are injected back into the patient. In some embodiments, the APCs are dendritic cells. In related embodiments, the dendritic cells are autologous dendritic cells that are pulsed with the neoantigenic peptide or nucleic acid. The neoantigenic peptide can be any suitable peptide that gives rise to an appropriate T cell response. T cell therapy using autologous dendritic cells pulsed with peptides from a tumor associated antigen is disclosed in Murphy et al. (1996) The Prostate 29, 371-380 and Tjua et al. (1997) The Prostate 32, 272-278. In some embodiments, the T cell is a CTL (e.g., CD8⁺). In some embodiments, the T cell is a helper T lymphocyte (Th (e.g., CD4⁺)).

In some embodiments, the present disclosure provides a composition comprising a cell-based immunogenic pharmaceutical composition that can also be administered to a subject. For example, an APC based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein. The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

Antigen presenting cells (APCs) can be prepared from a variety of sources, including human and non-human primates, other mammals, and vertebrates. In certain embodiments, APCs can be prepared from blood of a human or non-human vertebrate. APCs can also be isolated from an enriched population of leukocytes. Populations of leukocytes can be prepared by methods known to those skilled in the art. Such methods typically include collecting heparinized blood, apheresis or leukopheresis, preparation of buffy coats, rosetting, centrifugation, density gradient centrifugation (e.g., using Ficoll, colloidal silica particles, and sucrose), differential lysis non-leukocyte cells, and filtration. A leukocyte population can also be prepared by collecting blood from a subject, defibrillating to remove the platelets and lysing the red blood cells. The leukocyte population can optionally be enriched for monocytic dendritic cell precursors.

Blood cell populations can be obtained from a variety of subjects, according to the desired use of the enriched population of leukocytes. The subject can be a healthy subject. Alternatively, blood cells can be obtained from a subject in need of immunostimulation, such as, for example, a cancer patient or other patient for which immunostimulation will be beneficial. Likewise, blood cells can be obtained from a subject in need of immune suppression, such as, for example, a patient having an autoimmune disorder (e.g., rheumatoid arthritis, diabetes, lupus, multiple sclerosis, and the like). A population of leukocytes also can be obtained from an HLA-matched healthy individual.

When blood is used as a source of APC, blood leukocytes may be obtained using conventional methods that maintain their viability. According to one aspect of the present disclosure, blood can be diluted into medium that may or may not contain heparin or other suitable anticoagulant. The volume of blood to medium can be about 1 to 1. Cells can be concentrated by centrifugation of the blood in medium at about 1,000 rpm (150 g) at 4° C. Platelets and red blood cells can be depleted by resuspending the cells in any number of solutions known in the art that will lyse erythrocytes, for example ammonium chloride. For example, the mixture may be medium and ammonium chloride at about 1:1 by volume. Cells may be concentrated by centrifugation and washed in the desired solution until a population of leukocytes, substantially free of platelets and red blood cells, is obtained. Any isotonic solution commonly used in tissue culture may be used as the medium for separating blood leukocytes from platelets and red blood cells. Examples of such isotonic solutions can be phosphate buffered saline, Hanks balanced salt solution, and complete growth media. APCs and/or APC precursor cells may also purified by elutriation.

In one embodiment, the APCs can be non-nominal APCs under inflammatory or otherwise activated conditions. For example, non-nominal APCs can include epithelial cells stimulated with interferon-gamma, T cells, B cells, and/or monocytes activated by factors or conditions that induce APC activity. Such non-nominal APCs can be prepared according to methods known in the art.

The APCs can be cultured, expanded, differentiated and/or, matured, as desired, according to the according to the type of APC. The APCs can be cultured in any suitable culture vessel, such as, for example, culture plates, flasks, culture bags, and bioreactors.

In certain embodiments, APCs can be cultured in suitable culture or growth medium to maintain and/or expand the number of APCs in the preparation. The culture media can be selected according to the type of APC isolated. For example, mature APCs, such as mature dendritic cells, can be cultured in growth media suitable for their maintenance and expansion. The culture medium can be supplemented with amino acids, vitamins, antibiotics, divalent cations, and the like. In addition, cytokines, growth factors and/or hormones, can be included in the growth media. For example, for the maintenance and/or expansion of mature dendritic cells, cytokines, such as granulocyte/macrophage colony stimulating factor (GM-CSF) and/or interleukin 4 (IL-4), can be added. In other embodiments, immature APCs can be cultured and/or expanded. Immature dendritic cells can they retain the ability to uptake target mRNA and process new antigen. In some embodiments, immature dendritic cells can be cultured in media suitable for their maintenance and culture. The culture medium can be supplemented with amino acids, vitamins, antibiotics, divalent cations, and the like. In addition, cytokines, growth factors and/or hormones, can be included in the growth media.

Other immature APCs can similarly be cultured or expanded. Preparations of immature APCs can be matured to form mature APCs. Maturation of APCs can occur during or following exposure to the neoantigenic peptides. In certain embodiments, preparations of immature dendritic cells can be matured. Suitable maturation factors include, for example, cytokines TNF-α, bacterial products (e.g., BCG), and the like. In another aspect, isolated APC precursors can be used to prepare preparations of immature APCs. APC precursors can be cultured, differentiated, and/or matured. In certain embodiments, monocytic dendritic cell precursors can be cultured in the presence of suitable culture media supplemented with amino acids, vitamins, cytokines, and/or divalent cations, to promote differentiation of the monocytic dendritic cell precursors to immature dendritic cells. In some embodiments, the APC precursors are isolated from PBMCs. The PBMCs can be obtained from a donor, for example, a human donor, and can be used freshly or frozen for future usage. In some embodiments, the APC is prepared from one or more APC preparations. In some embodiments, the APC comprises an APC loaded with the first and second neoantigenic peptides comprising the first and second neoepitopes or polynucleotides encoding the first and second neoantigenic peptides comprising the first and second neoepitopes. In some embodiments, the APC is an autologous APC, an allogenic APC, or an artificial APC.

### 5. Adjuvants

An adjuvant can be used to enhance the immune response (humoral and/or cellular) elicited in a patient receiving a composition as provided herein. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5, and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Suitable adjuvants are known in the art (see, WO 2015/095811) and include, but are not limited to poly(I:C), poly-ICLC, Hiltonol, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®. vector system, PLG microparticles, resiquimod, SRL172, virosomes and other virus-like particles, YF-17D, VEGF trap, R848, beta-glucan, Pam2Cys, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998; 186(1):18-27; Allison A C; Dev. Biol. Stand. 1998; 92:3-11) (Mosca et al. Frontiers in Bioscience, 2007; 12:4050-4060) (Gamvrellis et al. Immunol & Cell Biol. 2004; 82: 506-516). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J. Immunother. Emphasis Tumor Immunol. 1996 (6):414-418).

Adjuvant can also comprise stimulatory molecules such as cytokines. Non-limiting examples of cytokines include: CCL20, α-interferon (IFN-a), β-interferon (IFN-β), γ-interferon, platelet derived growth factor (PDGF), TNFα, TNFβ (lymphotoxin alpha (LTα)), GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-I, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include: MCP-1, MIP-1a, MIP-lp, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-1, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll-like receptor (TLR). Examples of modulators of TLRs include TLR-9 agonists and are not limited to small molecule modulators of TLRs such as Imiquimod. Other examples of adjuvants that are used in combination with an immunogenic pharmaceutical composition described herein can include and are not limited to saponin, CpG ODN and the like. Sometimes, an adjuvant is selected from bacteria toxoids, polyoxypropylene-polyoxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolisable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

### 6. Methods of Treatment and Pharmaceutical Compositions

The neoantigen therapeutics (e.g., polypeptides or polynucleotides, APCs or dendritic cells containing the polypeptides or polynucleotides) described herein are useful in a variety of applications including, but not limited to, therapeutic treatment methods, such as the treatment of cancer. In some embodiments, the therapeutic treatment methods comprise immunotherapy. In certain embodiments, a neoantigenic peptide is useful for activating, promoting, increasing, and/or enhancing an immune response, redirecting an existing immune response to a new target, increasing the immunogenicity of a tumor, inhibiting tumor growth, reducing tumor volume, increasing tumor cell apoptosis, and/or reducing the tumorigenicity of a tumor. The methods of use can be in vitro, ex vivo, or in vivo methods.

In some aspects, the present disclosure provides methods for activating an immune response in a subject using a polypeptide, cell, or pharmaceutical composition comprising a neoantigenic peptide or protein described herein. In some embodiments, the present disclosure provides methods of prophylaxis of a subject comprising contacting a cell of the subject with a polypeptide, cell, or pharmaceutical composition comprising a neoantigenic peptide or protein described herein. In some embodiments, the present disclosure provides methods for promoting an immune response in a subject using a polypeptide, cell, or pharmaceutical composition comprising a neoantigenic peptide or protein described herein. In some embodiments, the present disclosure provides methods for increasing an immune response in a subject using a polypeptide, cell, or pharmaceutical composition comprising a neoantigenic peptide or protein described herein. In some embodiments, the present disclosure provides methods for enhancing an immune response using a polypeptide, cell, or pharmaceutical composition comprising a neoantigenic peptide or protein described herein.

In some embodiments, the activating, promoting, increasing, and/or enhancing of an immune response comprises increasing cell-mediated immunity. In some embodiments, the activating, promoting, increasing, and/or enhancing of an immune response comprises increasing T cell activity or humoral immunity. In some embodiments, the activating, promoting, increasing, and/or enhancing of an immune response comprises increasing cytotoxic T lymphocyte (CTL) or helper T lymphocyte (Th) activity. In some embodiments, the activating, promoting, increasing, and/or enhancing of an immune response comprises increasing Natural Killer (NK) cell activity. In some embodiments, the activating, promoting, increasing, and/or enhancing of an immune response comprises increasing T cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of T regulatory (Treg) cells. In some embodiments, the activating, promoting, increasing, and/or enhancing of an immune response comprises increasing anti-tumor activity. In some embodiments, the activating, promoting, increasing, and/or enhancing of an immune response comprises increasing immunogenicity. In some embodiments, the immune response is a result of antigenic stimulation. In some embodiments, the antigenic stimulation is a tumor cell. In some embodiments, the antigenic stimulation is cancer.

In some embodiments, the present disclosure provides methods of activating, promoting, increasing, and/or enhancing of an immune response using a polypeptide, cell, or pharmaceutical composition comprising a neoantigenic peptide or protein described herein. In some embodiments, a method comprises administering to a subject in need thereof a therapeutically effective amount of a polypeptide that delivers a neoantigenic peptide or polynucleotide to a tumor cell. In some embodiments, a method comprises administering to a subject in need thereof a therapeutically effective amount of a neoantigenic polypeptide internalized by the tumor cell. In some embodiments, a method comprises administering to a subject in need thereof a therapeutically effective amount of a neoantigenic polypeptide that is internalized by a tumor cell, and the neoantigenic peptide is processed by the cell. In some embodiments, a method comprises administering to a subject in need thereof a therapeutically effective amount of a neoantigenic polypeptide that is internalized by a tumor cell and a neoepitope is presented on the surface of the tumor cell. In some embodiments, a method comprises administering to a subject in need thereof a therapeutically effective amount of a neoantigenic polypeptide that is internalized by the tumor cell, is processed by the cell, and an antigenic peptide is presented on the surface of the tumor cell.

In some embodiments, a method comprises administering to a subject in need thereof a therapeutically effective amount of a neoantigenic polypeptide or polynucleotide described herein that delivers an exogenous polypeptide comprising at least one neoantigenic peptide to a tumor cell, wherein at least one neoepitope derived from the neoantigenic peptide is presented on the surface of the tumor cell. In some embodiments, the antigenic peptide is presented on the surface of the tumor cell in complex with a MHC class I molecule. In some embodiments, the antigenic peptide is presented on the surface of the tumor cell in complex with a MHC class II molecule.

In some embodiments, a method comprises contacting a tumor cell with a neoantigenic polypeptide or polynucleotide described herein that delivers an exogenous polypeptide comprising at least one neoantigenic polypeptide to the tumor cell, wherein at least one neoepitope derived from the at least one neoantigenic polypeptide is presented on the surface of the tumor cell. In some embodiments, the neoepitope is presented on the surface of the tumor cell in complex with a MHC class I molecule. In some embodiments, the neoepitope is presented on the surface of the tumor cell in complex with a MHC class II molecule.

In some embodiments, a method comprises administering to a subject in need thereof a therapeutically effective amount of a neoantigenic polypeptide or polynucleotide described herein that delivers an exogenous polypeptide comprising at least one antigenic peptide to a tumor cell, wherein the epitope or neoepitope is presented on the surface of the tumor cell, and an immune response against the tumor cell is induced. In some embodiments, the immune response to the epitope or neoepitope is increased. In some embodiments, the immune response against the tumor cell is increased. In some embodiments, the neoantigenic polypeptide or polynucleotide delivers an exogenous polypeptide comprising at least one neoantigenic peptide to a tumor cell, wherein the epitope or neoepitope is presented on the surface of the tumor cell, and tumor growth is inhibited.

In some embodiments, a method comprises administering to a subject in need thereof a therapeutically effective amount of a neoantigenic polypeptide or polynucleotide described herein that delivers an exogenous polypeptide comprising at least one neoantigenic peptide to a tumor cell, wherein the neoepitope derived from the at least one neoantigenic peptide is presented on the surface of the tumor cell, and T cell killing directed against the tumor cell is induced. In some embodiments, T cell killing directed against the tumor cell is enhanced. In some embodiments, T cell killing directed against the tumor cell is increased.

In some embodiments, a method of increasing an immune response in a subject comprises administering to the subject a therapeutically effective amount of a neoantigenic therapeutic described herein, wherein the agent is an antibody that specifically binds the neoantigen described herein. In some embodiments, a method of increasing an immune response in a subject comprises administering to the subject a therapeutically effective amount of the antibody.

The present disclosure provides methods of redirecting an existing immune response to a tumor. In some embodiments, a method of redirecting an existing immune response to a tumor comprises administering to a subject a therapeutically effective amount of a neoantigen therapeutic described herein. In some embodiments, the existing immune response is against a virus. In some embodiments, the virus is selected from the group consisting of measles virus, varicella-zoster virus (VZV; chickenpox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the virus is varicella-zoster virus. In some embodiments, the virus is cytomegalovirus. In some embodiments, the virus is measles virus. In some embodiments, the existing immune response has been acquired after a natural viral infection. In some embodiments, the existing immune response has been acquired after vaccination against a virus. In some embodiments, the existing immune response is a cell-mediated response. In some embodiments, the existing immune response comprises CTL or Th cells.

In some embodiments, a method of redirecting an existing immune response to a tumor in a subject comprises administering a fusion protein comprising (i) an antibody that specifically binds a neoantigen and (ii) at least one neoantigenic peptide described herein, wherein (a) the fusion protein is internalized by a tumor cell after binding to the tumor-associated antigen or the neoepitope; (b) the neoantigenic peptide is processed and presented on the surface of the tumor cell associated with a MHC class I molecule; and (c) the neoantigenic peptide/MHC Class I complex is recognized by CTLs. In some embodiments, the CTLs are memory T cells. In some embodiments, the memory T cells are the result of a vaccination with the neoantigenic peptide.

The present disclosure provides methods of increasing the immunogenicity of a tumor. In some embodiments, a method of increasing the immunogenicity of a tumor comprises contacting a tumor or tumor cells with an effective amount of a neoantigen therapeutic described herein. In some embodiments, a method of increasing the immunogenicity of a tumor comprises administering to a subject a therapeutically effective amount of a neoantigen therapeutic described herein.

The present disclosure also provides methods for inhibiting growth of a tumor using a neoantigen therapeutic described herein. In certain embodiments, a method of inhibiting growth of a tumor comprises contacting a cell mixture with a neoantigen therapeutic in vitro. For example, an immortalized cell line or a cancer cell line mixed with immune cells (e.g., T cells) is cultured in medium to which a neoantigenic peptide is added. In some embodiments, tumor cells are isolated from a patient sample, for example, a tissue biopsy, pleural effusion, or blood sample, mixed with immune cells (e.g., T cells), and cultured in medium to which a neoantigen therapeutic is added. In some embodiments, a neoantigen therapeutic increases, promotes, and/or enhances the activity of the immune cells. In some embodiments, a neoantigen therapeutic inhibits tumor cell growth. In some embodiments, a neoantigen therapeutic activates killing of the tumor cells.

In some embodiments, the subject is a mammal. In certain embodiments, the subject is a human. In certain embodiments, the subject has a tumor or the subject had a tumor which was at least partially removed.

In some embodiments, a method of inhibiting growth of a tumor comprises redirecting an existing immune response to a new target, comprising administering to a subject a therapeutically effective amount of a neoantigen therapeutic, wherein the existing immune response is against an antigenic peptide delivered to the tumor cell by the neoantigenic peptide.

In certain embodiments, the tumor comprises cancer stem cells. In certain embodiments, the frequency of cancer stem cells in the tumor is reduced by administration of the neoantigen therapeutic. In some embodiments, a method of reducing the frequency of cancer stem cells in a tumor in a subject, comprising administering to the subject a therapeutically effective amount of a neoantigen therapeutic is provided.

In addition, in some aspects the present disclosure provides a method of reducing the tumorigenicity of a tumor in a subject, comprising administering to the subject a therapeutically effective amount of a neoantigen therapeutic described herein. In certain embodiments, the tumor comprises cancer stem cells. In some embodiments, the tumorigenicity of a tumor is reduced by reducing the frequency of cancer stem cells in the tumor. In some embodiments, the methods comprise using the neoantigen therapeutic described herein. In certain embodiments, the frequency of cancer stem cells in the tumor is reduced by administration of a neoantigen therapeutic described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

The present disclosure further provides methods for treating cancer in a subject comprising administering to the subject a therapeutically effective amount of a neoantigen therapeutic described herein. In some embodiments, a method of treating cancer comprises redirecting an existing immune response to a new target, the method comprising administering to a subject a therapeutically effective amount of neoantigen therapeutic, wherein the existing immune response is against an antigenic peptide delivered to the cancer cell by the neoantigenic peptide.

The present disclosure provides for methods of treating cancer comprising administering to a subject a therapeutically effective amount of a neoantigen therapeutic described herein (e.g., a subject in need of treatment). In some embodiments, the subject is a mammal. In certain embodiments, the subject is a human. In certain embodiments, the subject has a cancerous tumor. In certain embodiments, the subject has had a tumor at least partially removed.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (i.e., a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, uterine cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is non-small cell lung cancer. In certain embodiments, the cancer is uterine cancer. In certain embodiments, the cancer is liver cancer. In certain embodiments, the cancer is melanoma. In some embodiments, the cancer is a solid cancer. In some embodiments, the cancer comprises a solid tumor.

In some embodiments, the cancer is a hematologic cancer. In some embodiment, the cancer is selected from the group consisting of: acute myelogenous leukemia (AML), Hodgkin lymphoma, multiple myeloma, T cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T cell lymphoma (CTCL).

In some embodiments, the neoantigen therapeutic is administered as a combination therapy. Combination therapy with two or more therapeutic agents uses agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some instances, an immunogenic pharmaceutical composition can be administered with an additional agent. The choice of the additional agent can depend, at least in part, on the condition being treated. The additional agent can include, for example, a checkpoint inhibitor agent such as an anti-PD1, anti-CTLA4, anti-PD-L1, anti CD40, or anti-TIM3 agent (e.g., an anti-PD1, anti-CTLA4, anti-PD-L1, anti CD40, or anti-TIM3 antibody); or any agents having a therapeutic effect for a pathogen infection (e.g., viral infection), including, e.g., drugs used to treat inflammatory conditions such as an NSAID, e.g., ibuprofen, naproxen, acetaminophen, ketoprofen, or aspirin. For example, the checkpoint inhibitor can be a PD-1/PD-L1 antagonist selected from the group consisting of: nivolumab (ONO-4538/BMS-936558, MDX1 106, OPDIVO), pembrolizumab (MK-3475, KEYTRUDA), pidilizumab (CT-011), and MPDL3280A (ROCHE). As another example, formulations can additionally contain one or more supplements, such as vitamin C, E or other antioxidants.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (e.g., cervical canal, eyelid, tunica conjunctiva, vagina, lung, oral cavity, skin, urinary bladder, tongue, larynx, and gullet), and adenocarcinoma (e.g., prostate, small intestine, endometrium, cervical canal, large intestine, lung, pancreas, gullet, rectum, uterus, stomach, mammary gland, and ovary). In some embodiments, a cancer to be treated by the methods of the present disclosure further includes sarcomata (e.g., myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure has a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a neoantigen therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the neoantigen therapeutic described herein include chemotherapeutic agents. Thus, in some embodiments, the method or treatment involves the administration of an agent described herein in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, Pa.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; punne analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6 mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present disclosure with a small molecule that acts as an inhibitor against tumor-associated antigens including, but not limited to, EGFR, HER2 (ErbB2), and/or VEGF. In some embodiments, an agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of Treg cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In another embodiment, the additional therapeutic agent shifts cells to a T helper 1 response. In a further embodiment, the additional therapeutic agent is ibrutinib.

In some embodiments, an additional therapeutic agent comprises a biological molecule, such as an antibody. For example, treatment can involve the combined administration of an agent of the present disclosure with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

The agents and compositions provided herein may be used alone or in combination with conventional therapeutic regimens such as surgery, irradiation, chemotherapy, and/or bone marrow transplantation (autologous, syngeneic, allogeneic, or unrelated). A set of tumor antigens can be useful, e.g., in a large fraction of cancer patients.

In some embodiments, at least one or more chemotherapeutic agents may be administered in addition to the composition comprising an immunogenic vaccine. In some embodiments, the one or more chemotherapeutic agents may belong to different classes of chemotherapeutic agents.

Examples of chemotherapy agents include, but are not limited to, alkylating agents such as nitrogen mustards (e.g., mechlorethamine (nitrogen mustard), chlorambucil, cyclophosphamide (Cytoxan®), ifosfamide, and melphalan); nitrosoureas (e.g., N-Nitroso-N-methylurea, streptozocin, carmustine (BCNU), lomustine, and semustine); alkyl sulfonates (e.g., busulfan); tetrazines (e.g., dacarbazine (DTIC), mitozolomide and temozolomide (Temodar®)); aziridines (e.g., thiotepa, mytomycin and diaziquone); and platinum drugs (e.g., cisplatin, carboplatin, and oxaliplatin); non-classical alkylating agents such as procarbazine and altretamine (hexamethylmelamine); anti-metabolite agents such as 5-fluorouracil (5-FU), 6-mercaptopurine (6-MP), capecitabine (Xeloda®), cladribine, clofarabine, cytarabine (Ara-C®), decitabine, floxuridine, fludarabine, nelarabine, gemcitabine (Gemzar®), hydroxyurea, methotrexate, pemetrexed (Alimta®), pentostatin, thioguanine, Vidaza; anti-microtubule agents such as vinca alkaloids (e.g., vincristine, vinblastine, vinorelbine, vindesine and vinflunine); taxanes (e.g., paclitaxel (Taxol®), docetaxel (Taxotere®)); podophyllotoxin (e.g., etoposide and teniposide); epothilones (e.g., ixabepilone (Ixempra®)); estramustine (Emcyt®); anti-tumor antibiotics such as anthracyclines (e.g., daunorubicin, doxorubicin (Adriamycin®, epirubicin, idarubicin); actinomycin-D; and bleomycin; topoisomerase I inhibitors such as topotecan and irinotecan (CPT-11); topoisomerase II inhibitors such as etoposide (VP-16), teniposide, mitoxantrone, novobiocin, merbarone and aclarubicin; corticosteroids such as prednisone, methylprednisolone (Solumedrol®), and dexamethasone (Decadron®); L-asparaginase; bortezomib (Velcade®); immunotherapeutic agents such as rituximab (Rituxan®), alemtuzumab (Campath®), thalidomide, lenalidomide (Revlimid®), BCG, interleukin-2, interferon-alfa and cancer vaccines such as Provenge®; hormone therapeutic agents such as fulvestrant (Faslodex®), tamoxifen, toremifene (Fareston®), anastrozole (Arimidex®), exemestan (Aromasin®), letrozole (Femara®), megestrol acetate (Megace®), estrogens, bicalutamide (Casodex®), flutamide (Eulexin®), nilutamide (Nilandron®), leuprolide (Lupron®) and goserelin (Zoladex®); differentiating agents such as retinoids, tretinoin (ATRA or Atralin®), bexarotene (Targretin®) and arsenic trioxide (Arsenox®); and targeted therapeutic agents such as imatinib (Gleevec®), gefitinib (Iressa®) and sunitinib (Sutent®). In some embodiments, the chemotherapy is a cocktail therapy. Examples of a cocktail therapy includes, but is not limited to, CHOP/R-CHOP (rituxan, cyclophosphamide, hydroxydoxorubicin, vincristine, and prednisone), EPOCH (etoposide, prednisone, vincristine, cyclophosphamide, hydroxydoxorubicin), Hyper-CVAD (cyclophosphamide, vincristine, hydroxydoxorubicin, dexamethasone), FOLFOX (fluorouracil (5-FU), leucovorin, oxaliplatin), ICE (ifosfamide, carboplatin, etoposide), DHAP (high-dose cytarabine [ara-C], dexamethasone, cisplatin), ESHAP (etoposide, methylprednisolone, cytarabine [ara-C], cisplatin) and CMF (cyclophosphamide, methotrexate, fluouracil).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), atoll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MED14736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the neoantigen therapeutic can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, gamma-IFN, IL-1, IL-2, IL-3, IL-4, IL-5, IL-6, IL-7, IL-12, IL-15, and IL-18.

In some embodiments, treatment with a neoantigen therapeutic described herein can be accompanied by surgical removal of tumors, removal of cancer cells, or any other surgical therapy deemed necessary by a treating physician.

In certain embodiments, treatment involves the administration of a neoantigen therapeutic described herein in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a neoantigen therapeutic described herein and at least one additional therapeutic agent can be administered in any order or concurrently. In some embodiments, the agent will be administered to patients that have previously undergone treatment with a second therapeutic agent. In certain other embodiments, the neoantigen therapeutic and a second therapeutic agent will be administered substantially simultaneously or concurrently. For example, a subject can be given an agent while undergoing a course of treatment with a second therapeutic agent (e.g., chemotherapy). In certain embodiments, a neoantigen therapeutic will be administered within 1 year of the treatment with a second therapeutic agent. It will further be appreciated that the two (or more) agents or treatments can be administered to the subject within a matter of hours or minutes (i.e., substantially simultaneously).

For the treatment of a disease, the appropriate dosage of a neoantigen therapeutic described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. The neoantigen therapeutic can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates.

In some embodiments, a neoantigen therapeutic can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose; a dosing regimen can comprise administering an initial loading dose, followed by maintenance doses of, for example one-half of the initial dose every other week; or a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, therapy must be discontinued, and other agents can be tried. However, many agents in the same therapeutic class display similar side effects and/or toxicities, meaning that the patient either has to stop therapy, or if possible, suffer from the unpleasant side effects associated with the therapeutic agent.

In some embodiments, the dosing schedule can be limited to a specific number of administrations or “cycles.” In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present disclosure provides methods of administering to a subject a neoantigen therapeutic described herein comprising using an intermittent dosing strategy for administering one or more agents, which can reduce side effects and/or toxicities associated with administration of an agent, chemotherapeutic agent, etc. In some embodiments, a method for treating cancer in a human subject comprises administering to the subject a therapeutically effective dose of a neoantigen therapeutic in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of a neoantigen therapeutic in combination with a therapeutically effective dose of a second immunotherapeutic agent, wherein one or both of the agents are administered according to an intermittent dosing strategy. In some embodiments, the intermittent dosing strategy comprises administering an initial dose of a neoantigen therapeutic to the subject, and administering subsequent doses of the agent about once every 2 weeks. In some embodiments, the intermittent dosing strategy comprises administering an initial dose of a neoantigen therapeutic to the subject, and administering subsequent doses of the agent about once every 3 weeks. In some embodiments, the intermittent dosing strategy comprises administering an initial dose of a neoantigen therapeutic to the subject, and administering subsequent doses of the agent about once every 4 weeks. In some embodiments, the agent is administered using an intermittent dosing strategy and the additional therapeutic agent is administered weekly.

The present disclosure provides compositions comprising the neoantigen therapeutic described herein. The present disclosure also provides pharmaceutical compositions comprising a neoantigen therapeutic described herein and a pharmaceutically acceptable vehicle. In some embodiments, the pharmaceutical compositions find use in immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a neoantigen therapeutic of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition. Exemplary formulations are listed in WO 2015/095811.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London). In some embodiments, the vehicle is 5% dextrose in water.

In one aspect, provided herein are pharmaceutically acceptable or physiologically acceptable compositions including solvents (aqueous or non-aqueous), solutions, emulsions, dispersion media, coatings, isotonic and absorption promoting or delaying agents, compatible with pharmaceutical administration. Pharmaceutical compositions or pharmaceutical formulations therefore refer to a composition suitable for pharmaceutical use in a subject. Compositions can be formulated to be compatible with a particular route of administration (i.e., systemic or local). Thus, compositions include carriers, diluents, or excipients suitable for administration by various routes.

In some embodiments, a composition can further comprise an acceptable additive in order to improve the stability of immune cells in the composition. Acceptable additives may not alter the specific activity of the immune cells. Examples of acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives can be combined with acceptable carriers and/or excipients such as dextrose. Alternatively, examples of acceptable additives include, but are not limited to, a surfactant such as polysorbate 20 or polysorbate 80 to increase stability of the peptide and decrease gelling of the solution. The surfactant can be added to the composition in an amount of 0.01% to 5% of the solution. Addition of such acceptable additives increases the stability and half-life of the composition in storage.

The pharmaceutical compositions described herein can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intra-arterial, intratumoral, subcutaneous, intraperitoneal, intramuscular (e.g., injection or infusion), or intracranial (e.g., intrathecal or intraventricular).

The pharmaceutical composition can be administered, for example, by injection. Administration can be intradermal injection, intranasal spray application, intramuscular injection, intraperitoneal injection, intravenous injection, oral administration, or subcutaneous injection. Compositions for injection include aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, and sodium chloride can be included in the composition. The resulting solutions can be packaged for use as is, or lyophilized; the lyophilized preparation can later be combined with a sterile solution prior to administration. For intravenous, injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as needed. Sterile injectable solutions can be prepared by incorporating an active ingredient in the required amount in an appropriate solvent with one or a combination of ingredients enumerated above, as required, followed by filtered sterilization. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation can be vacuum drying and freeze drying which yields a powder of the active ingredient plus any additional desired ingredient from a previously sterile-filtered solution thereof.

Compositions can be conventionally administered intravenously, such as by injection of a unit dose, for example. For injection, an active ingredient can be in the form of a parenterally acceptable aqueous solution which is substantially pyrogen-free and has suitable pH, isotonicity, and stability. One can prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Additionally, compositions can be administered via aerosolization.

When the compositions are considered for use in medicaments or any of the methods provided herein, it is contemplated that the composition can be substantially free of pyrogens such that the composition will not cause an inflammatory reaction or an unsafe allergic reaction when administered to a human patient. Testing compositions for pyrogens and preparing compositions substantially free of pyrogens are well understood to one or ordinary skill of the art and can be accomplished using commercially available kits.

Acceptable carriers can contain a compound that stabilizes, increases, or delays absorption, or increases or delays clearance. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (e.g., Fix (1996) Pharm Res. 13:1760 1764; Samanen (1996) J. Pharm. Pharmacol. 48:119 135; and U.S. Pat. No. 5,391,377).

The compositions can be administered in a manner compatible with the dosage formulation, and in a therapeutically effective amount. The quantity to be administered depends on the subject to be treated, capacity of the subject's immune system to utilize the active ingredient, and degree of binding capacity desired. Precise amounts of active ingredient required to be administered depend on the judgment of the practitioner and are peculiar to each individual. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations in the blood are contemplated.

In some cases, pharmaceutical compositions comprising one or more agents exert local and regional effects when administered topically or injected at or near particular sites of infection. Direct topical application, e.g., of a viscous liquid, solution, suspension, dimethylsulfoxide (DMSO)-based solutions, liposomal formulations, gel, jelly, cream, lotion, ointment, suppository, foam, or aerosol spray, can be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations can also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In another embodiment, local/topical formulations comprising a transporter, carrier, or ion channel inhibitor are used to treat epidermal or mucosal viral infections.

In some instances, an immunogenic pharmaceutical composition can include carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In another instances, the pharmaceutical preparation is substantially free of preservatives. In other instances, the pharmaceutical preparation can contain at least one preservative. It will be recognized that, while any suitable carrier known to those of ordinary skill in the art can be employed to administer the pharmaceutical compositions described herein, the type of carrier will vary depending on the mode of administration.

An immunogenic pharmaceutical composition can include preservatives such as thiomersal or 2-phenoxyethanol. In some instances, the immunogenic pharmaceutical composition is substantially free from (e.g., <10 μg/mL) mercurial material e.g. thiomersal-free. α-Tocopherol succinate may be used as an alternative to mercurial compounds.

For controlling the tonicity, a physiological salt such as sodium salt can be included in the immunogenic pharmaceutical composition. Other salts can include potassium chloride, potassium dihydrogen phosphate, disodium phosphate, and/or magnesium chloride, or the like.

An immunogenic pharmaceutical composition can have an osmolality of between 200 mOsm/kg and 400 mOsm/kg, between 240-360 mOsm/kg, or within the range of 290-310 mOsm/kg.

An immunogenic pharmaceutical composition can comprise one or more buffers, such as a Tris buffer; a borate buffer; a succinate buffer; a histidine buffer (particularly with an aluminum hydroxide adjuvant); or a citrate buffer. Buffers, in some cases, are included in the 5-20 or 10-50 mM range.

An immunogenic pharmaceutical composition can comprise a pH modifier. In some embodiments, the pH modifier is present at a concentration of less than 1 mM or greater than 1 mM. In some embodiments, the pH modifier is present at a concentration of less than 10, 20, 30, 40, 50, 60, 70, 80, 90, 100, 200, 300, 400, 500, 600, 700, 800, 900 nM, or 1 mM. In some embodiments, the pH modifier is present at a concentration of greater than 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, or 900 mM. In some embodiments, the pH modifier is a dicarboxylate salt. In some embodiments, the pH modifier is a tricarboxylate salt. In some embodiments, the pH modifier is a dicarboxylate salt of succinic acid. In some embodiments, the pH modifier is a disuccinate salt. In some embodiments, the pH modifier is a tricarboxylate salt of citric acid. In some embodiments, the pH modifier is a tricitrate salt. In some embodiments, the pH modifier is disodium succinate. In some embodiments, the dicarboxylate salt of succinic acid is present in the pharmaceutical composition at a concentration of 0.1 mM-1 mM. In some embodiments, the disuccinate salt is present in the pharmaceutical composition at a concentration of 0.1 mM-1 mM. In some embodiments, the dicarboxylate salt of succinic acid is present in the pharmaceutical composition at a concentration of 1 mM-5 mM. In some embodiments, the disuccinate salt is present in the pharmaceutical composition at a concentration of 1 mM-5 mM. The pH of the immunogenic pharmaceutical composition can be between about 5.0 and about 8.5, between about 6.0 and about 8.0, between about 6.5 and about 7.5, or between about 7.0 and about 7.8.

An immunogenic pharmaceutical composition can be sterile. The immunogenic pharmaceutical composition can be non-pyrogenic e.g. containing <1 EU (endotoxin unit, a standard measure) per dose, and can be <0.1 EU per dose. The composition can be gluten free.

An immunogenic pharmaceutical composition can include detergent e.g. a polyoxyethylene sorbitan ester surfactant (known as ‘Tweens’), or an octoxynol (such as octoxynol-9 (Triton X-100) or t-octylphenoxypolyethoxyethanol). The detergent can be present only at trace amounts. The immunogenic pharmaceutical composition can include less than 1 mg/mL of each of octoxynol-10 and polysorbate 80. Other residual components in trace amounts can be antibiotics (e.g., neomycin, kanamycin, polymyxin B).

An immunogenic pharmaceutical composition can be formulated as a sterile solution or suspension, in suitable vehicles, well known in the art. The pharmaceutical compositions can be sterilized by conventional, well-known sterilization techniques, or can be sterile filtered. The resulting aqueous solutions can be packaged for use as is, or lyophilized, the lyophilized preparation being combined with a sterile solution prior to administration.

Pharmaceutical compositions comprising, for example, an active agent such as immune cells disclosed herein, in combination with one or more adjuvants can be formulated to comprise certain molar ratios. For example, molar ratios of about 99:1 to about 1:99 of an active agent such as an immune cell described herein, in combination with one or more adjuvants can be used. In some instances, the range of molar ratios of an active agent such as an immune cell described herein, in combination with one or more adjuvants can be selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio of an active agent such as an immune cell described herein, in combination with one or more adjuvants can be about 1:9, and in some cases can be about 1:1. The active agent such as an immune cell described herein, in combination with one or more adjuvants can be formulated together, in the same dosage unit e.g., in one vial, suppository, tablet, capsule, an aerosol spray; or each agent, form, and/or compound can be formulated in separate units, e.g., two vials, suppositories, tablets, two capsules, a tablet and a vial, an aerosol spray, and the like.

The therapeutic formulation can be in unit dosage form. Such formulations include tablets, pills, capsules, powders, granules, solutions, or suspensions in water or non-aqueous media, or suppositories.

The neoantigenic peptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include a neoantigen therapeutic described herein complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the neoantigenic peptides described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The present disclosure provides methods of treatment comprising an immunogenic vaccine. Methods of treatment for a disease (such as cancer or a viral infection) are provided. A method can comprise administering to a subject an effective amount of a composition comprising an immunogenic antigen. In some embodiments, the antigen comprises a viral antigen. In some embodiments, the antigen comprises a tumor antigen.

Non-limiting examples of vaccines that can be prepared include a peptide-based vaccine, a nucleic acid-based vaccine, an antibody based vaccine, and an antigen-presenting cell based vaccine.

Vaccine compositions can be formulated using one or more physiologically acceptable carriers including excipients and auxiliaries which facilitate processing of the active agents into preparations which can be used pharmaceutically. Proper formulation can be dependent upon the route of administration chosen. Any of the well-known techniques, carriers, and excipients can be used as suitable and as understood in the art.

In some cases, the vaccine composition is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al., J. Clin. Invest. 95:341, 1995), naked cDNA or peptides, encapsulated e.g., in poly(DL-lactide-co-glycolide) (“PLG”) microspheres (see, e.g., Eldridge, et al., Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). Sometimes, a vaccine is formulated as a peptide-based vaccine, or nucleic acid based vaccine in which the nucleic acid encodes the polypeptides. Sometimes, a vaccine is formulated as an antibody based vaccine. Sometimes, a vaccine is formulated as a cell based vaccine.

The amino acid sequence of an identified disease-specific immunogenic neoantigen peptide can be used to develop a pharmaceutically acceptable composition. The source of antigen can be, but is not limited to, natural or synthetic proteins, including glycoproteins, peptides, and superantigens; antibody/antigen complexes; lipoproteins; RNA or a translation product thereof; and DNA or a polypeptide encoded by the DNA. The source of antigen may also comprise non-transformed, transformed, transfected, or transduced cells or cell lines. Cells may be transformed, transfected, or transduced using any of a variety of expression or retroviral vectors known to those of ordinary skill in the art that may be employed to express recombinant antigens. Expression may also be achieved in any appropriate host cell that has been transformed, transfected, or transduced with an expression or retroviral vector containing a DNA molecule encoding recombinant antigen(s). Any number of transfection, transformation, and transduction protocols known to those in the art may be used. Recombinant vaccinia vectors and cells infected with the vaccinia vector may be used as a source of antigen.

A pharmaceutical composition can comprise a synthetic disease-specific immunogenic neoantigen peptide. A pharmaceutical composition can comprise two or more disease-specific immunogenic neoantigen peptides. A pharmaceutical composition may comprise a precursor to a disease-specific immunogenic peptide (such as a protein, peptide, DNA and RNA). A precursor to a disease-specific immunogenic peptide can generate or be generated to the identified disease-specific immunogenic neoantigen peptide. In some embodiments, a therapeutic composition comprises a precursor of an immunogenic peptide. The precursor to a disease-specific immunogenic peptide can be a pro-drug. In some embodiments, the pharmaceutical composition comprising a disease-specific immunogenic neoantigen peptide may further comprise an adjuvant. For example, the neoantigen peptide can be utilized as a vaccine. In some embodiments, an immunogenic vaccine may comprise a pharmaceutically acceptable immunogenic neoantigen peptide. In some embodiments, an immunogenic vaccine may comprise a pharmaceutically acceptable precursor to an immunogenic neoantigen peptide (such as a protein, peptide, DNA and RNA). In some embodiments, a method of treatment comprises administering to a subject an effective amount of an antibody specifically recognizing an immunogenic neoantigen peptide.

The methods described herein are useful in the personalized medicine context, where immunogenic neoantigen peptides are used to develop therapeutics (such as vaccines or therapeutic antibodies) for the same individual. Thus, a method of treating a disease in a subject can comprise identifying an immunogenic neoantigen peptide in a subject according to the methods described herein; and synthesizing the peptide (or a precursor thereof); and administering the peptide or an antibody specifically recognizing the peptide to the subject.

In some embodiments, identifying an epitope expressed by a subject's tumor cells or an immunogenic neoantigen peptide comprises selecting a plurality of nucleic acid sequences from a pool of nucleic acid sequences sequenced from the subject's tumor cells that encode a plurality of candidate peptide sequences comprising one or more different mutations not present in a pool of nucleic acid sequences sequenced from the subject's non-tumor cells, wherein the pool of nucleic acid sequences sequenced from the subject's tumor cells and the pool of nucleic acid sequences sequenced from the subject's non-tumor cells are sequenced by whole genome sequencing or whole exome sequencing. In some embodiments, identifying an epitope expressed by a subject's tumor cells or an immunogenic neoantigen peptide further comprises predicting or measuring which candidate peptide sequences of the plurality of candidate peptide sequences form a complex with a protein encoded by an HLA allele of the same subject by an HLA peptide binding analysis. In some embodiments, identifying an epitope expressed by a subject's tumor cells or an immunogenic neoantigen peptide further comprises selecting the plurality of selected tumor-specific peptides or one or more polynucleotides encoding the plurality of selected tumor-specific peptides from the candidate peptide sequences based on the HLA peptide binding analysis. In some embodiments, the epitope expressed by the subject's tumor cells is a neoantigen, a tumor associated antigen, a mutated tumor associated antigen, and/or wherein expression of the epitope is higher in the subject's tumor cells compared to expression of the epitope in a normal cell of the subject.

In some embodiments, an expression pattern of an immunogenic neoantigen can serve as the essential basis for the generation of patient specific vaccines. In some embodiments, an expression pattern of an immunogenic neoantigen can serve as the essential basis for the generation of a vaccine for a group of patients with a particular disease. Thus, particular diseases, e.g., particular types of tumors, can be selectively treated in a patient group.

In some embodiments, the peptides described herein are structurally normal antigens that can be recognized by autologous anti-disease T cells in a large patient group. In some embodiments, an antigen-expression pattern of a group of diseased subjects whose disease expresses structurally normal neoantigens is determined.

In some embodiments, the pharmaceutical composition described herein comprises at least two polypeptide comprises at least at two polypeptide molecules. In some embodiments, the two or more of the at least two polypeptides or polypeptide molecules comprise the same epitope of the same length. In some embodiments, the two or more of the at least two polypeptides or polypeptide molecules comprise the same amino acid or amino acid sequence that is of a peptide sequence that is not encoded by a nucleic acid sequence immediately upstream or downstream of the nucleic acid sequence in the genome of the subject that encodes the epitope. In some embodiments, the two or more of the at least two polypeptides or polypeptide molecules comprise a different linker. In some embodiments, a first polypeptide of the at least two polypeptides or polypeptide molecules does not comprise a linker and a second polypeptide of the at least two polypeptides or polypeptide molecules comprises a linker. In some embodiments, the first polypeptide of the at least two polypeptides or polypeptide molecules does not comprise a linker on the N-terminus of the epitope and the second polypeptide of the at least two polypeptides or polypeptide molecules comprises a linker on the N-terminus of the epitope. In some embodiments, the first polypeptide of the at least two polypeptides or polypeptide molecules does not comprise a linker on the C-terminus of the epitope and the second polypeptide of the at least two polypeptides or polypeptide molecules comprises a linker on the C-terminus of the epitope. In some embodiments, a first polypeptide of the at least two polypeptides or polypeptide molecules comprises a linker and a second polypeptide of the at least two polypeptides or polypeptide molecules does not comprise a linker. In some embodiments, the first polypeptide of the at least two polypeptides or polypeptide molecules comprises a linker on the N-terminus of the epitope and the second polypeptide of the at least two polypeptides or polypeptide molecules does not comprise a linker on the N-terminus of the epitope. In some embodiments, the first polypeptide of the at least two polypeptides or polypeptide molecules comprises a linker on the C-terminus of the epitope and the second polypeptide of the at least two polypeptides or polypeptide molecules does not comprise a linker on the C-terminus of the epitope.

In some embodiments, the epitope is present in the pharmaceutical composition at an amount of from 1 ng to 10 mg or 5 pg to 1.5 mg. In some embodiments, the epitope is present at an amount from 1 ng to 10 mg. In some embodiments, the epitope is present at an amount from 1 ng to 100 ng, from 10 ng to 200 ng, from 20 ng to 300 ng, from 30 ng to 400 ng, from 40 ng to 500 ng, from 50 ng to 600 ng, from 60 ng to 700 ng, from 70 ng to 800 ng, from 80 ng to 900 ng, from 90 ng to 1 pg, from 100 ng to 2 pg, from 200 ng to 3 pg, from 300 ng to 4 pg, from 400 ng to 5 pg, from 500 ng to 6 pg, from 600 ng to 7 pg, from 700 ng to 8 pg, from 800 ng to 9 pg, from 900 ng to 10 pg, from 1 pg to 100 pg, from 20 pg to 200 pg, from 30 pg to 300 pg, from 40 pg to 400 pg, from 50 pg to 500 pg, from 60 pg to 600 pg, from 70 pg to 700 pg, from 80 pg to 800 pg, from 90 pg to 900 pg, from 100 pg to 1 mg, from 200 pg to 1.1 mg, from 300 pg to 1.2 mg, from 400 pg to 1.3 mg, from 500 pg to 1.4 mg, from 600 pg to 1.5 mg, from 700 pg to 2 mg, from 800 pg to 3 mg, from 900 pg to 4 mg, from 1 mg to 5 mg, from 1.3 mg to 6 mg, from 1.5 mg to 7 mg, from 2 mg to 8 mg, from 3 mg to 9 mg, or from 4 mg to 10 mg. In some embodiments, the epitope is present at an amount of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, or 950 ng. In some embodiments, the epitope is present at an amount of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 550, 600, 650, 700, 750, 800, 850, 900, 950 pg. In some embodiments, the epitope is present at an amount of about 1, 1.1, 1.2, 1.3, 1.4, 1.5, 2, 2.5, 3, 3.5, 4, 4.5, 5, 6, 7, 8, 9, or 10 mg.

There are a variety of ways to produce immunogenic neoantigens. Proteins or peptides may be made by any technique known to those of skill in the art, including expression of proteins, polypeptides, or peptides through standard molecular biological techniques, isolation of proteins or peptides from natural sources, in vitro translation, or the chemical synthesis of proteins or peptides. In general, such disease-specific neoantigens may be produced either in vitro or in vivo. Immunogenic neoantigens may be produced in vitro as peptides or polypeptides, which may then be formulated into a personalized vaccine or immunogenic composition and administered to a subject. In vitro production of immunogenic neoantigens can comprise peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, immunogenic neoantigens can be produced in vivo by introducing molecules (e.g., DNA, RNA, and viral expression systems) that encode an immunogenic neoantigen into a subject, whereupon the encoded immunogenic neoantigens are expressed. In some embodiments, a polynucleotide encoding an immunogenic neoantigen peptide can be used to produce the neoantigen peptide in vitro.

In some embodiments, a polynucleotide comprises a sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a polynucleotide encoding an immunogenic neoantigen. The polynucleotide may be, e.g., DNA, cDNA, single- and/or double-stranded, native or stabilized forms of polynucleotides, or combinations thereof. A nucleic acid encoding an immunogenic neoantigen peptide may or may not contain introns so long as it codes for the peptide. In some embodiments in vitro translation is used to produce the peptide.

Expression vectors comprising sequences encoding the neoantigen, as well as host cells containing the expression vectors, are also contemplated. Expression vectors suitable for use in the present disclosure can comprise at least one expression control element operationally linked to the nucleic acid sequence. The expression control elements are inserted in the vector to control and regulate the expression of the nucleic acid sequence. Examples of expression control elements are well known in the art and include, for example, the lac system, operator and promoter regions of phage lambda, yeast promoters, and promoters derived from polyoma, adenovirus, retrovirus, or SV40. Additional operational elements include, but are not limited to, leader sequences, termination codons, polyadenylation signals and any other sequences necessary or preferred for the appropriate transcription and subsequent translation of the nucleic acid sequence in the host system. It will be understood by one skilled in the art the correct combination of expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers.

The neoantigen peptides may be provided in the form of RNA or cDNA molecules encoding the desired neoantigen peptides. One or more neoantigen peptides of the present disclosure may be encoded by a single expression vector. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. Useful expression vectors for eukaryotic hosts, especially mammals or humans include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages. Suitable host cells for expression of a polypeptide are discussed in Polynucleotides section [0250]. Appropriate cloning and expression vectors for use with bacterial, fungal, yeast, and mammalian cellular hosts are well known in the art.

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance, and x-ray crystallography.

A vaccine can comprise an entity that binds a polypeptide sequence described herein. The entity can be an antibody. Antibody-based vaccine can be formulated using any of the well-known techniques, carriers, and excipients as suitable and as understood in the art. In some embodiments, the peptides described herein can be used for making neoantigen specific therapeutics such as antibody therapeutics. For example, neoantigens can be used to raise and/or identify antibodies specifically recognizing the neoantigens. These antibodies can be used as therapeutics. The antibody can be a natural antibody, a chimeric antibody, a humanized antibody, or can be an antibody fragment. The antibody may recognize one or more of the polypeptides described herein. In some embodiments, the antibody can recognize a polypeptide that has a sequence with at most 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a polypeptide described herein. In some embodiments, the antibody can recognize a polypeptide that has a sequence with at least 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide described herein. In some embodiments, the antibody can recognize a polypeptide sequence that is at least 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein. In some embodiments, the antibody can recognize a polypeptide sequence that is at most 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein.

The present disclosure also contemplates the use of nucleic acid molecules as vehicles for delivering neoantigen peptides/polypeptides to the subject in need thereof, in vivo, in the form of, e.g., DNA vaccines.

In some embodiments, the vaccine is a nucleic acid vaccine. In some embodiments, the nucleic acid encodes an immunogenic peptide or peptide precursor. In some embodiments, the nucleic acid vaccine comprises sequences flanking the sequence coding the immunogenic peptide or peptide precursor. In some embodiments, the nucleic acid vaccine comprises more than one immunogenic epitope. In some embodiments, the nucleic acid vaccine is a DNA-based vaccine. The methods of delivery are discussed in Polynucleotide section [0250].

The polynucleotide may be substantially pure, or contained in a suitable vector or delivery system. Suitable vectors and delivery systems include viral, such as systems based on adenovirus, vaccinia virus, retroviruses, herpes virus, adeno-associated virus, or hybrids containing elements of more than one virus. Non-viral delivery systems include cationic lipids and cationic polymers (e.g., cationic liposomes).

One or more neoantigen peptides can be encoded and expressed in vivo using a viral based system. Viral vectors may be used as recombinant vectors in the present disclosure, wherein a portion of the viral genome is deleted to introduce new genes without destroying infectivity of the virus. The viral vector of the present disclosure is a nonpathogenic virus. In some embodiments the viral vector has a tropism for a specific cell type in the mammal. In another embodiment, the viral vector of the present disclosure is able to infect professional antigen presenting cells such as dendritic cells and macrophages. In yet another embodiment of the present disclosure, the viral vector is able to infect any cell in the mammal. The viral vector may also infect tumor cells. Viral vectors used in the present disclosure include but is not limited to Poxvirus such as vaccinia virus, avipox virus, fowlpox virus, and a highly attenuated vaccinia virus (Ankara or MVA), retrovirus, adenovirus, baculovirus and the like.

A vaccine can be delivered via a variety of routes. Delivery routes can include oral (including buccal and sub-lingual), rectal, nasal, topical, transdermal patch, pulmonary, vaginal, suppository, or parenteral (including intramuscular, intra-arterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999). The vaccine described herein can be administered to muscle, or can be administered via intradermal or subcutaneous injections, or transdermally, such as by iontophoresis. Epidermal administration of the vaccine can be employed. The vaccine described here in can be administered via intradermal injection, intranasal spray application, intramuscular injection, intraperitoneal injection, intravenous injection, oral administration, or subcutaneous injection.

In some instances, the vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup, or elixir. The vaccine can also be a preparation for parenteral, subcutaneous, intradermal, intramuscular, or intravenous administration (e.g., injectable administration), such as a sterile suspension or emulsion.

The vaccine can include material for a single immunization, or may include material for multiple immunizations (i.e. a ‘multidose’ kit). The inclusion of a preservative is preferred in multidose arrangements. Aₛ an alternative (or in addition) to including a preservative in multidose compositions, the compositions can be contained in a container having an aseptic adaptor for removal of material.

The vaccine can be administered in a dosage volume of about 0.5 mL, although a half dose (i.e. about 0.25 mL) can be administered to children. Sometimes the vaccine can be administered in a higher dose e.g. about 1 ml.

The vaccine can be administered as a 1, 2, 3, 4, 5, 6, 7, 8, 9, 10, or more dose-course regimen. Sometimes, the vaccine is administered as a 1, 2, 3, or 4 dose-course regimen. Sometimes the vaccine is administered as a 1 dose-course regimen. Sometimes the vaccine is administered as a 2 dose-course regimen.

The administration of the first dose and second dose can be separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, or more.

The vaccine described herein can be administered every 1, 2, 3, 4, 5, 6, 7, 8, 9, 10, or more years. Sometimes, the vaccine described herein is administered every 2, 3, 4, 5, 6, 7, or more years. Sometimes, the vaccine described herein is administered every 4, 5, 6, 7, or more years. Sometimes, the vaccine described herein is administered once.

The dosage examples are not limiting and are only used to exemplify particular dosing regiments for administering a vaccine described herein. The effective amount for use in humans can be determined from animal models. For example, a dose for humans can be formulated to achieve circulating, liver, topical and/or gastrointestinal concentrations that have been found to be effective in animals. Based on animal data, and other types of similar data, those skilled in the art can determine the effective amounts of a vaccine composition appropriate for humans.

The effective amount when referring to an agent or combination of agents will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some aspects, the vaccine and kit described herein can be stored at between 2° C. and 8° C. In some instances, the vaccine is not stored frozen. In some instances, the vaccine is stored in temperatures of such as at −20° C. or −80° C. In some instances, the vaccine is stored away from sunlight.

### 7. Kits

The neoantigen therapeutic described herein can be provided in kit form together with instructions for administration. Typically the kit would include the desired neoantigen therapeutic in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

Kits and articles of manufacture are also provided herein for use with one or more methods described herein. The kits can contain one or more neoantigenic polypeptides comprising one or more neoepitopes. The kits can also contain nucleic acids that encode one or more of the peptides or proteins described herein, antibodies that recognize one or more of the peptides described herein, or APC-based cells activated with one or more of the peptides described herein. The kits can further contain adjuvants, reagents, and buffers necessary for the makeup and delivery of the vaccines.

The kits can also include a carrier, package, or container that is compartmentalized to receive one or more containers such as vials, tubes, and the like, each of the container(s) comprising one of the separate elements, such as the peptides and adjuvants, to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. A kit typically includes labels listing contents and/or instructions for use, and package inserts with instructions for use. A set of instructions will also typically be included.

The present disclosure will be described in greater detail by way of specific examples. The following examples are offered for illustrative purposes, and are not intended to limit the present disclosure in any manner. Those of skill in the art will readily recognize a variety of non-critical parameters that can be changed or modified to yield alternative embodiments according to the present disclosure. All patents, patent applications, and printed publications listed herein are incorporated herein by reference in their entirety.

### EXAMPLES

These examples are provided for illustrative purposes only and not to limit the scope of the claims provided herein.

### Example 1—Assessment of Enhanced Cleavage and Processing of Polypeptides

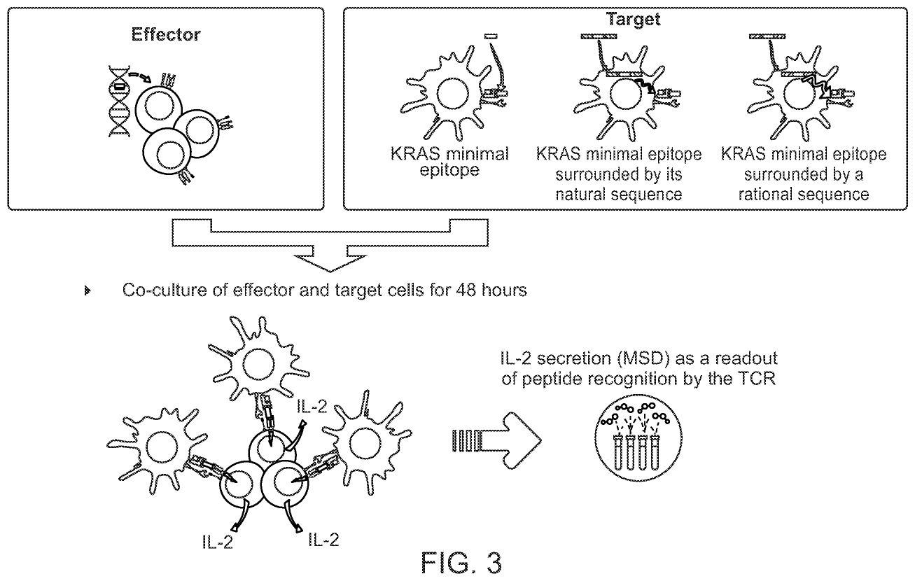

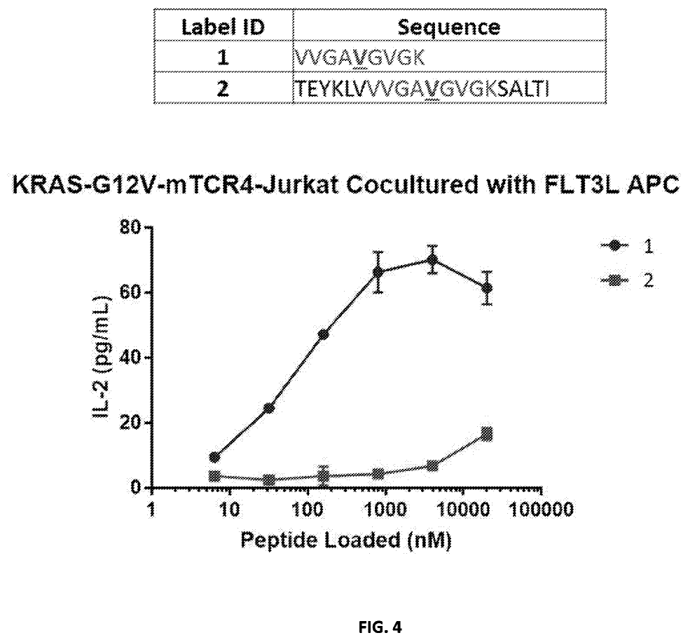

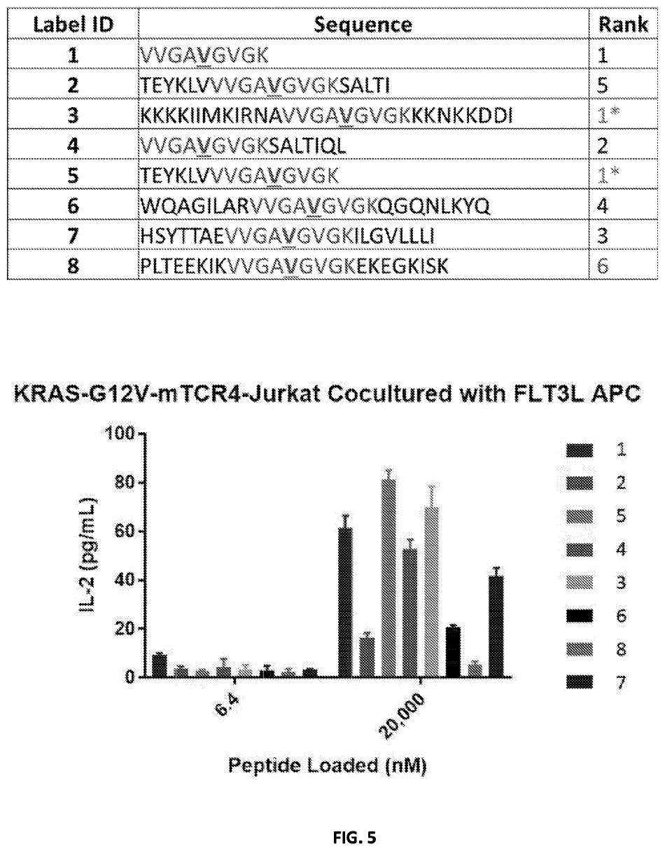

T cell receptor (TCR)-transduced cells were used to screen polypeptides in vitro for epitope processing and presentation. Engineered Jurkat cells expressing CD8 along with validated TCRs were prepared as effector cells. For target cells, peripheral blood mononuclear cells (PBMCs) with specific HLA alleles were stimulated with FLT3-ligand overnight, loaded with polypeptides containing the epitope of interest in different contexts for an hour, and matured with cytokines. Engineered Jurkat cells and PBMCs were co-cultured for 48 hours and the level of IL-2 secreted by engineered Jurkat cells was measured as a readout for peptide recognition by TCRs. The experimental design is shown in FIG. 3 and the results are shown in FIGS. 4 and 5.

### Example 2—Neoantigenic Polypeptide Immunogenecity

The immunogenicity of multiple polypeptides designed around specific epitopes, as well as the quality of the T cell responses to these polypeptides was studied. Eighty-four 8-12 week old female C57BL/6 mice (Taconic Biosciences) were randomly and prospectively assigned to treatment groups on arrival. Animals were acclimated for three days prior to study commencement. Animals were maintained on LabDiet™ 5053 sterile rodent chow and sterile water provided ad libitum. 12 animals in Group 1 served as unvaccinated controls. 12 animals in each group 2-7 received 50 μg polyIC:LC and 10 μg of each polypeptide (defined in Table 13; bolded sequence represents minimal epitope) or molar-matched equivalent of alternative peptide designs (defined in Table 14). Kif18b was used as a CD4 helper peptide and was used unmodified in all mice in groups 2-7. Blood was collected by retro-orbital bleeding on days 7, 14, and 21. Animals were weighed and monitored for general health daily. Animals were euthanized by CO₂ overdose at study completion Day 21, if an animal lost >30% o of its body weight compared to its weight at Day 0; or if an animal was found moribund.

TABLE 13
Peptides used in the study

| | | Restric- | | Source |
|---|---|---|---|---|
| Antigen | Sequence | tion | Allele | Model |
| Alg8 | AVGITYTWTRLYASV | MHCI | H-2Kb | T3 |
| | LTGSLVSKTKK | | | |
| Lama4 | IQKISFFDGFEVGFNF | MHCI | H-2Kb | T3 |
| | RTLQPNGLLFYYT | | | |
| Adpgk | GIPVHLELASMTNMEL | MHCI | H-2Db | MC38 |
| | MSSIVHQQVFPT | | | |
| Repsi | GRVLELFRAAQLANDV | MHCI | H-2Db | MC38 |
| | VLQIMELCGATR | | | |
| Irgq | KARDETAALLNSAVLG | MHCI | H-2Db | MC38 |
| | AAPLFVPPAD | | | |
| Obsl1 | REGVELCPGNKYEMRR | MHCI | H-2Db | B16F10 |
| | HGTTHSLVIHD | | | |
| Kif18b | PSKPSFQEFVDWENVS | MHCII | I-Ab | B16F10 |
| | PELNSTDQPFL | | | |

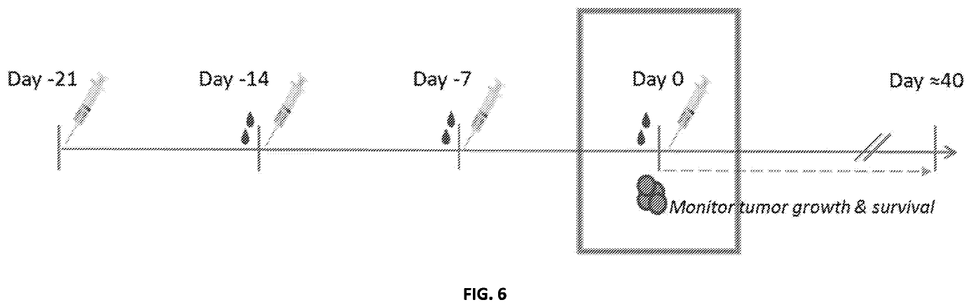

TABLE 14
Experimental Design (also see FIG. 6)

| | | | Antigens | Antigens | | | |
|---|---|---|---|---|---|---|---|
| | # of | | (Left Tail | (Scruff | Vaccine | Vaccine | Tissue |
| Group | Mice | Treatment | base) | of neck) | dose | treatment | collection |
| 1 | 12 | Untreated | N/A | N/A | N/A | N/A | N/A |
| 2 | 12 | Strong | Kif18b* | Alg8, | 10 μg each | Route: | RO bleeds: |
| | | SLPs + | | Lama4, | SLP | s.c. left tail | Days −14, −7, 0 |
| | | CD4 + | | Obsl1 | or | base in | |
| | | Hiltonol | | | Equimolar | 100 μL/ | |
| 3 | 12 | SLPs + | Reps1, | Alg8, | SSP | injection | |
| | | CD4 + | Adpgk, | Lama4, | 50 μg | Schedule: | |
| | | Hiltonol | Irgq, | Obsl1 | Hiltonol | Days −21, −14, −7 | |
| 4 | 12 | SSPs + | Kif18b* | | | | |
| | | CD4 + | | | | | |
| | | Hiltonol | | | | | |
| 5 | 12 | K4-SSPs + | | | | | |
| | | CD4 + | | | | | |
| | | Hiltonol | | | | | |
| 6 | 12 | K4-Val- | | | | | |
| | | Cit-PABC- | | | | | |
| | | SSPs + | | | | | |
| | | CD4 + | | | | | |
| | | Hiltonol | | | | | |
| 7 | 12 | K4- | | | | | |
| | | disulfide- | | | | | |
| | | SSPs + | | | | | |
| | | CD4 + | | | | | |
| | | Hiltonol | | | | | |

MIHC tetramers are manufactured on-site and are used to measure peptide-specific T cell expansion in the immunogenicity assays. For the assessment, tetramer is added to 1×10⁵ cells in PBS containing 1% FCS and 0.1% o sodium azide (FACS buffer). Cells are incubated in the dark for 15 minutes at 37° C. Antibodies specific for T cell markers, such as CD8, and for irrelevant cell types, such as CD4/CD11b/CD11c/CD19, are then added to a final concentration suggested by the manufacturer, and the cells are incubated in the dark at 4° C. for 20 minutes. Cells are washed with cold FACS buffer, immediately analyzed on an LSR2 (Becton Dickinson) instrument, and analyzed by use of FacsDiva software (Becton Dickinson). For analysis of tetramer positive cells, the lymphocyte gate is taken from the forward and side-scatter plots. Data are reported as the percentage of cells that were CD4⁻ CD11b⁻ CD11c⁻ CD19⁻CD8⁺/tetramer⁺.

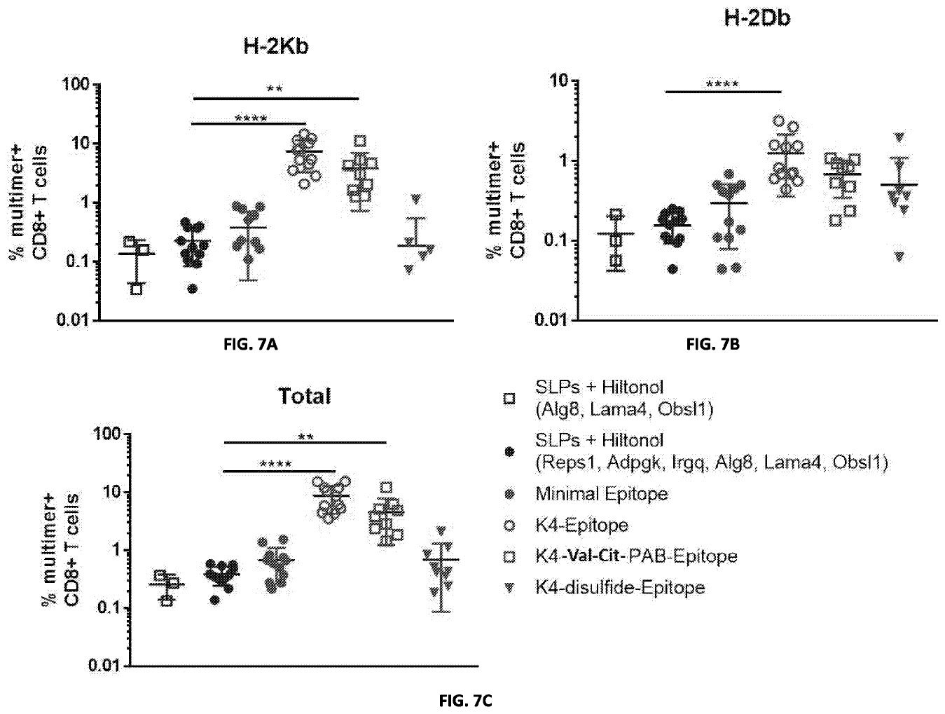

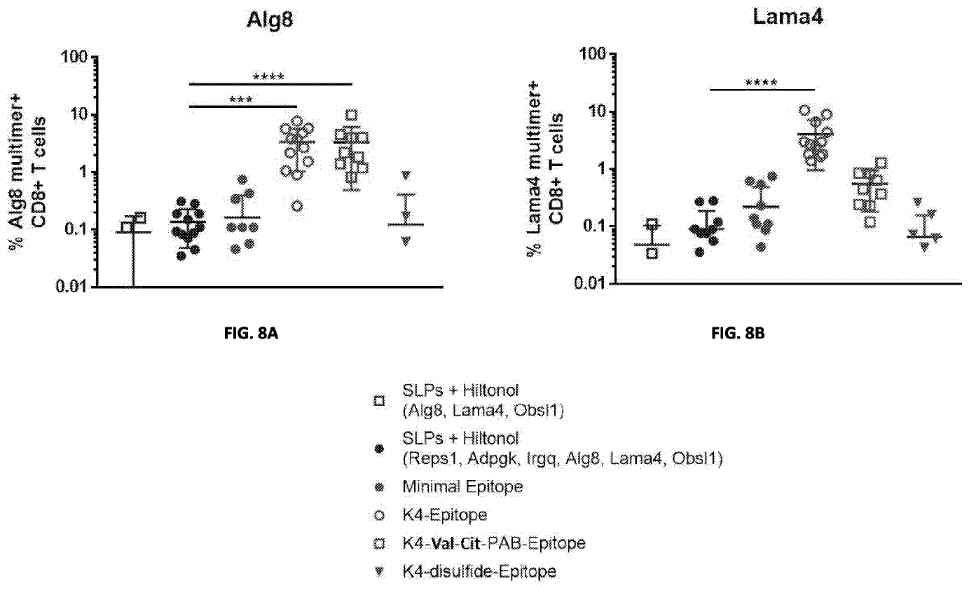

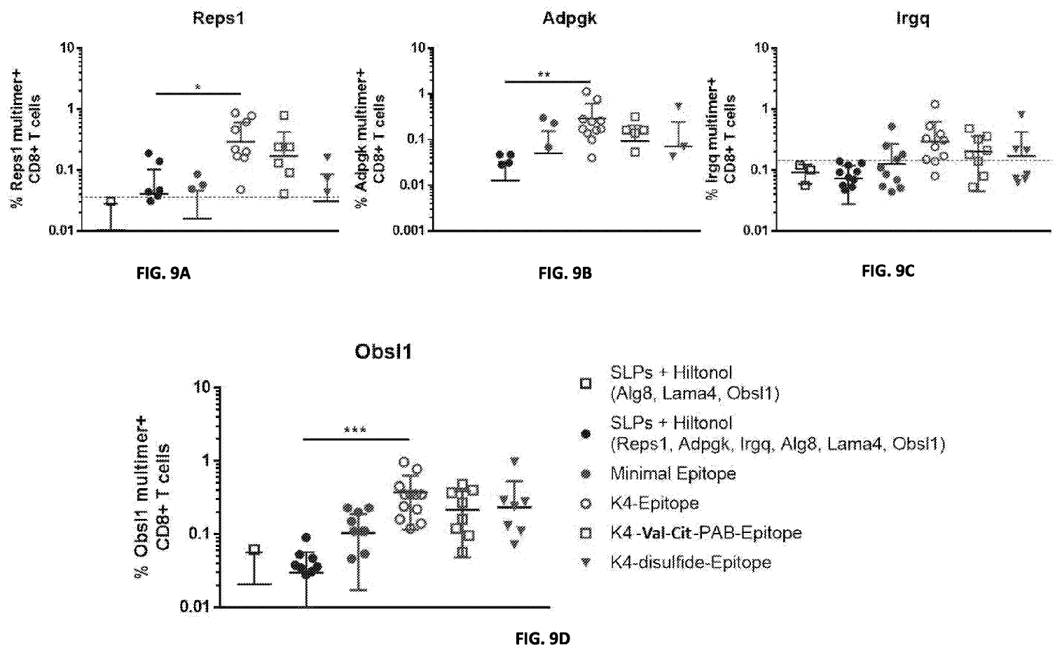

Immunization with K4-epitope significantly increased immune responses to 5 out of 6 epitopes assessed. Immune responses to Alg8, Lama4, Reps1, Adpgk, and Obsl1 are significantly increased. Immunization with K4-epitope increases immunogenicity of poorly immunogenic epitopes (e.g., Obsl1). K4-Val-Cit-PABC-epitope immunization increases Alg8-specific immune responses. The results are shown in FIGS. 7-9.

### Example 3—Synthesis of Disulfide Linker (Compound 5)

Step 1

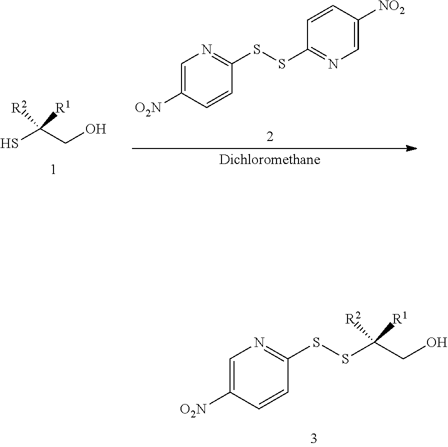

2,2′-bis(5-nitropyridyl) disulfide 2 (2 mmol) was suspended in 10 mL dichloromethane and the corresponding mercapto alcohol 1 (1 mmol, wherein R¹ and R² are as defined herein) in dichloromethane (4 mL) was added to the suspension. The resulting suspension was stirred at room temperature for 16 hours. The solvent was removed under reduced pressure. The resulting residue was re-dissolved in 5 mL dimethylformamide and purified using a C18-reverse phase column with a gradient of acetonitrile and water containing 0.05% TFA. The desired fractions were combined and lyophilized to give (5-nitropyridine-2-yl) disulfaneylalkyl alcohol 3 (˜65-82% yield, >90% purity, UPLC-MS/UV analysis at 220 nm).

Step 2

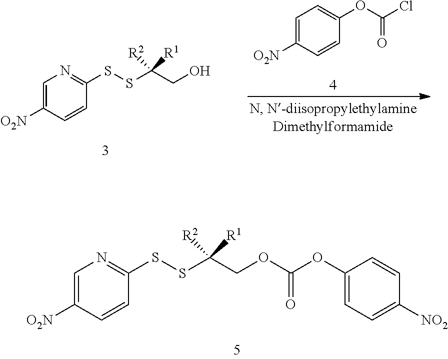

To a solution of (5-nitropyridine-2-yl) disulfaneylalkyl alcohol 3 (0.5 mmol, wherein R¹ and R² are as defined herein) in dimethylformamide (2 mL) was added N,N′-diisopropylethylamine (1.5 mmol) followed by addition of 4-nitrophenyl chloroformate 4 (0.55 mmol). This solution was stirred at room temperature for 16 hours, then purified using a C18-reverse phase column with a gradient of acetonitrile and water containing 0.05% TFA. The desired fractions were combined and lyophilized to give 4-nitrophenyl-(5-nitropyridine-2-yl) disulfaneylalkyl carbonate 5 (˜90-98% yield, >90% purity, UPLC-MS/UV analysis at 220 nm).

### Example 4—Synthesis of Disulfide Containing Peptides

Step 1: Formation of 4-nitro-2-pyridylthio Activated Disulfide Peptide 8

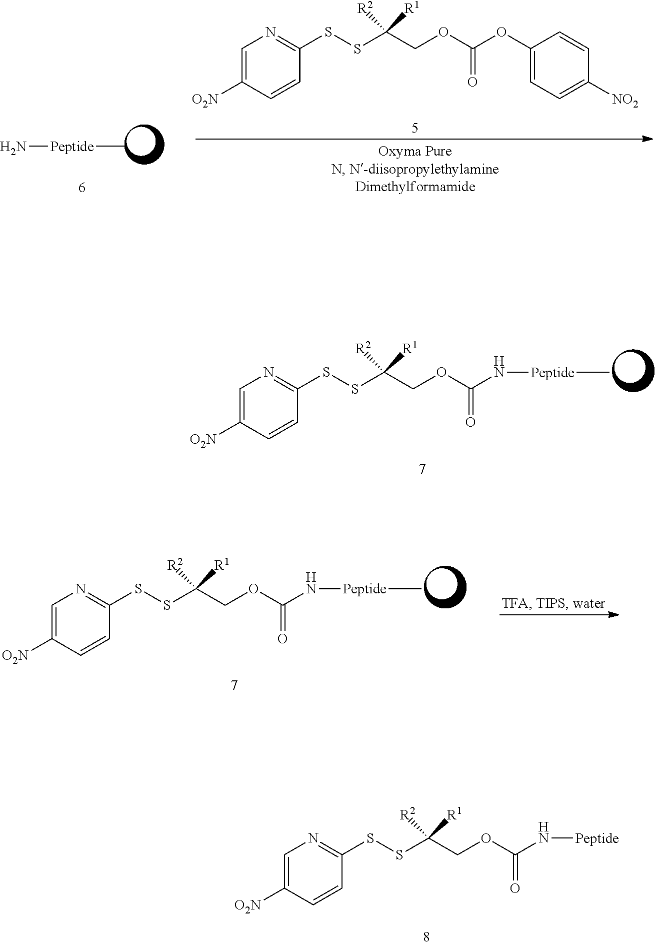

According to the above scheme, the N-terminus end of the peptide bound resin 6 (any resin made for solid phase peptide synthesis can be used) was acylated using Linker 5 (wherein R¹ and R² are as defined herein) manually, or programmed accordingly on an automatic peptide synthesizer. More specifically, resin 6 (0.05 mmol) was swelled in dimethylformamide for 5 minutes, and drained. The corresponding 4-nitrophenyl-(5-nitropyridine-2-yl) disulfaneylalkyl carbonate 5 (0.2 mmol) and Oxyma Pure Novabiochem® (also known as Oxyma Pure, 0.3 mmol) were dissolved in 1 mL dimethylformamide, added to swelled resin 6, and then N, N′-diisopropylethylamine (0.3 mmol) was added. The resulting resin suspension was agitated for 3 hours, drained, and resulting peptide bound resin 7 was then rinsed with dimethylformamide (5×, 5 mL), dichloromethane (5×, 5 mL), and methanol (2×, 5 mL). Peptide bound resin 7 was dried under reduced pressure for 1 hour, and cleaved using 3 mL 95% trifluoroacetic acid (TFA), 2.5% water, 2.5% triisopropylsilane (TIPS) at room temperature for 3 hours to form a cleavage solution (“A”) containing both unbound peptide 8 and the cleaved resin from 7. This cleavage solution A was then filtered and drained in a 50 mL conical tube, the cleaved resin from 7 was washed with a 95:5 TFA:water solution (1 mL), filtered, drained, and combined to result in a filtered peptide solution (“B”). Unbound peptide 8 was isolated from filtered peptide solution B by precipitating with ice cold diethyl ether, centrifuged at 3600 rpm for 5 minutes, and the diethyl ether was decanted. The resulting peptide pellet was then rinsed with 20 mL ice cold diethyl ether, to result in a suspension, which was then vortexed and centrifuged again at 3600 rpm for 3 minutes. This was repeated for a total of 3 washes to thoroughly rinse the pellet to result in 4-nitrophenyl-(5-nitropyridine-2-yl) disulfaneylalkyl carbamate peptide 8, which was carried into the next synthetic step without further purification.

Step 2: Disulfide Exchange Reaction to Form Disulfide Containing Peptide 10

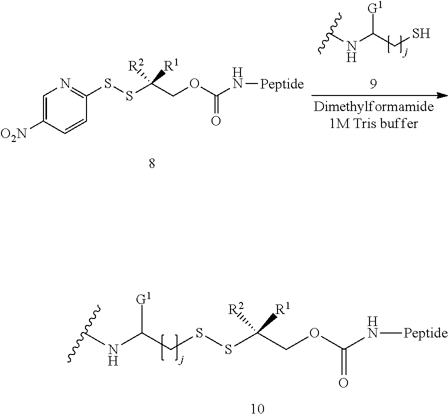

As described in the scheme above, crude 4-nitrophenyl-(5-nitropyridine-2-yl) disulfaneylalkyl carbamate peptide 8 (wherein R^{t} and R² are as defined herein) underwent the disulfide exchange with the desired thiol containing molecule 9 (wherein G^{t} and j are as defined herein). More specifically, the 4-nitrophenyl-(5-nitropyridine-2-yl) disulfaneylalkyl carbamate peptide 8 (0.05 mmol) was dissolved in dimethylformamide (1 mL), then a solution of desired thiol containing compound 9 (0.05 mmol) in 1:1 dimethylformamide-1M Tris buffer was added. The resulting yellow solution was stirred for 2 hours, purified using a C18-reverse phase column with a gradient of acetonitrile and water containing 0.05% TFA. The desired fractions were combined and lyophilized to result in disulfide containing peptide 10 (˜10-30% yield, >95% purity, UPLC-MS/UV analysis at 220 nm, starting from solid phase peptide synthesis).

### Example 5—Synthesis of PABC-Containing Peptide (13)

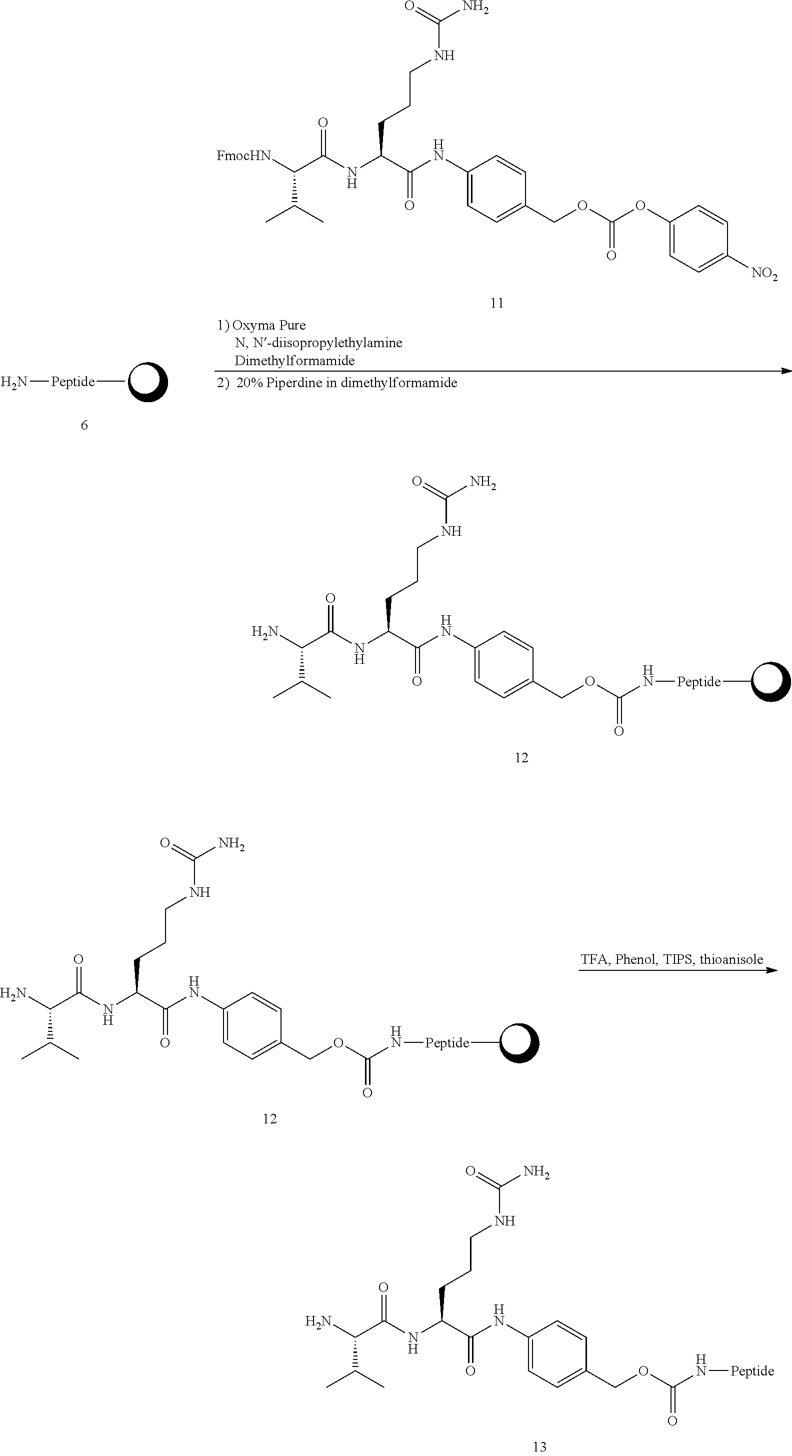

As described in the scheme above, the N-terminus end of peptide bound resin 6 was acylated using Fmoc-AA-AA-PAB-PNP 11 manually, or programmed accordingly on an automatic peptide synthesizer. More specifically, resin 6 (0.05 mmol) was swelled in dimethylformamide for 5 minutes, and drained. The corresponding Fmoc-AA-AA-PAB-PNP 11 (0.2 mmol) and Oxyma Pure Novabiochem® (also known as Oxyma Pure, 0.3 mmol) were dissolved in 1 mL dimethylformamide, added to resin 6, and then N, N′-diisopropylethylamine (0.3 mmol) was added. The resulting resin suspension was agitated for 3 hours, drained, and resulting Fmoc protected resin 12 was then rinsed with dimethylformamide (5×, 5 mL). The final N-terminal α-Fmoc was removed with 20% piperidine in dimethylformamide (2×, 5 minutes). At this point, the deprotected intermediate 12 may be optionally reacted with additional amino acid residues at the N-terminal end of 12 using standard Fmoc solid phase peptide syntheses, followed by N-terminal α-Fmoc deprotection(s) using an analogous procedure as discussed immediately above. After desired Fmoc deprotection(s) were completed, then resin 12 (or analogs with extended amino acids) was rinsed with dimethylformamide (5×, 5 mL), dichloromethane (5×, 5 mL), and then methanol (2×, 5 mL). Peptide bound resin 12 was dried under reduced pressure for 1 hour, and cleaved using 3 mL 70% trifluoroacetic acid (TFA), 10% Phenol, 10% triisopropylsilane (TIPS) and 10% thioanisole at room temperature for 30 minutes to form a cleavage solution (“A”) containing both unbound peptide 13 and cleaved resin from 12. This cleavage solution A was then filtered and drained to result in a filtered peptide solution (“B”) in a 50 mL conical tube. The cleaved resin from 12 was washed with 95:5 TFA:water solution (1 mL), filtered, drained, and combined with filtered peptide solution B. Unbound peptide 13 was isolated from filtered peptide solution B by precipitating with ice cold diethyl ether, centrifuged at 3600 rpm for 5 minutes and the diethylether was decanted. The resulting peptide pellet was then rinsed with 20 mL ice cold diethyl ether to result in a suspension, which was then vortexed and centrifuged again at 3600 rpm for 3 minutes. This was repeated for a total of 3 washes to thoroughly rinse the pellet to result in compound 13 (˜10-30% yield, >95% purity, UPLC-MS/UV analysis at 220 nm, starting from solid phase peptide synthesis).

### Example 6—Assessment of the TMPRSS2::ERG Epitope Processing

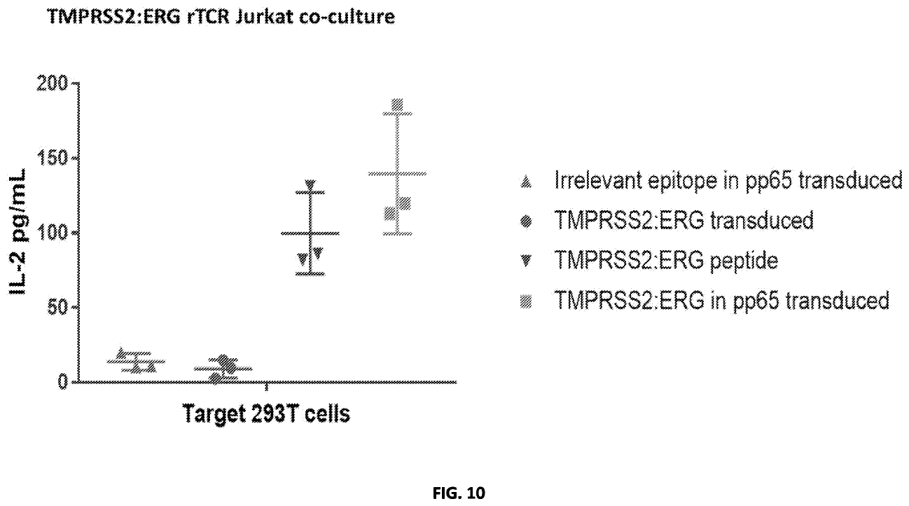

T cell receptor (TCR)-transduced cells were used to assess the TMPRSS2::ERG epitope processing and presentation on HLA-A02:01 in vitro. Engineered Jurkat cells expressing CD8 along with validated TCRs were prepared as effector cells. For target cells, 293T cells, which naturally express HLA-A02:01, were i) loaded with peptides containing the TMPRSS2::ERG epitope only for 24 hours or ii) stably transduced with a plasmid encoding a peptide containing the TMPRSS2::ERG epitope in different contexts (epitope in natural context i.e., the peptide additionally comprises an amino acid or an amino acid sequence that is naturally flanking the epitope sequence on the N- and/or C-terminus, epitope in non-natural context i.e., the peptide additionally comprises an amino acid or an amino acid sequence that is not naturally flanking the epitope sequence, e.g., CMVpp65 sequence) or a plasmid encoding a peptide containing an irrelevant epitope in non-natural context (as a control). Engineered Jurkat cells and 293T cells were co-cultured for 24 hours and the level of IL-2 secreted by engineered Jurkat cells was measured as a readout for peptide recognition by TCRs. The results are shown in FIG. 10.

### Example 7-Comparison of Enhanced Cleavage and Processing and Immunogenicity of Polypeptides

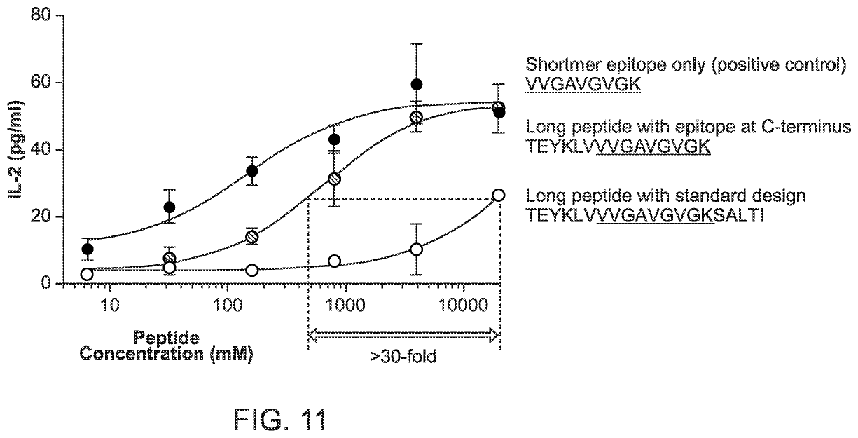

T cell receptor (TCR)-transduced cells were used for in vitro comparison of processing of the RAS-G12V-HLA-A11:01 epitope from peptides containing the RAS-G12V epitope only, the RAS-G12V epitope and additional amino acid sequence flanking the epitope on the N-terminus only, or the RAS-G12V epitope and additional amino acid sequences flanking the epitope on both N- and C-terminus for epitope processing and presentation. Engineered Jurkat cells expressing CD8 along with validated TCRs were prepared as effector cells. For target cells, peripheral blood mononuclear cells (PBMCs) with specific HLA alleles were stimulated with FLT3-ligand overnight, loaded with polypeptides containing the RAS-G12V epitope in different contexts for an hour, and matured with cytokines. Engineered Jurkat cells and PBMCs were co-cultured for 48 hours and the level of IL-2 secreted by engineered Jurkat cells was measured as a readout for peptide recognition by TCRs. The results are shown in FIG. 11.

### Example 8—Immunogenicity Evaluation of RAS Mutant Peptides with Differing Contexts Around Epitopes

Materials:

AIM V media (Invitrogen)
Human FLT3L, preclinical CellGenix #1415-050 Stock 50 ng/μL
TNF-α, preclinical CellGenix #1406-050 Stock 10 ng/μL
IL-1β, preclinical CellGenix #1411-050 Stock 10 ng/μL
PGE1 or Alprostadil—Cayman from Czech republic Stock 0.5 μg/μL
R10 media—RPMI 1640 glutamax+10% Human serum+1% PenStrep
20/80 Media—18% AIM V+72% RPMI 1640 glutamax+10% Human Serum+1% PenStrep
IL7 Stock 5 ng/μL
IL15 Stock 5 ng/μL

### Procedure:

Step 1: Plate 5 million PBMCs (or cells of interest) in each well of 24 well plate with FLT3L in 2 mL AIM
V media
Step 2: Peptide loading and maturation—in AIMV
1. Mix peptide pool of interest (except for no peptide condition) with PBMCs (or cells of interest) in respective wells.

### 2. Incubate for 1 hr.

3. Mix Maturation cocktail (including TNF-α, IL-1β, PGE1, and IL-7) to each well after incubation.
Step 3: Add human serum to each well at a final concentration of 10% by volume and mix.
Step 4: Replace the media with fresh RPMI+10% HS media supplemented with IL7+IL15,
Step 5: Replace the media with fresh 20/80 media supplemented with IL7+IL15 during the period of incubation every 1-6 days.
Step 6: Plate 5 million PBMCs (or cells of interest) in each well of new 6-well plate with FLT3L in 2 ml AIM V media
Step 7: Peptide loading and maturation for re-stimulation—(new plates)
1. Mix peptide pool of interest (except for no peptide condition) with PBMCs (or cells of interest) in respective wells

### 2. Incubate for 1 hr.

3. Mix Maturation cocktail to each well after incubation

### Step 8: Re-stimulation:

1. Count first stimulation FLT3L cultures and add 5 million cultured cells to the new Re-stimulation plates.
2. Bring the culture volume to 5 mL (AIM V) and add 500 ul of Human serum (10% by volume)
Step 9: Remove 3 ml of the media and add 6 ml of RPMI+10% HS media supplemented with IL7+IL15.
Step 10: Replace 75% of the media with fresh 20/80 media supplemented with IL7+IL15.
Step 11: Repeat re-stimulation if needed.

### Analysis of Antigen-Specific Induction

MHC tetramers are purchased or manufactured on-site, and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a LSR Fortessa (Becton Dickinson) instrument, and are analyzed by use of FlowJo software (Becton Dickinson). For analysis of tetramer positive cells, the lymphocyte gate is taken from the forward and side-scatter plots. Data are reported as the percentage of cells that were CD8+/Tetramer+.

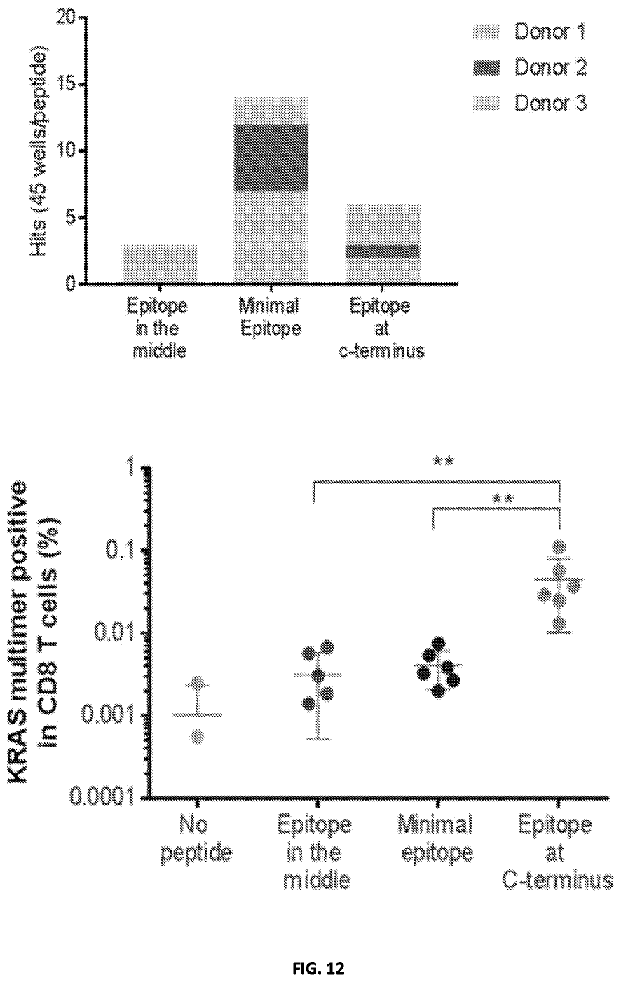

The peptide immunogenicity workflow (i.e., T cell induction and tetramer analysis) were used to evaluate the relative immunogenicity of the three peptide designs described in FIG. 11. Exemplary data showing increased immunogenicity of the peptide with the epitope at the c-terminus relative to the peptide with the epitope in the middle based on hit rate across 3 donors is shown in FIG. 12, top. The same three peptide designs were also evaluated using an in vivo mouse vaccination strategy as described in Example 2. Exemplary data showing the showing increased immunogenicity of the peptide with the epitope at the c-terminus relative to the peptide with the epitope in the middle is shown in FIG. 12, bottom.

### Example 9—High CD8 Hit Rate when APCs were Stimulated with mRNA Encoding Peptides

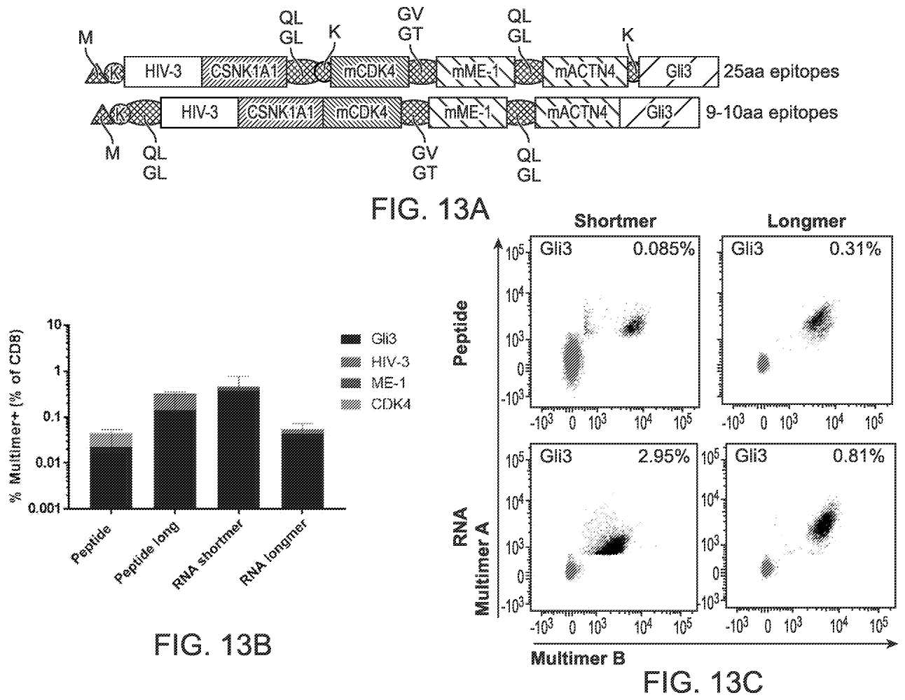

Shortmers (9-10 amino acids) or longmers (25 amino acids) were constructed in the form of a concatenated neoantigen string as shown graphically in FIG. 13A. Sequences for antigens are represented by colored boxes. Linker sequences (K, QLGL, or GVGT—represented as blue circles) were added in between antigen sequences as predicted by NetChop (an algorithm to predict cleavage of the human proteome). If a sequence was predicted to cleave within the antigen sequence, cleavage sites were added to promote cleavage between antigen sequences. PBMCs were then nucleofected with the aforementioned multi-antigen encoding mRNA constructs and were used to stimulate T cells. A side by side comparison was performed with pools of congruent peptides, and their length and sequence were the same as those encoded within the RNA strings. Short and long RNA sequences raise similar CD8⁺ T cell responsive to multimers (Table 15). Notably, robust CD8 responses were observed using mRNA encoding longmers and shortmers.

TABLE 15
Comparison of peptide and RNA longer and shortmer mediated
activation

| | | | Mean | | |
|---|---|---|---|---|---|
| | | CD8 | Neoantigen + | Diversity | |
| | | Hit | Frequency | of | |
| | | Rate | (% CD8 | responses | CD4 |
| | | (%) | cells) | (out of 6) | responses |
| Donor | Peptide Short | 7 | 0.03% | 1 | N.A. |
| 1 | Peptide Long | 19 | 0.09% | 2 | 0 |
| | RNA Short | 11 | 1.50% | 2 | N.A. |
| | RNA Long | 8 | 0.36% | 2 | 0 |
| Donor | Peptide Short | 11 | 0.03% | 2 | N.A. |
| 2 | Peptide Long | 17 | 0.21% | 3 | 1 |
| | RNA Short | 19 | 0.39% | 2 | N.A. |
| | RNA Long | 20 | 0.05% | 2 | 0 |

As shown in FIG. 13B, Gli3 epitope is well represented and presented by the peptides as well as mRNA, however, mRNA encoded Gli3 shortmer epitope loaded PBMCs resulted in higher Gli3-specific CD8+ T cells (as detected by a multimer assay). Representative flow cytometry results for a multimer assay are shown in FIG. 13C. In this string, the sequence preceding the Gli3 sequence is from a non-natural context. This may have enhanced the processing and presentation of Gli3 from the poly-peptide string, and have increased the response compared to peptide. Additionally, the mRNA shortmer string gave rise to a ME-1 T cell response, which was not present in the congruent short peptide pool. In our strings, ME-1 has cleavage sites before and after the epitope sequence, and this enhanced processing and presentation on the epitope could lead to superior T cell responses.

### PARAGRAPHS OF EMBODIMENTS

A polypeptide comprising an epitope presented by a class I MHC or a class II MHC of an antigen presenting cell (APC), the polypeptide having a structure of Formula (I):

Yₙ-Bₜ-Aᵣ-Xₘ-Aₛ-Cᵤ-Zₚ Formula (I),

or a pharmaceutically acceptable salt thereof,
(i) wherein Xₘ is the epitope, wherein each X independently represents an amino acid of a contiguous amino acid sequence encoded by a nucleic acid sequence in a genome of a subject, and wherein, (a) the MHC is a class I MHC and m is an integer from 8 to 12, or
   (b) the MHC is a class II MHC and m is an integer from 9 to 25;
(ii) wherein each Y is independently an amino acid, analog, or derivative thereof, and wherein:
   (A) when variable r of Aᵣ in Formula (I) is 0, Yₙ is not encoded by a nucleic acid sequence immediately upstream of the nucleic acid sequence in the genome of the subject that encodes Bₜ-Aᵣ-Xₘ,
   (B) when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or
   (C) when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and further wherein, n is an integer from 0 to 1000;
(iii) wherein each Z is independently an amino acid, analog, or derivative thereof, and wherein:
   (A) when variable s of Aₛ in Formula (I) is 0, Zₚ is not encoded by a nucleic acid sequence immediately downstream of the nucleic acid sequence in the genome of the subject that encodes Xₘ-Aₛ-Cᵤ,
   (B) when variable s of Aₛ in Formula (I) is 1 and variable u of Cᵤ in Formula (I) is 0, Zₚ is not encoded by a nucleic acid sequence immediately downstream of the nucleic acid sequence in the genome of the subject that encodes Xₘ, or
   (C) when variable s of Aₛ in Formula (I) is 1 and variable u of Cᵤ in Formula (I) is 1 or more, Zₚ is not encoded by a nucleic acid sequence immediately downstream of the nucleic acid sequence in the genome of the subject that encodes Cᵤ; and
   further wherein, p is an integer from 0 to 1000;
   and further wherein,
   when n is 0, p is an integer from 1 to 1000; and
   when p is 0, n is an integer from 1 to 1000;
(iv) wherein Aᵣ is a linker, and r is 0 or 1;
(v) wherein Aₛ is a linker, and s is 0 or 1;
(vi) wherein each B independently represents an amino acid encoded by a nucleic acid sequence in the genome of the subject that is immediately upstream of the nucleic acid sequence in the genome of the subject that encodes Xₘ,
   and wherein t is an integer from 0 to 1000; and
(vii) wherein each C independently represents an amino acid encoded by a nucleic acid sequence in the genome of the subject that is immediately downstream of the nucleic acid sequence in the genome of the subject that encodes Xₘ,
   and wherein, u is an integer from 0 to 1000;

and further wherein,

(a) the polypeptide does not consist of four different epitopes presented by a class I MHC;

(b) the polypeptide comprises at least two different polypeptide molecules;

(c) the epitope comprises at least one mutant amino acid; and/or

(d) Yₙ and/or Zₚ is cleaved from the epitope when the polypeptide is processed by the APC.

The polypeptide of paragraph [0492], wherein the epitope is presented by a class II MHC.

The polypeptide of paragraph [0492] or [0493], wherein m is an integer from 9 to 25.

The polypeptide of any one of paragraphs [0492]-[0494], wherein t is 1, 2, 3, 4, or 5 or more and r is 0.

The polypeptide of any one of paragraphs [0492]-[0495], wherein u is 1, 2, 3, 4, or 5 or more and s is 0.

The polypeptide of any one of paragraphs [0492]-[0496], wherein t is 1 or more, r is 0, and n is from 1-1000.

The polypeptide of any one of paragraphs [0492]-[0497], wherein u is 1 or more, s is 0, and p is from 1-1000.

The polypeptide of any one of paragraphs [0492]-[0498], wherein t is 0.

The polypeptide of any one of paragraphs [0492]-[0499], wherein u is 0.

The polypeptide of any one of paragraphs [0492]-[0500], wherein t is at least 1 and Bₜ comprises a lysine.

The polypeptide of any one of paragraphs [0492]-[0501], wherein u is at least 1 and Cᵤ comprises a lysine.

The polypeptide of any one of paragraphs [0492]-[0502], wherein Bₜ is cleaved from the epitope when the polypeptide is processed by the APC.

The polypeptide of any one of paragraphs [0492]-[0503], wherein Cᵤ is cleaved from the epitope when the polypeptide is processed by the APC.

The polypeptide of any one of paragraphs [0492]-[0504], wherein n is an integer from 1 to 5 or 7-1000.

The polypeptide of any one of paragraphs [0492]-[0505], wherein p is an integer from 1 to 4 or 6-1000.

The polypeptide of any one of paragraphs [0492]-[0506], wherein the polypeptide does not consist of four different epitopes presented by a class I MHC.

The polypeptide of any one of paragraphs [0492]-[0507], wherein the polypeptide does not comprise four different epitopes presented by a class I MHC.

The polypeptide of any one of paragraphs [0492]-[0508], wherein the polypeptide comprises at least two different polypeptide molecules.

The polypeptide any one of paragraphs [0492]-[0509], wherein the epitope comprises at least one mutant amino acid.

The polypeptide of paragraph [0510], wherein the at least one mutant amino acid is encoded by an insertion, a deletion, a frameshift, a neoORF, or a point mutation in the nucleic acid sequence in the genome of the subject.

The polypeptide of any one of paragraphs [0492]-[0511], wherein Yₙ and/or Zₚ is cleaved from the epitope when the polypeptide is processed by the APC.

The polypeptide of any one of paragraphs [0492]-[0512], wherein m of Xₘ is at least 8 and wherein Xₘ is AA₁AA₂AA₃AA₄AA₅AA₆AA₇AA₈AA₉AA₁₀AA₁₁AA₁₂AA₁₃AA₁₄AA₁₅AA₁₆AA₁₇AA₁₈AA₁₉AA₂₀AA₂₁AA₂₂A A₂₃AA₂₄AA₂₅, wherein each AA is an amino acid, and wherein one or more of AA₉, AA₁₀, AA₁₁, AA₁₂, AA₁₃, AA₁₄, AA₁₅, AA₁₆, AA₁₇, AA₁₈, AA₁₉, AA₂₀, AA₂₁, AA₂₂, AA₂₃, AA₂₄, and AA₂₅ are optionally present, and further wherein at least one AA is a mutant amino acid.

The polypeptide of any one of paragraphs [0492]-[0513], wherein r is 1.

The polypeptide of any one of paragraphs [0492]-[0514], wherein s is 1.

The polypeptide of any one of paragraphs [0492]-[0515], wherein r is 1 and s is 1.

The polypeptide of any one of paragraphs [0492]-[0516], wherein r is 0.

The polypeptide of any one of paragraphs [0492]-[0517], wherein s is 0.

The polypeptide of any one of paragraphs [0492]-[0518], wherein r is 0 and s is 0.

The polypeptide of any one of paragraphs [0492]-[0519], wherein Aᵣ and/or Aₛ is a non-polypeptide linker.

The polypeptide of any one of paragraphs [0492]-[0520], wherein Aᵣ and/or Aₛ is chemical linker.

The polypeptide of any one of paragraphs [0492]-[0521], wherein Aᵣ and/or Aₛ comprises a non-natural amino acid.

The polypeptide of any one of paragraphs [0492]-[0522], wherein Aᵣ and/or Aₛ does not comprise an amino acid.

The polypeptide of any one of paragraphs [0492]-[0523], wherein Aᵣ and/or Aₛ does not comprise a natural amino acid.

The polypeptide of any one of paragraphs [0492]-[0524], wherein Aᵣ and/or Aₛ comprises a bond other than a peptide bond.

The polypeptide of any one of paragraphs [0492]-[0525], wherein Aᵣ and/or Aₛ comprises a disulfide bond.

The polypeptide of any one of paragraphs [0492]-[0526], wherein Aᵣ and Aₛ are different.

The polypeptide of any one of paragraphs [0492]-[0527], wherein Aᵣ and Aₛ are the same.

The polypeptide of any one of paragraphs [0492]-[0528], wherein the polypeptide comprises a hydrophilic tail.

The polypeptide of any one of paragraphs [0492]-[0529], wherein Yₙ-Bₜ-Aᵣ and/or Aₛ-Cᵤ-Zₚ enhances solubility of the polypeptide compared to a corresponding peptide that does not contain Yₙ-Bₜ-Aᵣ and/or Aₛ-Cᵤ-Zₚ.

The polypeptide of any one of paragraphs [0492]-[0530], wherein each X of Xₘ is a natural amino acid.

The polypeptide of any one of paragraphs [0492]-[0531], wherein the epitope is released from Yₙ-Bₜ-Aᵣ and/or Aₛ-Cᵤ-Zₚ when the polypeptide is processed by the APC.

The polypeptide of any one of paragraphs [0492]-[0532], wherein the polypeptide is cleaved at Aᵣ and/or Aₛ.

The polypeptide of any one of paragraphs [0492]-[0533], wherein the polypeptide is cleaved at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ; and/or wherein the polypeptide is cleaved at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ.

The polypeptide of any one of paragraphs [0492]-[0533], wherein the polypeptide is cleaved at a higher rate when n is an integer from 1 to 1000 compared to cleavage of a corresponding polypeptide of the same length that comprises Bₜ-Xₘ wherein t is at least one and r of variable Aᵣ in Formula (I) is 0; and/or wherein the polypeptide is cleaved at a higher rate when p is an integer from 1 to 1000 compared to cleavage of a corresponding polypeptide of the same length that comprises Xₘ-Cᵤ wherein u is at least one and s of variable Aₛ in Formula (I) is 0.

The polypeptide of any one of paragraphs [0492]-[0535], wherein the polypeptide is cleaved at Aᵣ at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ; and/or wherein the polypeptide is cleaved at Aₛ at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ.

The polypeptide of any one of paragraphs [0492]-[0536], wherein epitope presentation by the APC is enhanced when n is an integer from 1 to 1000 compared to epitope presentation of a corresponding polypeptide of the same length that comprises Xₘ and at least one additional amino acid encoded by a nucleic acid sequence immediately upstream of the nucleic acid sequence in the genome of the subject that encodes Xₘ; and/or wherein epitope presentation by the APC is enhanced when p is an integer from 1 to 1000 compared to epitope presentation of a corresponding polypeptide of the same length that comprises Xₘ and at least one additional amino acid encoded by a nucleic acid sequence immediately downstream of the nucleic acid sequence in the genome of the subject that encodes Xₘ.

The polypeptide of any one of paragraphs [0492]-[0536], wherein epitope presentation by the APC is enhanced when n is an integer from 1 to 1000 compared to epitope presentation of a corresponding polypeptide of the same length that comprises Bₜ-Xₘ wherein t is at least one and r of variable Aᵣ in Formula (I) is 0; and/or wherein epitope presentation by the APC is enhanced when p is an integer from 1 to 1000 compared to epitope presentation of a corresponding polypeptide of the same length that comprises Xₘ-Cᵤ wherein u is at least one and s of variable Aₛ in Formula (I) is 0.

The polypeptide of any one of paragraphs [0492]-[0538], wherein the APC presents the epitope to an immune cell.

The polypeptide of any one of paragraphs [0492]-[0539], wherein the APC presents the epitope to a phagocytic cell.

The polypeptide of any one of paragraphs [0492]-[0540], wherein the APC presents the epitope to a dendritic cell, a macrophage, a mast cell, a neutrophil, or a monocyte.

The polypeptide of any one of paragraphs [0492]-[0541], wherein the APC presents the epitope preferentially or specifically to the immune cell, the phagocytic cell, the dendritic cell, the macrophage, the mast cell, the neutrophil, or the monocyte.

The polypeptide of any one of paragraphs [0492]-[0542], wherein immunogenicity is enhanced when n is an integer from 1 to 1000 compared to immunogenicity of a corresponding polypeptide of the same length that comprises Xₘ and at least one additional amino acid encoded by a nucleic acid sequence immediately upstream of the nucleic acid sequence in the genome of the subject that encodes Xₘ; and/or wherein immunogenicity is enhanced when p is an integer from 1 to 1000 compared to immunogenicity of a corresponding polypeptide of the same length that comprises Xₘ and at least one additional amino acid encoded by a nucleic acid sequence immediately downstream of the nucleic acid sequence in the genome of the subject that encodes Xₘ.

The polypeptide of any one of paragraphs [0492]-[0542], wherein immunogenicity is enhanced when n is an integer from 1 to 1000 compared to immunogenicity of a corresponding polypeptide of the same length that comprises Bₜ-Xₘ wherein t is at least one and r of variable Aᵣ in Formula (I) is 0; and/or wherein immunogenicity is enhanced when p is an integer from 1 to 1000 compared to immunogenicity of a corresponding polypeptide of the same length that comprises Xₘ-Cᵤ wherein u is at least one and s of variable Aₛ in Formula (I) is 0.

The polypeptide of any one of paragraphs [0492]-[0544], wherein anti-tumor activity is enhanced when n is an integer from 1 to 1000 compared to anti-tumor activity of a corresponding polypeptide of the same length that comprises Xₘ and at least one additional amino acid encoded by a nucleic acid sequence immediately upstream of the nucleic acid sequence in the genome of the subject that encodes Xₘ; and/or wherein anti-tumor activity is enhanced when p is an integer from 1 to 1000 compared to anti-tumor activity of a corresponding polypeptide of the same length that comprises Xₘ and at least one additional amino acid encoded by a nucleic acid sequence immediately downstream of the nucleic acid sequence in the genome of the subject that encodes Xₘ.

The polypeptide of any one of paragraphs [0492]-[0544], wherein anti-tumor activity is enhanced when n is an integer from 1 to 1000 compared to anti-tumor activity of a corresponding polypeptide of the same length that comprises Bₜ-Xₘ wherein t is at least one and r of variable Aᵣ in Formula (I) is 0; and/or wherein anti-tumor activity is enhanced when p is an integer from 1 to 1000 compared to anti-tumor activity of a corresponding polypeptide of the same length that comprises Xₘ-Cᵤ wherein u is at least one and s of the variable Aₛ in Formula (I) is 0.

The polypeptide of any one of paragraphs [0492]-[0546], wherein Yₙ and/or Zₚ comprises a sequence selected from the group consisting of poly-Lys (polyK) and poly-Arg (polyR).

The polypeptide of paragraph [0547], wherein Yₙ and/or Zₚ comprises a sequence selected from the group consisting of polyK-AA-AA and polyR-AA-AA, wherein each AA is an amino acid or analogue or derivative thereof.

The polypeptide of paragraph [0547] or [0548], wherein the polyK comprises poly-L-Lys.

The polypeptide of paragraph [0547] or [0548], wherein the polyR comprises poly-L-Arg.

The polypeptide of any one of paragraphs [0547]-[0550], wherein the polyK or polyR comprises at least three or four contiguous lysine or arginine residues, respectively.

The polypeptide of any one of paragraphs [0492]-[0551], wherein Aᵣ and/or Aₛ is selected from the group consisting of a disulfide; p-aminobenzyloxycarbonyl (PABC); and AA-AA-PABC, wherein each AA is an amino acid or analogue or derivative thereof.

The polypeptide of paragraph [0552], wherein AA-AA-PABC is selected from the group consisting of Ala-Lys-PABC, Val-Cit-PABC, and Phe-Lys-PABC.

The polypeptide of any one of paragraphs [0492]-[0551], wherein Aᵣ and/or Aₛ is

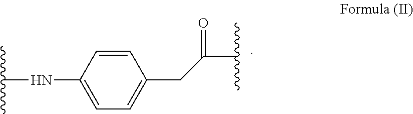 [SMILES: *Nc1ccc(CC(*)=O)cc1]

The polypeptide of any one of paragraphs [0492]-[0551], wherein Aᵣ and/or Aₛ is

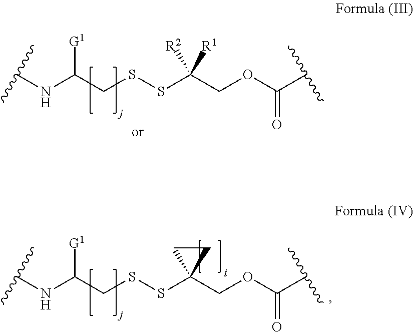

wherein,
R₁ and R₂ is independently H or an (C₁-C₆) alkyl;
j is 1 or 2;

### G₁ is H or COOH; and

i is 1, 2, 3, 4, or 5.

The polypeptide of any one of paragraphs [0492]-[0555], wherein the polypeptide is ubiquitinated.

The polypeptide of paragraph [0556], wherein the polypeptide is ubiquitinated prior to cleavage.

The polypeptide of paragraph [0556] or [0557], wherein the polypeptide is ubiquitinated on a lysine residue.

The polypeptide of any one of paragraphs [0492]-[0558], wherein the polypeptide is not cleaved before processing by an APC or before internalization by an APC in a subject.

The polypeptide of any one of paragraphs [0492]-[0559], wherein the polypeptide is not cleaved in blood in a subject before processing by an APC or before internalization by an APC.

The polypeptide of any one of paragraphs [0492]-[0560], wherein the polypeptide is not cleaved by a protease in blood.

The polypeptide of any one of paragraphs [0492]-[0561], wherein the polypeptide is not cleaved by plasmin, plasma kallikrein, tissue kallikrein, thrombin, or a coagulation factor.

The polypeptide of any one of paragraphs [0492]-[0562], wherein the polypeptide is stable in human plasma.

The polypeptide of any one of paragraphs [0492]-[0563], wherein the polypeptide has a half-life of from 1 hour to 5 days in human plasma.

The polypeptide of any one of paragraphs [0492]-[0564], wherein the polypeptide is cleaved in a lysosome, an endolysosome, an endosome, or an endoplasmic reticulum (ER).

The polypeptide of any one of paragraphs [0492]-[0565], wherein the polypeptide is cleaved by an aminopeptidase.

The polypeptide of paragraph [0566], wherein the aminopeptidase is an insulin-regulated aminopeptidase (IRAP) or an endoplasmic reticulum aminopeptidase (ERAP).

The polypeptide of any one of paragraphs [0492]-[0565], wherein the polypeptide is processed by a trypsin-like domain of a proteasome and/or an immunoproteasome.

The polypeptide of paragraph [0568], wherein the trypsin-like domain comprises trypsin-like activity, chymotrypsin-like activity, or peptidylglutamyl-peptide hydrolase (PGPH) activity.

The polypeptide of any one of paragraphs [0492]-[0565], wherein the polypeptide is cleaved by a protease.

The polypeptide of paragraph [0570], wherein the protease is a trypsin-like protease, a chymotrypsin-like protease, or a peptidylglutamyl-peptide hydrolase (PGPH).

The polypeptide of paragraph [0570], wherein the protease is selected from the group consisting of asparagine peptide lyase, aspartic protease, cysteine protease, glutamic protease, metalloprotease, serine protease, and threonine protease.

The polypeptide of paragraph [0572], wherein the protease is a cysteine protease selected from the group consisting of a Calpain, a Caspase, Cathepsin B, Cathepsin C, Cathepsin F, Cathepsin H, Cathepsin K, Cathepsin L1, Cathepsin L2, Cathepsin O, Cathepsin S, Cathepsin W, and Cathepsin Z.

The polypeptide of any one of paragraphs [0492]-[0573], wherein the subject is a mammal.

The polypeptide of any one of paragraphs [0492]-[0574], wherein the subject is a human.

The polypeptide of any one of paragraphs [0492]-[0575], wherein the epitope binds to a MHC I class HLA.

The polypeptide of paragraph [0576], wherein the epitope binds to the MHC I class HLA with a stability of 10 minutes to 24 hours.

The polypeptide of paragraph [0576], wherein the epitope binds to the MHC I class HLA with an affinity of 0.1 nM to 2000 nM.

The polypeptide of any one of paragraphs [0492]-[0575], wherein the epitope binds to MHC II class HLA.

The polypeptide of paragraph [0579], wherein the epitope binds to the MHC II class HLA with a stability of 10 minutes to 24 hours.

The polypeptide of paragraph [0579], wherein the epitope binds to the MHC II class HLA with an affinity of 0.1 nM to 2000 nM, 1 nM to 1000 nM, 10 nM to 500 nM, or less than 1000 nM.

The polypeptide of any one of paragraphs [0492]-[0581], wherein n is an integer from 1 to 20 or 5 to 12.

The polypeptide of any one of paragraphs [0492]-[0582], wherein p is an integer from 1 to 20 or 5 to 12.

The polypeptide of any one of paragraphs [0492]-[0583], wherein the epitope comprises a tumor-specific epitope.

The polypeptide of any one of paragraphs [0492]-[0584], wherein the polypeptide comprises at least two polypeptides, wherein two or more of the at least two polypeptides have the same formula Yₙ-Bₜ-Aᵣ-Xₘ-Aₛ-Cᵤ-Zₚ.

The polypeptide of paragraph [0585], wherein the polypeptide comprises at least at two polypeptide molecules.

The polypeptide of paragraph [0585] or [0586], wherein Xₘ of two or more of the at least two polypeptides or polypeptide molecules are the same.

The polypeptide of any one of paragraphs [0585]-[0587], wherein Yₙ of two or more of the at least two polypeptides or polypeptide molecules are the same.

The polypeptide of any one of paragraphs [0585]-[0588], wherein Zₚ of two or more of the at least two polypeptides or polypeptide molecules are the same.

The polypeptide of any one of paragraphs [0585]-[0589], wherein Aᵣ and/or Aₛ of two or more of the at least two polypeptides or polypeptide molecules are different.

The polypeptide of any one of paragraphs [0585]-[0590], wherein r=0 for a first of the at least two polypeptides or polypeptide molecules and r=1 for a second of the at least two polypeptides or polypeptide molecules.

The polypeptide of any one of paragraphs [0585]-[0591], wherein s=0 for a first of the at least two polypeptides or polypeptide molecules and s=1 for a second of the at least two polypeptides or polypeptide molecules.

The polypeptide of any one of paragraphs [0492]-[0592], wherein the polypeptide comprises at least 3, 4, 5, 6, 7, 8, 9, 10, or more polypeptides or polypeptide molecules.

The polypeptide of any one of paragraphs [0492]-[0593], wherein the epitope is a RAS epitope.

The polypeptide of paragraph [0594], wherein the epitope comprises a mutant RAS peptide sequence that comprises at least 8 contiguous amino acids of a mutant RAS protein comprising a mutation at G12, G13, or Q61 and the mutation at G12, G13, or Q61.

The polypeptide of paragraph [0595], wherein the at least 8 contiguous amino acids of a mutant RAS protein comprising a mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation.

The polypeptide of paragraph [0595] or [0596], wherein the mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation.

The polypeptide of any one of paragraphs [0492]-[0597], wherein Yₙ and/or Zₚ comprises an amino acid sequence of a protein of CMV such as pp65, HIV, or MART-1.

The polypeptide of any one of paragraphs [0492]-[0598], wherein n and/or p is 1, 2, 3, or an integer greater than 3.

The polypeptide of any one of paragraphs [0492]-[0599], wherein the epitope binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM.

The polypeptide of any one of paragraphs [0492]-[0600], wherein the epitope binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes.

The polypeptide of paragraph [0600] or [0601], wherein the HLA allele is selected from the group consisting of HLA-A02:01 allele, an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, an HLA-A74:01 allele, and/or an HLA-C08:02 allele and any combination thereof.

The polypeptide of any one of paragraphs [0492]-[0602], wherein the epitope comprises an amino acid sequence of GADGVGKSAL, GACGVGKSAL, GAVGVGKSAL, GADGVGKSA, GACGVGKSA, GAVGVGKSA, KLVVVGACGV, FLVVVGACGL, FMVVVGACGI, FLVVVGACGI, FMVVVGACGV, FLVVVGACGV, MLVVVGACGV, FMVVVGACGL, YLVVVGACGV, KMVVVGACGV, YMVVVGACGV, MMVVVGACGV, DTAGHEEY, TAGHEEYSAM, DILDTAGHE, DILDTAGH, ILDTAGHEE, ILDTAGHE, DILDTAGHEEY, DTAGHEEYS, LLDILDTAGH, DILDTAGRE, DILDTAGR, ILDTAGREE, ILDTAGRE, CLLDILDTAGR, TAGREEYSAM, REEYSAMRD, DTAGKEEYSAM, CLLDILDTAGK, DTAGKEEY, LLDILDTAGK, ILDTAGKE, ILDTAGKEE, DTAGLEEY, ILDTAGLE, DILDTAGL, ILDTAGLEE, GLEEYSAMRDQY, LLDILDTAGLE, LDILDTAGL, DILDTAGLE, DILDTAGLEEY, AGVGKSAL, GAAGVGKSAL, AAGVGKSAL, CGVGKSAL, ACGVGKSAL, DGVGKSAL, ADGVGKSAL, DGVGKSALTI, GARGVGKSA, KLVVVGARGV, VVVGARGV, SGVGKSAL, VVVGASGVGK, GASGVGKSAL, VGVGKSAL, VVVGAGCVGK, KLVVVGAGC, GDVGKSAL, DVGKSALTI, VVVGAGDVGK, TAGKEEYSAM, DTAGHEEYSAM, TAGHEEYSA, DTAGREEYSAM, TAGKEEYSA, AAGVGKSA, AGCVGKSAL, AGDVGKSAL, AGKEEYSAMR, AGVGKSALTI, ARGVGKSAL, ASGVGKSA, ASGVGKSAL, AVGVGKSA, CVGKSALTI, DILDTAGK, DILDTAGREEY, DTAGHEEYSAMR, DTAGKEEYS, DTAGKEEYSAMR, DTAGLEEYS, DTAGLEEYSA, DTAGLEEYSAMR, DTAGREEYS, DTAGREEYSAMR, GAAGVGKSA, GACGVGKSA, GACGVGKSAL, GADGVGKS, GAGDVGKSA, GAGDVGKSAL, GASGVGKSA, GCVGKSAL, GCVGKSALTI, GHEEYSAM, GKEEYSAM, GLEEYSAMR, GREEYSAM, GREEYSAMR, HEEYSAMRD, KEEYSAMRD, KLVVVGASG, LDILDTAGR, LEEYSAMRD, LVVVGARGV, LVVVGASGV, REEYSAMRDQY, RGVGKSAL, TAGLEEYSA, TEYKLVVVGAA, VGAAGVGKSA, VGADGVGK, VGASGVGKSA, VGVGKSALTI, VVVGAAGV, VVVGAVGV, YKLVVVGAC, YKLVVVGAD, YKLVVVGAR, or DILDTAGKE.

The polypeptide of any one of paragraphs [0492]-[0603], wherein Yₙ comprises an amino acid sequence of IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, TEYKLV, WQAGILAR, HSYTTAE, PLTEEKIK, GALHFKPGSR, RRANKDATAE, KAFISHEEKR, TDLSSRFSKS, FDLGGGTFDV, CLLLHYSVSK, KKKKIIMKIRNA, or MTEYKLVVV.

The polypeptide of any one of paragraphs [0492]-[0604], wherein Zₚ comprises an amino acid sequence of KKNKKDDI, KKNKKDDIKD, AGNDDDDDDDDDDDDDDDDDKKDKDDDDDD, AGNKKKKKKKNNNNNNNNNNNNNNNNNNNN, AGRDDDDDDDDDDDDDDDDDDDDDDDDDDD, SALTI, SALTIQL, GKSALTIQL, GKSALTI, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

The polypeptide of any one of paragraphs [0492]-[0593], wherein the epitope is not a RAS epitope.

The polypeptide of any one of paragraphs [0492]-[0606], wherein the polypeptide is not KKKKKPKRDGYMFLKAESKIMFAT, KKKKYMFLKAESKIMFATLQRSS, KKKKKAESKIMFATLQRSSLWCL, KKKKKIMFATLQRSSLWCLCSNH, or KKKKMFATLQRSSLWCLCSNH.

The polypeptide of any one of paragraphs [0492]-[0593], wherein the epitope is a GATA3 epitope.

The polypeptide of paragraph [0608], wherein the GATA3 epitope comprises an amino acid sequence of MLTGPPARV, SMLTGPPARV, VLPEPHLAL, KPKRDGYMF, KPKRDGYMFL, ESKIMFATL, KRDGYMFL, PAVPFDLHF, AESKIMFATL, FATLQRSSL, ARVPAVPFD, IMKPKRDGY, DGYMFLKA, MFLKAESKIMF, LTGPPARV, ARVPAVPF, SMLTGPPAR, RVPAVPFDL, or LTGPPARVP.

A cell comprising the polypeptide of any one of the paragraphs [0492]-[0609].

The cell of paragraph [0610], wherein the cell is an antigen presenting cell.

The cell of paragraph [0611], wherein the cell is a dendritic cell.

The cell of paragraph [0610], wherein the cell is a mature antigen presenting cell.

A method of cleaving a polypeptide comprising contacting the polypeptide of any one of paragraphs [0492]-[0609] to an APC.

The method of paragraph [0614], wherein the method is performed in vivo.

The method of paragraph [0614], wherein the method is performed ex vivo.

A method of manufacturing a polypeptide comprising linking Yₙ-Aᵣ and/or Aₛ-Zₚ to a sequence comprising an epitope sequence, wherein the epitope sequence is presented by a class I MHC or a class II MHC of an antigen presenting cell (APC); and wherein
(i) each Y is independently an amino acid, analog, or derivative thereof of and wherein Yₙ is not encoded by a nucleic acid sequence immediately upstream of a nucleic acid sequence in a genome of a subject that encodes the epitope, and wherein, n is an integer from 0 to 1000;
(ii) each Z is independently an amino acid, analog, or derivative thereof of and wherein Zₚ is not encoded by a nucleic acid sequence immediately downstream of the nucleic acid sequence in the genome of the subject that encodes the epitope, and wherein, p is an integer from 0 to 1000; and
(iii) Aᵣ is a linker and Aₛ is a linker, wherein at least one of r and s is 1;
and further wherein,
(a) the polypeptide does not consist of four different epitopes presented by a class I MHC;
(b) the polypeptide comprises at least two different polypeptide molecules;
(c) the epitope comprises at least one mutant amino acid; and/or (d) Yₙ and/or Zₚ is cleaved from the epitope when the polypeptide is processed by the APC.

A method of manufacturing a polypeptide comprising linking Yₙ to Bₜ-Xₘ and/or Zₚ to Xₘ-Cᵤ, wherein Xₘ is an epitope sequence presented by a class I MHC or a class II MHC of an antigen presenting cell (APC); and wherein
(i) each B independently represents an amino acid encoded by a nucleic acid sequence in a genome of a subject that is immediately upstream of a nucleic acid sequence in the genome of the subject that encodes Xₘ,
and wherein t is an integer from 0 to 1000;
(ii) each C independently represents an amino acid encoded by a nucleic acid sequence in the genome of the subject that is immediately downstream of the nucleic acid sequence in the genome of the subject that encodes Xₘ,
and wherein, u is an integer from 0 to 1000;
(iii) each Y is independently an amino acid, analog, or derivative thereof of and wherein Yₙ is not encoded by a nucleic acid sequence immediately upstream of a nucleic acid sequence in the genome of the subject that encodes Bₜ-Xₘ,
   and wherein, n is an integer from 0 to 1000; and
(iv) each Z is independently an amino acid, analog, or derivative thereof of and wherein Zₚ is not encoded by a nucleic acid sequence immediately downstream of a nucleic acid sequence in the genome of the subject that encodes Xₘ-Cᵤ,
   and wherein, p is an integer from 0 to 1000;
and further wherein,
(a) the polypeptide does not consist of four different epitopes presented by a class I MHC;
(b) the polypeptide comprises at least two different polypeptide molecules;
(c) the epitope comprises at least one mutant amino acid; and/or
(d) Yₙ-Bₜ and/or Cᵤ-Zₚ is cleaved from the epitope when the polypeptide is processed by the APC.

The method of paragraph [0617] or [0618], wherein when n is 0, p is an integer from 1 to 1000; and when p is 0, n is an integer from 1 to 1000.

The method of any one of paragraphs [0617]-[0619], wherein each X independently represents an amino acid of a peptide sequence comprising any contiguous amino acid sequence encoded by the nucleic acid sequence in the genome of a subject, and wherein (a) the MHC is a class I MHC and m is an integer from 8 to 12 or (b) the MHC is a class II MHC and m is an integer from 9 to 25.

A pharmaceutical composition comprising the polypeptide of any one of paragraphs [0492]-[0609] and a pharmaceutically acceptable excipient.

The pharmaceutical composition of paragraph [0621], further comprising an immunomodulatory agent or an adjuvant.

The pharmaceutical composition of paragraph [0622], wherein the immunomodulatory agent or an adjuvant is selected from the group consisting of poly-ICLC, 1018 ISS, aluminum salts, Amplivax, AS15, BCG, CP-870,893, CpG7909, CyaA, ARNAX, STING agonists, dSLIM, GM-CSF, IC30, IC31, Imiquimod, ImuFact IMP321, IS Patch, ISS, ISCOMATRIX, Juvlmmune, LipoVac, MF59, monophosphoryllipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam2Cys, Pam3Cys, Pam3C-SK4, and Aquila's QS21 stimulon.

The pharmaceutical composition of paragraph [0622] or [0623], wherein the immunomodulatory agent or adjuvant comprises poly-ICLC.

The pharmaceutical composition of any one of paragraphs [0621]-[0624], wherein the pharmaceutical composition is a vaccine composition.

The pharmaceutical composition of any one of paragraphs [0621]-[0625], wherein the pharmaceutical composition is aqueous or a liquid.

The pharmaceutical composition of any one of paragraphs [0621]-[0626], wherein the epitope is present in the pharmaceutical composition at an amount of from 1 ng to 10 mg or 5 pg to 1.5 mg.

The pharmaceutical composition of any one of paragraphs [0621]-[0627], further comprising DMSO.

The pharmaceutical composition of any one of paragraphs [0621]-[0628], wherein the pharmaceutically acceptable excipient comprises water.

The pharmaceutical composition of any one of paragraphs [0621]-[0629], wherein the pharmaceutical composition comprises a pH modifier present at a concentration of less than 1 mM or greater than 1 mM.

The pharmaceutical composition of paragraph [0630], wherein the pH modifier is a dicarboxylate salt or a tricarboxylate salt.

The pharmaceutical composition of paragraph [0630], wherein the pH modifier is a dicarboxylate salt of succinic acid, or a disuccinate salt.

The pharmaceutical composition of paragraph [0630], wherein the pH modifier is a tricarboxylate salt of citric acid, or a tricitrate salt.

The pharmaceutical composition of paragraph [0630], wherein the pH modifier is disodium succinate.

The pharmaceutical composition of paragraph [0632], wherein the dicarboxylate salt of succinic acid, or the disuccinate salt, is present in the pharmaceutical composition at a concentration of 0.1 mM-1 mM.

The pharmaceutical composition of paragraph [0632], wherein the dicarboxylate salt of succinic acid, or the disuccinate salt, is present in the pharmaceutical composition at a concentration of 1 mM-5 mM.

The pharmaceutical composition of any one of paragraphs [0621]-[0636], wherein an immune response to the epitope is increased when administered to a subject.

A method of treating a disease or a condition comprising administering a therapeutically effective amount of the pharmaceutical composition of any one of paragraphs [0621]-[0637] to a subject in need thereof.

The method of paragraph [0638], wherein the disease or the condition is a cancer.

The method of paragraph [0639], wherein the cancer is selected from the group consisting of lung cancer, non-small cell lung cancer, pancreatic cancer, colorectal cancer, uterine cancer, and liver cancer.

The method of any one of paragraphs [0638]-[0640], wherein administering comprises intradermal injection, intranasal spray application, intramuscular injection, intraperitoneal injection, intravenous injection, oral administration, or subcutaneous injection.

A method of prophylaxis of a subject comprising contacting a cell of the subject with the polypeptide, cell, or pharmaceutical composition of any one of paragraphs [0492]-[0613] or [0621]-[0637].

A method comprising identifying an epitope expressed by a subject's tumor cells and producing a polypeptide comprising the epitope, wherein the polypeptide has a structure of Formula (I):

Yₙ-Bₜ-Aᵣ-Xₘ-Aₛ-Cᵤ-Zₚ Formula (I),

or a pharmaceutically acceptable salt thereof,
(i) wherein Xₘ is the epitope, wherein each X independently represents an amino acid of a contiguous amino acid sequence encoded by a nucleic acid sequence in a genome of a subject, and wherein, (a) the MHC is a class I MHC and m is an integer from 8 to 12, or
   (b) the MHC is a class II MHC and m is an integer from 9 to 25;
(ii) wherein each Y is independently an amino acid, analog, or derivative thereof, and wherein:
   (A) when variable r of Aᵣ in Formula (I) is 0, Yₙ is not encoded by a nucleic acid sequence immediately upstream of the nucleic acid sequence in the genome of the subject that encodes Bₜ-Aᵣ-Xₘ,
   (B) when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or
   (C) when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and
   further wherein, n is an integer from 0 to 1000;
(iii) wherein each Z is independently an amino acid, analog, or derivative thereof, and wherein:
   (A) when variable s of Aₛ in Formula (I) is 0, Zₚ is not encoded by a nucleic acid sequence immediately downstream of the nucleic acid sequence in the genome of the subject that encodes Xₘ-Aₛ-Cᵤ,
   (B) when variable s of Aₛ in Formula (I) is 1 and variable u of Cᵤ in Formula (I) is 0, Zₚ is not encoded by a nucleic acid sequence immediately downstream of the nucleic acid sequence in the genome of the subject that encodes Xₘ, or
   (C) when variable s of Aₛ in Formula (I) is 1 and variable u of Cᵤ in Formula (I) is 1 or more, Zₚ is not encoded by a nucleic acid sequence immediately downstream of the nucleic acid sequence in the genome of the subject that encodes Cᵤ; and
   further wherein, p is an integer from 0 to 1000;
and further wherein,
   when n is 0, p is an integer from 1 to 1000; and
   when p is 0, n is an integer from 1 to 1000;
(iv) wherein Aᵣ is a linker, and r is 0 or 1;
(v) wherein Aₛ is a linker, and s is 0 or 1;
(vi) wherein each B independently represents an amino acid encoded by a nucleic acid sequence in the genome of the subject that is immediately upstream of the nucleic acid sequence in the genome of the subject that encodes Xₘ,
   and wherein t is an integer from 0 to 1000; and
(vii) wherein each C independently represents an amino acid encoded by a nucleic acid sequence in the genome of the subject that is immediately downstream of the nucleic acid sequence in the genome of the subject that encodes Xₘ,
   and wherein, u is an integer from 0 to 1000;
and further wherein,
(a) the polypeptide does not consist of four different epitopes presented by a class I MHC;
(b) the polypeptide comprises at least two different polypeptide molecules;
(c) the epitope comprises at least one mutant amino acid; and/or
(d) Yₙ and/or Zₚ is cleaved from the epitope when the polypeptide is processed by the APC.

The method of paragraph [0643], wherein identifying comprises selecting a plurality of nucleic acid sequences from a pool of nucleic acid sequences sequenced from the subject's tumor cells that encode a plurality of candidate peptide sequences comprising one or more different mutations not present in a pool of nucleic acid sequences sequenced from the subject's non-tumor cells, wherein the pool of nucleic acid sequences sequenced from the subject's tumor cells and the pool of nucleic acid sequences sequenced from the subject's non-tumor cells are sequenced by whole genome sequencing or whole exome sequencing.

The method of paragraph [0643] or [0644], wherein identifying further comprises predicting or measuring which candidate peptide sequences of the plurality of candidate peptide sequences form a complex with a protein encoded by an HLA allele of the same subject by an HLA peptide binding analysis.

The method of any one of paragraphs [0643]-[0645], wherein identifying further comprises selecting the plurality of selected tumor-specific peptides or one or more polynucleotides encoding the plurality of selected tumor-specific peptides from the candidate peptide sequences based on the HLA peptide binding analysis.

The method of any one of paragraphs [0643]-[0646], further comprising administering the polypeptide to the subject.

The method of paragraph [0647], wherein administering comprises intradermal injection, intranasal spray application, intramuscular injection, intraperitoneal injection, intravenous injection, oral administration, or subcutaneous injection.

The method of any one of paragraphs [0643]-[0648], wherein an immune response is elicited in the subject.

The method of any one of paragraphs [0643]-[0649], wherein the epitope expressed by the subject's tumor cells is a neoantigen, a tumor associated antigen, a mutated tumor associated antigen, and/or wherein expression of the epitope is higher in the subject's tumor cells compared to expression of the epitope in a normal cell of the subject.

1-48. (canceled)

49. A polypeptide comprising an epitope presented by a class I MHC or a class II MHC of an antigen presenting cell (APC), the polypeptide having a structure of Formula (I):
Yₙ-Bₜ-Aᵣ-Xₘ-Aₛ-Cᵤ-Zₚ Formula (I),
or a pharmaceutically acceptable salt thereof,
(i) wherein Xₘ is the epitope, wherein each X independently represents an amino acid of a contiguous amino acid sequence encoded by a nucleic acid sequence in a genome of a subject,
and wherein, (a) the MHC is a class I MHC and m is an integer from 8 to 12, or
(b) the MHC is a class II MHC and m is an integer from 9 to 25;
(ii) wherein each Y is independently an amino acid, analog, or derivative thereof, and wherein:
(A) when variable r of Aᵣ in Formula (I) is 0, Yₙ is not encoded by a nucleic acid sequence immediately upstream of the nucleic acid sequence in the genome of the subject that encodes Bₜ-Aᵣ-Xₘ,
(B) when variable r of Aᵣ in Formula (I) is 1 and variable t of Bₜ in Formula (I) is 0, Yₙ is not encoded by a nucleic acid sequence immediately upstream of the nucleic acid sequence in the genome of the subject that encodes Xₘ, or
(C) when variable r of Aᵣ in Formula (I) is 1 and variable t of Bₜ in Formula (I) is 1 or more, Yₙ is not encoded by a nucleic acid sequence immediately upstream of the nucleic acid sequence in the genome of the subject that encodes Bₜ; and
further wherein, n is an integer from 0 to 1000;
(iii) wherein each Z is independently an amino acid, analog, or derivative thereof, and wherein:
(A) when variable s of Aₛ in Formula (I) is 0, Zₚ is not encoded by a nucleic acid sequence immediately downstream of the nucleic acid sequence in the genome of the subject that encodes Xₘ-Aₛ-Cᵤ,
(B) when variable s of Aₛ in Formula (I) is 1 and variable u of Cᵤ in Formula (I) is 0, Zₚ is not encoded by a nucleic acid sequence immediately downstream of the nucleic acid sequence in the genome of the subject that encodes Xₘ, or
(C) when variable s of Aₛ in Formula (I) is 1 and variable u of Cᵤ in Formula (I) is 1 or more, Zₚ is not encoded by a nucleic acid sequence immediately downstream of the nucleic acid sequence in the genome of the subject that encodes Cᵤ; and
further wherein, p is an integer from 0 to 1000;
and further wherein,
when n is 0, p is an integer from 1 to 1000; and
when p is 0, n is an integer from 1 to 1000;
(iv) wherein Aᵣ is a linker, and r is 0 or 1;
(v) wherein Aₛ is a linker, and s is 0 or 1;
(vi) wherein each B independently represents an amino acid encoded by a nucleic acid sequence in the genome of the subject that is immediately upstream of the nucleic acid sequence in the genome of the subject that encodes Xₘ,
and wherein t is an integer from 0 to 1000; and
(vii) wherein each C independently represents an amino acid encoded by a nucleic acid sequence in the genome of the subject that is immediately downstream of the nucleic acid sequence in the genome of the subject that encodes Xₘ,
and wherein, u is an integer from 0 to 1000;
and further wherein,
(a) the polypeptide does not consist of four different epitopes presented by a class I MHC;
(b) the polypeptide comprises at least two different polypeptide molecules;
(c) the epitope comprises at least one mutant amino acid; and/or
(d) Yₙ and/or Zₚ is cleaved from the epitope when the polypeptide is processed by the APC;
wherein the subject is a human, and wherein Yₙ-Bₜ-Aᵣ and/or AS-Cᵤ-Zₚ enhances solubility of the polypeptide compared to a corresponding peptide that does not contain Yₙ-Bₜ-Aᵣ and/or AS-Cᵤ-Zₚ.

50. The polypeptide of claim 49, wherein
(a) the polypeptide is cleaved at a higher rate when n is an integer from 1 to 1000 compared to cleavage of a corresponding polypeptide of the same length that comprises Xₘ and at least one additional amino acid encoded by a nucleic acid sequence immediately upstream of the nucleic acid sequence in the genome of the subject that encodes Xₘ;
(b) the polypeptide is cleaved at a higher rate when p is an integer from 1 to 1000 compared to cleavage of a corresponding polypeptide of the same length that comprises Xₘ and at least one additional amino acid encoded by a nucleic acid sequence immediately downstream of the nucleic acid sequence in the genome of the subject that encodes Xₘ;
(c) epitope presentation by the APC is enhanced when n is an integer from 1 to 1000 compared to epitope presentation of a corresponding polypeptide of the same length that comprises Xₘ and at least one additional amino acid encoded by a nucleic acid sequence immediately upstream of the nucleic acid sequence in the genome of the subject that encodes Xₘ;
(d) epitope presentation by the APC is enhanced when p is an integer from 1 to 1000 compared to epitope presentation of a corresponding polypeptide of the same length that comprises Xₘ and at least one additional amino acid encoded by a nucleic acid sequence immediately downstream of the nucleic acid sequence in the genome of the subject that encodes Xₘ;
(e) immunogenicity is enhanced when n is an integer from 1 to 1000 compared to immunogenicity of a corresponding polypeptide of the same length that comprises Xₘ and at least one additional amino acid encoded by a nucleic acid sequence immediately upstream of the nucleic acid sequence in the genome of the subject that encodes Xₘ;
(f) immunogenicity is enhanced when p is an integer from 1 to 1000 compared to immunogenicity of a corresponding polypeptide of the same length that comprises Xₘ and at least one additional amino acid encoded by a nucleic acid sequence immediately downstream of the nucleic acid sequence in the genome of the subject that encodes Xₘ;
(g) anti-tumor activity is enhanced when n is an integer from 1 to 1000 compared to anti-tumor activity of a corresponding polypeptide of the same length that comprises Xₘ and at least one additional amino acid encoded by a nucleic acid sequence immediately upstream of the nucleic acid sequence in the genome of the subject that encodes Xₘ; and/or
(h) anti-tumor activity is enhanced when p is an integer from 1 to 1000 compared to anti-tumor activity of a corresponding polypeptide of the same length that comprises Xₘ and at least one additional amino acid encoded by a nucleic acid sequence immediately downstream of the nucleic acid sequence in the genome of the subject that encodes Xₘ.

51. The polypeptide of claim 49, wherein Yₙ and/or Zₚ comprises a sequence selected from the group consisting of lysine (Lys), poly-Lys (polyK) and poly-Arg (polyR), wherein the polyK or polyR comprises at least two, three or four contiguous lysine or arginine residues, respectively.

52. The polypeptide of claim 49, wherein the HLA allele is selected from the group consisting of HLA-A02:01 allele, an HLA-A03:01 allele, an HLA-A11:01 allele, an HLA-A03:02 allele, an HLA-A30:01 allele, an HLA-A31:01 allele, an HLA-A33:01 allele, an HLA-A33:03 allele, an HLA-A68:01 allele, an HLA-A74:01 allele, and/or an HLA-C08:02 allele and any combination thereof.

53. The polypeptide of any claim 49, wherein the epitope comprises at least one mutant amino acid and wherein the epitope is a RAS epitope.

54. The polypeptide of claim 49, wherein the epitope comprises a mutant RAS peptide sequence that comprises at least 8 contiguous amino acids of a mutant RAS protein comprising a mutation at G12, G13, or Q61; wherein the at least 8 contiguous amino acids of a mutant RAS protein comprising a mutation at G12, G13, or Q61 comprises a G12A, G12C, G12D, G12R, G12S, G12V, G13A, G13C, G13D, G13R, G13S, G13V, Q61H, Q61L, Q61K, or Q61R mutation.

55. The polypeptide of claim 49, wherein the epitope comprises a mutant RAS peptide sequence that comprises an amino acid sequence of VVVGAAGVGK, VVVGAAGVG, VVVGAAGV, VVGAAGVGK, VVGAAGVG, VGAAGVGK, VVVGACGVGK, VVVGACGVG, VVVGACGV, VVGACGVGK, VVGACGVG, VGACGVGK, VVVGADGVGK, VVVGADGVG, VVVGADGV, VVGADGVGK, VVGADGVG, VGADGVGK, VVVGARGVGK, VVVGARGVG, VVVGARGV, VVGARGVGK, VVGARGVG, VGARGVGK, VVVGASGVGK, VVVGASGVG, VVVGASGV, VVGASGVGK, VVGASGVG, VGASGVGK, VVVGAVGVGK, VVVGAVGVG, VVVGAVGV, VVGAVGVGK, VVGAVGVG, or VGAVGVGK.

56. The polypeptide of claim 49, wherein Yₙ comprises an amino acid sequence of K, KK, KKK, KKKK, KKKKK, KKKKKKK, KKKKKKKK, KTEY, KTEYK, KTEYKL, KTEYKLV, KTEYKLVV, KTEYKLVVV, KKTEY, KKTEYK, KKTEYKL, KKTEYKLV, KKTEYKLVV, KKTEYKLVVV, KKKTEY, KKKTEYK, KKKTEYKL, KKKTEYKLV, KKKTEYKLVV, KKKTEYKLVVV, KKKKTEY, KKKKTEYK, KKKKTEYKL, KKKKTEYKLV, KKKKTEYKLVV, KKKKTEYKLVVV, IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, TEYK, TEYKL, TEYKLV, TEYKLVV, TEYKLVVV, WQAGILAR, HSYTTAE, PLTEEKIK, GALHFKPGSR, RRANKDATAE, KAFISHEEKR, TDLSSRFSKS, FDLGGGTFDV, CLLLHYSVSK, KKKKIIMKIRNA, or MTEYKLVVV.

57. The polypeptide of claim 49, wherein Zₚ comprises an amino acid sequence of K, KK, KKK, KKKK, KKKKK, KKKKKKK, KKKKKKKK, KKNKKDDI, KKNKKDDIKD, AGNDDDDDDDDDDDDDDDDDKKDKDDDDDD, AGNKKKKKKKNNNNNNNNNNNNNNNNNNNN, AGRDDDDDDDDDDDDDDDDDDDDDDDDDDD, SALTI, SALTIQL, GKSALTIQL, GKSALTI, SALTIK, SALTIQLK, GKSALTIQLK, GKSALTIK, SALTIKK, SALTIQLKK, GKSALTIQLKK, GKSALTIKK, SALTIKKK, SALTIQLKKK, GKSALTIQLKKK, GKSALTIKKK, SALTIKKKK, SALTIQLKKKK, GKSALTIQLKKKK, GKSALTI, KKKK, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

58. The polypeptide of claim 49, wherein the polypeptide comprises an amino acid sequence of KTEYKLVVVGAVGVGKSALTIQL, KTEYKLVVVGADGVGKSALTIQL, KTEYKLVVVGARGVGKSALTIQL, KTEYKLVVVGACGVGKSALTIQL, KKTEYKLVVVGAVGVGKSALTIQL, KKTEYKLVVVGADGVGKSALTIQL, KKTEYKLVVVGARGVGKSALTIQL, KKTEYKLVVVGACGVGKSALTIQL, KKKTEYKLVVVGAVGVGKSALTIQL, KKKTEYKLVVVGADGVGKSALTIQL, KKKTEYKLVVVGARGVGKSALTIQL, KKKTEYKLVVVGACGVGKSALTIQL, KKKKTEYKLVVVGAVGVGKSALTIQL, KKKKTEYKLVVVGADGVGKSALTIQL, KKKKTEYKLVVVGARGVGKSALTIQL, KKKKTEYKLVVVGACGVGKSALTIQL, KKTEYKLVVVGAVGVGKSALTIQLKK, KKTEYKLVVVGADGVGKSALTIQLKK, KKTEYKLVVVGARGVGKSALTIQLKK, KKTEYKLVVVGACGVGKSALTIQLKK, TEYKLVVVGAVGVGKSALTIQLK, TEYKLVVVGADGVGKSALTIQLK, TEYKLVVVGARGVGKSALTIQLK, TEYKLVVVGACGVGKSALTIQLK, TEYKLVVVGAVGVGKSALTIQLKK, TEYKLVVVGADGVGKSALTIQLKK, TEYKLVVVGARGVGKSALTIQLKK, TEYKLVVVGACGVGKSALTIQLKK, TEYKLVVVGAVGVGKSALTIQLKKK, TEYKLVVVGADGVGKSALTIQLKKK, TEYKLVVVGARGVGKSALTIQLKKK, TEYKLVVVGACGVGKSALTIQLKKK, TEYKLVVVGAVGVGKSALTIQLKKKK, TEYKLVVVGADGVGKSALTIQLKKKK, TEYKLVVVGARGVGKSALTIQLKKKK, or TEYKLVVVGACGVGKSALTIQLKKKK.

59. The polypeptide of claim 58, wherein the polypeptide comprises an amino acid sequence of KKKTEYKLVVVGADGVGKSALTIQL.

60. The polypeptide of claim 58, wherein the polypeptide comprises an amino acid sequence of KKKTEYKLVVVGARGVGKSALTIQL.

61. The polypeptide of claim 58, wherein the polypeptide comprises an amino acid sequence of KKKKTEYKLVVVGAVGVGKSALTIQL.

62. The polypeptide of claim 58, wherein the polypeptide comprises an amino acid sequence of KKKKTEYKLVVVGACGVGKSALTIQL.

63. A method of preparing antigen-specific T cells comprising stimulating T cells with antigen presenting cells comprising the polypeptide of claim 49 or a polynucleotide comprising a sequence encoding the polypeptide of claim 49.

64. The method of claim 63, wherein the method comprises stimulating T cells with antigen presenting cells comprising at least 4 polypeptides or a polynucleotide encoding the at least 4 polypeptides, wherein the at least 4 polypeptides comprise:
(a) a polypeptide comprising an amino acid sequence of KKKTEYKLVVVGADGVGKSALTIQL,
(b) a polypeptide comprising an amino acid sequence of KKKTEYKLVVVGARGVGKSALTIQL,
(c) a polypeptide comprising an amino acid sequence of KKKKTEYKLVVVGAVGVGKSALTIQL, and
(d) a polypeptide comprising an amino acid sequence of KKKKTEYKLVVVGACGVGKSALTIQL.

65. A pharmaceutical composition comprising the polypeptide of claim 49 or a polynucleotide comprising a sequence encoding the polypeptide of claim 49.

66. The pharmaceutical composition of claim 65, wherein the pharmaceutical composition comprises the polynucleotide comprising a sequence encoding the polypeptide of claim 49, and wherein the polynucleotide is an mRNA.

67. A polynucleotide comprising a sequence encoding the polypeptide of claim 49, wherein the polynucleotide is an mRNA.

68. A method of treating a cancer in a subject in need thereof comprising administering a therapeutically effective amount of the pharmaceutical composition of claim 65 to a subject in need thereof.


SEQUENCE LISTING (395 sequences)

SEQ ID NO: 1 - protein, 4 aa, Artificial Sequence
  Description of Artificial Sequence Synthetic peptide
KKKK

SEQ ID NO: 2 - protein, 4 aa, Artificial Sequence
  Description of Artificial Sequence Synthetic peptide
RRRR

SEQ ID NO: 3 - protein, 5 aa, Artificial Sequence
  Description of Artificial Sequence Synthetic peptide
KKKKK

SEQ ID NO: 4 - protein, 10 aa, Artificial Sequence
  Description of Artificial Sequence Synthetic peptide
GADGVGKSAL

SEQ ID NO: 5 - protein, 10 aa, Artificial Sequence
  Description of Artificial Sequence Synthetic peptide
GACGVGKSAL

SEQ ID NO: 6 - protein, 10 aa, Artificial Sequence
  Description of Artificial Sequence Synthetic peptide
GAVGVGKSAL

SEQ ID NO: 7 - protein, 9 aa, Artificial Sequence
  Description of Artificial Sequence Synthetic peptide
GADGVGKSA

SEQ ID NO: 8 - protein, 9 aa, Artificial Sequence
  Description of Artificial Sequence Synthetic peptide
GACGVGKSA

SEQ ID NO: 9 - protein, 9 aa, Artificial Sequence
  Description of Artificial Sequence Synthetic peptide
GAVGVGKSA

SEQ ID NO: 10 - protein, 10 aa, Artificial Sequence
  Description of Artificial Sequence Synthetic peptide
KLVVVGACGV

SEQ ID NO: 11 - protein, 10 aa, Artificial Sequence
  Description of Artificial Sequence Synthetic peptide
FLVVVGACGL

SEQ ID NO: 12 - protein, 10 aa, Artificial Sequence
  Description of Artificial Sequence Synthetic peptide
FMVVVGACGI

SEQ ID NO: 13 - protein, 10 aa, Artificial Sequence
  Description of Artificial Sequence Synthetic peptide
FLVVVGACGI

SEQ ID NO: 14 - protein, 10 aa, Artificial Sequence
  Description of Artificial Sequence Synthetic peptide
FMVVVGACGV

SEQ ID NO: 15 - protein, 10 aa, Artificial Sequence
  Description of Artificial Sequence Synthetic peptide
FLVVVGACGV

SEQ ID NO: 16 - protein, 10 aa, Artificial Sequence
  Description of Artificial Sequence Synthetic peptide
MLVVVGACGV

SEQ ID NO: 17 - protein, 10 aa, Artificial Sequence
  Description of Artificial Sequence Synthetic peptide
FMVVVGACGL

SEQ ID NO: 18 - protein, 10 aa, Artificial Sequence
  Description of Artificial Sequence Synthetic peptide
YLVVVGACGV

SEQ ID NO: 19 - protein, 10 aa, Artificial Sequence
  Description of Artificial Sequence Synthetic peptide
KMVVVGACGV

SEQ ID NO: 20 - protein, 10 aa, Artificial Sequence
  Description of Artificial Sequence Synthetic peptide
YMVVVGACGV

SEQ ID NO: 21 - protein, 10 aa, Artificial Sequence
  Description of Artificial Sequence Synthetic peptide
MMVVVGACGV

SEQ ID NO: 22 - protein, 8 aa, Artificial Sequence
  Description of Artificial Sequence Synthetic peptide
DTAGHEEY

SEQ ID NO: 23 - protein, 10 aa, Artificial Sequence
  Description of Artificial Sequence Synthetic peptide
TAGHEEYSAM

SEQ ID NO: 24 - protein, 9 aa, Artificial Sequence
  Description of Artificial Sequence Synthetic peptide
DILDTAGHE

SEQ ID NO: 25 - protein, 8 aa, Artificial Sequence
  Description of Artificial Sequence Synthetic peptide
DILDTAGH

SEQ ID NO: 26 - protein, 9 aa, Artificial Sequence
  Description of Artificial Sequence Synthetic peptide
ILDTAGHEE

SEQ ID NO: 27 - protein, 8 aa, Artificial Sequence
  Description of Artificial Sequence Synthetic peptide
ILDTAGHE

SEQ ID NO: 28 - protein, 11 aa, Artificial Sequence
  Description of Artificial Sequence Synthetic peptide
DILDTAGHEEY

SEQ ID NO: 29 - protein, 9 aa, Artificial Sequence
  Description of Artificial Sequence Synthetic peptide
DTAGHEEYS

SEQ ID NO: 30 - protein, 10 aa, Artificial Sequence
  Description of Artificial Sequence Synthetic peptide
LLDILDTAGH

SEQ ID NO: 31 - protein, 9 aa, Artificial Sequence
  Description of Artificial Sequence Synthetic peptide
DILDTAGRE

SEQ ID NO: 32 - protein, 8 aa, Artificial Sequence
  Description of Artificial Sequence Synthetic peptide
DILDTAGR

SEQ ID NO: 33 - protein, 9 aa, Artificial Sequence
  Description of Artificial Sequence Synthetic peptide
ILDTAGREE

SEQ ID NO: 34 - protein, 8 aa, Artificial Sequence
  Description of Artificial Sequence Synthetic peptide
ILDTAGRE

SEQ ID NO: 35 - protein, 11 aa, Artificial Sequence
  Description of Artificial Sequence Synthetic peptide
CLLDILDTAGR

SEQ ID NO: 36 - protein, 10 aa, Artificial Sequence
  Description of Artificial Sequence Synthetic peptide
TAGREEYSAM

SEQ ID NO: 37 - protein, 9 aa, Artificial Sequence
  Description of Artificial Sequence Synthetic peptide
REEYSAMRD

SEQ ID NO: 38 - protein, 11 aa, Artificial Sequence
  Description of Artificial Sequence Synthetic peptide
DTAGKEEYSAM

SEQ ID NO: 39 - protein, 11 aa, Artificial Sequence
  Description of Artificial Sequence Synthetic peptide
CLLDILDTAGK

SEQ ID NO: 40 - protein, 8 aa, Artificial Sequence
  Description of Artificial Sequence Synthetic peptide
DTAGKEEY

SEQ ID NO: 41 - protein, 10 aa, Artificial Sequence
  Description of Artificial Sequence Synthetic peptide
LLDILDTAGK

SEQ ID NO: 42 - protein, 8 aa, Artificial Sequence
  Description of Artificial Sequence Synthetic peptide
ILDTAGKE

SEQ ID NO: 43 - protein, 9 aa, Artificial Sequence
  Description of Artificial Sequence Synthetic peptide
ILDTAGKEE

SEQ ID NO: 44 - protein, 8 aa, Artificial Sequence
  Description of Artificial Sequence Synthetic peptide
DTAGLEEY

SEQ ID NO: 45 - protein, 8 aa, Artificial Sequence
  Description of Artificial Sequence Synthetic peptide
ILDTAGLE

SEQ ID NO: 46 - protein, 8 aa, Artificial Sequence
  Description of Artificial Sequence Synthetic peptide
DILDTAGL

SEQ ID NO: 47 - protein, 9 aa, Artificial Sequence
  Description of Artificial Sequence Synthetic peptide
ILDTAGLEE

SEQ ID NO: 48 - protein, 12 aa, Artificial Sequence
  Description of Artificial Sequence Synthetic peptide
GLEEYSAMRDQY

SEQ ID NO: 49 - protein, 11 aa, Artificial Sequence
  Description of Artificial Sequence Synthetic peptide
LLDILDTAGLE

SEQ ID NO: 50 - protein, 9 aa, Artificial Sequence
  Description of Artificial Sequence Synthetic peptide
LDILDTAGL

SEQ ID NO: 51 - protein, 9 aa, Artificial Sequence
  Description of Artificial Sequence Synthetic peptide
DILDTAGLE

SEQ ID NO: 52 - protein, 11 aa, Artificial Sequence
  Description of Artificial Sequence Synthetic peptide
DILDTAGLEEY

SEQ ID NO: 53 - protein, 8 aa, Artificial Sequence
  Description of Artificial Sequence Synthetic peptide
AGVGKSAL

SEQ ID NO: 54 - protein, 10 aa, Artificial Sequence
  Description of Artificial Sequence Synthetic peptide
GAAGVGKSAL

SEQ ID NO: 55 - protein, 9 aa, Artificial Sequence
  Description of Artificial Sequence Synthetic peptide
AAGVGKSAL

SEQ ID NO: 56 - protein, 8 aa, Artificial Sequence
  Description of Artificial Sequence Synthetic peptide
CGVGKSAL

SEQ ID NO: 57 - protein, 9 aa, Artificial Sequence
  Description of Artificial Sequence Synthetic peptide
ACGVGKSAL

SEQ ID NO: 58 - protein, 8 aa, Artificial Sequence
  Description of Artificial Sequence Synthetic peptide
DGVGKSAL

SEQ ID NO: 59 - protein, 9 aa, Artificial Sequence
  Description of Artificial Sequence Synthetic peptide
ADGVGKSAL

SEQ ID NO: 60 - protein, 10 aa, Artificial Sequence
  Description of Artificial Sequence Synthetic peptide
DGVGKSALTI

SEQ ID NO: 61 - protein, 9 aa, Artificial Sequence
  Description of Artificial Sequence Synthetic peptide
GARGVGKSA

SEQ ID NO: 62 - protein, 10 aa, Artificial Sequence
  Description of Artificial Sequence Synthetic peptide
KLVVVGARGV

SEQ ID NO: 63 - protein, 8 aa, Artificial Sequence
  Description of Artificial Sequence Synthetic peptide
VVVGARGV

SEQ ID NO: 64 - protein, 8 aa, Artificial Sequence
  Description of Artificial Sequence Synthetic peptide
SGVGKSAL

SEQ ID NO: 65 - protein, 10 aa, Artificial Sequence
  Description of Artificial Sequence Synthetic peptide
VVVGASGVGK

SEQ ID NO: 66 - protein, 10 aa, Artificial Sequence
  Description of Artificial Sequence Synthetic peptide
GASGVGKSAL

SEQ ID NO: 67 - protein, 8 aa, Artificial Sequence
  Description of Artificial Sequence Synthetic peptide
VGVGKSAL

SEQ ID NO: 68 - protein, 10 aa, Artificial Sequence
  Description of Artificial Sequence Synthetic peptide
VVVGAGCVGK

SEQ ID NO: 69 - protein, 9 aa, Artificial Sequence
  Description of Artificial Sequence Synthetic peptide
KLVVVGAGC

SEQ ID NO: 70 - protein, 8 aa, Artificial Sequence
  Description of Artificial Sequence Synthetic peptide
GDVGKSAL

SEQ ID NO: 71 - protein, 9 aa, Artificial Sequence
  Description of Artificial Sequence Synthetic peptide
DVGKSALTI

SEQ ID NO: 72 - protein, 10 aa, Artificial Sequence
  Description of Artificial Sequence Synthetic peptide
VVVGAGDVGK

SEQ ID NO: 73 - protein, 10 aa, Artificial Sequence
  Description of Artificial Sequence Synthetic peptide
TAGKEEYSAM

SEQ ID NO: 74 - protein, 11 aa, Artificial Sequence
  Description of Artificial Sequence Synthetic peptide
DTAGHEEYSAM

SEQ ID NO: 75 - protein, 9 aa, Artificial Sequence
  Description of Artificial Sequence Synthetic peptide
TAGHEEYSA

SEQ ID NO: 76 - protein, 11 aa, Artificial Sequence
  Description of Artificial Sequence Synthetic peptide
DTAGREEYSAM

SEQ ID NO: 77 - protein, 9 aa, Artificial Sequence
  Description of Artificial Sequence Synthetic peptide
TAGKEEYSA

SEQ ID NO: 78 - protein, 8 aa, Artificial Sequence
  Description of Artificial Sequence Synthetic peptide
AAGVGKSA

SEQ ID NO: 79 - protein, 9 aa, Artificial Sequence
  Description of Artificial Sequence Synthetic peptide
AGCVGKSAL

SEQ ID NO: 80 - protein, 9 aa, Artificial Sequence
  Description of Artificial Sequence Synthetic peptide
AGDVGKSAL

SEQ ID NO: 81 - protein, 10 aa, Artificial Sequence
  Description of Artificial Sequence Synthetic peptide
AGKEEYSAMR

SEQ ID NO: 82 - protein, 10 aa, Artificial Sequence
  Description of Artificial Sequence Synthetic peptide
AGVGKSALTI

SEQ ID NO: 83 - protein, 9 aa, Artificial Sequence
  Description of Artificial Sequence Synthetic peptide
ARGVGKSAL

SEQ ID NO: 84 - protein, 8 aa, Artificial Sequence
  Description of Artificial Sequence Synthetic peptide
ASGVGKSA

SEQ ID NO: 85 - protein, 9 aa, Artificial Sequence
  Description of Artificial Sequence Synthetic peptide
ASGVGKSAL

SEQ ID NO: 86 - protein, 8 aa, Artificial Sequence
  Description of Artificial Sequence Synthetic peptide
AVGVGKSA

SEQ ID NO: 87 - protein, 9 aa, Artificial Sequence
  Description of Artificial Sequence Synthetic peptide
CVGKSALTI

SEQ ID NO: 88 - protein, 8 aa, Artificial Sequence
  Description of Artificial Sequence Synthetic peptide
DILDTAGK

SEQ ID NO: 89 - protein, 11 aa, Artificial Sequence
  Description of Artificial Sequence Synthetic peptide
DILDTAGREEY

SEQ ID NO: 90 - protein, 12 aa, Artificial Sequence
  Description of Artificial Sequence Synthetic peptide
DTAGHEEYSAMR

SEQ ID NO: 91 - protein, 9 aa, Artificial Sequence
  Description of Artificial Sequence Synthetic peptide
DTAGKEEYS

SEQ ID NO: 92 - protein, 12 aa, Artificial Sequence
  Description of Artificial Sequence Synthetic peptide
DTAGKEEYSAMR

SEQ ID NO: 93 - protein, 9 aa, Artificial Sequence
  Description of Artificial Sequence Synthetic peptide
DTAGLEEYS

SEQ ID NO: 94 - protein, 10 aa, Artificial Sequence
  Description of Artificial Sequence Synthetic peptide
DTAGLEEYSA

SEQ ID NO: 95 - protein, 12 aa, Artificial Sequence
  Description of Artificial Sequence Synthetic peptide
DTAGLEEYSAMR

SEQ ID NO: 96 - protein, 9 aa, Artificial Sequence
  Description of Artificial Sequence Synthetic peptide
DTAGREEYS

SEQ ID NO: 97 - protein, 12 aa, Artificial Sequence
  Description of Artificial Sequence Synthetic peptide
DTAGREEYSAMR

SEQ ID NO: 98 - protein, 9 aa, Artificial Sequence
  Description of Artificial Sequence Synthetic peptide
GAAGVGKSA

SEQ ID NO: 99 - protein, 8 aa, Artificial Sequence
  Description of Artificial Sequence Synthetic peptide
GADGVGKS

SEQ ID NO: 100 - protein, 9 aa, Artificial Sequence
  Description of Artificial Sequence Synthetic peptide
GAGDVGKSA

SEQ ID NO: 101 - protein, 10 aa, Artificial Sequence
  Description of Artificial Sequence Synthetic peptide
GAGDVGKSAL

SEQ ID NO: 102 - protein, 9 aa, Artificial Sequence
  Description of Artificial Sequence Synthetic peptide
GASGVGKSA

SEQ ID NO: 103 - protein, 8 aa, Artificial Sequence
  Description of Artificial Sequence Synthetic peptide
GCVGKSAL

SEQ ID NO: 104 - protein, 10 aa, Artificial Sequence
  Description of Artificial Sequence Synthetic peptide
GCVGKSALTI

SEQ ID NO: 105 - protein, 8 aa, Artificial Sequence
  Description of Artificial Sequence Synthetic peptide
GHEEYSAM

SEQ ID NO: 106 - protein, 8 aa, Artificial Sequence
  Description of Artificial Sequence Synthetic peptide
GKEEYSAM

SEQ ID NO: 107 - protein, 9 aa, Artificial Sequence
  Description of Artificial Sequence Synthetic peptide
GLEEYSAMR

SEQ ID NO: 108 - protein, 8 aa, Artificial Sequence
  Description of Artificial Sequence Synthetic peptide
GREEYSAM

SEQ ID NO: 109 - protein, 9 aa, Artificial Sequence
  Description of Artificial Sequence Synthetic peptide
GREEYSAMR

SEQ ID NO: 110 - protein, 9 aa, Artificial Sequence
  Description of Artificial Sequence Synthetic peptide
HEEYSAMRD

SEQ ID NO: 111 - protein, 9 aa, Artificial Sequence
  Description of Artificial Sequence Synthetic peptide
KEEYSAMRD

SEQ ID NO: 112 - protein, 9 aa, Artificial Sequence
  Description of Artificial Sequence Synthetic peptide
KLVVVGASG

SEQ ID NO: 113 - protein, 9 aa, Artificial Sequence
  Description of Artificial Sequence Synthetic peptide
LDILDTAGR

SEQ ID NO: 114 - protein, 9 aa, Artificial Sequence
  Description of Artificial Sequence Synthetic peptide
LEEYSAMRD

SEQ ID NO: 115 - protein, 9 aa, Artificial Sequence
  Description of Artificial Sequence Synthetic peptide
LVVVGARGV

SEQ ID NO: 116 - protein, 9 aa, Artificial Sequence
  Description of Artificial Sequence Synthetic peptide
LVVVGASGV

SEQ ID NO: 117 - protein, 11 aa, Artificial Sequence
  Description of Artificial Sequence Synthetic peptide
REEYSAMRDQY

SEQ ID NO: 118 - protein, 8 aa, Artificial Sequence
  Description of Artificial Sequence Synthetic peptide
RGVGKSAL

SEQ ID NO: 119 - protein, 9 aa, Artificial Sequence
  Description of Artificial Sequence Synthetic peptide
TAGLEEYSA

SEQ ID NO: 120 - protein, 11 aa, Artificial Sequence
  Description of Artificial Sequence Synthetic peptide
TEYKLVVVGAA

SEQ ID NO: 121 - protein, 10 aa, Artificial Sequence
  Description of Artificial Sequence Synthetic peptide
VGAAGVGKSA

SEQ ID NO: 122 - protein, 8 aa, Artificial Sequence
  Description of Artificial Sequence Synthetic peptide
VGADGVGK

SEQ ID NO: 123 - protein, 10 aa, Artificial Sequence
  Description of Artificial Sequence Synthetic peptide
VGASGVGKSA

SEQ ID NO: 124 - protein, 10 aa, Artificial Sequence
  Description of Artificial Sequence Synthetic peptide
VGVGKSALTI

SEQ ID NO: 125 - protein, 8 aa, Artificial Sequence
  Description of Artificial Sequence Synthetic peptide
VVVGAAGV

SEQ ID NO: 126 - protein, 8 aa, Artificial Sequence
  Description of Artificial Sequence Synthetic peptide
VVVGAVGV

SEQ ID NO: 127 - protein, 9 aa, Artificial Sequence
  Description of Artificial Sequence Synthetic peptide
YKLVVVGAC

SEQ ID NO: 128 - protein, 9 aa, Artificial Sequence
  Description of Artificial Sequence Synthetic peptide
YKLVVVGAD

SEQ ID NO: 129 - protein, 9 aa, Artificial Sequence
  Description of Artificial Sequence Synthetic peptide
YKLVVVGAR

SEQ ID NO: 130 - protein, 9 aa, Artificial Sequence
  Description of Artificial Sequence Synthetic peptide
DILDTAGKE

SEQ ID NO: 131 - protein, 10 aa, Artificial Sequence
  Description of Artificial Sequence Synthetic peptide
IDIIMKIRNA

SEQ ID NO: 132 - protein, 30 aa, Artificial Sequence
  Description of Artificial Sequence Synthetic polypeptide
FFFFFFFFFFFFFFFFFFFFIIFFIFFWMC

SEQ ID NO: 133 - protein, 30 aa, Artificial Sequence
  Description of Artificial Sequence Synthetic polypeptide
FFFFFFFFFFFFFFFFFFFFFFFFAAFWFW

SEQ ID NO: 134 - protein, 30 aa, Artificial Sequence
  Description of Artificial Sequence Synthetic polypeptide
IFFIFFIIFFFFFFFFFFFFIIIIIIIWEC

SEQ ID NO: 135 - protein, 30 aa, Artificial Sequence
  Description of Artificial Sequence Synthetic polypeptide
FIFFFIIFFFFFIFFFFFIFIIIIIIFWEC

SEQ ID NO: 136 - protein, 6 aa, Artificial Sequence
  Description of Artificial Sequence Synthetic peptide
TEYKLV

SEQ ID NO: 137 - protein, 8 aa, Artificial Sequence
  Description of Artificial Sequence Synthetic peptide
WQAGILAR

SEQ ID NO: 138 - protein, 7 aa, Artificial Sequence
  Description of Artificial Sequence Synthetic peptide
HSYTTAE

SEQ ID NO: 139 - protein, 8 aa, Artificial Sequence
  Description of Artificial Sequence Synthetic peptide
PLTEEKIK

SEQ ID NO: 140 - protein, 10 aa, Artificial Sequence
  Description of Artificial Sequence Synthetic peptide
GALHFKPGSR

SEQ ID NO: 141 - protein, 10 aa, Artificial Sequence
  Description of Artificial Sequence Synthetic peptide
RRANKDATAE

SEQ ID NO: 142 - protein, 10 aa, Artificial Sequence
  Description of Artificial Sequence Synthetic peptide
KAFISHEEKR

SEQ ID NO: 143 - protein, 10 aa, Artificial Sequence
  Description of Artificial Sequence Synthetic peptide
TDLSSRFSKS

SEQ ID NO: 144 - protein, 10 aa, Artificial Sequence
  Description of Artificial Sequence Synthetic peptide
FDLGGGTFDV

SEQ ID NO: 145 - protein, 10 aa, Artificial Sequence
  Description of Artificial Sequence Synthetic peptide
CLLLHYSVSK

SEQ ID NO: 146 - protein, 12 aa, Artificial Sequence
  Description of Artificial Sequence Synthetic peptide
KKKKIIMKIRNA

SEQ ID NO: 147 - protein, 9 aa, Artificial Sequence
  Description of Artificial Sequence Synthetic peptide
MTEYKLVVV

SEQ ID NO: 148 - protein, 8 aa, Artificial Sequence
  Description of Artificial Sequence Synthetic peptide
KKNKKDDI

SEQ ID NO: 149 - protein, 10 aa, Artificial Sequence
  Description of Artificial Sequence Synthetic peptide
KKNKKDDIKD

SEQ ID NO: 150 - protein, 30 aa, Artificial Sequence
  Description of Artificial Sequence Synthetic polypeptide
AGNDDDDDDDDDDDDDDDDDKKDKDDDDDD

SEQ ID NO: 151 - protein, 30 aa, Artificial Sequence
  Description of Artificial Sequence Synthetic polypeptide
AGNKKKKKKKNNNNNNNNNNNNNNNNNNNN

SEQ ID NO: 152 - protein, 30 aa, Artificial Sequence
  Description of Artificial Sequence Synthetic polypeptide
AGRDDDDDDDDDDDDDDDDDDDDDDDDDDD

SEQ ID NO: 153 - protein, 5 aa, Artificial Sequence
  Description of Artificial Sequence Synthetic peptide
SALTI

SEQ ID NO: 154 - protein, 7 aa, Artificial Sequence
  Description of Artificial Sequence Synthetic peptide
SALTIQL

SEQ ID NO: 155 - protein, 9 aa, Artificial Sequence
  Description of Artificial Sequence Synthetic peptide
GKSALTIQL

SEQ ID NO: 156 - protein, 7 aa, Artificial Sequence
  Description of Artificial Sequence Synthetic peptide
GKSALTI

SEQ ID NO: 157 - protein, 8 aa, Artificial Sequence
  Description of Artificial Sequence Synthetic peptide
QGQNLKYQ

SEQ ID NO: 158 - protein, 8 aa, Artificial Sequence
  Description of Artificial Sequence Synthetic peptide
ILGVLLLI

SEQ ID NO: 159 - protein, 8 aa, Artificial Sequence
  Description of Artificial Sequence Synthetic peptide
EKEGKISK

SEQ ID NO: 160 - protein, 10 aa, Artificial Sequence
  Description of Artificial Sequence Synthetic peptide
AASDFIFLVT

SEQ ID NO: 161 - protein, 10 aa, Artificial Sequence
  Description of Artificial Sequence Synthetic peptide
KELKQVASPF

SEQ ID NO: 162 - protein, 10 aa, Artificial Sequence
  Description of Artificial Sequence Synthetic peptide
KKKLINEKKE

SEQ ID NO: 163 - protein, 10 aa, Artificial Sequence
  Description of Artificial Sequence Synthetic peptide
KKCDISLQFF

SEQ ID NO: 164 - protein, 10 aa, Artificial Sequence
  Description of Artificial Sequence Synthetic peptide
KSTAGDTHLG

SEQ ID NO: 165 - protein, 10 aa, Artificial Sequence
  Description of Artificial Sequence Synthetic peptide
ATFYVAVTVP

SEQ ID NO: 166 - protein, 30 aa, Artificial Sequence
  Description of Artificial Sequence Synthetic polypeptide
LTIQLIQNHFVDEYDPTIEDSYRKQVVIDG

SEQ ID NO: 167 - protein, 30 aa, Artificial Sequence
  Description of Artificial Sequence Synthetic polypeptide
TIQLIQNHFVDEYDPTIEDSYRKQVVIDGE

SEQ ID NO: 168 - protein, 24 aa, Artificial Sequence
  Description of Artificial Sequence Synthetic peptide
KKKKKPKRDGYMFLKAESKIMFAT

SEQ ID NO: 169 - protein, 23 aa, Artificial Sequence
  Description of Artificial Sequence Synthetic peptide
KKKKYMFLKAESKIMFATLQRSS

SEQ ID NO: 170 - protein, 23 aa, Artificial Sequence
  Description of Artificial Sequence Synthetic peptide
KKKKKAESKIMFATLQRSSLWCL

SEQ ID NO: 171 - protein, 23 aa, Artificial Sequence
  Description of Artificial Sequence Synthetic peptide
KKKKKIMFATLQRSSLWCLCSNH

SEQ ID NO: 172 - protein, 21 aa, Artificial Sequence
  Description of Artificial Sequence Synthetic peptide
KKKKMFATLQRSSLWCLCSNH

SEQ ID NO: 173 - protein, 9 aa, Artificial Sequence
  Description of Artificial Sequence Synthetic peptide
MLTGPPARV

SEQ ID NO: 174 - protein, 10 aa, Artificial Sequence
  Description of Artificial Sequence Synthetic peptide
SMLTGPPARV

SEQ ID NO: 175 - protein, 9 aa, Artificial Sequence
  Description of Artificial Sequence Synthetic peptide
VLPEPHLAL

SEQ ID NO: 176 - protein, 9 aa, Artificial Sequence
  Description of Artificial Sequence Synthetic peptide
KPKRDGYMF

SEQ ID NO: 177 - protein, 10 aa, Artificial Sequence
  Description of Artificial Sequence Synthetic peptide
KPKRDGYMFL

SEQ ID NO: 178 - protein, 9 aa, Artificial Sequence
  Description of Artificial Sequence Synthetic peptide
ESKIMFATL

SEQ ID NO: 179 - protein, 8 aa, Artificial Sequence
  Description of Artificial Sequence Synthetic peptide
KRDGYMFL

SEQ ID NO: 180 - protein, 9 aa, Artificial Sequence
  Description of Artificial Sequence Synthetic peptide
PAVPFDLHF

SEQ ID NO: 181 - protein, 10 aa, Artificial Sequence
  Description of Artificial Sequence Synthetic peptide
AESKIMFATL

SEQ ID NO: 182 - protein, 9 aa, Artificial Sequence
  Description of Artificial Sequence Synthetic peptide
FATLQRSSL

SEQ ID NO: 183 - protein, 9 aa, Artificial Sequence
  Description of Artificial Sequence Synthetic peptide
ARVPAVPFD

SEQ ID NO: 184 - protein, 9 aa, Artificial Sequence
  Description of Artificial Sequence Synthetic peptide
IMKPKRDGY

SEQ ID NO: 185 - protein, 8 aa, Artificial Sequence
  Description of Artificial Sequence Synthetic peptide
DGYMFLKA

SEQ ID NO: 186 - protein, 11 aa, Artificial Sequence
  Description of Artificial Sequence Synthetic peptide
MFLKAESKIMF

SEQ ID NO: 187 - protein, 8 aa, Artificial Sequence
  Description of Artificial Sequence Synthetic peptide
LTGPPARV

SEQ ID NO: 188 - protein, 8 aa, Artificial Sequence
  Description of Artificial Sequence Synthetic peptide
ARVPAVPF

SEQ ID NO: 189 - protein, 9 aa, Artificial Sequence
  Description of Artificial Sequence Synthetic peptide
SMLTGPPAR

SEQ ID NO: 190 - protein, 9 aa, Artificial Sequence
  Description of Artificial Sequence Synthetic peptide
RVPAVPFDL

SEQ ID NO: 191 - protein, 9 aa, Artificial Sequence
  Description of Artificial Sequence Synthetic peptide
LTGPPARVP

SEQ ID NO: 192 - protein, 10 aa, Artificial Sequence
  Description of Artificial Sequence Synthetic peptide
VVVGAAGVGK

SEQ ID NO: 193 - protein, 9 aa, Artificial Sequence
  Description of Artificial Sequence Synthetic peptide
VVVGAAGVG

SEQ ID NO: 194 - protein, 9 aa, Artificial Sequence
  Description of Artificial Sequence Synthetic peptide
VVGAAGVGK

SEQ ID NO: 195 - protein, 8 aa, Artificial Sequence
  Description of Artificial Sequence Synthetic peptide
VVGAAGVG

SEQ ID NO: 196 - protein, 8 aa, Artificial Sequence
  Description of Artificial Sequence Synthetic peptide
VGAAGVGK

SEQ ID NO: 197 - protein, 10 aa, Artificial Sequence
  Description of Artificial Sequence Synthetic peptide
VVVGACGVGK

SEQ ID NO: 198 - protein, 9 aa, Artificial Sequence
  Description of Artificial Sequence Synthetic peptide
VVVGACGVG

SEQ ID NO: 199 - protein, 8 aa, Artificial Sequence
  Description of Artificial Sequence Synthetic peptide
VVVGACGV

SEQ ID NO: 200 - protein, 9 aa, Artificial Sequence
  Description of Artificial Sequence Synthetic peptide
VVGACGVGK

SEQ ID NO: 201 - protein, 8 aa, Artificial Sequence
  Description of Artificial Sequence Synthetic peptide
VVGACGVG

SEQ ID NO: 202 - protein, 8 aa, Artificial Sequence
  Description of Artificial Sequence Synthetic peptide
VGACGVGK

SEQ ID NO: 203 - protein, 10 aa, Artificial Sequence
  Description of Artificial Sequence Synthetic peptide
VVVGADGVGK

SEQ ID NO: 204 - protein, 9 aa, Artificial Sequence
  Description of Artificial Sequence Synthetic peptide
VVVGADGVG

SEQ ID NO: 205 - protein, 8 aa, Artificial Sequence
  Description of Artificial Sequence Synthetic peptide
VVVGADGV

SEQ ID NO: 206 - protein, 9 aa, Artificial Sequence
  Description of Artificial Sequence Synthetic peptide
VVGADGVGK

SEQ ID NO: 207 - protein, 8 aa, Artificial Sequence
  Description of Artificial Sequence Synthetic peptide
VVGADGVG

SEQ ID NO: 208 - protein, 10 aa, Artificial Sequence
  Description of Artificial Sequence Synthetic peptide
VVVGARGVGK

SEQ ID NO: 209 - protein, 9 aa, Artificial Sequence
  Description of Artificial Sequence Synthetic peptide
VVVGARGVG

SEQ ID NO: 210 - protein, 9 aa, Artificial Sequence
  Description of Artificial Sequence Synthetic peptide
VVGARGVGK

SEQ ID NO: 211 - protein, 8 aa, Artificial Sequence
  Description of Artificial Sequence Synthetic peptide
VVGARGVG

SEQ ID NO: 212 - protein, 8 aa, Artificial Sequence
  Description of Artificial Sequence Synthetic peptide
VGARGVGK

SEQ ID NO: 213 - protein, 9 aa, Artificial Sequence
  Description of Artificial Sequence Synthetic peptide
VVVGASGVG

SEQ ID NO: 214 - protein, 8 aa, Artificial Sequence
  Description of Artificial Sequence Synthetic peptide
VVVGASGV

SEQ ID NO: 215 - protein, 9 aa, Artificial Sequence
  Description of Artificial Sequence Synthetic peptide
VVGASGVGK

SEQ ID NO: 216 - protein, 8 aa, Artificial Sequence
  Description of Artificial Sequence Synthetic peptide
VVGASGVG

SEQ ID NO: 217 - protein, 8 aa, Artificial Sequence
  Description of Artificial Sequence Synthetic peptide
VGASGVGK

SEQ ID NO: 218 - protein, 10 aa, Artificial Sequence
  Description of Artificial Sequence Synthetic peptide
VVVGAVGVGK

SEQ ID NO: 219 - protein, 9 aa, Artificial Sequence
  Description of Artificial Sequence Synthetic peptide
VVVGAVGVG

SEQ ID NO: 220 - protein, 9 aa, Artificial Sequence
  Description of Artificial Sequence Synthetic peptide
VVGAVGVGK

SEQ ID NO: 221 - protein, 8 aa, Artificial Sequence
  Description of Artificial Sequence Synthetic peptide
VVGAVGVG

SEQ ID NO: 222 - protein, 8 aa, Artificial Sequence
  Description of Artificial Sequence Synthetic peptide
VGAVGVGK

SEQ ID NO: 223 - protein, 7 aa, Artificial Sequence
  Description of Artificial Sequence Synthetic peptide
KKKKKKK

SEQ ID NO: 224 - protein, 8 aa, Artificial Sequence
  Description of Artificial Sequence Synthetic peptide
KKKKKKKK

SEQ ID NO: 225 - protein, 4 aa, Artificial Sequence
  Description of Artificial Sequence Synthetic peptide
KTEY

SEQ ID NO: 226 - protein, 5 aa, Artificial Sequence
  Description of Artificial Sequence Synthetic peptide
KTEYK

SEQ ID NO: 227 - protein, 6 aa, Artificial Sequence
  Description of Artificial Sequence Synthetic peptide
KTEYKL

SEQ ID NO: 228 - protein, 7 aa, Artificial Sequence
  Description of Artificial Sequence Synthetic peptide
KTEYKLV

SEQ ID NO: 229 - protein, 8 aa, Artificial Sequence
  Description of Artificial Sequence Synthetic peptide
KTEYKLVV

SEQ ID NO: 230 - protein, 9 aa, Artificial Sequence
  Description of Artificial Sequence Synthetic peptide
KTEYKLVVV

SEQ ID NO: 231 - protein, 5 aa, Artificial Sequence
  Description of Artificial Sequence Synthetic peptide
KKTEY

SEQ ID NO: 232 - protein, 6 aa, Artificial Sequence
  Description of Artificial Sequence Synthetic peptide
KKTEYK

SEQ ID NO: 233 - protein, 7 aa, Artificial Sequence
  Description of Artificial Sequence Synthetic peptide
KKTEYKL

SEQ ID NO: 234 - protein, 8 aa, Artificial Sequence
  Description of Artificial Sequence Synthetic peptide
KKTEYKLV

SEQ ID NO: 235 - protein, 9 aa, Artificial Sequence
  Description of Artificial Sequence Synthetic peptide
KKTEYKLVV

SEQ ID NO: 236 - protein, 10 aa, Artificial Sequence
  Description of Artificial Sequence Synthetic peptide
KKTEYKLVVV

SEQ ID NO: 237 - protein, 6 aa, Artificial Sequence
  Description of Artificial Sequence Synthetic peptide
KKKTEY

SEQ ID NO: 238 - protein, 7 aa, Artificial Sequence
  Description of Artificial Sequence Synthetic peptide
KKKTEYK

SEQ ID NO: 239 - protein, 8 aa, Artificial Sequence
  Description of Artificial Sequence Synthetic peptide
KKKTEYKL

SEQ ID NO: 240 - protein, 9 aa, Artificial Sequence
  Description of Artificial Sequence Synthetic peptide
KKKTEYKLV

SEQ ID NO: 241 - protein, 10 aa, Artificial Sequence
  Description of Artificial Sequence Synthetic peptide
KKKTEYKLVV

SEQ ID NO: 242 - protein, 11 aa, Artificial Sequence
  Description of Artificial Sequence Synthetic peptide
KKKTEYKLVVV

SEQ ID NO: 243 - protein, 7 aa, Artificial Sequence
  Description of Artificial Sequence Synthetic peptide
KKKKTEY

SEQ ID NO: 244 - protein, 8 aa, Artificial Sequence
  Description of Artificial Sequence Synthetic peptide
KKKKTEYK

SEQ ID NO: 245 - protein, 9 aa, Artificial Sequence
  Description of Artificial Sequence Synthetic peptide
KKKKTEYKL

SEQ ID NO: 246 - protein, 10 aa, Artificial Sequence
  Description of Artificial Sequence Synthetic peptide
KKKKTEYKLV

SEQ ID NO: 247 - protein, 11 aa, Artificial Sequence
  Description of Artificial Sequence Synthetic peptide
KKKKTEYKLVV

SEQ ID NO: 248 - protein, 12 aa, Artificial Sequence
  Description of Artificial Sequence Synthetic peptide
KKKKTEYKLVVV

SEQ ID NO: 249 - protein, 4 aa, Artificial Sequence
  Description of Artificial Sequence Synthetic peptide
TEYK

SEQ ID NO: 250 - protein, 5 aa, Artificial Sequence
  Description of Artificial Sequence Synthetic peptide
TEYKL

SEQ ID NO: 251 - protein, 7 aa, Artificial Sequence
  Description of Artificial Sequence Synthetic peptide
TEYKLVV

SEQ ID NO: 252 - protein, 8 aa, Artificial Sequence
  Description of Artificial Sequence Synthetic peptide
TEYKLVVV

SEQ ID NO: 253 - protein, 6 aa, Artificial Sequence
  Description of Artificial Sequence Synthetic peptide
SALTIK

SEQ ID NO: 254 - protein, 8 aa, Artificial Sequence
  Description of Artificial Sequence Synthetic peptide
SALTIQLK

SEQ ID NO: 255 - protein, 10 aa, Artificial Sequence
  Description of Artificial Sequence Synthetic peptide
GKSALTIQLK

SEQ ID NO: 256 - protein, 8 aa, Artificial Sequence
  Description of Artificial Sequence Synthetic peptide
GKSALTIK

SEQ ID NO: 257 - protein, 7 aa, Artificial Sequence
  Description of Artificial Sequence Synthetic peptide
SALTIKK

SEQ ID NO: 258 - protein, 9 aa, Artificial Sequence
  Description of Artificial Sequence Synthetic peptide
SALTIQLKK

SEQ ID NO: 259 - protein, 11 aa, Artificial Sequence
  Description of Artificial Sequence Synthetic peptide
GKSALTIQLKK

SEQ ID NO: 260 - protein, 9 aa, Artificial Sequence
  Description of Artificial Sequence Synthetic peptide
GKSALTIKK

SEQ ID NO: 261 - protein, 8 aa, Artificial Sequence
  Description of Artificial Sequence Synthetic peptide
SALTIKKK

SEQ ID NO: 262 - protein, 10 aa, Artificial Sequence
  Description of Artificial Sequence Synthetic peptide
SALTIQLKKK

SEQ ID NO: 263 - protein, 12 aa, Artificial Sequence
  Description of Artificial Sequence Synthetic peptide
GKSALTIQLKKK

SEQ ID NO: 264 - protein, 10 aa, Artificial Sequence
  Description of Artificial Sequence Synthetic peptide
GKSALTIKKK

SEQ ID NO: 265 - protein, 9 aa, Artificial Sequence
  Description of Artificial Sequence Synthetic peptide
SALTIKKKK

SEQ ID NO: 266 - protein, 11 aa, Artificial Sequence
  Description of Artificial Sequence Synthetic peptide
SALTIQLKKKK

SEQ ID NO: 267 - protein, 13 aa, Artificial Sequence
  Description of Artificial Sequence Synthetic peptide
GKSALTIQLKKKK

SEQ ID NO: 268 - protein, 23 aa, Artificial Sequence
  Description of Artificial Sequence Synthetic peptide
KTEYKLVVVGAVGVGKSALTIQL

SEQ ID NO: 269 - protein, 23 aa, Artificial Sequence
  Description of Artificial Sequence Synthetic peptide
KTEYKLVVVGADGVGKSALTIQL

SEQ ID NO: 270 - protein, 23 aa, Artificial Sequence
  Description of Artificial Sequence Synthetic peptide
KTEYKLVVVGARGVGKSALTIQL

SEQ ID NO: 271 - protein, 23 aa, Artificial Sequence
  Description of Artificial Sequence Synthetic peptide
KTEYKLVVVGACGVGKSALTIQL

SEQ ID NO: 272 - protein, 24 aa, Artificial Sequence
  Description of Artificial Sequence Synthetic peptide
KKTEYKLVVVGAVGVGKSALTIQL

SEQ ID NO: 273 - protein, 24 aa, Artificial Sequence
  Description of Artificial Sequence Synthetic peptide
KKTEYKLVVVGADGVGKSALTIQL

SEQ ID NO: 274 - protein, 24 aa, Artificial Sequence
  Description of Artificial Sequence Synthetic peptide
KKTEYKLVVVGARGVGKSALTIQL

SEQ ID NO: 275 - protein, 24 aa, Artificial Sequence
  Description of Artificial Sequence Synthetic peptide
KKTEYKLVVVGACGVGKSALTIQL

SEQ ID NO: 276 - protein, 25 aa, Artificial Sequence
  Description of Artificial Sequence Synthetic peptide
KKKTEYKLVVVGAVGVGKSALTIQL

SEQ ID NO: 277 - protein, 25 aa, Artificial Sequence
  Description of Artificial Sequence Synthetic peptide
KKKTEYKLVVVGADGVGKSALTIQL

SEQ ID NO: 278 - protein, 25 aa, Artificial Sequence
  Description of Artificial Sequence Synthetic peptide
KKKTEYKLVVVGARGVGKSALTIQL

SEQ ID NO: 279 - protein, 25 aa, Artificial Sequence
  Description of Artificial Sequence Synthetic peptide
KKKTEYKLVVVGACGVGKSALTIQL

SEQ ID NO: 280 - protein, 26 aa, Artificial Sequence
  Description of Artificial Sequence Synthetic peptide
KKKKTEYKLVVVGAVGVGKSALTIQL

SEQ ID NO: 281 - protein, 26 aa, Artificial Sequence
  Description of Artificial Sequence Synthetic peptide
KKKKTEYKLVVVGADGVGKSALTIQL

SEQ ID NO: 282 - protein, 26 aa, Artificial Sequence
  Description of Artificial Sequence Synthetic peptide
KKKKTEYKLVVVGARGVGKSALTIQL

SEQ ID NO: 283 - protein, 26 aa, Artificial Sequence
  Description of Artificial Sequence Synthetic peptide
KKKKTEYKLVVVGACGVGKSALTIQL

SEQ ID NO: 284 - protein, 26 aa, Artificial Sequence
  Description of Artificial Sequence Synthetic peptide
KKTEYKLVVVGAVGVGKSALTIQLKK

SEQ ID NO: 285 - protein, 26 aa, Artificial Sequence
  Description of Artificial Sequence Synthetic peptide
KKTEYKLVVVGADGVGKSALTIQLKK

SEQ ID NO: 286 - protein, 26 aa, Artificial Sequence
  Description of Artificial Sequence Synthetic peptide
KKTEYKLVVVGARGVGKSALTIQLKK

SEQ ID NO: 287 - protein, 26 aa, Artificial Sequence
  Description of Artificial Sequence Synthetic peptide
KKTEYKLVVVGACGVGKSALTIQLKK

SEQ ID NO: 288 - protein, 23 aa, Artificial Sequence
  Description of Artificial Sequence Synthetic peptide
TEYKLVVVGAVGVGKSALTIQLK

SEQ ID NO: 289 - protein, 23 aa, Artificial Sequence
  Description of Artificial Sequence Synthetic peptide
TEYKLVVVGADGVGKSALTIQLK

SEQ ID NO: 290 - protein, 23 aa, Artificial Sequence
  Description of Artificial Sequence Synthetic peptide
TEYKLVVVGARGVGKSALTIQLK

SEQ ID NO: 291 - protein, 23 aa, Artificial Sequence
  Description of Artificial Sequence Synthetic peptide
TEYKLVVVGACGVGKSALTIQLK

SEQ ID NO: 292 - protein, 24 aa, Artificial Sequence
  Description of Artificial Sequence Synthetic peptide
TEYKLVVVGAVGVGKSALTIQLKK

SEQ ID NO: 293 - protein, 24 aa, Artificial Sequence
  Description of Artificial Sequence Synthetic peptide
TEYKLVVVGADGVGKSALTIQLKK

SEQ ID NO: 294 - protein, 24 aa, Artificial Sequence
  Description of Artificial Sequence Synthetic peptide
TEYKLVVVGARGVGKSALTIQLKK

SEQ ID NO: 295 - protein, 24 aa, Artificial Sequence
  Description of Artificial Sequence Synthetic peptide
TEYKLVVVGACGVGKSALTIQLKK

SEQ ID NO: 296 - protein, 25 aa, Artificial Sequence
  Description of Artificial Sequence Synthetic peptide
TEYKLVVVGAVGVGKSALTIQLKKK

SEQ ID NO: 297 - protein, 25 aa, Artificial Sequence
  Description of Artificial Sequence Synthetic peptide
TEYKLVVVGADGVGKSALTIQLKKK

SEQ ID NO: 298 - protein, 25 aa, Artificial Sequence
  Description of Artificial Sequence Synthetic peptide
TEYKLVVVGARGVGKSALTIQLKKK

SEQ ID NO: 299 - protein, 25 aa, Artificial Sequence
  Description of Artificial Sequence Synthetic peptide
TEYKLVVVGACGVGKSALTIQLKKK

SEQ ID NO: 300 - protein, 26 aa, Artificial Sequence
  Description of Artificial Sequence Synthetic peptide
TEYKLVVVGAVGVGKSALTIQLKKKK

SEQ ID NO: 301 - protein, 26 aa, Artificial Sequence
  Description of Artificial Sequence Synthetic peptide
TEYKLVVVGADGVGKSALTIQLKKKK

SEQ ID NO: 302 - protein, 26 aa, Artificial Sequence
  Description of Artificial Sequence Synthetic peptide
TEYKLVVVGARGVGKSALTIQLKKKK

SEQ ID NO: 303 - protein, 26 aa, Artificial Sequence
  Description of Artificial Sequence Synthetic peptide
TEYKLVVVGACGVGKSALTIQLKKKK

SEQ ID NO: 304 - protein, 9 aa, Artificial Sequence
  Description of Artificial Sequence Synthetic peptide
ALNSEALSV

SEQ ID NO: 305 - protein, 10 aa, Artificial Sequence
  Description of Artificial Sequence Synthetic peptide
  MISC_FEATURE (1)..(10): This region may encompass 5-10 residues
  See specification as filed for detailed description of substitutions and preferred embodiments
KKKKKKKKKK

SEQ ID NO: 306 - protein, 7 aa, Artificial Sequence
  Description of Artificial Sequence Synthetic peptide
  MISC_FEATURE (1)..(7): This region may encompass 4-7 residues
  See specification as filed for detailed description of substitutions and preferred embodiments
RRRRRRR

SEQ ID NO: 307 - protein, 8 aa, Artificial Sequence
  Description of Artificial Sequence Synthetic peptide
RRRRRRRR

SEQ ID NO: 308 - protein, 10 aa, Artificial Sequence
  Description of Artificial Sequence Synthetic peptide
  MISC_FEATURE (1)..(7): This region may encompass 5-10 residues
  See specification as filed for detailed description of substitutions and preferred embodiments
RRRRRRRRRR

SEQ ID NO: 309 - protein, 62 aa, Artificial Sequence
  Description of Artificial Sequence Synthetic polypeptide
MTEYKLVVVGACGVGKSALTIQLIQNHFVDEYDPTIEDSYRKQVVIDGETCLLDILDTAGQE

SEQ ID NO: 310 - protein, 9 aa, Artificial Sequence
  Description of Artificial Sequence Synthetic peptide
LVVVGACGV

SEQ ID NO: 311 - protein, 62 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQE

SEQ ID NO: 312 - protein, 10 aa, Artificial Sequence
  Description of Artificial Sequence Synthetic peptide
KLVVVGADGV

SEQ ID NO: 313 - protein, 9 aa, Artificial Sequence
  Description of Artificial Sequence Synthetic peptide
LVVVGADGV

SEQ ID NO: 314 - protein, 62 aa, Artificial Sequence
  Description of Artificial Sequence Synthetic polypeptide
MTEYKLVVVGAVGVGKSALTIQLIQNHFVDEYDPTIEDSYRKQVVIDGETCLLDILDTAGQE

SEQ ID NO: 315 - protein, 10 aa, Artificial Sequence
  Description of Artificial Sequence Synthetic peptide
KLVVVGAVGV

SEQ ID NO: 316 - protein, 9 aa, Artificial Sequence
  Description of Artificial Sequence Synthetic peptide
LVVVGAVGV

SEQ ID NO: 317 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGHEEYSAMRDQYMRTGEGFLCVFAINNTKSFEDIHHYREQIKRVKDSEDVPM

SEQ ID NO: 318 - protein, 10 aa, Artificial Sequence
  Description of Artificial Sequence Synthetic peptide
ILDTAGHEEY

SEQ ID NO: 319 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGLEEYSAMRDQYMRTGEGFLCVFAINNTKSFEDIHHYREQIKRVKDSEDVPM

SEQ ID NO: 320 - protein, 10 aa, Artificial Sequence
  Description of Artificial Sequence Synthetic peptide
ILDTAGLEEY

SEQ ID NO: 321 - protein, 10 aa, Artificial Sequence
  Description of Artificial Sequence Synthetic peptide
LLDILDTAGL

SEQ ID NO: 322 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGKEEYSAMRDQYMRTGEGFLCVFAINNSKSFADINLYREQIKRVKDSDDVPM

SEQ ID NO: 323 - protein, 10 aa, Artificial Sequence
  Description of Artificial Sequence Synthetic peptide
ILDTAGKEEY

SEQ ID NO: 324 - protein, 101 aa, Artificial Sequence
  Description of Artificial Sequence Synthetic polypeptide
AGGVGKSALTIQLIQNHFVDEYDPTIEDSYRKQVVIDGETCLLDILDTAGREEYSAMRDQYMRTGEGFLCVFAINNSKSFADINLYREQIKRVKDSDDVPM

SEQ ID NO: 325 - protein, 10 aa, Artificial Sequence
  Description of Artificial Sequence Synthetic peptide
ILDTAGREEY

SEQ ID NO: 326 - protein, 23 aa, Artificial Sequence
  Description of Artificial Sequence Synthetic peptide
TCLLDILDTAGHEEYSAMRDQYM

SEQ ID NO: 327 - protein, 9 aa, Artificial Sequence
  Description of Artificial Sequence Synthetic peptide
AGHEEYSAM

SEQ ID NO: 328 - protein, 9 aa, Artificial Sequence
  Description of Artificial Sequence Synthetic peptide
LDTAGHEEY

SEQ ID NO: 329 - protein, 10 aa, Artificial Sequence
  Description of Artificial Sequence Synthetic peptide
DTAGHEEYSA

SEQ ID NO: 330 - protein, 23 aa, Artificial Sequence
  Description of Artificial Sequence Synthetic peptide
TCLLDILDTAGREEYSAMRDQYM

SEQ ID NO: 331 - protein, 8 aa, Artificial Sequence
  Description of Artificial Sequence Synthetic peptide
DTAGREEY

SEQ ID NO: 332 - protein, 9 aa, Artificial Sequence
  Description of Artificial Sequence Synthetic peptide
AGREEYSAM

SEQ ID NO: 333 - protein, 9 aa, Artificial Sequence
  Description of Artificial Sequence Synthetic peptide
TAGREEYSA

SEQ ID NO: 334 - protein, 10 aa, Artificial Sequence
  Description of Artificial Sequence Synthetic peptide
LLDILDTAGR

SEQ ID NO: 335 - protein, 10 aa, Artificial Sequence
  Description of Artificial Sequence Synthetic peptide
AGREEYSAMR

SEQ ID NO: 336 - protein, 9 aa, Artificial Sequence
  Description of Artificial Sequence Synthetic peptide
LDTAGREEY

SEQ ID NO: 337 - protein, 23 aa, Artificial Sequence
  Description of Artificial Sequence Synthetic peptide
TCLLDILDTAGKEEYSAMRDQYM

SEQ ID NO: 338 - protein, 9 aa, Artificial Sequence
  Description of Artificial Sequence Synthetic peptide
AGKEEYSAM

SEQ ID NO: 339 - protein, 9 aa, Artificial Sequence
  Description of Artificial Sequence Synthetic peptide
LDTAGKEEY

SEQ ID NO: 340 - protein, 23 aa, Artificial Sequence
  Description of Artificial Sequence Synthetic peptide
TCLLDILDTAGLEEYSAMRDQYM

SEQ ID NO: 341 - protein, 11 aa, Artificial Sequence
  Description of Artificial Sequence Synthetic peptide
DTAGLEEYSAM

SEQ ID NO: 342 - protein, 11 aa, Artificial Sequence
  Description of Artificial Sequence Synthetic peptide
CLLDILDTAGL

SEQ ID NO: 343 - protein, 9 aa, Artificial Sequence
  Description of Artificial Sequence Synthetic peptide
LDTAGLEEY

SEQ ID NO: 344 - protein, 9 aa, Artificial Sequence
  Description of Artificial Sequence Synthetic peptide
AGLEEYSAM

SEQ ID NO: 345 - protein, 10 aa, Artificial Sequence
  Description of Artificial Sequence Synthetic peptide
AGLEEYSAMR

SEQ ID NO: 346 - protein, 8 aa, Artificial Sequence
  Description of Artificial Sequence Synthetic peptide
GLEEYSAM

SEQ ID NO: 347 - protein, 23 aa, Artificial Sequence
  Description of Artificial Sequence Synthetic peptide
MTEYKLVVVGAAGVGKSALTIQL

SEQ ID NO: 348 - protein, 9 aa, Artificial Sequence
  Description of Artificial Sequence Synthetic peptide
LVVVGAAGV

SEQ ID NO: 349 - protein, 10 aa, Artificial Sequence
  Description of Artificial Sequence Synthetic peptide
KLVVVGAAGV

SEQ ID NO: 350 - protein, 9 aa, Artificial Sequence
  Description of Artificial Sequence Synthetic peptide
YKLVVVGAA

SEQ ID NO: 351 - protein, 23 aa, Artificial Sequence
  Description of Artificial Sequence Synthetic peptide
MTEYKLVVVGACGVGKSALTIQL

SEQ ID NO: 352 - protein, 23 aa, Artificial Sequence
  Description of Artificial Sequence Synthetic peptide
MTEYKLVVVGADGVGKSALTIQL

SEQ ID NO: 353 - protein, 10 aa, Artificial Sequence
  Description of Artificial Sequence Synthetic peptide
VGADGVGKSA

SEQ ID NO: 354 - protein, 23 aa, Artificial Sequence
  Description of Artificial Sequence Synthetic peptide
MTEYKLVVVGARGVGKSALTIQL

SEQ ID NO: 355 - protein, 10 aa, Artificial Sequence
  Description of Artificial Sequence Synthetic peptide
GARGVGKSAL

SEQ ID NO: 356 - protein, 10 aa, Artificial Sequence
  Description of Artificial Sequence Synthetic peptide
RGVGKSALTI

SEQ ID NO: 357 - protein, 8 aa, Artificial Sequence
  Description of Artificial Sequence Synthetic peptide
KLVVVGAR

SEQ ID NO: 358 - protein, 23 aa, Artificial Sequence
  Description of Artificial Sequence Synthetic peptide
MTEYKLVVVGASGVGKSALTIQL

SEQ ID NO: 359 - protein, 10 aa, Artificial Sequence
  Description of Artificial Sequence Synthetic peptide
KLVVVGASGV

SEQ ID NO: 360 - protein, 10 aa, Artificial Sequence
  Description of Artificial Sequence Synthetic peptide
SGVGKSALTI

SEQ ID NO: 361 - protein, 23 aa, Artificial Sequence
  Description of Artificial Sequence Synthetic peptide
MTEYKLVVVGAVGVGKSALTIQL

SEQ ID NO: 362 - protein, 9 aa, Artificial Sequence
  Description of Artificial Sequence Synthetic peptide
AVGVGKSAL

SEQ ID NO: 363 - protein, 9 aa, Artificial Sequence
  Description of Artificial Sequence Synthetic peptide
YKLVVVGAV

SEQ ID NO: 364 - protein, 23 aa, Artificial Sequence
  Description of Artificial Sequence Synthetic peptide
MTEYKLVVVGAGCVGKSALTIQL

SEQ ID NO: 365 - protein, 9 aa, Artificial Sequence
  Description of Artificial Sequence Synthetic peptide
VVGAGCVGK

SEQ ID NO: 366 - protein, 23 aa, Artificial Sequence
  Description of Artificial Sequence Synthetic peptide
MTEYKLVVVGAGDVGKSALTIQL

SEQ ID NO: 367 - protein, 9 aa, Artificial Sequence
  Description of Artificial Sequence Synthetic peptide
VVGAGDVGK

SEQ ID NO: 368 - protein, 10 aa, Artificial Sequence
  Description of Artificial Sequence Synthetic peptide
ALNSEALSVV

SEQ ID NO: 369 - protein, 10 aa, Artificial Sequence
  Description of Artificial Sequence Synthetic peptide
MALNSEALSV

SEQ ID NO: 370 - protein, 71 aa, Artificial Sequence
  Description of Artificial Sequence Synthetic polypeptide
MTEYKLVVVGADGVGKSALTIQLIQNHFVDEYDPTIEDSYRKQVVIDGETCLLDILDTAGQEVVGADGVGK

SEQ ID NO: 371 - DNA, 30 bp, Artificial Sequence
  Description of Artificial Sequence Synthetic oligonucleotide
ggcggcagcggcggcggcggcagcggcggc

SEQ ID NO: 372 - DNA, 30 bp, Artificial Sequence
  Description of Artificial Sequence Synthetic oligonucleotide
ggcggcagcctgggcggcggcggcagcggc

SEQ ID NO: 373 - DNA, 12 bp, Artificial Sequence
  Description of Artificial Sequence Synthetic oligonucleotide
ggcgtcggcacc

SEQ ID NO: 374 - DNA, 12 bp, Artificial Sequence
  Description of Artificial Sequence Synthetic oligonucleotide
cagctgggcctg

SEQ ID NO: 375 - DNA, 12 bp, Artificial Sequence
  Description of Artificial Sequence Synthetic oligonucleotide
caactgggattg

SEQ ID NO: 376 - protein, 6 aa, Artificial Sequence
  Description of Artificial Sequence Synthetic 6xHis tag
HHHHHH

SEQ ID NO: 377 - protein, 26 aa, Artificial Sequence
  Description of Artificial Sequence Synthetic peptide
AVGITYTWTRLYASVLTGSLVSKTKK

SEQ ID NO: 378 - protein, 29 aa, Artificial Sequence
  Description of Artificial Sequence Synthetic peptide
IQKISFFDGFEVGFNFRTLQPNGLLFYYT

SEQ ID NO: 379 - protein, 28 aa, Artificial Sequence
  Description of Artificial Sequence Synthetic peptide
GIPVHLELASMTNMELMSSIVHQQVFPT

SEQ ID NO: 380 - protein, 28 aa, Artificial Sequence
  Description of Artificial Sequence Synthetic peptide
GRVLELFRAAQLANDVVLQIMELCGATR

SEQ ID NO: 381 - protein, 26 aa, Artificial Sequence
  Description of Artificial Sequence Synthetic peptide
KARDETAALLNSAVLGAAPLFVPPAD

SEQ ID NO: 382 - protein, 27 aa, Artificial Sequence
  Description of Artificial Sequence Synthetic peptide
REGVELCPGNKYEMRRHGTTHSLVIHD

SEQ ID NO: 383 - protein, 27 aa, Artificial Sequence
  Description of Artificial Sequence Synthetic peptide
PSKPSFQEFVDWENVSPELNSTDQPFL

SEQ ID NO: 384 - protein, 6 aa, Artificial Sequence
  Description of Artificial Sequence Synthetic peptide
  MOD_RES (6)..(6): Cit
KKKKVX

SEQ ID NO: 385 - protein, 4 aa, Artificial Sequence
  Description of Artificial Sequence Synthetic peptide
QLGL

SEQ ID NO: 386 - protein, 4 aa, Artificial Sequence
  Description of Artificial Sequence Synthetic peptide
GVGT

SEQ ID NO: 387 - protein, 20 aa, Artificial Sequence
  Description of Artificial Sequence Synthetic peptide
TEYKLVVVGAVGVGKSALTI

SEQ ID NO: 388 - protein, 29 aa, Artificial Sequence
  Description of Artificial Sequence Synthetic peptide
KKKKIIMKIRNAVVGAVGVGKKKNKKDDI

SEQ ID NO: 389 - protein, 16 aa, Artificial Sequence
  Description of Artificial Sequence Synthetic peptide
VVGAVGVGKSALTIQL

SEQ ID NO: 390 - protein, 15 aa, Artificial Sequence
  Description of Artificial Sequence Synthetic peptide
TEYKLVVVGAVGVGK

SEQ ID NO: 391 - protein, 25 aa, Artificial Sequence
  Description of Artificial Sequence Synthetic peptide
WQAGILARVVGAVGVGKQGQNLKYQ

SEQ ID NO: 392 - protein, 24 aa, Artificial Sequence
  Description of Artificial Sequence Synthetic peptide
HSYTTAEVVGAVGVGKILGVLLLI

SEQ ID NO: 393 - protein, 25 aa, Artificial Sequence
  Description of Artificial Sequence Synthetic peptide
PLTEEKIKVVGAVGVGKEKEGKISK

SEQ ID NO: 394 - protein, 5 aa, Artificial Sequence
  Description of Artificial Sequence Synthetic peptide
QLGLK

SEQ ID NO: 395 - protein, 5 aa, Artificial Sequence
  Description of Artificial Sequence Synthetic peptide
KQLGL